SEQUENCE LISTING (74 sequences)

SEQ ID NO: 1 - DNA, 5000 bp, Artificial Sequence
  nucleic acid probe
agtctgtccacggcagaggaaattctagctccaggtttccagtgtgaaggcaaaagggtgaaattgtccacgtgttaccaatctcaagtctccctgctttttcaaaataaaaccctgcagaaagcaatggctccatctaaaagaatgactctccattgtttgaatgtcaagcactgtggttcactggagcagggcctcccttgggggaccagcatttatgataccctggaatgacactgcttaggaagccacctttttggtgcctttcagttgatttcaggcagtcattcatgcactgtttatatggaaaaggacacttgtcacatgaaattcatgagctcatgtctatacctcactgcccagtgttgtattatttactctgcggtatgagtgtctcttttagagaaatgccctcagcttctggatgtctgcagtttctagtcacaacggctgtgattctttgaagtggccagtaaaccagatttttgacaggtgtggagagccccggcccctgctgctctgcagataagtgccaggtatgaaatgagggccggtcctcggaattgtgcattgacaggcagggttgaagcctcaggaatggattcaggcaccacaaagccagatctctgtgcttccctgctgtgccccaattccagtttgggtgattttacatcaggtcctttctctcttactttcccatatacgaatataaacacatagcccctaggtctggagtcaatggctgctttcccagagttaagcacaggtcaaaataggtggcaggaggtgctagctacatgcttcaagtgtctcagcaaacctgagaaatgcagtgaagaagctctcttaggacacagagagccctacgtttatccagacagacactccattagccgaaacgaccagacacacgaagttttaagagtccccgctgtcagaattccaaatggttagtcttaaactgacttagggcgcagcgctgcctctttacaaagagctgagcagaagtgtccagtgttagcgctggcaccttaccgcctatctctgtggcactattaggcctgaggaatgattgagggctgatgtcgctgtgcccaggccacctgcaatgattccatcaaacattgacttctcgtggaactttcacaaagccactactgggaaaacacatgtgtatccatgtttgaaagtttatcattttgaaatgggtccgggctacctgttgctgcggagtcctaacagaaccgctacaggaatgggcccaccctacctaagaaaaactgagggctggagcacgggtaccccaccaccatcagggaattcgaggggagactggcagttatccagggatcccagaaggagtcaactgggagttaagagtcctaacaaccctagaaaaactgctcctttctgcgagctggtttcacactcttaacctgatgcaaagcgccgagagctgctggggaatgatatcactaacccaaagctttggtccttaggctaagcacctgtaacagatgaagcaaagttatcaggccccgcaagttgctctctacagtttataatccccatcttccatttgagggagaactagatgctatcatttcttttttttaatcccccaggtgtgatctaccatgcattgtctcagaaagaggcgaatgactccgatgtccaggtcagttcttggcagggagtccggctggatcaaagtctgtagttctcattacctgttcccacgtgccagcctccttttctgttgtgccaaagtcaagtttggtaaaatgaggctttccttaaaccaggctgtgcccacgtggtgggcagcatcattcaaccttgtcactgtgccctctgcacttctgcagaaggtgtgcaatcacatatcataagtgaaattatagtcacaatgacgagagtgaattaatttttagttagcatctacaggtgcaaagctgaagcagagttcccaccatcatactgtgagctccctcgaaggccatttttcccagaagtcctctctaagagcctgtgctcattcatggtaagttgacgccctgctctcagtgacacccttcagagagagcgagcctggggcctcctagttttgctgttttctgtagtgttaggatcacttcctccatagcttagtgctactctgagctcactttcatctggtcatgcctggttgaatatatttgaagtgagcgtttgacacgctgttgtgtgtgacaaacggtcatgaattggtgtgtgagtgtgacttacatctcggcagctgtgggtagcagatgcatggcggtaacccgagctccccacgtctttctgcccgcggtgcttacccttcttggtgtgtgaacttggctgagtggctggggaggcagcctggctccacctaagaccttcaggagtaagacccgtgcaagtacatctgttgcccatggcaacaggctgtttgtccttcggctttgttagacagaaaagaggaactgatatgtttggagatcaacggccaaatagcctcccacgctccagaagacgcttaaagtatgccatgctgttcccggccccactcaggagatcagctgggcctcaggtggtgctctttggggtggatctcaagcctctgatcattgtaaatgcggcttcaggaaccagcgttggtattgcctggctgcagacggttaaagacctagttgcctgtttaccctgcaggcagccacagatgattcaggccaagctcttaaaggcagatcttcacggggctattatttcaggtaatttacctaagagcgtgtagctcgctggctgtcttagccagtccggccatttgctggacacaccctaagtggtctctgtgtaccttctccacacagggacccctgttggtaccctaaagggtgctgggcacatatgtgacctttaattccagaatcctgcccaagacccctgtggccacagaccctccgaaggacacctcataggaacagtccagaggctgcacattttcaaaattatcaccaaagaagaccgcctgaaaggtatgtaggtgtttctgagggcattgcttgcgggctgtggctcccagctactgcgttagagcttggtttttcttgttgcttcccttattttcctacatatccttctacctcacattgtaaacctcggaaagtggtgtaatagtttctccacagagcatatgacccctcaagtcagcgcagattgcagatacttgctgaacctgcattaaagcctgcagagaccatcccctgcacccgtggctcccccaagaggtgtgtgtgtgactagattgacctgtggcagaataaccacaggcaaccctaatgcccctccccttgtgggtttcccgtttcctattagcttatttttttcattcaattgtggaaaccgatggctttatttcctacatttacgggagcaaattccagcttcggtcaagtgaacggtctgcgaagaagttcaaagcgaagggaacgattgcgaaaggcaggccacaaatctgttctcccccaggagaagtagcagtgccagggctttgcgtttctggtaacttccacccaaaccacaccagagagaaacctccccaaagctcagtgcacccagagtctcacagggaccaacacggtgcctcggtcctgcttccatccatgcaagtgtcctccgctcaccagggtgttctcggatgcagttcaaagtgcaggatgtgctgcctttattacctgctttcccgctgagcaaatccctgtcttgagtgggaagtggcatctattcccccagccatggagggtaatgtggtgctttttggcccgatctctcccatgtccactgtccaaggagcaggaccctggcagagctgaggccttggccatgtggtcccacgactgatctcccaggttccaaagagtaaacaggaacatccctccacaaagccgtaaattctggtaaaaaagctcgctttctggacaatggctttagctttgaggaggatataggacctcacccagagactcacaaaacgccagatagatgcacagactgggtgaaatgcaacagactgggatagggaaggatttcctggggcccaggatgtgcctctaataatgttgaataagggttggcacacagcaactgcaagtggtgctggtcagtgacccctgtctgggccagatgagcccgtgtccagctatgcagaccgaggaggttatgctccatcaacagctgtgacgtgcagcaagtgtggcccgcgcttctctgcccctttcagtttcccccatgaagccaggaaacccaagttcaaggcccaagttccccgagaccttcggttccaaccacagagaacgtgaagaaagggagtgtggtgtttattgcaggcacatttattttaacagctccctcctttcaaaatacgggctcttccgctaccccagaactccagggaccaagtgctctgcagccctgtcactagggcgtggttgccacgctgaggacactgagatgaattatccccggatcacatggccagtgggtgagtcacatggcaagtcccctctcaccaaatcagctattcttccagaacttgactgttagctcctcagggggcagaccgatgttttaggccttcccagaccaccctaagtccaccagtcaccatctccacagtgctggagagagaagctgaccagccccattctggctccattgagtaaaaggtaagttgtggccctaaataatgaagacagaagagctaatgggctcggtgaagaagggcagctcaggcgcgccatttgctgtcctggtgctttctgttgttgaatggacagttcagaacctccagaaatcaccctgagaatctcaggctttgtaacagcagtgtctcacccagacaccaggagcctggatttgccaccactttctatgctgtgttataaacacaaacatgtgtctgcctccttgactataagctctgatgaattcgcccc

SEQ ID NO: 2 - DNA, 5000 bp, Artificial Sequence
  Nucleic acid probe
gctcggagaatgagtaagtgaatcagtgaatgtgcccgcagctggtgtgatcaggaacttttgggtcatttcagggccgtctctggggcaggatggtgctaccagcagtgcctcctacagagcactgattaagtcactactgagtgctctacaggtaaccccttcaaatcttagcaacagcttcaagaggcaattaggaagctgcctttttttgaagtggtttaagatctctgctcttgattgcaatcttccaagagctgattttggccacttggctctcatttctgaagtgatgatgggcctaacttgcaattcagagccaggaatcacacttatggccaccctgtgtctgaccttaatgagagaggctgtggctttaggtccttagcagctcatctcatcagagctttggtgactgtcacccaggcccctgtccaagttctggcctcatggcactaatcccccacgtagccagtggctagctgtgcaacctagagccaccttccagaacattagccctgccccctgtggagtccctgagaagagaaatgccccacgagtgacaaagaaggcaaagtatctcctcactgtgtgctggagcggccggcacagcgactggggccatacatctcagcttccactcctgagttctggacttctcccacaccctttgtgtggctggaggttttatgagaaggcccaaaaaatgggtccccgtacacagcgaatgggatgtttcagcagagacacaaacaagaaggatgcgagggacccttacttggagatattttctcctgtccatttgggaaactgggagacaagaatggcgaagaaaaggagtctcgaagatccgtcaccatcagctaggaggatggaacaagcagatggagaggaaggagttccagacaaagacagaacatgaatgaatgaccatataggaacttgccagagagaagcagacgtgccccaggtaaggtgaggcctggttcatgcacatgaaacaggatatagatattccaaaatcacaccctggtcacaacacaggaaacagtaataagagtaactgatccttacgcatggttgctatgtgcgtatgctgccatttgctcccaagacagaagtttgctctctttatccattttctcagaataaaagacctccccgcttctgtcttccaaggattataagcaaggaggttttaacatcatgagagggcacctcctgcaggaactgcaaatgcaaggcaaacttgtcagcctccagaggcctcagggagggcagccaaggtgggtgacagcagtctggaagtcaggcctctcctccacttctctgaatactcaggtgcagtccctccagagggcagggctcattgcccttgcagtggatcccatctctccagcaaaataccacaagtgtatacagtaaatgggaatctccctcccacccattttcccttccagaggcaaacccatgaccagtttattgtgaattctgccagcaccagaccccaaaggctgacactgaaatgccaaatggttctccaagatgtgtcatccaaccaagacagcttctcctgcacagacccctgccctctactcggcaaaagcctcttggatgtactccattgacattaggtgggtctaatactctcaggttatattggcctgcaacatttatttcacatctcgaaggcatttttctcctttccccctttggcctggttggagcctcaaggggaaaatccatgctgggaaacaaaaacaataatgcccgtgaacatgaagtggatgtgcagacagcgcatgtagtagtttgggaatcagacaaatttagggtcagtttgaggatctacccaagctaaacagtaggacctctgtttcctttgtttttctttaatttgggcattgagcacagaggtggttgccagaatccctaacctgaaaatgccgagccccactgcttttctgaaacccgggttcccgcagtctggaagccagctttccacagctatgtggcttggctgactcaacttctgcctgcccctgagtcacgagtttttcctctcttatttggaggagcgaggccctccaccctccacccgagaccagtgcgtgctgcctccagggcgtttcttggagcgccattcagatccatagttggaaggaaggacagaagaggagactgaggtccaagtgggcccgcaggacaaggctaggaggaacatcctccctctagtttcccagtcatgaaaaggcttccttctcccgaggcctcctctgtacggacaaggtgttagatgctccttcctccgcagcctgacttcacccattccaccttgagctgctccccaggaccggagctctgcccctagatctttgcacagctgacttcaggctggagttttcttcacagtgtttagcactggctctcagaaaagaccctgtgaatgtacttctagtctcactggcaagaagtgtgcattcctgaagggcctggtctcttgccaagaaaaacctacaggcaaggtcaaaccaggatgtgaagttgtggtcatgtagagaaggtgagcatgccggggcagctcccaggaggatgttgtcaccttatagacgaaagtggaatccaatctgagccctccattcctggtcacatctatgtcctctccccaggctacttactcctgactttctccttcctatcgttgtttgccgtcagaccacattgatctgtatctctcattgcgtgagtagccccgggtttttatccttttccctgctggtaggcattttggttgtctctggtggaatgcagttctggttttgtcagattgtccctcagccaggtactcggtgatcagtcatggtgggacaggtcagttctgtcactggtaatcccctaggaggctcagaactgcagctcagtgacccagaaacactatgggtgtgatcagctcagccccaggcgtgccgtgtgtaccggaatgcatgtgtgcacgtggtgttctggactcccagtgcctttttgctggacactgtgtccttatggcttcactgcaggtaatgcctttcccttcaggaagccccagaacacccacaggtctcagccgggtgaggactctgtcctgtgtcacctctctccaggtgtccagctgtctcatgcctttttgtcctgtcctcagctctccgtgtggtcagcgaaaccaaggacacgggaaccaactgtcctgccagccgagtgctgctgtccacctctcaaatcttcctcccagctgtgcctggactccatgtccctgccagaagcggtggcaaaaatctaccactgtcccccacactgggagttcttgtgtatgctggctgcctgaaggagacagacgctcatgctgtgccttggctgggctcccctttagagctctgcaggatgcagtgttcccacgggacccagaggctgcagacactaaggagactttcacaaaggtcaagtagcccctacctgtgggctttcagagggtttccgctggcctggccacttccctgcccagcctgttctttgaacaaggccagaaagaggcctaggagaaggcaaagccgctcctgggctcagtgtgccccccaaacctggccttggcatctgagctccttgaaagcacaggtgtttctcatttgtttttggcctccatggcccccaacccatggtcctgcacaatgacagcattggctttgcccaacccgcttgtcctgcctctgccaacacctacacctgggcaacccagccaaaccctaaggtgtctctcgaccctttctaatccttggagcatcagctcagggttcctgccagacattctgaccctgaaacacaggctttctggagaagagggcctgcccactgtggtcccaggctggggcagaggcctttgaacctcgcctgccccagccataaccacttgctggcacaacccaatccctgagggtttctttcccccacgccagagctggagttggtaacggttttaaatcagttacagagaaaaccacccagcaatgagcaactccaagatgtgaaggggagcattcagtagcacaatggacacagtgactggcgccggagcaagcggttagggaaatcagtaagtcctcaaaggagtactgggagaaattagcatgggggccggacacacagttcagagagtggggacagccagcccaaagaggggccacactgtttggaaatacacgtgcctgccctttttagttcagctctgctgaggggcttgtgtgtttgctgaaaagattatctcaacattgcacagccggccctcctgccttggtcagtgggaacaagcacatcagatcaagctctcatgggtctcaggatctgggatggtgctttggaactgaatgttgatacataaataactcacattttaaacactagaactcaggcatgagacgcccatgcctgcctgtctgttccctctgagccatgccttcatcgggtgggagaaactgaaggcacatcaggcaatgccctcttctgcatggcttagatgagtcctgcatttcttaattcccatatgttcgttgttgggattgagagacattctaggacccagctagttccagtgtcttcactcagcaaggcctgctccatcggacacaaaactgatcaggagttggaccatctcctccctgggcatttttccccagaaggatttgctgtaagcaaccggagaagttggcatttgttcaacatggaaaaagtgtcccccaactttgaagcactgaaccaaacatctttgttgagtctgagctgggaccacctgtgtcccctgggcctgacacagaccaggacctgatgcacgagtaagaatgaatggggcccaatcgcctaacaaaggcaactccacgatatctgcaaatcaacgttttttccttcacactaaaaacgggcaactttagagcccactaaagctatacaaaattgggagggggcatgctctgatt

SEQ ID NO: 3 - DNA, 5000 bp, Artificial Sequence
  Nucleic acid probe
gtgagactgaatttcctccaccaggcagctgatatgctccttgcccctgaacttagaaaatcttcagtgagcccatgtggttaaaggacctggggacagattctgggacttaaagaccttgtatccacggaaagttctacctgaagaggccacgtgcagaatatggtgcagagaagcggaaggtgccctagggaaggggtagccttgaatcgcatggaccttccgaggcccaggacacagaagagctggacagcttggttcctatggagtggcagtggctacttcacgccagttcattgcctaatttacaagttttactgaacttttccccatgacaccagtgaaatgctccaagtcgacaagagttgcactcatgtgcactaaacctttatgagaacatatgctcccgcccatgaccagggactggaagtagtgaatcccccgggctctcttagtggacatcctgttctctaccagcttttactcaatctgaataaaatagatgcacagtgtcatcgaggaagaaggaaacagccttgtctcccccatctccggttccttcccggatggcaagatgctgaagtcaggcctcactaattcgaaggtctggggccacccacacatttatttttctcctcagtgtgagcctcagcttaacgaaatgtccttgaatgtcaatcccactacagaagggaagaacctctctgactccatctatggtttgggccacagccccaggccaggtagaccttctgaggctcaggacacaggaattcttccttccccaagccaaagaaagtttttcaggaaagctcagcagctccaagcgcctgtatgacagacagcatccagacggtggaaagttcccacagacaggacaggcagcagttagtgaataagtcacatgaaatggccacaccatatgtgctgagaaggtgcatggctcctcccagcctcctccctggtttcctttctgcagggacctcacagtcatgaaggccttctttgccctctgattaacttctgacttacgacagattaaggagcaacccggccacatgttctgctggttcatcacgagggaggtggtctgacccttccctgcaggctcccgacccaggatggctctgaacgaacactgttactaacacacacactgggctgccttaagcaactgttcttaaaagtcaacacagttgtttccctttctcaccagcacttcccatttcattctaggtggaaatattgaaacaggtgctacagcaattcaactgaggaagttatggatagattctattgcccctactgtaaaattgctctcccaatcctgggatataattttgagcccagagaacaaacccatgaatccatgagaggaccgcaaagccaggtcctcagaaaaaacctagcttccgacctgctccgaatgacttgaggtgaaaatctacagaggctaaagagatgtccttgcaaaaatgctacttccagctctacctgctcagaggaagtcactgtgttttaatagtccagcaccacgggtagagatgttgtggctgactccatccaaggttcaatttctttgtagtaatcatttcagaagtcctgagcccttgctaaaaagctcagcttcctgaattctccaggctcaccagattgagagggaagcccaccctgctccttctcccaccgcaaggctgcctcacacctatttcacactcagatttatccacataccaggccttgatcacaggaagcccatctgccagccacagggaaggcaaatgccaactggaagccccgaggtacagagagacggagacaagcccatctcaccgagagcggcctagctttctgagtttggaccagattgtaagagataagggagaggaaggcttcagataacgctcttaataccaagcttccccaggccttggctgggataaaacaaccccatttattcctgaatcggagcttttgcttgagtttgggtgactttcaagggcccaacaatgccctagggtgctaggtggtgttcacacggccagccagagtcaaaactcagacggggccagcgttctatgccctcactttctcccaaagaacttccatggctccttcaggctaaagccacccacctagaccagggctggatgatccgcaatgtgggccacacccaaaaaccctatagcttcttttgttgtaaaaaaaatccaccttgtcttgcatttcagatctcactgaagagttcttctgtgcctggaagacttattttcagtctgagaagaatgatttttcaatggttgtcttttcttctattggagctttggtctgtttgaggttttgttctatagcagaatgttctctttcgctccagtgtacataaatgaagtcacccatggtgtgatcatgaaaacagaggagctgaaacttctaggcacattcaggctaggtcacatcctatttcaggtggtagctccaccttgaaaagcagggggctggaagagctgggattcaggaatgggcttgctgactccaacagtgaaggtcttcccctccaagagaccccacccgtggggaagggtgaccctcaaatcttggacagggctctgagaagtcccagaatatctgcccacagcaatctagcatcatcagaacccaccttcacgtgtagccaccgtgtctccatgttcctggtaatgctaacacaatcagaacagaaatctagggctcttggccttcaagcctctgttcttcgcttaacacacggctggcagagatggggagacctgagagggccctggaaaatcagagaggaggaaatgtcagggactcctccagcacggaaaacatcctgatagtttggagcgccgggaagagtgggcaccttggaatctgcaaggttacccatcttatagccgggccgtccctgcaaaattccagctgcatgggggaaccacacttctgacttcagaatgaaaccagtgttttgcacacattcatgcggccctgcgtgtgccagaggcttgcagagtgatctgagcacgccaagagattgctgtcaagtaagctcctccacaaatctgcagaaacctcaaagatttgtatgtggatttgagaccaacgggaaacgcgctcccaaacaatgctcgttgtttgcaccgagccgcttttctgctaagtttcatcaagtaatttgcatgtggactcagggccttcccagtgccagctcccccttcagactggggaatagaacagtagccaagagagataaggtccccttctggcatccctgacttttcaggaagggaatattgagacctcctggttcttgagagttacttgggcccagccaggctctcagcatcttacagggtctcctgctactctcttatctgtggacacaatttgttgtccatgcggtaactttaggggctgtctgagatggtctgttcctaaaagactgtggatgggagaggtgacagtaaatcctggagagtggacctcacttaaaatcaaattctcgcccttaatacttcctttcagaactgcaccgaaaatgacgatgtcttctcatgcatatgaattatccaaagtgtaggaccactggagtacgctgaagcctttaacccaagtacatttaatgctgcgaagccccgagtgaggcaaaggtgtctttttattttagaagacatttaggacataaagagagcttatgctccccaaatctgtttccgagccaggtaggatgatgaattctgaggtgggactgagggggaacggtggcagctctctcccctgcaccgtttggacattggcgtgacaccctccttccctgttcatctgtgaaaatgtgcagcgttttcatttaaaacctcctgctccaacatggggccttctgcaacctgatgacttctccttagagaaaaagcaataaaaggagcctcttctgtcacaaatttccacttagcatggtttgtgcagaggcagaatttcaggcttttggactcagagccccacatggagtctgctgacctgtcttgggattgaagggtgataggtgagaaggatggagaatttgtaaagggccggcagtctccctggccttcagtcattggaggttggatttccctgtttccaactcttagggagtctctactggggccagtgtgtcagcatcaccttggccccttctataccttatgaagggtaagctgggatccctttggccccacgaagcattaaagccttcatgtcctctcatcaacacaccccattccagggcttctgtgcctccccagcccttgggacatttcagtgtcctccacctttataccaaagactgtgcccctgtcactatatccatgtcagcctctaagcccccggctcccaggaccacatgcgccccaggtttgctgcccaggcccaagctacccacgggcagtgagaaaggtaccatctcccagaatgggaccctcttgcctgtgtggtcagtgccctttccctggaggcccccccttggaactggaaatagtcctggataccatgattgaagcaacttcaggctgtactgagcccacgcctggctctggggttgccctccgctcctgctacccactgtgcacttgttccaggtgcattcccaagggccgacttccttggttcattttgacaaactttgaggttgctcgtcatagcccttagatggagtggtacagtcatcccctggcaccaagcagcccattagcccagaatggttatctgatcagatgcctgtctcctatggacctatgggagctggacagcgaaggctctggcatctgtagccatgagaggaatgtttgccgagtgaccccatgagcgagtgatgccagcataaaggccagtgcacccaaggctggccttgttggcatctcaggctgatttgcgtcccctggggccactctgataacttggctaaacaaatacaggaggtgctgtagggacaggctcacagggccaga

SEQ ID NO: 4 - DNA, 5000 bp, Artificial Sequence
  Nucleic acid probe
ggaaacttggcgtaactgcagagaccacggcttcttcagagcctcacatctcctgtgttggtgccatcgcagctgcacagcccccagccctaacacccagagggaaaagagaaggaaaggaccaactgccttggcccattcagtgatctggccatgaaaccaggggctggctgaaatgtcattgtccaagaaaaggtgctggactggctcacctgagttccacaggaaagacagactgtcctgagaacagacactcgctcattttcatcactgttcctcaaacaagtagattttgttttaataaacagcgttgtcaagttggtcaagtttgttgtttcttacccaaacttcccctttaaatcttcggcaggacagaggaggaccctgggcttcttgtcaaatttgtaattgtcacttgtctagtgttttggtgcgcacatttacagaacgaagcctgctgaaaagctttaaagtacagcacactaatgattcctgagataagacctctagcatccacccccaggcttttccactgcactgtcgacactgaaggtcaagttatgccccagactcctactgctggaggggcttgggccatatgtggcctttcatagcacctagaatgcccctgctcactcatctgtgaagcaaggacgccactctcactctgcctgtcttgctgtcactcaacactcaacagttcctgatggcccaaccttgcctgttcacaccagctctgggcctgtcccccatggaccaagctctggattccttgaaggccaccagtctccagcctccagccagctgggcctgtctgcttttactccaacaccatcaggagctacaaacaagatttacaaaagccagcaagtttacacgggcctgaatagcagattctcttcacttcttctgctcctttagtctagtatttaggaaaactttcgtccaaatgcaagcttccaagatgttctccctctggtctgccacacctccttttcttgcgtttgcaaacccatttcggaagacaggaaatgcttgatttacctagggcacccatcaggttctgtatttcagatcaaagtagataaatggtaaggcaaaaagatggacgctatttaaggtggttggacattgttttcatggaggctacatggagctacgggccctagtcaatgtcatgtctgagcctttgttgaacaccacaaatcttgcgttcctaaatgcaaccctcccttcctgaattaactaagccatcttagcaactatatcctctgtgggttttacctgtttggacacagggaaggtgctctcataacagccaacatgtggcccactctgccaccattaggagcctagcttgtccaactggtccagcctccactgagcagtcaggtcctgctgctcctttctcgcaggggacccagactttaaaacgcaacccatgcaaatgaagcaagaaggcagaagtcaagcccctggactcggctgttttcattggtcagattagatcacatgcactctagccatgacccactcagagaggccagagaatgccgtttgctgattggctgagcgtgggagcgaggaccccgctttgttcggtcagagaccaacagtaggccctgcttcagtttcctcatcctctagaatcaaggaggacccacgactgcctccagttcacccttgaggcttctggggctcagaactctgagcgggcctcccgggctgctcagctatcagtaaaggagttagttcccggtgcccatgtgctctgtaccccactgcataggggagagggcaggcgccaggcatccttccatccttgtccttgatgtcactttaacaccatgggaaccttgacatccttcacacatccttctatagacagtgctggggcttcgaatcacctatcctcatcctaagaccgtgaagtcacagtcctgatgcgctactcttttctgagtgcatgtgcgcacctcttggaactgttaaaataagcatgctgccccctcaagagaaccgtgaccagaaacttagtccttgggaaatatgcgctactgcagaacttgtcaaatggcaaagaatcaaggaaggaataaatcaagcactttctatacttcatctggtggttttaaagcttccgtggttcagaggaaaatttgcatctgaaaatgcggcttttcccctagctgcctgatgtggatgagaaaatatcagggttgtagtccttagaagtctccttccagattagtaatgggaacagaattatcccaagtgaatgactctggtggtgcctctcctagtatgtggcagtgaagaagagtggggtacacctatctgtacaacgtaactgttgggctggaagagaatgccatgcacgttccaacaccttatttttgtatctggaaggcaacaagtccagttgatgacagaggctacctctgcggagggtactgggtgatgctgatcaaagagaactttaaacccatgggtaatgtctcgggcctggacagaattctccaattaactgagacctggtgacatttgtggtactatgcgatgctcacagcacagatgcacgctccctcttctgattccagcagtatgcgtcagcattgcctgtgcaccctggtgacacctggacctccttctaccaaacaaagacactgcagtaaggtggaagcctcctttgacccctcttggtgcccccatacctggtactgaatcacacatgttcttccttttcttgccttttcccccaaggcgctcactggagctctttctttgcccattacattctcctgagctctcccaggcttgggaaaatctcccttcctctccctgctctgttattccaattccccaggacgccagaaactcagacacctttgtgcaagggcccttagggcaaatccagaatcccaggcccccgtgctctacacaaaccatctctgagacaagggagggcctgcttcataccctgtttcactccccaccaaataatcaaccgtgatttgttatcctgcagggttgctcataccaagagtgcagttcatgaccacgcttcctgcacacgtttcgctgaaggagtggttccatgcattttagtggatcctgctcacttgggtttctatggcagtcatagcttcactcagttaagattgaggcatgccctgcctcctacctggaccccatggaagcatcttctcctccagtagtctaagcagttctcagcccagggattccagcaagtgtctcaaagccacacactcggagagacgacggccacatgacctggctaatggcctgaaagctgcgagaatggcttaacacacctgaagaagggacatgtatatgctgggggctggcactctcccacattagctcttttgcaccattgctcactgagacggtgatgcttaactcaggtatgtgtctaatgacagggtcttgcagaagggaccagcctgagggactaggactgcctcacctttgcaagtaacaggggtattttcttttactgcgcagaaccgaggggcataaaaccctgattcgtcttctgtggctgggtttgctctctgcgtgctcatatcctttaattccgtttcccctcggctgctcccagaagtttccataaaaacagatgtaaggtaatcaaagacttcccagccccgggcctctctgctcctgaaggcttccataataatagtaataacacctttgctgagcttggtgcctttttcaatgactctcagagccatgccgtgagatcccggtggcatcgcaatggctgatgacagacagcctagattgcacatgtgtgatattggtgggtctccagcccctgcctccatcctgagaagcatcctttttggggaatttcctgatgggtagagtcttagccatctgtccccagagcctccatacttgaggagtaagctggtcttgcttgagagagcactgtggtttcttggatctaagagtggaagtgggatggaagccagaaaatgtccagcacccatgatcgggggcctttcttacttccccaaagtgctctctgctcacgccagaaatcatcccaggaggaccagccccagccctgtaatgagcggaaatggtattccatagtaatccatcccaactgactcgctattgtacacgcacctgctaacattttttaagctgggactttatggaaagttgcttctgaagtttgctgagcaattccccctctctctcaggcatacggtctttgtgctggaaatttccctcgtggctccatgcgcggactggcagacgggagatcacggagccgtgagtctttctttccactgcaggacctgttgtggtccacgccatcaggaaagtggtagtcagggcagtctataacccagcaacgaaatgctaggaggctggcttctgccctcacgtacctgatcacagtgatatgggacggtccactcaacccggaccttcatgaaatcctcctgattatttctgtgccatgtttgcctggtgggttacctagcccacttcacttcctgccagcacttgggtttgtaggccccgcttcatagtacacctggatgtgcatttgtctccaaataaaatagagggaataaactgttactcaggtgttaagcccctatgcaccaggaatcttgaggatgcagggagatgatgagaaagagttggagtggtgtcaaggggatcaccaaccctgttggaacttcccccggcctagtaccagccccctaaacctgtgagccatgacaattcttctcaaagatagctcaaatattttaagcccagaataattctgaccaaatcaataaagaggactcgggtcatgccccccaggagaaccctgtgtatttgctagcatcagaagtcatggacccaagtgggaaatagctctaggaatggaggcttctgtgggtcctttgctcttggaaacacctccttcctgccaccttccccagtgccacacgtggcacctt

SEQ ID NO: 5 - DNA, 5000 bp, Artificial Sequence
  Nucleic acid probe
cctctctgaggacttagtttccccaacttaacagactgccctggtgcctcctggggatgtttttggacactgagtaaaatggtgatagaagactacagggcaagtaaggacctgacaacaacctgaccagctgcacgttacaaagggcagatacaaagataagaaacgagggccaactacggcagcatttgcatccttctgacatcagtttccgttgttgaggatggtgtgtgaatcacatcttgcgctagagaatggtgcgtctaaggctcagcacacagcgatacctcgggaaatgttaggcagtgcacatttttaaggaaaatgaaagacatggagttggatgaacatagggctgtctctgtgttcagggggccggcatgacggtgcaggagcagtgcagacccacctggctatggtctgcaacctttggctgttcttgcttaaggaacaggaagatgtgactttagcatgacccaaactgcaggtttacacgttggcaaagaagctctcgctcctaaagcagaaagttccacagcttctaggtatgcatgaacgcatgtccttgatggtgaggacagggcccagccttcatatgcctccctgcccaagtccttgtcagcctctactggtcccaagaccatgggcctgcatgctcattaggccatgggcagcccgccttctctctgcttgcaaacctgaggaagcatttattccagcatgtgagctgtatttcaaggcaaccaaatagccctgattgatatggaaaatttctttccggaggactctagtgaatcaacagcaaaagaagatgctgagaggaaattggtgagaggtgaaacataaaataacagcactgagctggagacaatggaatttagagattgtggccaggaaaggtttgggggttaggattttaaatgtacagatgtggtggaagcttcggtgacttcgctctctgcttctctcctcttcctgagctgcttagggacccctgctgacccggtatgatggcactataactgatttccctgttacccaccagcatgaagggcatgaagacagggaacttcattagctgtccccaaacacaggaaattaaaattaatcagggtaaacttaagaagggactccttgtgggaaccaaggaacagaccctgaaaatatagaaaatatagctatcttctccctaaaggccagctcctgctgttgctctgcacacgtgtgggctcaactgtgccaaatcatcctattgtcccctgagccagaggccctcccgtatccaggaattcacagctttgagaggccagggcctttgctatcccagcccgtgctctgtcttgcagctgtgcctgtcttcagaaatcccaaaatgagatttaaatgaaagctgagggtatagcctccatctgaagaagcgatggctaggcttgctccccaatgtaagaggaggttaggccgggacagaagcaccctttcactggttttcatccttcccactctagtatttccatgtgtagcctcacaagataactggctcatccaggtggccacagatgtccagcttggccccacagcaaggccttagccagctccactgttggtcttccttctccttaacctttatgtcctggtgcccgtcctccatcatcagagcctgaaccatataggaagctaagcaaataacccccaaatctcttgttgccctccctacacagcagctggttcaccacctctgatgcaatccacagtctccctcaaggctgggccaatgctcgggagctctggacttgaggcaaggtttataagcaacaactttccccctatctacacctatttttcttgtcatggtgtttaaggacctcctccgtcttctgtaggtcagatttcaaattcttctccctccagcaattacctgcattgatccccagctcagcctgacctcagcaagagtatcagtggaacattcagtgtaatgataggtggtgtctcaatacattgctgaacagaaacagtgttcgacctgagtgcagggatcccagctaggttagcagagaagcccctcactttgcagagcagcagccagggtagttcatctggtttcccagctgtgttttgaatagaaaagaaacttccagctgaagatgaagcagggccgtccctgttgcattcttggagttttagaagcatgtctgttctcggatagctgtgctgccggaatagccataccatttcccactagaaggcattttcgtggttatccagagattaatggcgacagatttctatacgttttgtagtttttccctgctcacatacagaatttttctctggagttatcataattccccccagtacgacagtcttgagctacttggagccattgtgccaaactcttgttttatctggctttgctcttctaactgaaagtgcttgggcttcctctgaaggcggccaatgtgttgtggtcgccaccatgtgctgtgtcttcgaggtagcatatttgaattaccctgcaagactcaggagattcagaggtcttaaggagtgtctaggctagcttcctgtagacttcatagtatgacctgtgaccatcacttttatcaaagggtatatgggcccaactttcaaatgtgttaaaggtactgaaaaggggatatttcaagagtacccacaatgagtcaaagttccatccatttaacaaccattggtgtggcttctgtgttaattccatcagtgcgccctctctctttccatgccagggtactgagaagtcattttggatgcattataaattgagtctccgtaggtaagttggctttccagtgcatgcacaaacaaggtgtactaagcttacatccagttgtcagcctcagattaagcccacgatgtcttcactggtcatatgtcagctgagcgcatgatgcagcctgttcgctgagcactgatggagtcaccaagtgccttgctaatcattgctgcccacttacaggcctcgtgctcacatgactttttactgaacattcttcaaatggtggtcttttggagtcttgtgtttgactaacagtgcttcatcacgtgtgctagtgcatacaaggaatgctgtgtttctggaagtcaaagctttttaagtggcagtgttgtcacaggcagctcttaattattggtgctaataaaggctaagtcctgaatgggtaaatttaatcaatcatgtctatactcaggaaccctaagagctgctagtaagtagaaatcaaaattcccctgtaagaggagctttttccacttgctagtaatccctaccccttgaccaagtcgcttatgttctgctattcagctaaagccagatgaggccacaagagacactggggtgaaaatggcagctcagtcaacttgggtgagagatggagaaaaagcgagtgattcaccaaccctggagaccctgaactgtggttgagcagaccatatcctcatatcccgcctcagctcacagaattcacaaatacatgatgcagaggttcacactgacttttagtttctaaggcaggtagagccatggagccctttaagattcagagcatggcacccaatacgtgtccacgcctgcgacaatagagtcttaaatataatttgctgcagatacttcagttcctgcaccgacagacaattgcagtctgtatccaatgagcacagattttttagttgcataggcttaccaggtactctttcctgggagcctcacatgggagggcaacactatcctttattcaataaacagcatcccccaccttttaaatcatccaaaagttgtgtggtatctgattatgcagtgcggttcaagtcaaatgctggcttccttggggttggtgagtgaggtgccctcgatggagtccctgctaaaaatcagaatactacaaacacgtacaacatagaaactgatgattccccagagatgccgttcttaagagttggagtgttaaaacagtgtacattgctctagtaagattagaaagcgagacattaacagaaattaatcaagcccctgtccaggttctagtccttttttgagcaaaggtgccatacaccacggaatcccagttcctggcggggttaccgggggcatggcatccaacagcccagtctgcaatgcaccattagacacacagacaaatccatcccctctactttggggcagcactgttctattccctaggcctcctggtgatccctgaggtgacctttggccctcagtccctctaaggcacatcaaggccggaggtgaggcaggtgtgattccaggcactgaggcggactttgctcttccttgaagggaaccctgcactgtgctcccgggaaaccccctctctgattccaccactgtgatggagaaaactgaggctctgaaaaggatggctcatctgcggcttgagatagatgcagacgtcaataaggaattcacggaaacacaaaaggcgggctgggtggcttttattaattataaaaaggtctgtctgcccaagctagtcagccctcgcatgagcctggagatcagcaaagccctagaagcccttaccagggccccctcactgccatttttgcacctgcgttcttctttgctatagtcccacgtagaggagttctgacctcagtattgagtcctctctttgggtggaggtagaggcagggtatgtgtgtattggagcaacggctgaaagactcacagattatttgcccgcttctgtcatcactctccacccccagatcctagggtgaactgtcagggatggcatcgaacagtttacctgcagctggcgacattgcgggagccacttacccgcaggtaggtttcacctttgatgagccatctgacctggagccaccaaggctcatccagaaatggacttttcgcagttgaggtctgcctcacccctaaggcccctagtttccctctctggtgtctcttagatctccctgtgtttggatttccattttcctcttcc

SEQ ID NO: 6 - DNA, 5000 bp, Artificial Sequence
  Nucleic acid probe
caggtctgggcttctcatgcacaaagcctctgtctgccacccacaaacccattcggggtagagttgccctcttgtagtggaataaaggacaagtgtggccgtcgctgctctgatgtgtctcagtgagtgcaccgggcaagacgctgtttgaaggagcttccgtggaaacatccataaaactaaccttgagggaagaactgacaaaatgactttgttccagcttctcagaagtgagaccagagatgacatttgcatctgccttggagcgttttgtagatttctgagcctttgtcactgatcctcctgccttgcccccaataaatgcctttgaatttaggtagaaagaaagttggaagacgcccgcagggtgtctaagaagggttaggcatattttcactatcaagtggcaaatgaagtcatcagctccaggtagttcaagtcccagccccacctggctttgaggtgaggttgtgctttgagccggtaccattttggatgttgtgtggtctggtaactgcgactcaggtggaagcatggctttctcagctgatgcctacaagtgtagatgtctcaactgtaaccagtggatttccacatcaggagggaagtctagaagattccaaagtctgttctcgctcgcacacttacactaagtgctttttacgaacttctgaggccacaaacttctgccctgacaagattttctcaaagttccggatccaggattcacgagtgttgcttgccctgtgccaggcacttcctttaccttttgtatctagttattttatcccaagagatcagtctgcactgtgagcttaaaatactctagtgagcggatgctttcatgcagcagatagccaggtggagctctccctgccaatcttacaacgttaattaatcgtttgtcattgtggttaatttttcctcaaaaagaggccccctatctctctggcccaatgcatatttataaagggcagggaagtcaggcagaggatgctggtcccagggtcccggagcactaaacaaaaaggagctgaaggtcaccacactttggccatttgcagggagcccttccccacacaggggcggcatgttcccttgaagcatgaggaatgtgtgtgttccacagtctagagaagcaccagggctatcactgcttatccttccccttgtggcagagagtgggtgggagggtatttcaccattcctcccagttcactgttagcatttatcctggacaaatgcatcagcatttttgatggtcagcctctcaataatcgtagaaataccccatagtacatagctgaagccatcaattaccttttgcaaaacacagtagtattgcagaagtcagatgtgaagtgaagggtgtcttgtcatgatatcacacaattttattaagagccccaggcccaggcaagagctcgaagagtattagcttttgctgttgtaaaccccatggggtggtctgcaaactgcttcccatactcgctccgtcattggattagcgtgtcttgtctattccgggagaaagttgtcctacattctaaaaatagagctgggagtctggacatgggcatgcacagtcaggggaggctttccaaaagaggccgtgctccactccaggggatgctccgtgacctctgaagtgaggacaccctctcaaatcagaggcacgggcctctctcctccatagagggctcattgactctcctctctgttttcctcttgatatatcctccccacttctctgctgcagaagaggcgaggtgagtaccattcacctctcccaagtggtcccagcccctctggccagcatcaagcccttattgagagctccaactgtgcacccagcgtcagaaatgatttggggtgcctgtgtccggagcatacagtcctgcctcctctgaactactctaagtgaagccaatcaggattactttcatttcatcacttcaaggacttcctcctcggagtcttttcatccttctcctgccttgtgacatttttcttccctcattaagaaagatgcccctggtgagatacctgtgctgggctcagagcagaaaataaatatcgctggcatccactttcctatacgccctaacacttccctttggggacttttcctttccattgcggttttgcttttcccccttcacacgtctcttatcccagctgtagttggctgtcttgtttgggtcacagttactgtagcccagggggattctgtgagagacagatgggctaatttggctcgttcgtcatttttaatgccctaatttcctgttttacacgcagccgttcaggagctggctggccgactgtcccagtacctccttggtcaccttcatacggagctttggtccttcaactttggaaagggttttgattaccgaactgtgactaatcagcgctcacccataagaaatggtgtgcatctgcaaatgtcgtggtgtcccttgcaatgccctatttcaggtctgcagtgggtgagggaaagcctgggtgcaaggcccctctgtcacctctggagtacttgcctccatcaaagaagggagcagctgcctcctgccggtgttagccaggcataggctctctcatcagcacttatggaaatagcacaccaagttgtcaactgcccgtaaaccctttgccatccctccggtggcagcatgttccccgctggtggggagaaagagaaactggtcccgtctgccgtctttccagatagttccttctggagcagaagtagtccaaattccaaacctctgattactgacatttgcggttagtttttctgaataatgcaactttacacagacactctcaccactgggcctcaccaagtcctgattttcaaaacactcagagcccttcctgagctcacctgagagatgttttaaacctgctcactggctggctctcttctcgacttcttgggacctgagttgtccttcatctctgtgacctacctgacaggtgtttctggggtccagcccagctcctgagcctgtcaaccctggctatgcacagccctcgcctgagaagcgtcttcaaaattctagtgccttctcctcccttcaattccaggataatggctgtgtgtcaaattcaccaagatttagctccagacccagctgatctgcagccaggatgctattgggagcatcactgcttccgtctttgacccatctttcggaggatatcaatgaacatgaacacatgcgagagttcctgtacggagctgtggaccataaaccacgcatgctgtcccggtggcaagtcagggggctttcttgtgaatagagctgaccacagtagcacaggtctcaccgtaggcctccaggaacaccctgcaacctcagtctgcccctccacattcatttccaggaaactgtccctgaaacatgcctgcaagaaccaccccttggctcagcagctttgcagtgatgaatccaacctctgcagaaaaggcaccctggtgttctaattcctggcttttctggcccttaccctgttctgctgtccactcctgatcccccatttcctccctccagacttctttccaggatgctgcattcctaccacgtgtgctggagatacagagttaagcacaagatcttagcggaagatgaaaacctcagccaaatataagtgggggactctgcagtgaggcaagtggacacctcagaggacaggtagtgctcttggccttggtgcatatctgtgacccaggtcccctatccaaccatgagcctccaacgcaggcaagaagccggcctcttccttccagcctcctggcaccatcaggaagggttgcttggcaggccacctccggactttgatgcagaatgtccccaggttctataagcctccaccctcgagttcccaaaggcttgcaagccagcttttttttagccttgtagggtccagacccgatgggtgaaccatagtctgtctctttgcaagcagtcaggcctggacctgtgctctcattattttgtgtggctgcagaatttgtgcacagccctggtggcatcatagttactcctgccacccacctggcaggcctccattgcttctaaatgcggtccagggaaaccagaatctatttcaagtcaagcagaacttctccggaagctggggtagcatcccatgtccccacaatttgctgcagggggagctggggcaccccaaaggccccccggcccacgtgctcaaccactctgcttcactctagaaagaagagaaccatttagatgactgactgaattttcctggaaatctggccctccattgtaagctgttgaggcaccatcaaatcccacatcacaaagagatgcacccgttttaaatgaacttagagaagatttggcgaatccatactccagtctactcaatgaactggtgatggggagtaagtgctatttgaatccaatctccacccttgttattaacaacgaaagctcaccagcgtgcccccaccccccaccatccaccaggtcaataatatatgcaaccggcaaaagaaaacatcgacctcatttcctccagagagcagacaactgtaaccctggagtcctggtttgcatgtaaaccggggcccattaaatcctgaccaaagtgctgaagagttgccccacaaatgatttggctacactgggaatcacccggaaggaggccaagggtgtgtaattgcagaggctgattcctagctattattctctgaccttccatgtcgcagaaattatgatggtaatcagaactggatcatgtttcctctctagtttggaggagcctcgagagccactctgcacctgctgtctgcactagctacagaagggtccagatgagtgtctgcacaggtaggtcttgccgctgaagactaagccttgcatctggatctttggaacaggtctgccctgaagtggtgttttatgggcctgggcactctggcacaagccctttctgggcacaaggcgaagttgatgagactc

SEQ ID NO: 7 - DNA, 5000 bp, Artificial Sequence
  Nucleic acid probe
gactcttctcccaactcctctgtgcatggcctgaacttgcccttgcaacctggcatggatcaagctggcttcctagtttctgccccccagctccattcacccagggagcaataggcagctgtcacccacaaacgacatggctcccacaatctacttcccagatcatagtcctgcctagctgggactggaggtaggatacacccttcaaggaagccacgtttctaagagggctctggcatatgcattggtgagttgaagcttgacagtgttatagcttctctgctgttagtgtgaggtttgctttcacaaacacctgggtgcttgcggactcaccttttacaatggttttaccagcctcctccacaagcacaggcaaagtcagtttcaaggacaggagccctcattttttagccacctcgctttcatccctggtcctcatgaccccttgatttctttctgaggaattccctccctcccactgtgagaacagaggggccaggggttgggaagttttgtggctccctaccaagggatgacgtgcctgtctggcaggagctaggctcatgcatggctcgggagagcacctgcttgtactgagtccatttaagtggcagggactttgaaatcagtgtgtcagtccctgatgcttgtttattttcaaagcaacataggagaatggatgtggaggggaagcggggagtcaaggtgacacccagaactcatttagggacatttgcagactcccaagttccttcaccagcaaagtaggaaacctctcaggaatcctcaggctcttcctcaatctcctgatggatgctgccataggaagcacaagagggctgaaaccaatacaggagtctgtgctaatccctcaggactcaaaaatgccccaatttttggtctgatacacatggggctacctgctttgcacttcctggccaaggggatagactgattccttatgggagcacttttgaagaatgaggaagaaccttctcagaccttcccagaagcctccaggaagtctctgtttggacgacatcgcacacccattcttgaaccaactatttagaagggagatgcatcaccccctttgcctgtgtggaacgaacacgacccatctttactgctagcgactgcagcagataaggcaaggccactttcacgcagttaggttaataggaggctgtacaaagggctggttttgatagaacttaaaaaaccctcgtttgcttggaaaccacctgttgggatgaactgcgggaatctttctttgttctggttggtttaaattattttgattctttcagagaatgtgacacagaccctcagaggatggtgtttctattgcctttcctctgctgcagtgggttaatttcacctccttcagtgtataatacttttattctgatgttagtgtgactgaaatgcaaaggatactttttggggtgctctgaacagctttctggtatcgtgttgagttacattggaagagagagtgttaatgttaattcggcacacaaaataaccacgtatatctgtcatgggtgtgtaaaattatccccagttcaggaaaaagcgcgtcagtatttaatctatcttttgtgaagcaatttatttaggctttaaagctggcccagttgtgggagtacagtgagttctagtaaagtagaagcgtagtgacatcctgagtgtagaagtggaatgttactttaattgcaaaacactttctgagcctgaaaaatggctcttggctggtttgaatgccggttaggcaggtttatttcttggttacttcagaaatggaagcttaacctgtctagactgtgacctataccataaaatgtgtggtctaggactgttgtggagtgtcttgggctgttcagggaggtttccttgtatttcttgtgataaactaaactgcacatagtttaaagaactgtcagccacgtaggccatataacagctattctttccaagcctagaatccatgccatagataagaaaggcacagttgtaagaatatgtttgcttgtatagccgaaggcacattccgttttgtaggtaggtgagttttcagaactcattacttgtaagtttaaccattacagaatgcctgctttaagaacctatttctggtgtcttcagggtttttttctcctagcctctgacctcaagaatttaaaaaacttacaggagtgttttcttgtacatttatgttgtttcctgtaccagctgctttagatggttggttagcttgtgcagatcctatagtggtctttcttttaggaccagaatcttgtccaaagtaaatgaggttggtccagttagcagcaggtatctatcctctccctttattgtggctacagacggagaaatgggattctttacccattctttggcagggtttctacgaggaaggcatgtgaatggagaagaaatgattgataatctctgataaaacagtgggcatgcaaggtggaattcaggcagtatttcactttcagccagaacagttagagttcctggatctctagttcaagtcacccccaaaaccctccaaacatgtaaacgtcttcattcttagaagtattactgaaccgcctccattgaactctttgcagttcgttttgtcacatgcagtgttggcatatactcctgttgttattgctcataggttaaagacagttttgtgttgcatacaagagacaatattggtatttagagcagtgaactagagcagcgttttgcaaaccagataatgctgctctctttttctcatggcctagtcttaagcatttatatccatgctgtgcttacgaaggttttactgagagaacaacaggagctgtcttgattattgtctgcagggttattgcaaaaatcatggtaactgccccgacttcattaattttcaataaatgcatcgttgtagttgcttccactggtggtgtaagtgtgttagcatttccataatggaccattcgcctttcattatactagtgagcagtgacagaaatgtgacatttacttcatggcaggttcatgtgaccctcaatttcacttttcagttttgtaaaaccctctaaactgtagaagcgagatatgaagcaactacaggagttcagaggctggagccgttcttagggtgtctcacacactacttttaaaagcatgttgctttaattcctacttaaccctaggtcctacacacacaaaaaggaagtctccgtcctttatgttgactggtgagaatgaaatgaaatagtacatgtgatgcctggcagttcatctttatgtaactcttaagcacagagtcctgtttgaggatgaagatgtgaatattgcaacaaccacagtgcttgtctgagaagtgcatcacttggtgataatcctgagtaaaagattagtaatgagtgaagaaacaggcacctgtgataactgacaagtcttcaaacacatgtgtatggttgttgtagagagaacagtagaacagattttttatgcaaatgagctgtttcccataggttctgtcttgatcttaatcctttaagggtaagggctaacttgtggttgtgttaatggtcttcatcgctgctgagagttgaaggcggaacctgatctataaggatccattatgttgaagccaaagaagcaggtctagctattctaagtaagtaggctggagaggtgtttcttaagagatactatttggagatcactgtttgactaagccctgaacatttccacgaatgtagcctttagagagagagggttcctcaagctgtagtttcttccaggaattgtaggtagtttgagtagaaccaacatttgagaatgagctggagaatggagcatccgggttggaaatgtcacctacattggcatgttttcagaagttgtgaaaaggtgctacctaaaacagcacccttatcctttttcagttgtttgagttctctgcatccctttattagggcagctcttgagttttgtttatccgagtcaggacacggaataggatacaagatactgctctcaggactcagctcctatcaaaacctccctcacactgccccttggtggtcacaccctacggctgcaatgacttgaaattctagtatctaggcaccatgtcttagaatttttggctgtgacaacaggcatttggccacatttctagtcattagtccagtgattaatatcgctgttatcagtatcttactcattattccagggatattctgtgcatttacagccatagacttatgcattcctaccccaccctatacacctccactcagcttaccattctgctgtattttatgttattgtgagcaccaggttcgtttgccttgcggttgcccagctttcttttgtcttgtcttaaattttctgtatagctgactgagtttactgaagcaaaaacattccataaccttaaccttttccctgaccccctgcttttaacaactgccgacatagttttatgctgttatgtgatttgctgccgtgagaaaaggtctaattgttgcagaaatttaatgatctagagcgacatcagggctgtgagattttatgaactgtttacaccgtaattttcagtgattgtgttttatttaaaatagcattgcatagcatgagagccgtatcggagaacatgtgccactctcgagaagcccttcatatgtggtgatatttcacattcatcattggtagagaaagaactatgtacttttctcctccatcctcttctgatctgcttactgattgagtgcctgttgtaaaacagtaggaaatcacttgtaagctgtttttggaagaatgaatgaatgtgggtgtatttcagccattcctcttatggtgcctgcagtggctttccctgcgcaggttgttcccatctttgtttggggtctggaaggggatcagatttgagtggcaactactgtaacttcacctgtccttcattcagagagttttcttaagaatcctggagttaatta

SEQ ID NO: 8 - DNA, 5000 bp, Artificial Sequence
  Nucleic acid probe
cactgggaattcgtctaattcgtctttctcagggaaatttaaggcctagaagacatagatctggttggtaacaaaacaggaactctttcgcctctgagtgtattgtggaaagtatgctctaagtgctgactagggatagaaaaataacatgttaaccccatgtgaaccctgaacaggcttggtttttatttttttactcccatttttgtttccagtgggctaggtccctacgtttctaccacagccgtaacatgcttggcatgcagtttaccttttcagagtagaaattatactgttgcatttcatgttcacagacttgattttgagcctgtctatgttacatctttcagataggttttccctcatagaacaagatggtagtagaagcagataaagtgttaataccagaagtgttccaggagctgtacactgagagttataacttgggagtcatagtatcttctttgaggtaagaaaactggcaactgcttcacgtcagaagatattttcttgggctccaagattttcttggtaatgtctatttgggatcttggaagctaaactgagggtaagaatggactgtcagtgatgatttgttggatgttgtggcttagggcagacttggctattctaagatacagagcggtgtctaggctagatccacctgtagtcacaaagacgtaagtttaatttttttacatgcaggttttccacacggccaggactcagacctgtgtgtgtcttctgtgcggttgttgcacactccaaatctggtctgtgtgggttccttaataagaaaggctctctcaactttgaaatgtactgaccaggccacactagtaagttgtggagcctgaatatatggcagctgatgaatacaatgatgatggtagacatagccggaaaccatcagtcagtcgacacctttctggtaacctgggcgtggttcattacccaggtggcattctcttgctttggaaatttaaaaaatggcagatttgctggtggtttttttctctgcataccccgctgtttgcttgtaactaattttgcatgccagtaggtagcagtggtgagcagaggcacagacagaaagctgcttctgaggtttgttgcccccctgctgctccaggtttgaggttagttacagcatcataagacgctctcaagcatagagaactcaggctgggcttaaaaagccgtggagatgtgaaatgtttatccatttacaaggcagaagtcacaaacccagacacaaggcatccgatctcgtcctctgctagatgattacagacatgttccagcgctaggcatgaccgatgagttccttgtctgtgaaccatgactgggctcttggacttctggagctgcttatacttcctgcaggtgcctgctgtgtagccactgctaacaactctatgcctttagccttccctgggtttctggaggaaagctgccttctgccctggacccagctccatcctgctccggaagtggccctccagtttcttgaaaacatctcaaaggtactaaacataagtgctcagtcaccctgttagatgtgggcttatggagtgattcataaaaatgaaatatggactaaagacatgcaatgaagtcgttatttacttatggacaactacccttgcaaaacaaagtcaggaaaagtgggtgatgttcaaatgaggccatttgaactgaaagactacgttcaatacatagaagatgatacatggccagccatgtcatctgcctgtgattccagaactcatctatctgggcccttttcacaatgacattgcgattacatttttgtttaaatgtggagcacatttcaatgacggtagagttgccggccgttatttacgcatctgctaagggaaaggagactatttgtcagtgaaatgtcaacagcaaacctcatcagcaggcctcactgccgtgcaatcaagacagcgccatagctccatataaacaaatgtatataatgtgcctgtgttaaacagtcccttcgacagcctttaaatgttaaggaatcagtgctgtgtatattacccagagagtctcagggagcgtggactcaaatgtgactggcagctgtttggggagtttcatgttttggagccatgaaccagccattgcactgaacttgaaagtttttcctcagaaccctgatctgggtgttcatccacagccatctacccagaagacctgcaagtgaatggaggctcggatccaaaagctaccccgagaccccagggtcttcactcctttatggcagtgtgagtggggcagacaggatggagaggctcagaactgaggcccctgtgtttgtgctggccacagctctcctttcctcccagctcattgcaacctcagaaggcctgtcgagtgcctgtccccatctgaggaaactgtcctttcaatatctgcaaaagggccatccttgtttgtatcaattgctcctctccataccctagccatggtatcgtttcttttattggacctgagtacatagaatggctttatcacaatacttggttcacatacagtcttccttgctctatgcatggcccatacacagtataattatttaaatggccaggtagctcttctagctgcttctgtttattctccttttctcttagcagagtattggaatgaggctgttgagagctgggctgaaggaatgttttgggaacagaatgctcatgacaaatgaacaaccacagaggcaaagccaggctaaaaatcgtcacaaaacagaacagatgttcccaccctctgaagacagagttgtatgctaaggagaggagccgtcattgcaatgtgaatagaggcaactgggaattgcattaaagggacttcaaggatctcccagcttcattctgcggacagcccctgcctcagtcccaatctatgtaacattcatctctaaccctttcttggcctcgtggtagctttcctccaatactccaattggggcctttgccatgatgaacagtgaaaacaaccttccacaagctcccggaaactccctgatgcctttctctgaggtaaaggtctttagatggtacctagttttgtgttagaagctttccatccagagaggtgatgacagtcgtctccatgaccattgattaatgaaaacagggctagttggacccagagtagatggttataaatggtgctgtcagagaattcattatggtgattgaatcctcttcaatagagaacggtttaaagagagcacaggccccgtgcgtctcactcagagttaaggcgcataggttaaaagctgttggaaagaaaagctcatttttataggtggtgccacctccacaatttatactttgcccatgaaataccaaaaccagggagtcaattttccaaggaagaaaacacttttattgatggaagagatctgcagagacggtcgccatctgcaaggaagcgaaggctcttgcaggtgcgggttgatccagggctgcccaagcgtcggaagtgaatatttgaaacctacctggtcttcccaaggtggcccagttattactgtgtgacgacttcttcctggcttccctggtgaaaagctgttccgtcgttaaaaacataatcacccataaaagcctgttgctccccagagtggtgaggtatctaagggatgccccggggtgctcttagtagaaagcggggggtctgcacaccatccctgcactggggttgcctcttgcttacagcccccatccaaatcctaccctctatggaggcccaacagaaccaccctcctccaggcagccttcacatgggcacaacggacaatctcatcaggagagaggggacttgcagtggcacctgcatggaaatcaaaagagcaccagctgtgtgctgcagaggaggggggccgtgggcagcttctcctcactcacattccacgagggcgtctgtgtccacagaagctcctgcaaaaccaatcaggagctgtgacatccctcaagcttgctaccacggggcagtgtaggacaaaccacgcctctgtagcgaagtgcatggccgtccccagaaaagcatggggtagcaggaggtatgtggagggaggagggtccttctagggtcttctagcctcatgcagccaatcccagtgaagccataagcatcacataccctgtccccaagccaggctgagcctggggaatctggatgtgcaggagctggtgagcccaggacaaggaagaaactgaggtgaggctatgcagccatgctggctaatgcctgcagtgatcaggataggcttcctggtggatcccattttcaaccttccccaggagagtctcttgcacaacctggaaagtgattgggctcagaccttcacctggaacccaggtgcagccatggggctgagggccttgtcaccacaacccctgcactctgccagagagtctattcctggaaatgaaatttcaaatctgcactcttaggcttcccatttctcgagcacccctgcccagcttggcctcagagctgcaggaaaagcctgcccgggccttgtttatttttggagagttttaactggttgggcgaggaagtgagccttgttaaaagggcggccctgttggtagcttctccatcacgccgcacaagcggctgagtcccaagatgaccttttaccagagcgggtagagttcacagaatcggatcccatcagatggtgggtatttccaggccacaaagtcctctgacctttgaacagttgccgccgaatttcaataatgaaagggccttttttgaatatgtacaaatgagtccatacattttatttccagcctcatctgcgaatctaatattgacccggagttatctgtgatggggaagttattaattctgacagatataagggagaagctgtatgtggggcacacccaaacaacctgcctcagcacgtcttgatggaatattatttttatttcctgcagaccgtgggtgctaggagttgggtgagatgg

SEQ ID NO: 9 - DNA, 5000 bp, Artificial Sequence
  Nucleic acid probe
cactcctagcctctggcattcctggaaaactgggctctcagcaggaacttccaacaggttctaccccagagagctggccccgactccacctggatccaagttcgtgagccaaggctgagtttattgtcagaaagatgactaagaaatccccgacacagtgtatcatcagcattatgattggagaaacaacacctacaaaagtcccaatgcccacctgtggtaacaccttcctttctctcaacctggcctttctcacccacaaggtgggaatggtgatttccacctctcgggagaagagagcgtctctgggaagggctcaccaaggtaaggtggtcaggacccagtgagcctttcccagagagggttcaaaagaatgtctggggccaagttcaggtctgggcatgaaaacagagaggtacttgcatggcgactcttccccttgtacccagaagaacacgactgctggatagctctgtgccagtcccttcctgtcctttggagctcttctaaggtctccggaaaccttcatagactctgcagaatgaagcaccacagcttggtccagggcatctgcagattgagacggccctctcttcctcccccttgtccggtgtgacctggcaggtgagtccctgccaaggccttagggtgatggggtactgtagaaagatgggtttgtgctcccctgttaatgctcctgaaaaatggtgatttagaggccgcccccactcgacgggctttcttagccgaagacgttcagctaatgaacctcaagcagattcatggccgggtgtgaagtcaaaaacttcaggatcgcctgaccctgtaaactgaaaattcatgtctctcgcccggggacaaatgcattctttgtaaaggcggcccgtggccagccttgggaggtgtctggaccatggcgaggtttgcgtttgccgttatctgggttgcattggcgaagctgccagtcagggtctgcctgcctgtcgttaaactttccacattttgtttctaggaaatggatcaattcttgccctcgtgttcactgaatgaagtaacttacagccccagctttgcagccagacagagccctgggcaagttactcagctgtaacgtgggttgcagacacccgtgttgccaatgttattaccatgattgttgttttcttccactggcagatgcacaggaaatggctctctctgcagtcataaaacagcataacgatgagatgcttttccccccacagccctgtgatgccactttattgcttaggaagtgatggtacatgaaatgttcagtggcttgcccatggcctcaagagagcaaagctggccttcccacctcaccacatgacactggggtaagatggacagcgggatctgttgcctgtgctcctgtgagcgttccttggtttgtcaaacaaactctccggcttgaactaagtgcctttctttcagatttcagaatggatgggcctcagccatggcaaatctgagagtcttccgggtgatggcacctgttttcatgaatgacacagcaatctccacttcagtaagggccgaccatactgttgattcctgccctgagacccttctcccctgcagtgccaaccagcccccagggaaccgtactgccttggcagcatgtcactcactctctcccagggctgcatcgcttcacacttccagtcctctgtatgtcttccaacaggctgtcgtctggcttcctgggccctgcatccagccctgaattagccacgtgtcatgctacactcaggcctagccataatttggaaggggacacccatgatgtgcagggctaatgttaaaaggtaaaattgtgtgcgggttcccaggcaatcataattagagtaattgtcctgtgttgcaggcacccctctttcctcctcccagctcctgcagacctttgaaggtactgacgagcctaaataaaccttcctgtagataacaggtggtggtggccaagagaactaaccaaaccaaattgtttaagtagctctcttgctggtgtggttttatggctagcctctgctgaactatagacaccccaacccacggggcagatgggcagtgggacccccagagcaggagtgtccttagcaaatgttgtacccaaccaggagaagggacgggccaagccctgtaaggtgcatcaacatttacacactcatcacaacacagggtaggagagggttaaacccaaatggcacggcccttccggctggtccctggttactaagctgtgcctggattacagcacctggggccatggggcccccacctgcggccagcatgattacatcgtccgtccctcgcagcctcccaaagaccacggtccaccttgactgcgtctgctgccaaggatgaggccttttgagcatcggaggtctcaggctcctggtgaagccccctgagcttcaggaaggtaagtgcagccatttgaacagccctcattggccgccttcccattgtggagaattagcatcaaaatatattctctagctgtagggaacgtctcagaattgttaaccttctccccaggtattagaggtaacacactgtgagattagatgtatattttcctcactcccatggcctccctaactcccgtcctggctgagcccattgtggtccacacaatcctgtcctctgtggctcctgagagtcttggaccctcaattctcagcttgcctgggcctctctgcctgctaagaagcttccaccagtggccagtgcccctctcccaccctgcagtttctggtatttggagggcaagtgcttgtgttgagtggaacaaatgttctggctataaccaagctgcaaagctgttggaaaagcacaggtcaggaatggacacggcttccagggaagccagggctcaggaagaggcagtcgccttcctttacttcctccagatcagtcttcgagctccggcacatggggagatcacgttggctgttaatggggtgtctcttttgtgtttcaggctctgtctcctgggtcaggctgggtccttggcaatctctgcctgtccccttcagtaggtttgaaaaaccaagcctggagggatgcggtggtgatagcagcataccaacactgtttttgttcttttcaacacctcattttgacgcaagagcttctcacacaccttggaaggggcttcagcccagatgaggggttttgacgccgcagctgggctctgaaatggcttagaattttaggttttcagctaaatggtgaccagggcggtcagtgtgtggccaaactcattggaggagaagccagtgagcatgcatctctggtctgtgcctctgtgggttggctgcagtagctctgctccatgcacctccttctggggccagttacatgggcttgatcttctgtgccagcaactcccaaatatgtttctaggccaggctgtgaccttcagactcactccacacctcccttgacttctacacttgactctcctgaaggctacaactcagtgcatctggtcttcccttgaagactcagtttccctgtttgtcatgtgagcaggttgattagatgagctactagttctgagagcaagtgtgggaccgtcactctagactttcttttggctttatgcccccttgggatttacctgatggttgagaacatgaacatttagccaatgacccatatggggagatggaaggagttcagagagacaggactgaacttggaggctctaagctcagaaagtaggatctgtggggccacactttgggcaatgtctggccctgcagttccaccgccctctgtcctgaaacaggcttcccggagccctgcgcaccccaaacaaaagctgcctctgactcctcagcctttgtgcacatctgcattattagcaatgctagttcacaaaggcccagaatcccagcatgttttacaggggggaacactgagtataagatgggtagtggttagagagtcaaggtcacccccaggccagctcacttctttgaaaaaacattttcagcacagctgaagaagggaggttctaaattgagaatctgatgaggtacccctggccccagtttaaaatagtcaatgatggtgaatccctctcaccttctgtttctcccaggtccacctcatccttcctgaccaagttcttcacctctcctgaggagccttccctccctaccccagggctcctcgaaactctgactctggagtaaggtttggtggacagagctctgttgcctcctcaggcccattgtgagttaggatctgttttcttgcctgccatcttcagctgccgaaagcacctgcctggggcctggaccctcgccaaatgtcaacatgatgttggggaccaacaggaacactggaagcacagtgttgaggagacttggctcttagcaacatgtttcttgcattctgctcattctgtttcacatttcacacttccccaggctgagcgagccatccatctagtgcaaacgcttctgagctggcgattgctctgaacagaccttcctcctcttttcattccagtctctctgatcacctggggagatacagaccttgcaccttctccctcacagcccaggagggcacttatttggacctgcactggggctctaagcgcttggaggaagccatgacttgtgtcttccattttgcaatagttctcattagcgcttggtgcctggagagaccaagctcctgccctctagtgtgctgaagacgagggcttccaggaacccaaatttgtgtatggggcagtataggctcagaaaacagctttgaacttcccctttataggggcatcctctcagagctgagagaccagaactgcagacagcctgggcatcccctggttcttaagctccccctataatttccccacactagcatcctcttttgcagaaggttctagcagatgttgtggttgcaacaattggcatcagtgacttggagctgaaacctggcttgtggggtgctagggg

SEQ ID NO: 10 - DNA, 5000 bp, Artificial Sequence
  Nucleic acid probe
acaatgggaaatgcaagttgctgggtccccagagctcagataatgagcatgagaactgtgggatgcattaagagggacctcctaaaaataagaacctgaattattctcccatgccaggaataaattgcactaaaattttcacacagtgccccctccactttcactgccttggctctcgggggagctgttttctcaggctcaggagctcttctgagaagcccctcctggcgagacgttgttgtgagcctgctaatgtcttccacttaagcagcagccaccgaggtgtgccgcttcctggtgggagaagggttctaggtagccttggtgtgatcttcaaagtgttaggtagagagtgctcagtttcccactctggtgaaagacaaggaggcacagaagggctcttgcggcaagttctctttggaagaaaagccaacctccagtttggtgaaagtgtcagcgttttggggtgcagagcatgtgaccctaatgtaagccagcacctggccaaaattcaaatccccactttcctgatgcagaacggactgccagagctggattttcaacacagggccgccaagactggggccagctcccgtcccagatgcagagaaatgtcactgctgctacacaaaagtgcggcattatcagccccttgcaaaggcaagtggattaaaatgccattaacacagcaaattaagctgaatgacacccagtcgcagatgcttttcttgaggctctgattataggttctgacctgcgcttggggcactttttaggggccatggcttccctgtggggatataggcaaggggttcgcttgctaaaatacccttctatttccctagcttggctccaactgtctggtctgtggccaccagggtctctggaggcctgacacccaacgggtccaactctgaagagttctgcattcctctgttgatttttcccctctccctccctcaggttccaaatccaattctagctcctggccaactggtagcaccgagaggctcatagttcagcatccaaagccgaggaaatcgctctccgcctgccctggtattgttttgcaagcctcgctggaaccaccttttgagctcgtgcaagctcagatgccagaatcccctggcacctctcatcggttcatccacctacaactgacaaatggagctcccttccccactgcacaacagggaaggatggctgcaaccctcttctgccctccggacatgtggctccaccccacccatctctctggatgcctgagctcctgagtctgggctctgacagccactttccactggaggcctggccctcttgctgcctggttaggaccctctttccaaggcaaacacagtccccaccctctgatctagatgactgaccatcagccaagggcactggggacctagaatgaaaattttaacaactcagaagctgtgcactgaaggcgcccctgctgcaggttctcagaagggtattccaactaaattgcagaaacatcttcctcgcttttgatgagctatcactgcgcagagcccgaatccccaagtggcaaggtgtttttccactcaatgggagaacagccatcttcggttggatgaagatgcccggcagaaaaaatagggttttacaggataagtcattacagtgctatccatccacagggcctacacacaccagctaactcttaagcttgggtaaaaccttcagcatcagagccaggcacaatgtggaggagacccaatccagggctctatgggatgtatacctttccctctccaccaaggctgtgaagtcaccgtcccctcatggagctgcatttctccggccttgggcctgagcctggtttcacagccttcttcagatacctgcggagacacttccaagacagcaccgtacgattttctgatacctgtgtgctgccaggcatgatgtttgaggcataaacccagacctttgccgaggacagggagactcggcttgagcaagacaggagtcttcttgaagggtctttctttgtctacttatcagaaatatgtagccttcctactaccagcgtttttgatacacatgtgatacaggcataataagatggctagctgggacagcccttccatcgatatctgtggaatgagaacttggtttgctctagagaaaggggcttcccaacatcgcctgaacatacacagcaagctctgagccagggactgaccacgggcttggaggtcatgttctatcttccctgttgcttcccctcctccttgtaccaacagggccggctaattaggcaaggcctgttgcactgggttctatcactcaaaagggcactctggatgggcctattcagaggccattgtctggccagcaaagctgagcatgtggctggagggcattagcagtgacaagtccaggtcaagctgagaattcccaggacatggctctgtctaagcattgggccttcccctttctggacccagtcatgagaggcctacgaagtaaggcctgggagcgtccagagctgggtctcggccatcccgcagttgtagtatcaggaggggaagataaattaagatgagtgcattgcacagttagacattttctgttaatgacaattttgccccatctgttccaacatgaaaatgaggctccttgcagcagacaagatagatctctaagagtttaagaaggagaaatcttttgaatcgtcatattacagtaagtcatttccgtaaggcctggtgagcttctgtctgctgaggggccaaaactcagtctgggttttcagctctggaagaaaattgcctctcacacaggctgacattaagttgagttactgttctgccgtagacttctccaggaagtcaaaaagacagttgagagaatcaggctgaaaatcctcatttagtgtgcaaatgcctcctctgcagctgtgcagagctgaggaaccaccatttcctccaatagcccagctgaggacgaagccgcggaggggctagtgccctgtctagatacatctagaaggatgtgcacataggcatacatgcagggtagatgccaggccaggagaacccaccagcagtcgactggaggatctccaagtaaatcacaggtaacccaaaggttatttgaagataaattccctaggagccaggatactttgaatttgcctgcttctgggcttggggcagaggactccattttatcaaaccttagcacaatgtcgtatctgccccagatttttttcaggaactgagacgttaccgcagcagtgtgccctggtgtccctcttgcccagggccccaggaagggaaagaaggaaggttccaaatgaaggacacacatttgacccgaaggcccagcaagaattgcatgttgcaatgtgcactgtgatctgcagcccctcgtgtgccttttccaaatggctgaaaagtacctcagttgtctcctggaattcgttttagcagcaattcatttctgaccacgcaggacttgggattgttaatagtgagtgtattacctcttagccatcgggccgtatttatgggctgtttcttcccttccaatcatggctgactcctgcaggaatatcatactttttttacttttcccttcagatgggaccctatatttggaggccccatggagagagaaccccaggaacccttgctcgaccttcccagatggccaggttggggacttttcaacctggagcgagcagtcacagtaacttaaatgtggaaatctgtcagagcgtccatggggctgtggcgcgattggttttggcctcctgccatttcctagctgggggtgttggaatcgcaagtcacaggtggctgtctcaagaacccataccctgatagggtagtggaccccatggggtacagctgccctggagtgctctgagcagcctccatacatcggttgtggagtgaggatccatcttcaccctttgatatgctctggccccaccttgagctcaatcagtgcgggtgacccttctgctccacattccttcttgagtgatggctcaggacctcagcatagctctctagactcccaacgcccaccacacaagggcgccagcattcagaaacacgatgtgggcttagaaatcacaaagtgataagtgcctgtgctgcgtgccagctttgtcatccacagtcccctaagaagctccccactgggtggaaggggacatcgcctgtcactgctggctcgtccggggttaggacataatcggaaaatgagaattccaagtcggatcaggaggagtggaaggaggaggctgtgactgtgaggcctcccaaggtcagcctcgcccctatggtgccaggcccattctttcctgctccccgtttccacagctctctaggtgtaacccagccctggctcggagcctccccggggtccctgcccctccacatctcaggagagctcagatgctttgccagccccgggagggtggcattctgcacccagtagtttccatggtgaaaatgtgggatcccagtgggctttggctcagggatttgtgaaagtgtggctcttgcaatgggcacgtgggataggagggtggtaatagaataatgatcacagtgttcctgcaagcatccttccagtgcctgccaggcattctcccagcaactctgccttgatccccaaatcccagtccattgctgatcggcctcagactctgtgcagcaatcagcctcatctctgacggatgtactttagcctggaagcctctatgcatcctgtttcacagacaagcatctaacctcaaaaaaggcagggattcccccataaataccgcagcgcctttggtttagagatggtttgaatggggagaacagctcgaatgcactgttttttcctccaagtaaggtactataaaactctgaactgcctggcgcctgccaagagatggtgactccaagctgtggatttggtctgcgcccctccgagtgggtttccagcatgcgtctgattctctcctt

SEQ ID NO: 11 - DNA, 5000 bp, Artificial Sequence
  Nucleic acid probe
aagcaaaggagatatttcagacagaatttgttaggacctaatgtggaaaagctgaagtaggacaagagcctctctaagatggcatatatatggcttacttaggagcctgagaggacctgattacaacacaccatgatgtgttcctgggtctgtttctgcattctgcccaacagcctcacagcagcagccatactttacatcagaggagtgtctttcccacattattcttctgagctgagagggaacatgcatccgccctcaggtagcctagtgggggttcttccacgctgtctatgtcacccttgtatataagtgggccattgtgtctgtagcttctaaggacccttctggttgctcctgttcttgaagttccattgccatacccatggctttagagagttaacaacttggacttgtacagattaaacctcagaataccacaatacagtccatgccttgcttcctaacaccacaagataggattcttgttagtgaaaaaagcagcataattttctgtatgtttaaacacactctagcctccaggtagcagcccatccattccgtattgatgtcccatctacagactgtgtgcagaccccagcaattacttgaacccctaacaactgaatggtaaaggctgccgtctctctaggtgtcaagcagttcaaaagtgaggcccaggcaggacatcccaggctgtaatttgactccatgtggtgtattgaccttgaatagagtaaggggtcaggccaagagaaaagaagagatctgataataatggctcagatttaggtagaagctgtgaaggatgacttgggagcagttcctaaaaccatcagagctctgcctatcacgcttttgggtagagacaatttagggaacggaccatccagggcaagttcccatcccattctcacatcctgaagaacatggctttgtggctcttaggagatgccaacatggcgtgagggcaccatcactagtgggagcagtgatgcttaatgtattgaagccactcttgcttcaaaaatcaatgacaaagggaatgaagaagagggggcaagtttgcaaacatacttaggaaggcaaataatcgaaagctgctctcacaaaaagacgaaggaggaatggagctctcactcccttcacgcttgccacccgaaggacctccctttccttcctcccttctcacctgtgtcacccaacccagtctgcagccctccttagggctgggttgcagctgcaaacagtgtttttgtctacagtgccctctctgaaaatagttgtctttgttgtgacttaacggcccccccctcaaatctaatttcactttcctctccttgtgtctttctttgggctgaatgactacactgtatcattgctgaaatgactgtctttaatacctttgtcatttcctttctgtctgcaaaaattctcctgttcttagtgtccagtgtgacttctggctctccttaaactattttgtctccattagtttagatgctgacctttgctgttggtcaaggtcctccagggcgtactgcattctcagggctttgtctctcttgagtcaatgaggcccctaaaacgtgggtttaagaggtcagttattgccatcccacaaagcacaaactatacaatctattggcaacactgcccatccagtgtggccctgactttggttacttacatttctgtaccacaggacagaagctgaagccaggagttcatggccaaagcatccatgagctctctggctatggctggcatatcgctgaacaccaatttttgctctacttgtgagggcttacaccagacagctgaaggaggcatgcagaaggcggcgatgagctcaggcaccaaaagaaaggatttctagaaaggacaatatttgataaggtgttgggaaacagaagggttaggaaaatgttaggctgtgacttgctccttgagtgaacgtggttctcattaactcacaggaatcagaaagtaaagaacaccacatgttcagtgggactggctcttttagagcccaaagaaggtggaataggcatttgagcaacagacaggttttgaacaataactattcccagtagttgactatgggggaaatgttacagagtgggactctgaggagcacaagcccagagccagagaggccatttggggggctattgcaatggagataagagttgtaaggatgggatacaatgaaatggcaagaatgcaagagagttcaatgagaaagcatccaaccaccagctgtactcagtctagaaggaagaaagtgaatgaggctcgtgggggagcagagggagtaccatggccaagagtcagagtgtgcacgtcatgttcaaaagcttccagagctgcactctggcctcagcacggagaacaaggctctcagttgagctcaccatttctttgctttcccaggcaccatactcttgtcttcccggatgcctctctcccaggccttccctcctagcttagcaaacttactaaaatatagagggtgctaacataccttcctgccagaaaccagaggaatcatgctgccagaatgtaggctgtcagccccatagttcacaaatgtgagatttgtagcaggactcgctgatgcaaaacgagcacaagagtcaaggaatcaaccctttcagtggggtagtaatgaggctgcttgaggtgttggtgcctcactgaggttaattgaagacccagcaacaataatgagctgagagaagccagtgatgtagcaaatggtgcagggcatttttgatgttttaccacatggatggtattaccacaaagcagatcaagctggcaaatgtaccaacaggcccctcggtccttctgcgcagtgtcctctcacagctgctcccacaaacacacttccccagtttgctccacaccccagttctgtttccaacttatcgccctcaccttatttcatcttctcccttagctgagctaattatgagcttgatcaaggacagatgttaatatagagagatttatgcctcgggaaactccttctttcagatgtgtgtaattgggatagcaggtacatggtgttacatttggtgtgatgtgtacttggtgtactgatggactcagcctcacagttctttgctcactcaggccctgcttgttaagaacattgttgcctgctgtcagttggccaggtcattaccaatactaactcacgctacagaaagcaaaaccctaatggggctcttcagatctgtcccataataagctccaactgaccctggagagttccaggccttaatatgcctaccctcaaagctgtgcaccccaggcttccacagagaccttacattctgttgccatcacgcatctttatggagccttttgatttgggcataaaagttctttatcaaatgaatccagttccctctgtagtactcctaaaacctggaagcctcatttctggattctgtttgaacaacaatgcctctgcacataacctcaatgtcagacactagaggtctcaataatgtggatggccagtgtccaccactgggctttgtgaccagcaggaaatctaagagttcaaggaagaacaagacaagcaagagaaaacaagtgggaggtcatagataacaggtgccattgggtattagaagggaaatgtggaatggtcaatgacttgagacaattaagaagacaaccaccaataaaccaaatcttatgagaaacagatcaaagcaaaaggcagaagaagtgggtaaccccaagtctagtatgggaaggagacagtgtagaagtgctgtgggagctgagtgtattttcaggaacagccatttgttatatcctccaggttcaagccagctctctctgtgacaccagggatgtttcttccaataactccacaggcaaaccactgactctgggtaagaagaaaactccatagctccaacctcctacctggacaggaaaacattctattttattctagagaaatagccactcaggtcgtgcatgtttttctctgtcgtccctgttcaatgcgtgacttcactttgctcctccatgccaatttacccttctctctcctatgtttaatgtcacaactatgtcaactgtagcatggaaagaagaacttctcaacctcctcttcaatgttttaaggctggcatcttttgttgagagagtagaaaaggtagctataggaatgtaaagatgaggtacgtttccagggtgaagtcaatatttcaccacccttacccaacagcaggaataactctgccagacttcatacagttgtgtatctaaattttgattagtatcagtaattattaccactgaaggctaggcaaacacccctcttgagagaaaaaggttgattgaaagcaaggtagacgcatttctagaaacaggttttatgctcctagccatccaatgaggaggcccaaccaccaagactaagaccacagcagctgcaggcattattcttcaaaaagctattcatctcatgaaactccagatacaaagaactaggcgaacccaggtgtaggcaacagagcagagcctggactcggctgcccttggttaggctgggacaaagaacacatttagaagagaaagaactgacgacgccccttgagtgccttctcctgcaggcccttgactgttgaggatacccagttgtttcagcttcagcagacaccctccttaatagaagaccattggagacctctgataagcagtgaaagaaaccagggttgtggttcagtgataagcaaatgtggagagtggcaccgtgaaaatatgattgctagacccatcacaacaaagcatggttgtagctgcctagtaaggggaagtaaaggaagagccaaccaggtgaggagaagacctctgtgagtgtgagtcaccaaatgcaaacctgtagtgaactcatcttaattggaaagtgggagaggaaagaggactcagagatgaagacaaataattgatgtcagagtcagagagag

SEQ ID NO: 12 - DNA, 5000 bp, Artificial Sequence
  Nucleic acid probe
agtgcctgtcatggtgctgtccaccgtacagatccccaggacacatcctcatatttgaactgaactgccttattccacacatgacggtttccattgcctgtcttaactatgtcctcccaatctctctccccaagcgcacttcagagccatcactaactgactcatggaaaaaggaggagaaacagaacatggagaaaacagacagactggcaaggcctgaatccttgctcctttggctttctgggatctccctagaatttaacatgggatggactagagtcttgaaataaagctggggtcatgctatttccaaacacaggtggcattttagcaaagcttcttcatctggtcccatgatacaacaaaaccaacgaaaggcagctaacccctttaaaaatagtgttgccatagagtgagccaactgggaaaatgataggaatggcagccaatgtctacatcaaagagatagtgcctcaaacttgtgcaagaaaactacttgagagctcagatgcatgaaaatgtctctcccgtgctagcatggtcctgggagagggaactcgtgcacttctcatgggctgatcatgccttgtcaagctctcttcacagtgctatctggccatctgagaagagggatttccatcatgcagcgactaccagaagctcatagccctttgaggcaccttctgtctttgagcaatctggcacttaaaatacaaataacaggagaccaaagtcaaagatgtgatccctgaggtttgggcatttaacctggaaaagaaaagagtttgacttcagaagcagaatcataaaacactatggaaaattccaggagcctccattcctgaccagtctggaaaaccaggttcctgccccggccccttgccttcaccatttgacaatgaatagttgccttcctgttggcaaagaaatcataagggaaacaacatgcgaagaccctgctgtttctcccacccatgactgtttttcttttgattcgtttctatcatgtctgtggtgtgttttcctgctactcctagaaggttgcttgcaaatgagtagtaaatgaagcagcagttgatcacagtacccccaagagcagcagcatgaggaattggatgtgcatggaagatgagcgactagagatgaataatggacatagggaagcagaaaggaagacacaggaagtccaaactactaaaacagtctggctgtggcatgagtgccagagagcagggccctacaaatgcatagcattctgcaatctcccctgtgctctaggtgagctgcccccctgccttgatatacccataggctacgttggcaaggctgtgtcttaacagggttgctggcacagatggcccaaaagatggaaatttaagtaaccctgttccagcagggggtgcatgcgtttcagccatgagagtgacagaggccagaacacgtggctagactctcgccaagtccatcagaccgttgttaagagggctggaagagccaaacagcaagtggggccacaggcctcagagctgatgtccacgtgattcctttattctctttccaccactgatctcaaattacagttcctgtgcagatatttaatatcagaatagctcatccgttcacctattaatcacaggaggaccactgaatgtccagattgctgcaggccacctcctacgacacagaagatccttctgtgaggtgaaagttccaagacgctgcatcttcaggtcactctggggtcaaagatagtttcccatgaggcttagtggccaattgcagttcagaaataaataaaaagccacagaaaatgagtttaatctgtgaatgtgatttatggctgtcggggcagcaaacttcatcatgcagccatagcacacaataaaatatgttgcaatactgtatccatgtgcatcctcattcctacatacaggatctattccccacaccctagtagacttggaagccccatgttcaatgtgttaagattccctgctcagggggagactggtcttgggcagaaacagagactaaagagcagaggtttgctgagcagacaggcaaataataaggaaggcaaggaattgtttcatagtttatatactccccctcggaaaccccctttctcctgcatcattcaggccactacaacagcaggtggatcttcatgatgttttttgttagtctctgatggagaagacatgcatgggatggcttattggtgaatttgagctgtttcaggccgattttcttataatcccacatgtgctcaccagctgacccgtatcttgatatcatctcctagtaagtccttcacgtgtgtggccctaagcgttctgtctattaaaggcagctggatttgaaaccccgtaggacccaacttgcaaagataaggaaactaggaaagaaaatgaagcgagcactggtcaaaacaaatgtcattctactcagcagcaaagcatgtaaggaaggcattaagcctcatcttccaacatcatagaatgggaaggcctgggaaatgaaagaacagaagggagaggcaagtaaaacactgaaacacgaagcccacctttctccataaggctctgccttcaggagccaacacaggctttggagaacgcaaatgtgtgccgaacctgttttgggctaaccatgcctgtggtatttggaaagttacatgaactctaaatcagagatgttttccccaaagctgagaacatcactgtccagagttaacacgcaatgaagacgtgagcaatgaatgaatggctgtttcctttctcttaagtatcatttttcacaatctttaaaacaccactctgctgttttctttattcctcacacttccattcctgttgtctcagacttggagtattatgatggattccccacccacacccaaggaggactaatttggtcataaagctggctcagagggcaatagacaatggccaggatacccaaagggaaattatgataggagatctgattttggtgagaaggtgactctgaaagtctgactcaacacagaggttacaggtgatcagttaattggccagtcttggacattagcgtggggcagggagataagggtgaactgaggtcttgccaaagttactactacactgcgaaatataatgtactagcagcaacagagcctacagatctgcttcatacacttacagattctaagagggcatctcctccaaggacaccagagaactgcagccacgggcctggctttgactgtgaactggcaggaaggtctacagatagcctttctgatttcctcagactctatcaaaaccacctcaacccagaaaggagcttctgtgagaatcacaaggcttggaaattcaggttggagggtgggatttgcgggtgagggatgaagtggaacgatgtgttccctttcaattgtcaacagctaaaacatagagcaaccatcacacccggtgtaacttccatccattatcagtgggctgagaccaaaactgtttagtcatgctacgtatacaggcttatatccaaattttccctctccacaatggcacatgagcatttcctatcactctcacatggtcagcagtggagtcctgatgttaggtgagatgactgctccagcagctccagcgagacagggaagcccagaaacggaaggccatggtgctattggcattttgatgctacatgactttgagcataaagccaaatcctgaggccatttagtttagggaacaagagtcctctgaagctgactgcgtcatagtgcaatcgattttaataatgagtagttcagactaaagaaggctggctgaccacatttgaggcagcccgggagaggaataattatattctagtgtcagcacatcctctttccacaatggccatacacatagagaaagtaacatcccagtgggagaaaaacaatttgagcagaaattaggtcacccacaaagcatagccttgaccaaatcaaaaggaaatctatctcttaacctccatcatcatgtgacctgacagagatggaatctcccacatctggggacagcatcccccaacatcagttcacaggaagcctgtcccgcaaagcatacagttgtgtttgtgtgtccacactcccaaaggagtaatggcagcacatccaggagacaatcagaaggtacatcatccattagtttaagttccactgagaaatcatattccagggctcagactggagagaaagtagaatgagaggtggctctcaaaggtttgcaaagtcttctcaggctgagcaatgaagtgtgccttcttcctgcataaaaatgttggtgtaacaaatggctatttaaagtcctccttcgggagcagatgcctctttgccctacaggctccttgaaagtaggcacctgtctgagtaggtgctgaatttggctcattcccactgggacaaggattcaagggcagcctttcagagcacatggctttctctttatgtcgcataggttacaatgcagagtgacccataaggaagtggaagccatggggcttacagactcaccataccaggccaagaattggcgtggcttgcagagctcaaatcagatccacatccatttaattttcatagagaaactgccacagaggatgccacatactctgcctacttgatttctatttggagggcagcactttgtaatgaaaatcatgttgggcctctgaaatccgccttctccccacagaacaggactgctacctcccctactctgggtactgcatgcctatccgtgaggctacatcacatcttggaaagacagatgagtagagcgcccagctcagctgctgggccctgacagtagctgcttaattcaggttcaccctctccccttctcctctttcactcttgactcatttacccagaatactgttttcaactttaccctgaactcccagatcccacaaaacatggaaacagtgcttggtaccagccaggaatgtcagtctcatctgccaggata

SEQ ID NO: 13 - DNA, 5000 bp, Artificial Sequence
  Nucleic acid probe
ggcacagcagtgaagaatttgcccttctctttgtaagcacaaaatagggctcagaaaagattcagggtttttccagcccatttcccccatcattgtatctctctttaaggtctgtggttctccaagattccatcctgcacctcatttcccagttgtcatcctacaattgtcatccttccctgacgttcatcatctagggagctacttcccatggtcagtttatctgcccaggtcctgaaagccttgaaggttaggactcctttttgtctcagtcactttgtcccactgtctgcccaaaccagcatacttttttccaatcccaaagcatctcttctctgtagccaaactgacagggacaacagtcatgatataatcttacacagaaaacaggatgctggcaccttagctgctatgcacataaagaatatgcacgcagaactaggcctcaggagaaaagtcgtgctggatcttaattcatggtatgtggttaaacatgttttgctaatagccccagatgtggcctagtccacacaatccaaacacgcgggtctgccctggacccagctcggttcaggaaatcacagaattaaatggcgatttgaagcttttagtttttgcacaactctgtcatccctacagtgtgcactcacctaaaggggcactggctctgaaacgactctcacagcaggtgcggctccatggcagcatgcggaaattccaaataaaaatcagtgagaaaggcttcctcccaccagaaggtcttgcataggagaccaagcagggaagggagccgcagacacaggatatgcagaaactcaccctgaggcctttgctaaatcctcagacacaaagaaagctaaatgagagacaaagtttggcgggaggatcagagaagcccggtccaatgccctgccatctcttcctcattcccctatttccaccctcaccaatggtctcaccttctctttataagatggttgtttccaaaatggcagctgcaccttcccaggcatccccgcatggagagccctgcttcaccttgatgcaccatttgaatgacaccctttaggaagccttcctggattctttcaaacccctcctatcctgaaggcaagaagagccacagaagggtttgatggaggtaagggatgtgcctggctttacattttggatcatttggaagggaatgagaatgccatagaactggctattgttctgcatatcacatgcttccccattatttctctttccatgtccacactgaaatttggtcgcagcgagtgggcaggtaaagggacccaaacaaaggaaaaccagaaaacacaatgcacctatgtctactgccaaagacctgggataatctagtagagcctcaatcaccccttttcctcccatgatatagaaaagggaatgttttgcagacatcctcatctttgcaaatgccctctctagtcatcaagtcccctacaacattaggatctccagaaaaggaagataaacaacacccaggaacagttctgggaactcctggtacaaccgtatgcaaatcatttcacaggcgtgctggcaggctgagttcccagagtaaatgattttcattttgataaattgcagccatttcctttttctgatttggatcaaaccgtcttgctcactgatctcctctggctttctcatgggcagatctactctcatttttcttcctcttgttcatgcttatttctacctgtagctgcagttccccaagtcgaagtcaatccattcactgtggtcccttgctcaaagcaataggctcaggagcagtagctcgtttaggcaacacttcctgaggcttgtggtgggccacactctatactaggtactgtccccagttgagtgctgaattggagaaacaagctttagatcaacaaaatacgattccgtacctaatgagcaccactcaaaatacagttgcaaatctgggcacaatgtctgaattattcaccatgagccatgtaaatgcttcagctctctaggaggctgccacgtcctctctctgtctttggggacagccctggagcctgacttgtctcccaaatgaccaatcaaggcatttgtagtgaaagtgcccagaggacctgaacgcatgggcttgtgaatgttctcccatggacgtcagctgccatcaggtcttcagctgaaataggctggagggaaattccaactcctggctcctttgcttaactcttccatcctctccaatatctgggcagacggccaagatctttgccattttagtggctcaaggagtctaccagatactcttaagccctgatcataccccaggctgattttataggagagctggataagacagcaaggactatgatcccaaggtaggaatacatgtgaaaaacccagggtggcccagacaagtgataaagttccaaatctaccttctctgcccttcctcgtcctgaggggtccagaggtcagaaatctgtagctgcattactaataagtttcacttgtctgactccacttcccttcagcaacccctccagggtttgtttgtttctgatctggttcattactcttaagtcttgccacagccacattcccaaatgctcaggcctcatgtgtgctctctggcagcgagctgtgcttttgtggaaaattctccccttctcgcacgtcctttctttgacataccaaatgttgttttcaaatctaggatttatcaaatgtgacagcctgacaatcccagagacaagaattccacaggcacgtgcggcacaggacaggaatccctgccactgtgacctcagttgccaccttcatcacagttgttatcaattcccaactgactgacctgttctcaccatgactatctggaggcactctttggatgctctaattgccacaaatccaatccatatgttcccaaacaatgcaatgagatccacgggcccagaaagctcgtaccttcattcccaaagaaatccttggtgtaaaatccatggtccgcataagcagtgtttgagtttgcttctcaggacatctgtaagttggctgttgtgactggttatggcaaaccgaactacctctttgtgtaaagtgtcaaggttctctggtcatctctattactatgatcacagtgcttcattgtggggtggggaagggggtgaatgcagtaggggaggttgagttagtttttgtttgaagacagcattcattcctcaaacacctactctgttccttagtttagatcctggctctgggcttgacttacctttgtgaaattacttcgtaaatattttgtgtgttcactggatcacacagctgagcaactgcacaatcccctctgcgcagcagcccaggttcctgcagctgtgcacattcctgagtggagtcgccactaaagattattccctaagctgggactgcaagtgttttgtctaggacagtttcgaatgactcagatgagaagggctccacgtcattctgtggaaaatgaggctgactttacctgtgccctgatccccataagggcctgcaaccaccaggagaggcacctacatgatgtctgggtatagagtggaaggatctgcatgcaaggccgcaaaaagtgttactatgagtattttgatgttaaaggggggctttttgtgcacagagaccataaaccgaagtcaccagatgtttttaactaattcaaattccgctgaggcaaactcctcctccaacctcatagacacaggaggtattgaccagccctggtggtgaaactagattttagcaactggaatttatcttacacttctacatcattagcgccacagagtttgcagtacacagcaagtgccctggataaatgatgtgaggctgatggaggaacaatgggaaactagtgtaatggaacatcccctgtgagcatgtatttcctgagggccttctgtgcctctgaaggtcacgtgtacctagaggaaggcgatctgggctgctgtttgtgcacttctgactaacctatcttgcatccatgggccctgtccttcttctgctttgggctgctttttatcccccatacaccagggctcagttgtcaagttcccaacattaccccagcatggactgatacgatcactggagccttggaggtgagtttgaaagatgtggatgagatttgttggggagaaggagtggtatgcaaggaaaatttaccaaagcaaacatagtgagggcccctgcatttgaaggctcacccactgcaaatccaaatttccccagtgcaccagttcattaaagcctatttcctctggctctctgaactcattccccctacagttctctgaggcttttctcctcccaaaatgccagagcatgagcttatgcgtctctacttattattttgtagctcagaatgtggccatcaattcaaatcagccatacaagacagcttgagaggccccagggaaaccagaggacagtggatgagaatagctggaagtcaaaagaaggtaggtccaaaatcgacaaggttatcatgagagacaggagtagccagtggccacagtccacacaacagagagaagatatcctctggaggatcaaagagaaacaggcccggctctaaccaggaaggcagcctcaggcacgtaggtgccttcacgcatcccaggcctgaactgtttcatctgtaaaatagagttgatagcagactccaatcctggtccttgagtctaaattcctgtccatctctcaccagtgcagccttccaccaaaagtgtcctgctcttttctaactccttgctattgatcatcggtgtgaacactgctgctgccagatgctcagcaccggaaggaaagccaagtagagctcgtctggcacatctgctacagacacagaaaattaatccattgtatagaaatgatatcaccacatcaggacttctgctccaggggcaccagggtatgggcaaagtaagagctgtgtgtgtttccatcttcctacagacctacta

SEQ ID NO: 14 - DNA, 5000 bp, Artificial Sequence
  Nucleic acid probe
atgcactcacagcctttaaacattctccaaatgtctgtcacactagctatgcctgcctcatgcgcaccaagagtaaatattatcctcgcgttaaaataaattaagctccctttcatgacaaacacattttgtcgggagaggagactggaacacagagaggtccacttccattgtttaatcattgtccttcaggacttgggattaaaaatactgacaaattattcagaggaaggaagatggtgaagttgggggagtgcttaccagaattagagtaataaggggaagttgagagagagacagagctaagtgacagtgatttttgtacccacacataaacaacaaccactacttgaatcatcagctccttggggaaaatctcattgttacaaatgtctttcaaaaacagcacagagagtctccagagcacagagaattgtaaactgctgggattcagcttgcaggccaatagtcacgcatgcccccttcactggtttacccagaaagataccatcgaaaccactgagcttcagatacagtacttaggcagtctgcagccttgtgggcacattgaagcaaggctgatactcccacaggtgtcccagcaccaaagtgaagagccagccccttcatcagagagaatcttccctttgggctgttcaggtcacttacacagactcaagtaaatttagaacctatttatcgggagctcacaggggaccttaagtaatacagctaggcagagggctgtgcccaggccatgtttcccagctgatcactcagagcttcccaagttcaaggccagataagggtgtgctctccttggctctcctctactaacacatccttttaccactgaagatcctgcctccccattcctccgccatctgcaaagggatgtggaatgctaacttctggggtcaatgctcagtgacccgcagggcttctgtgggacttggctagaaatggacaagagagttgtgtgatatcctaaaggtaaagtacagataacagatactactgatacaaagatcaccagggcaagccaccaaaaagaaaatgtcagacacaaaaaatgataagggtgttgcaaggctgtcatcacctcaactatgttaaaaaatcacagggctctgagtttacacatcaaaagcagaggggctccgtatctcccttttgttccttgataaaaatagccaaatgtgtttctttttctgtaacaagtctgactttgttaaacgtggctcattcagtcctttctcaaacttaaagtgtcatgtaacttctgtgacttctactcttgtggacatgcacttggaggttagatcttagtgcttgctccttcgctgcttgtgcaggggtgacctacgtccaatgttaggaaggtgccaaagaccaggggtccttgtggccacacatacatccttatccatgcggggtccttgtcagcaattcctggctgatccactacagagagatgctaaggtgcatgcacggcgattgggagaatcactgctttttatgagtagaggatggctttcaagaacaatgctctcctgggtgagataacacattgacctgacatcaagtgagggggcagggaaaatgtggattaggtaagaaagacacccagagaaaggggacccttggtagtccctaaggctagcccaggcgtgaaacactgtgtatccacagccaggcaggcctcccaggaagggtcttggatgctccactacactccgtcatccaagacaacactgaaaagactcccagatctttctgtgagtgagaaaagagaccagcgctgagcagaatggagcctactttcctaaacagacgtcaaactaatgtcctggcctcctgcagttacaaaagctcccttacagcacaactaactcagcccgccaaatactgggggcttaaatctgcaccccatttacaaatgcaatgaaaggcagtagggaaaacacaacagtgcaaaaacacaattttccatgcaatttatgacagcactgaagcagcttaccccttggcctggattcttgccttcttcgtctaaagggaacaggctgttgctttgcacagggtgggcactccacgcatcgctgcctcctttggggctccatccactggctgacccactcatcatctcttgccagatgcattccaacctgtgttctccttcttaaattgcctttccccaagccgcctatgccctgagcagcctctatgctcttcagatgaccatggctcacaggcgtctttccctccaccagtatcttgcacttaatagacattaagtgtctctgcctcccttgtacaacaactacgctcaatgacagaagcaccttgatattagcactaccatggattcactctaccaaatcactgttaaacacaatcgccggagagccctgttggtgctgtagctgcaggaaggctaacgaggaaaagttagcagagctagcagggctattgtcacccatggttgagcctgcaccagggcttttccttcctgtcacagtgcacaactgggaacccacagagaagtagtcaaaacagccggtgtgcatgcatctctgccggtcacggccccccagaggctccccttttcctgtccgaagcactctccaccaagcaatacactccacagacttgcccaggagtgggctctgagtgctctgtgtggatggctcatactgcgctagaaccttgctcttgaaagaagccccaacattggacagtcctgggcagatctgcacttacacatctgcaaagacagaaatccacagactggaggggcaccccactgtctggcctggatcctggactcccaggaatgggcaagggctcaagtcaaaaaacatgtatggggcacttcttacaaggactatattgaggaagctttcgggatgtgtggcaccttttctcttaagtgccaaggggctccttgcatgtcttaaatgactctatccacctgcccaccttccctccagcaccatcagcctagatctcaagttaatacccagccataatcatttttctgacatgccttcataacatggggcctctctcgcacactcaaacaccaacttttgaattggcatgaagaaaaaggagacaaaaaaatctcaaaccgtgtccagttgggaaagagccatccagccttagagtcatgctatcgaatattctggaaataaattttttttatgaagccacctgttcttctcctgctcacttggtgtaattcttcacttctgtttctactccacctagtacctgttgccacaactcaatatgtatcatattgctctcatcttgaatcatttgttttacatgggggaaacaggatgagttggcaaaaggaaaaagactgaaaaaatgtctgaggctaacatgccctcctgtaaacatagggctgttcctccgtgacagcgaaggccagtcatctcctgggaagagaagaagctgaaagcagaagctgactcttccagggtggttttgccctggttgtcaggaaacagtaggaagagtctaggtccttaatatcatcactcttgcctgagtccaagtctttacccaccatctgtcaatggattattgcaccacatagaaactgatgcataaaatatcttccattctcctttccagggcctctgtgctgtctactgtggaaccaggtatacaatgggtgtcccattcacatccccgcagttgttccattgaagatattccacttgggtcatgacctccctctccgaatccacttggacaatccagacagcattccagattcttgcactgtaaatgaataaaactagtaaaggtttcactaaacacagctaaggacaccgccaatctataacaggtgaaacgcactcgcacacacacccccagaagtctggcaaggatgagaagatgcttagtgcagagccaacagacccaagcggaactgtggctcctaccatcaactgcagtcaccaacatgaaatggcaagcaatgcacatttctcctatagttgttttccaacctcctcccaggacaatcttcctccaaataatccaggcccttcaaagtgtcctggaacccttgttacacaatcagatatgagaacgataaaatgaacccaaaagtcagaggcacccaatagccaggtggaaagctcctctggtcctttgaagaaggtggtaggggaagaacaggcaacccgacaagcagagagcggcctaacaccactgcagtcaatcagggcttccttggcaggatttttctgaagtgctggtgcatatcacataacatctcttcaaaaaatgcaaaatgacacacacaagactggaatatcgagtacggttgtgcttggcatctgcagggcactggtttcaggagctctgaggccaagcttatgttgtggtctgttggtttgtttgcttaggtagcgaatgcagcagctgactgcaatgctcatttgcaatctgcagacgcaggtccctccaggagggaacgaaggatagaaagtttccacactcacctctactagaggacatgccagccccccatgggaggggcaagaccagtctttggactttgctgtaaaattaaggtaactatgggaaaacccactggaaaccttcatgctgagagtgagataagcagaatgcagacctgtcctctctagccactgtccctggggctgcccttgctgcagcccttgaatcccttcttaactaaaagacatgtgatccctgcctgtacccttccccaatgttttcttggagctctatgccacttatttaactctacgtcacaggacgccagggccctccctgtccaaaggccccactgcagtggagatttgtggtggcacatgctgtttgaagtatgagagcccccaagtggggctcgatgccaggaagccaggtcttttaaagcactgtcacctcagatgagccgctcaggagcgctcacacctcaggacccagccctattcttcacaagtcttcaa

SEQ ID NO: 15 - DNA, 5000 bp, Artificial Sequence
  Nucleic acid probe
cgcggtcagcatgtgagcctccctgcacaggcagattctgtgacataggaacaaataagtggctggggagtgagtcataaataaccgatgtcgaggtacaagggcgtttcaccgaggcccaacttcccatcctcgtgcctgtcgatggctcaaatggtaagtgggcaaaccctgagtgccacgtgccctcatctgggagcgcctctgccaagtccaggccagtccacatttgcccggaaaataggccaggttgcaaatgtgagtcatgatagcaaagctgatagccacagatgacagaagttcaactcttcaccagactcgcagtaattatagtcaacaggaaagtcgaactcagccaaagaggcaaggcaacaggaaattgtggggaaattttaaaaacgttcagcttctcaatctcactctggaattttataaatgcccctggggggttggcagaaggaaaagggcttctgtgaaatggtgaggcatttcccagagcccaccttaaactcaatgggaagcccaatggccttgatcatggtagagaataccaccagaggtggcaaggctgctgcagggagaagacgctgccagggatgtatagaaaataatagatgtccgttgtggctactactacaacacggttggcctttcatctcccctttgtctccccaaagacacatacttctgccatccaacaatccactgattcctctgtctcccttccaagcacagtgcagcattcctgacattcttcctggttctcctctttcataatgcttctattccccgtttgacattagtgaaaatgtccctttggtttttcctcagcctgtccaagctcccatgattcactgaggaaacggccttattaagggataataggttatgtggtcccctcaacacagagagcccctggaaattacagtgcattcaggatttcccaatttaccacaggccttcagagccacatttggcaacagcctatttccaatgtattattctttccttcaaggcatctactgaatgttaatgtgcacgtttgaaagctatctgtggtaggctgtacactatttcctcacaattttcccttctctctcagccaatgcagagtacatcttcctcaagcagcccatcaaatgaccgcaggctctgaggacacccctgacaaagcctccaagccagaagaggtaaccagccccagcaggagagccacagttcagagggcaaccacttcagacacatgccctcgctgtaagtaggcctcggtctatatctcagtggctgatggtgatatcaagaaagctaagttgtctgctggcaaactttcaagctccatttcttcatgcttgatagaccacaaaccagctccatgagccggagagaagctgcaattaatacagagaaaaatcgcaaaaccacagagaagtgcagcactaaagacaaacagacaacgtgggactccactctctggatatgagaagtttacagtgtaggagagaagtttggtgagctagattttaagtggcccccaacccagggaaacatacaccatgtgctccaggcttggtggggatggtgcgtggatgagagcagacagactgatgtcccctcgttcattctaaatcaccccacacatttatctccctatcaagcttcccagaatcaggatctcttacctatgtcatgtaaacctccaaaacacaatttcattatgggtttaggggccagaagaaaaattagaagggcacaacagaggtggaatctaatccaagaccacctatataaaaacagaactctagtcttccaagtatctgggagatgcacaatggcccatcagttggctccagttgtcaccactggcacattcactcacctgtatcacactccccactgtgatgtggcatttttcttggcattgagactggttattccaaacacactcttatcagccttgcaactgagccaagccgtttatacatgtcctcaaccattattcgtgccctttattcacagttcctcacacctgctgcagcaagctagcctgtgggaatccaaatcccaccaatcagctgaatgcacctgtgttgtcttattcttgacccacacagtgggccacatttccatcccagccctgacagggagcatctccctctcccttctctacttgttccagtagagcctgttctttaaaaccaggacacagctaaaacccatcttaagaagtcatttaatcatgccctggcgcaaaaaagagaagctgctataatatttggtcagaccaactctgaataagctgctctgaccaatctgctcccatacccaagctttggcttcagcctgatccaaacctccaagtaccacccgcttccctgacagtgttccctcctcacattgacaagccctactcagggttcacggtcattgctccatccaggactggtctgccgtcacttaagaggcgactaataaggatttggggggcaacagagctgccacctccctctttgaaatagtgctcctgaaacggtggggccacctcaggattttcgtcacgtcagtgtcaaccacatcattccccgtcttgattgactttcttaataacttaaaatattaggcagctgagacaacattagacaaccccagactgccattagtggctccgctgatcccaggaagcatatgtaacagttcttctttttggaattacagatccatgcccaaaattagggcaatggactaagaagaaagatacctgggaaactgtagcacgctcataagagttattctggagatctggctcagtataacaagcatcacaggtgagagaaactgccctgggaagccccacaggagaaatgggtttgcaaggggaaatgacactgggtgactatacttgaaggacctctcttgctcacaggaataatgcagaactcagcggcccaaaatggaaagagagtagagcagagaaaagtcctaaccaaaccacagagcatggaggtgagcacaaaagctcagacttggggatcattaaaaaatagatagtggtcaaaagtgcaatagcagcccccaccacgaaagacaggtgtggagagagagcaaaacaaaaatttggccttaggaacttgcgagtcatctctgtgatccttccagctgtgctttgcactgggggtaataaatcacgcgtaaataataatcacagtgtagagtcccacatatctttgcacagctgctcacatcagctacgtttttcaaccaaatacggcaagtccactcaaagcagagccgacgtgaatcatcaccatggtcctccctagtgatgggggacaaagtggacacaaccataaattgtgtgggctaaaggcaggaagtaaggcagatcaatagcagaggtacagccagccagaatgagaccaccgctcgctttctcattcacaaaatccatttcctgtctcaggtccagaacttccagcgtttggttctgttcatttccccctggatactaggagagggggtgcaaaaagaagcacaagtcagaataaccctggagaaggtcctatccatttcagagaagacatgtgagaccacagtgagcattcaagagcccaattcacatcaaacaggagaacactgcaacccaacggtgtattttcccttgcgcactttttcctttcaaaagcattcatttcaggaatgatgcggttttactatagtgcatgctcgttacagaaaacatggaggacactgagaagaggggccgactgcccacctccctatcaaccagtgctgttagtgtgagttgccatggaaacaatgggccatgccttctctacagagctgaggattaagaggcctgaggcctcgacactatggctcactgtattcaggctacggggagaagatttatccaaggagaaacggaaacccgttgctaccagatttcctcccagtcaggcctgcaccctccctcttttgggtcccatggagctctctggtccacatcagataccgaagtatcccaggcgctcaaaagctgtctctcacatcagggaggtgttcaagttaggcacaaaatgttcagcctgcctccctttgcggagaccgtgactacccagaaccagaattagaaatcaaagtgaggtccaggacgtactctggaatgcagactcctccctccctgctccccaggctcagtatagagattgttcattgctaccgtaagtagcacaaaccttagtaaatcctaggcctccctgagcaatgtacacacactttctcattaaaatttcactcccacgccaagggctgggggtcatgaggcttggagacctcgctcactcaagttgtgctattccaagctgctccctggaaagccagccaggggttccagcaatgcccacggcacagaagctaaaatccacatagacaaaggccgagagatgatagggccaggaggtatcaagaggaagccatcgggtagcagagggcaagactggaggacctggagacatttgtaatagatgagaaaggagcaggaagatgaaagggggcttgctgctgtagtcaggaactgcatctattaaagggttaacagcagttctcagtacaactgagtaccagccatgaaagcagcatcagacatttataatctagctggatgacacagccgagcagaaggaatagtgtagtgtaatacaagaggatggggctcacccactagcctcagagtggatgacaggaaggcttttctgatccaaatgtgagaaggagaccttgcctgccttgccctgccctaacctctctgcttctggttttcatgaccttcccatgactacagtcacttggcattctccagatccctccacagtattgaactgtcaagattcacctaaaattaagtcttttatactccagtttacctggtattgttgaaacagcactggacgcaaagttagaaaagctcgtttcaagtctcactaatgtcagtcttcagctaaacc

SEQ ID NO: 16 - DNA, 5000 bp, Artificial Sequence
  Nucleic acid probe
ggagccggtctccagccctcagttctgacagatgggtcaagttggcctccttcccaagcactggtttggattctttcagcttctactttttcagttggattctcacacctccctttaaaaatagggggtcttagctcagggagacggacgtccacagagcgaggctgccactcactgctctgtctctaggctcacgtccactctcagagctgcctgatgagatcccattgcagtgttttccgaaggaaggagctcaactcccctttctcacagcgtctgtcgcttactctcccaggcagcaatctttcaagattttgctcaccttaactcagatctgctgtgcctggaattgtgcaactcagtggtcctacgagagtgtgcttggtcaaagacacccccacatgccctatccagattccaagccattcttgtcccatagagacctccttgggtgacccaagatctggagccacagctccctatggaggggtgtggcccctcgtgtctgaatgggagggagggcatggaaacgtcagggagaaattcacatgggtccttctgcttctttatctccctcctgacacagctctgaatggatcaagctcaccccactgggtctccttgtcaaagtgcagatcagtctgctgccttgcttgttaggccatatgctccccctttgtctggggaagatgactcactaggataccagcgcttctcctggaagccctttgtgaaatctttggaggtccaagtgataaagtttggaagccacatgccatctttggctgtgcaaatgcaagtacgcaggtttgcacagagcctctagcactgcgccaagcatcacgggtaccaaggtggatccagcacaatctccatccttggcttgctccagcggcatcctgcaatgtccttgtgccccttgccatctgccaaatctactctcactcttcagggctctgctaacctcctcagaggctctcctatgcagtttatccctctgtatgtttttagtctatctcaagcaccaggatgaaggctcccaggctctgtcccatttgtcttggtatattccgcctcccccaaattctggcatgtcagtaaggcagagaaaccaccattgagtctcagtggaccacttcttatggacccagttcattggctctgataacctcgtcttccaaagttctgctttcttagaaaaatttccccacagccatttttggcatggccatccctgctatgagatggctggcatgtcttcggatgccgccccatctacctccctagcctcactgtctccacgtatgctatatttctcctgttttggaatcaaggttaagactaagagactcctttacctgctgacccacgtggtggtaacttgaaattacagaggaaaaaattgtataaggcctcctgccttcatgacaaaacacacaggcttttcacaggagcccaggttgcaacattctgacactagaggcaaggtgcactctctgactggggcaaattacaacatgtcccacaggtgtccaagtcatccatccggagaagctccaaaaagggcaaatgatgcctgaaatcactttggtctcaaacaaatggaacgtccacttgtattttattgcttgtcacaaaccctcaaaccacagcaacagtcacagtttatgttagaaacagcaaaatcgatgtgaattctgccgctgtctgtgcagtgtgactcgcgggagctcctcctctcattcagcaggaagtctttgcaaagtgcctgcgtgctttagaggctttgcagggccctagtgtcatccaaggagtgaccgttggctgcgatgcttgctagcagaggagggtggacagccgaggatccctacactcccgttctcttctgccctccaccgggatcaagcactggaaggcctgtgagtcctccaaatgaccctggtcatcactggcccagtctgaggggtaggggataccacagtttacttggaatgctcaaaggctgactccagtacagccaccaggagctcgacaggtttttgagtagcaggaggaagcactccaaagctatagaccagcaaagtgggtttacatcccctgtatccaaaaattgttctggtgaatgataaattgcagccagtctctacccaatgccaaaacagcaagaagaaaatgcattacttgtctagtgttctgaatcggaatttccctgaaagttcttatttcaagtatgacacaggcagggagggttggttgccttaatggcaggagattccagtaaaataaactccaggaggcctaagatgaaacaaaaagagtcaagtgcaactgctgagatatcttcagaacgtggccaagagccgtggagtccatcctcttccctagccacactggtttcctatcaaaactcggcttcaactgctggatgcccaagtgagtagggctgctccaggagctgcaatgcttcctaataaacagagaaccatgataggcaaatgattccaggctttttgttccaccctcagacccccttgtacctaaaaatggcaccatcgcgttcacccagaatggcagggccacattctctcccttcctccccaggatccatcctcccctcatcgtttcatttgtaggcttgatatatgctggtccatggacttctggcttgaatatcttcttaggtagcacacactcttccatgacaaactgtaagttagataagattcaagttctacctcaactctgacttgggttgctctgtggccttccatgactgattgacattagattcagcttcttctctcacgctcctcaaggttctatggtctgcagacaatgagtgcttagagcatgctatggcgcagttaggaattcttagccaacccctctgtgcattgtctagcagatcacagaatataccacatccaccaggctcaacagaacacaccattatccctctggagttaaactgggagtggcagccaagacaggtggtcatacatggatgcccagctcagaatggctgactcaggaaagaacagcccgcagctctggaggttagtaagccctctgccagccaagttcccagggaactccaagcgcaatttccttctccaaagcctattgcaaaatccactatccttgcctcaaccatgcagggaaaatagcccttctgtgttatgaataataggttccagtaaccatgtagggatgaggcctgaacacctccagatttgtccttctatctggttgaggttttttgaaagatccttcctgtgaaattctgtccttccacgtgtcatcagaagccaggcacaaggccacgccctttccattaactggatcagttagctgccagagccacagggagctgctctgaggactttgtaaatcagatggatagctaatgctggcaagaagacgggggtaaaggggcatgtaaaaccttcaaacctgggtcctacaaccagataagatggaaatattctctgagctgttaaatgagccagagcctggactgggagctgatttacttctgtccgtttcctctcaccccatgtgcttgatcctcatctttggtggagtggggagggtggacggtttctcctttctccctggctgaaagtaaagattatcgcaaagctccaagcaacccttccagtggcagcaggtagaaacaggaggataggcaggaatggagtctcaagggccttcatggcctggatctgcatgataattgaggaggatgtgacttggatagactgtcttgggactctcaaagctgcatgtccttgaggtgagaccagcagggaataaagcagagctcaaaagaggatggaatccccaggacccaggaggtgggtaggaaagagaccagaaggacagagtcccagggactatcccaggtgtgtcagtcacttcagttggatatgctcttacctgcggcgtcagacatgctctcactgtttacgtgtcagatacctgtttcttgagactctgtaggccgcagaggacggatgaagtcagcttcctttgcagttcctgggttctgagcagcgacttttccctcttctcagataaccgcagaatgtccgcatagagctgaggcagctcccagtttctacttcactcccagcaaacacgggcaaaagccctatcacagtgtattttcttcttcccctgcctcccatatgctcaacagaagcaggaaggatatgtgaccaggatttacactctccacaggaagacccactcctgtgttcagagttcccttccaagcctttaatgatcaattttacttacttctgcagaccctagccacagtgatgtggctatgagagcggggagggcaagaaggggacaggacccagaatgtttaggaagcacggatcaggggacacgagaacagaaggaagggatgccaaagtggacatacgaaggctgtcgagagagcactgaggtgagcccaaacggagacaagcctgcttgcatgggcagccatgagttcccattagcagacttacaaatttactccaagcaggtgaattaggagcagcacagtcgctggccttggagaagtccctccatttcctgtgacttcagggatggtaaagaagcgagatcgtgtgagtgcgaagactttattctcacacttctgctaccatgatctgggctctagggaggaacacatgaacgttcctctggggttgctacaggcttgcagttggagtctggacctaccttaaaagcccccaaaccaggggcatgagtccaagagagcagataccttaataagtcatgaggagtcccagagccagggaaggcactgcttcgaagagttctgttggcagcaattcatgcaaaggtgccagcaagaaagcatggggactgtgcatgccatctggtctctccttcctccacagctctctgggggcaccgcattgcctgccctgaaaagtcgatgcttcactgtggtgagaaacgggacttgtaaggctgtg

SEQ ID NO: 17 - DNA, 5000 bp, Artificial Sequence
  Nucleic acid probe
cttcgcattcaaagactatgagaccaccaattagattccaaacagtagacaaaggcacagtatcaaacgtgggtaggctgttctgccagaatctggcttttttccccaaaactgaaataccagactctgttgccttctttggaaagggttagggaatagagtaggaaggtgtgttgagtgattgtggaggatgcaaacagcctgccctgagcagaaagaaatcatctctaagctcctgtcttgggtgacatgttccctgacacagggttgcttttcctcacctgtctaaactaagtaaagcaagctaaaattaaagctggccgtctacataaaccgtccatggagcggcaaggatttgcgctctgccgcctgcccaagagttccaccggagaccagatacattgtcagcctgtcctcttaagcaaaagttctgcaggagagccagcggagaagcttgagaaggagccaggaagggagtgagttccacatgccccatgctgaaaatgatcaatgctctggaataaagaggtgtcttttccagccatgccccaatctactcatgagaagcaggggctggggaacaaatctccaataggaagacctcaaaagtcctttgggtggacacagaggccagttggggcacttgccacagcttcggggtgcatcttgtgtcaagaccttggcacgctgaagaagccagtctgcactgcagaggatgcttcagccagaggatcaattctgtactctctgcaaagagctctggagctgggggtgtgcctggagacgccagcacttttctgtctcctgcttcagtcccttatcctggcagattaaaaaaaaaagagtcaacagaacgatgctgcccactgctcaggcaaaggccccttggagtaccttggggggaggccctggcttcgagaacactgttctagtgtgcgctgaaaagatgtccctatttagatcgtgcctttcacgtggctattcttctctaaagttatttaaaaagagtgctctgggggtatgagaagagcagaggacgagagcagaaacagttttgtaatgaggaaggtctgggcatttgagctccagccatggataagagcagtgctgagttccaggcctgccacagctgagtcacaggtcactctccagtccccagtcctcttggcagtgaaacagtcccaactccacaactcctgaaggataggcctgaagttgttatcatgaacaggaaccagaatgttccacgacattttgcaaacaatatcgctaaaaaagaggctagtgctacagacagtaggtggtccactattttccttgatccaactccttctacagcatgctcctagctcctgggacttttttgagccttttggacaaaactagaaattttagtaagacgcccataaacaaccagtaacacactggaaacactggctgtgttggagaaagtgcttcccagagaggtgagaagtctacagggctttgaatgccatacatcgaaaagttagtttcccaatgtgctttgtgcagccttgattcaggaagccggcactataggcagactttatacatttctgctaatgtctctcaacattttggtgttactgatagcttcataggaagatgcaactgagaagaccaacaagcaaagactcttgcccacacgtcctgccagctagcacaatcccttgggttaacaattagggaatgttggcaaaatttattcagctccttatttctaccctaagccccaattccttcctcaaattcacagtaagatgtaagtatacttgccagttgctgcctgtccactccatggactatccagcatgtgaacgaataatgaggatggtagcaggtgccgaaactcctcgcgtgctatggagtcaaaccatccagaaattccatctgcacagactgggctaggtaaacctttctgattccaagccaccaaaagtagcccctatggatgaattcttcatcttttctcagtcccatgcagttctcttatatatgctgatctaggcttattgaaggcaggtgcgctctttaatatatgtgggcatagggacccccttccatcccataaaagaatacccatcaaagcaggacgtgcgtatggcccttcagttgtccaggaaaatgaagcagctccgacagatgcattgttgtgtgtattagactgagctatgaacaatgtcttaacctttgatttctgtgccccaagaagcatctacctggatgtcccaaattcaacgtgaacagaattgaacccattactccttccaaagccacaaaaattccaaatttatttttttctccccatcagggtcccacttcaccccttgtgagccagctgggttcaggagtaaaggagggtccattgcgcagtgtgactctaagcccagtgatgatcctgtgtgtcaggaagaaatcgttttcatcctaaggaacagccgcatgaagtgcacacactccctgcacatgacaggttctccacattgctacttaattccaactaagactcacatttgctatcactaccagatgcccctacttgtccagttaaagcatttgtgtgtgtggagaatgttcattcatctaagggactagaagaaatgggatatcctgtgaggttcagaaaagcccagcaagggtcaggggacaccactgcagaagaatcattgtcaaatcatcgggagtcaccatgctgggaggtctggagtaagatctgtgccatctgccttgcccaatcctgtaccagtgcctgtgtttcaaaatctcaaaataaaagtgaatcattttaccaggggcagatgtgtatctgtcctctccgtttcactagtgcaggctggctttactttggtccatcaacatacttttgtcattctactacagggactgggcccttattactgtgctgctcaaccaatgaatattaaaagtcacgtccatgaaccatgagatctcaattgcacagctttgatgagttctattcagtaagcatcctggatggtttccccagtagagccagggtcccagaaactcctgggaatgccatcttaaaaccatagatgagctaggagtctgcagggggcgtaaaattcataccaaggagaggagttagtccttgaaagattacagaggaggggggacataccagtcaggtgaaacactcactgaatttatgtagtaacgattttcagtaagtggcaaaatcacaagggattaacatttgaaaggccactgactttccaacctcctccccagaaagtatcgaatcttggaattttattcttgccaagcaggagtcctcaatgctggaagcaccccaatagtcgtaggaggtctcttctacctgatttggggagcaaaggtgtctgccggtagccctttcctatttgcaaagttgtactaacaggacactggagcataggaggagatttgggggaagccttgcacgtggtaatgagccattccctctactggctgagctagttcttcctaagtgtagcctacaaattcataatgtctacatggatctccttttaggcgaaagcccggcaaaatatagttctttaaacctctggacaaggctcaacatttgcccagattctaatttgagacaatgtaaaacttacatctgcgaccacccaaagctgttccaagtatattcatgatgttcagattctatgtttgccaatggagaatgcatggaaatctggacacagctatgggctcttcacttcttggggcctttaactcacaaggcccatttctcaccataacaggaagagcatttctcaacttccctagaaatcattcagtcacacgttcaacaaatgcctactagcagccagtgagagaaaggggcagggggaagggctagaaagcatagtccattcaaactattcagaccaaatggtacacttctctaggatcccctgacacgggcatcaaggaaattctggcggaatattgtgagtggtgtctgaatccaaagcagagaggagaccagtaggctaaccacaactctgctgcattttctgcccccggcttctggaatttactgcgaccaaaaccagatttttcaccttccttagtttagcttctgcccagctaaacctccacgtcaactaggaacattggctgctttccctgacccttcgagttactccctggcctccccacttctgacccgtttcaatatggcgccagcatcatcactcactttgtctgttgcacagctcttcacccattcgccagggcaatagagtcaatactttgtaagcaaaggggcagagtccacgtttgattgaactttcattcatctcattgctgtctgcagttttgccctttttcgtgaaaacagctgctcatgcagccggagtaagcctaaaatcgtttaggagtccatagcaaaaggctaggacaagacgataaggattgaataaaggtaatcccaaggaggtgtgggaagggaatctgtacaagaatcccagcattccaggactactccagcttgtgcacaggaaagcagagtattcagttggcagtccaggctcagaagaaggctgccttattttgtcatgtcatggctaatgatagagaaatcagagctacattccattggcagaaagggctcatccttgctgcatgtggaattacagaaagatatttcttttcacacatgcacagtcccgttacctggaggctgccatctgagcagaatttggcacaagcttagcacaatgcctcacacctaaaagtctgaaagtgttgcttgactgactgatgcaggaccttccagcagggcaaacgtcctgaagaaacgcaggagctatcagcaccgttccgtgttgggcttggctgtgagaaaagacagcctgcgtgctgcatctacacagtaacagacctgccctcgttcactcactgaccccaggaaagctgtagactggacagcatcgaacgtcaggggagtctactcatcataccat

SEQ ID NO: 18 - DNA, 5000 bp, Artificial Sequence
  Nucleic acid probe
attttgggagctcatgaaaccagttagtagactatggagtgggatctcgagcactggagcaaggtagattattcagtaagacacagaaaacatatttcaaagcatctcagcaatcagaccagatttagagccttacgtcgagcaacgaactccacattttgagatgaggctgtgagtccaggcgggctcagaaaattaccggtgtggtttgtcgctcacacgtgtccctctactgttccgttggttaattccagtgagcattcacttgcactcctgaagtgatgaccttaacgtttcaccttcgcaagccaccactgtgtcccaagaaacaccagctctctacacagggcagcccctgatctaatgtcaccagggggccacccatgggatgtaatcacaattaacactggtccagaaaactgggtttggaatccagtaatctgctcatcctgatctctctcaattccaacctccagtagtttccaaggaatagccactctccctggaatgatcctgggatctggaagaaagaagcaggttaggacaactgtcaaatgaaaacaagtaactctcttgatggcaggaatgatggacaagctaaaacaatcgggcctttgcccactgaacagtggcataattgatactttctattctagggacaaggaggtgttgaggaaatatgggaagagcagaaatcagccccctccccagaggtcctgggctcagatatttgcacacatctgcgtgggagctccttgcagtggatagtggaaggatgggacaaaagagaaacagaaaaaaccctgtgttctccactgtgccctaaagaggacagagatccatggaaactaacatggacacaaaacactaattgtcatggaaggagtggccaagtggagtacaagagggatgttgctatgcaatttctgtctgcgcaacttgcattcattattggggatgagattgatagggctgaactgtaaagctagcaaatggaagattcgtttcaggatcatccttgctggtaagtagtaagtagagaatcaaacccaccagatcctaacttgtgaggccagaggcaccccccccccgacactgaggaagatgggattgcaacagatgaaccgggtcaaggagaaagggctgttcagcagggaccgcacgatgttcaggtgcctgggacagtacagttggctggaggagctgactcagcgcggagattgtgaattctgctattgatgctgtgtcgagttgaatgaaggagctgccgcaggctgcacagcatggaatggagtgaagacacaaatataaaacaggagaaaagttatgaaaatcctccaggccagtgatctgcatagaacctcatggtcattgtccccattcagtttacctttttataaagaaacagatttgtatgtagaagaattggccattgctaagaaatcctgagtcagaactccctaaagaaccaaagaggtaggaaagtcctctgtgagccacaagcaagatgggcaaaacacagccatgtgaacagcaggtcacttagcaacaagatgggaagctctctgtcagtcacttgactcaaatatttcgtaaagaagggacccaccaagggaagattctaggttctactacagagaaatggaaagtagaacaccgtttaaccaatcctagtcttaggggatgctaaatagttttgcacggcaaatgttccattcacatctcctgaatgaagtctcttacactatctcaaggcaggctgtcccaactgcaaacaagttgtccaaatcagcaaaggagagggacctaatgtggactcatctccaatcatctggactcagctccgtcacaaacaagctctgatgagttttgtgctgccacgtggtacctcgggattcaaaccttagaacagcactcctggtatcctgtctctcaccagacgtgggctaatgctaacacgtccctccatgctccacctgaatgcaaatttgtatgtcaccagtttgcaggaggcattcaattcacaaactcaatgtcacttccaccagaagaatcccagcaatctggtcagtgtgccttgtggggaagtctgccatgacctgacagaggtcatcaccaggcccaggggaccaccctgcaccagggaacctaattgttggcgaacgtgggcacttcctctttaatccacactgtgaatattttttacagacatatcagaattcatggagtaaccttcagagaagcaataggtaacaccaaaggcagccctaaaaaagcaaagacacaggaaggttgccttgaatttcagtggagagagttgatgaaggtcaccacggtgaccacgcattttggtctccatggtgacacagctgataaagacccttaaatttcattttcttgaatttgtattagtcttttctcctcccattccctggacaggaactcacaaaggctccattagccacacaattaccagggaaatgtaagcaaatgagagaagttcccacatggaaagggggttgactcacctgcgggaggaaacccatgcaggttctgccttcacctgtcacagagcagagggccagagcctaggagcaggcagtgcgcaaggcatggctgggtacacagagaagccgcctgcagaaggagttccctggtcagcttggccagtgaacacactggtgattaagagtctttaaagtttgtaatatggggaacacaacagagcacacttgaggagctgttttgtgtgacagacgcccaggctgagatgcttggaaagtgagtgggtggtgggcagcatttttgtccttggctttccaggcaacactaaaataacccaaactggatgtgtgaagcaaggtgtagctgcggagtgggccactaagatgaacaacggatcctaccttaatatgagcctattcaatccacctcaaagtcacagtgtcagacgtgtagaagacactgtatcagcccatgagggttaaataaaaagaaagtacaagggatcagtaggaggtttaaggaaatcaactgggactctgaccaatccactgccttccatggattttttccactgcctaatgcagacacaagagtacaaacacgatcattatagacacgtaaatagtttcagccagccgactctagaaggaaagcatgggacatgggagaatagaagaaatgaattcagatggaaaaataagcaaaacctcctgagaggagctaccaccgtttcaggccagcttcaaagttggaagtgttaggattaatctacatttcaacagctagggggcatggtttaaaatgtgtatcagagcaaagtgactgcgactgggtgatcgtttcggcagcgactgtcatagtggtagatgcgctccatgcatttagcacaggcccacccacatatcccataatcagaacccagatgaagcttccatctacttaaagtccttaaacgtaactccgaccaagcccttttgccagagtgcccgtcaactacctaccattcagggtggtgaaacaggggcatgaggaacacgatcagatcggaaccatccctcgggccacaagcggagaatgggtgtttgagatgagcagtgctagatctctgagagggtcagtctagcacttggaagccaggcctgggtgaaggattacgtttggtgtgctagcgcatcgctagtaccaggctcgtggcccatgtaaatttaagatttgtactcaaggaacagccccctgttgctgaagtcctggtgttggcaggagaatgcccccagacccgacagccaccctcccatggcactccccactgttgcacgtggaggtctacagagaaattcacaataaaccgaggcacgacctgctaccaaaggaagatggggggaggaaggcccactctaaaaagccagatgaagaaagaattgtgatgtcaatgtacaacccagctctgctgatctgattgctaagactgctaagcacctattacctaggtcacttcccaatttctcttccaatctgtcccctttcaaaaagtgtcaattcctcctcatgaccccgcggtatctgtcctttccatgacatctgctgagggctgtggatgctcctggcccttcttgctttggcggatacacatgggccagatgcttgggccaagtcctaacccaggtgattagaatctgactctttcgcttctggtcctagcaccagtgacacagaggagtcaacatcggacaaatctgcagtggctcttgcagaaggatttggaattggtatcacctgctcaaaggcctttggggacaaatttgtaatcatctattttcagagatggagttcactgctgcccctgagggaatctgcaaatgtggcatcctatgtgctgtcaggaatctagtcactcctgaatcccaagtgtctgcaatagccctagacagaagtatgtggccatctcatagcaaaggacatcatgaatgtctgtattcaaagtgagggctaggacccaacctaagcacaagagctgagagaaaagaagttaacctctgaggcttacccaggggccagaactgaagactaagtggtggagctgagaacatttctttcaagataaagaaggtaataaagataagacggggtgtgaacctgaatgcaggcctcggagcccagacaggtgtgcaagatacaagaacgcttatctaagacacaaaaagagatgactcctagggaattctctcttctccccaaacccaaaataatccaatcagcacattcccaggtcaaaaaaatagaagccgggttgaaagaatctcggtccttttaacattgcagtccacgtcacagcaacgtggcctctgctatcagaacaacacgagaggacctaaatcatctttttgcaaacagaggaggcaccaggcaaagcaaacggggtctatgtgtcctctgcactttaggggcttataatagaatgctgcatatactcacatacacagtcagctgccaggcat

SEQ ID NO: 19 - DNA, 5000 bp, Artificial Sequence
  Nucleic acid probe
ttatcatggacttgggtaaatgctgtgctaaatcttgggttagatttggagcttgatgctgtgaacagcagagggattggaatatgggggtcctagcttcagaagccatctgcattgaggagctgtgcagcaatctgggcccaatccaacctggcaaagccattcttagaattaaagagatggaagagcccgctaaacaaccccctggacaggaaattggatcattacctcattgattctggctcctggtgggacagctgatttcacggtgggcacacagtgtcatggctaagaataaatctctacctagggctctcaacaggagagtgagaagtttcgagcaaggccagtcacaggcggcccctaccctcctgtataccccaatgtctctacatagatggaacttcaaatttcacattattcaaacccaccataatcttccctaggccagctcttcttactcatgcctataattcagatattgccaaacccattccccctctttgctcacgtctgagtctcttagtaaccgtattttttggtcttcaataatcacccagaagccctgctagtcagactgtggactgaacctttctcattcttcttagataccattttcacatcgctgttgtactccgaaacccttaacagctttctagtgcctcacaattaccatctaaacatttctgcctgacttgtacctgttctctgaatgtctacagcatttccagcctgagcccacgcaatctggcacttgtttgtaacatcttctaaaagtctctgacattgcatatgctttatcagtggtcctctgaggatctacgttttctcaggatgaaggaatgaggcccagacagtgcttgctggggctcctcggtgagcaaacgccgaagctgaacctgagatatgagttagtcaagccgatacagcagcagaagggctaatgcagttgtgggctttgttcccaacggtttcatcagcggagaacaggggtgagagggcagatgggatgctctattagaatttgtcccgcaggctaacgggaagttcctaatgcaaatagggtggcacaggcacagtgaaatttaactgagggtagagaggctcgcccagggccattgcatcacctctctggaatgtggtgaaggtaagtgagattgtgcctgcccactgctttgcattccaagaggcaccacggctaggagaatgtccttgatgggaaaacatcagtctatagttgcaaggaaagggggctgacttagatttggactccctgacatgccaggatcacaacagcatccatttaggaccacatggaatacagaatggggccaaaacacagaaatctgcttttgggctcatggatatggattagaggtgactagactatattttcagtcaggtgtcctaatttctttggatgtgggcatcccagtcctacagtttcttgagcaggtaccatgtgtctatagattcattgacatagaactcagcttggtatgaccacaaacagtggatctaaatttggcacatttcaaggtcaagtgatgcagcaaacagaatctgacgtcaaacaatatgactttgtccattcacccgttccttaatgcacatcagcaggagggcattgccatgtactccaacttggaccacagaaaggcaagacatcgtcacaataagtcatgcgcttctagctcatagtccactggtctaggtaaaaaatgtgtgaatttctggtgccgtttagtcctagtcccttctctcggggtcccatctttatttaatgcaacatagtatcgctgttcccatttttcctagagcagctatgtggtgaccacactgggagggagagcctgtgcctgtggcaaggctcagatataacagctttaacaccagtataacacgccccaggcaaaaatgtgtgcattgaagctggcccctagagaaataaggtgacaggacttactggcctttttgctcttctcactgttcaactgaaaactgaccttttcactgccagttaacactgccctatttccctagcaggctattagagcatcgttcacgcacacacataactcagaaaataaagccaattatgctcaccaaccatgtggtacccagtctgctgccaaggacagaacctgccaaatataccacaggacaaactagacaattgagaggtcttccttggaagctgttatgctgacaccaccttttgatagattaatagaaatggcccttcccctcctcaacactgtggtgggctatgtctaattctctcaaggataaggtgacctacttagcaccataaccttttagtcctcgggggaagccctctgcaacgggggtgtcagggatgcagacatttttctcagccggttgtcacagtgccactgaagaggaaagaccacagaatccggagacatccagaagcctccatgtttctaaaccctgcagcaaagttgccttcttggctgaaaaggtttctctactgagaagccactgtcacctggttatccttgcagaccctaacttacaagctgatcagacttctccagtgtgcctcaaggctcaggatgaggggaagcaggagtcagattcctgagcatgttcctgggaacccacgacaatattcctccaaattcgcatcccagacgggaaatgtctctgctgagcaactaataatttaaaataatcaccctgcccagagcttcgtgcctatgcagtgttctcccacactggcaccgcagatacctagaaggcaaagggcataatctgcggggccacaagttccctcggggaacaagtcatcctgaggtgctctgcagaacaggcaccctgagctcacaggaagctgcaccaagcgccaaggctggatttacagtgaggcagcatctacctgccccacaatatggaacaccatgcaagaaacttctttgactcatgtgtgtctgcccagcagaaaaaactgccccaaatgaggtatttactaaaggatacaattctgggcaaaataatgtaagtgctacaaagggatacaaacgagaaatgttagcacctcccccatctctgcgccatcctccagtgtcacttttgcccaagtgtgtaatctaacatcctctcgaaatttcaacctggcagcgtattttaattgtgacatttgaagggtcacgtgacctaacctgcctgcccttcctactcctcattctcactgccacacgaggcatgataaccacaaacccagtgggaaatatgagattccagcccctgaggaacaagactttcactctcaccttcacacaggccaattatgcatagaaatggaagatggtgattcctccttcaactggagacctaagtacacagcagccatacaagcgagatactgtgaggccacacagctgggcttcccagaaaaggggaaagatggtgctctccagctgtctacctggctgccgtgtcccacagtattgtcactggcactccgtgggtctacaaaacatggagataaagctggatgaagtccctccagtacttactgatccatacagctcccccttctcattcatcccgaggtagagtccactgtccacgcctctcacccagagaagctcgaaccagggctgccagatcaaccatctcagatcctgcaccggggtaagttcggatctgtagcaacctgagcacatctcatagacagtttgggtgctcatctgaggtcctgatgaagaccggcctttcagagctacctaaccccagccaaaaagggaacagacttaggtttcctaaacaagttaagcagcgagtggagacggccggagcaatttcacactactaggctcagctgtgggcttaggctcaggggtgagcatgaggagcaaatcctgggcctcgtggaaaatatgactttagtcctcccaacaggacaatccgtcagcaaccttcgcctatggggctttcagaccctcacaggggagaataaacaatgcacttaatagagtcggagggaaggggagttgatttaatggactcggatggccatggcagtttctcttccattcttcggccacacacacagaagcgcaaactatgtttcatgatccacctttcatccgcatccctaactccagctgcaagaagcttttgctgcttcctcgccacttcaccatcagatttctcttgcttggttttgttggtaaacagaacaaatgctaagcagtgttgaataacaatcatgaaatcctctgcctggaacttgagtaactctcattagaatcacagtctttacctttcccaaaggctaaaattgtaccctgtatgaaccttctggcaggttaactccgtgacttatgctggtgtgtaggctcacctcctaacatctatcctggcttctcaatataacgtaaaattagcaaaaagaggcagtacagcaaggtagaaggagcgtattcttcagaggttctcccatccggcagattgagggagctctttatttgtgtaccttgagatataactcatcccagcctataaatttgaacccagttagagtgctcaggtcctcacttactaaacccataaaatagaatcatatggccagcttactcctggctgtacctcaggactatgtgtggagtgacataaaattacagttgcatgggacccatctcagacttactgctgagatttccctccccttctctgatattgacaaaaatattcttcccgtaccatgtggtatgcatgaactgcaacaaactccaaagaaacactggttcttattgccaggatatgacttttagaccctaacttcaggggacaacaaaatcacagagattacagaccttcatgctaaacagagagtggcccttctgaactgaagcctccccatcctggacaccagctcttgccgctggagtgaatccagccttccttatggactagcgtcaggtgaacgcccttctgtcttttgcccctccattttcccttccgaagtgctg

SEQ ID NO: 20 - DNA, 5000 bp, Artificial Sequence
  Nucleic acid probe
ttaactcagctgacgcacctgtgagttaccataaccccatgacctcgccaagggcccatatacatagtgcctcctttgctggaacttccctcaatgcctgttcaataattgaccctagcatgtcgctggccctgagcaggtgccctataaatattagtttccctctcttctccctgctcctctgctatcaaagctgttggtcttacaacacagcccagagaagcgatgcctctggtgaataaattgacacagaagtgtgaggtacagactggcccccaaaatattgttaccaggccatcagctacaagaataggtaaagggcagcattgccccagggataaatgttaacattaacctagcccgcacaaagggaggggagtcgacttcagatacaatggttgtccatagtttgaagcgcctacccactggagcacaactcttatttcacgtggctccccaacatatctgttcatcagaattgaattcgttgccttcgagcactcttcatctctgagcctaagaacctaaccctgactgctcttcaaaattccaaaaatgcattttcactacctgctggacttccctacatcattattacaatggtgtctagtgtgctcatcactctgccctctggtcccagactgtctttgatcttcctcacggctcatcccatctgctgcctggaggagctggaaggaccatagaatgttttcctgagcggctgggttctaggaaggattggcggggtaaggtggtgtgtacccactgctcatagaaaatgtgagtcaaaaatatgggtttaagttgcctttcaatagacagaagatgtattaattgtcagaggctctgaggtactgagccattcattatccctggccaaccatgagaatctctccctccttaccagggattagttgtcagctccgggccagaccctccaggaatggtacacaggctacttgtcggggttgtcatgaaaggaggaagtaaaatatgacaaacagcaccttctgtatggactggtggcctttaggatgcagagctgccttcccatccaatactggcagccttaagatagggctaatgtcatgtccttatctaaggtagaaaactagaataatgcagatcagaaagcccacacaattactagacatgagccacagagtcagaactgaggaaaacatgatcacaaccaagatgctttgcctctgccaaacacgtcctggcccctaggtctttgcccccatctgaagcactcccaggaaactctcaacacccaaaggttgccatgaagtcctcaaagtgcaaaagaaactcagggcaacccagtcaattggagaaagaagctctggagaaaagccagatcctgctgccagatgcccagcctcctgccctgctgagacgaagcaaaggcacatgttaagagatggtcacaataatcacagggcaaggtaatgggagctgcactgcgaggaagggaaggtaataagatgcagacgaacaaatgtgcagagggtcaagggggctgacaactgttccacagactggagatcattgtgtggcccctgcctggccctcaggacactccaagccagtccccgggggttggtttataaactgcatggttgaaaatacatcccagcccaggactttacagctgatgatgaagaaatgaacaaagctggagcccaaaaagaccacaggttaagatgggctagtgatctggaatcatggacattcagtcaagctcatttgtgttcacagggcagaagtaaaccaaaccttgaccacagtaaacgcatgcaacagccaggactggaggtatcgttctaaatctgcactggggaaaacctctgggtccctgatgatagagacatggggctggtgtgctgctgtctctgcctcgcctcttgctacctctctggtcagtgaagtggcattcacatccgcaggtggtttgcgtccagttacccctgtgggatatacctttgttaagctttttcagagccttttatagtcacactctgacctgacactactcaggcaccttcccactaaagtgattcactcactatccctagaacaagacttaaataccgccgagacagtggttctcatccttttgccaccacggtggacattcctttcagcatcagtggctgaatacaggaaggattgagaggacctataggttgaaaaccccctggagctgccatggaagctgttcattaaagctgtttggaagccacccaccagtgtgtcctgaaggtgcagctgaagtcactatgcctgcctaccagagggatcttagtctgctactggagcgggtaacccagatagttagggggagaggggataatttacaaaactgtgaaactgaaccatgagagctgagatgcatgatggtagtagtactgctttgactgttgctttagcgggagccccatgcatggagctgtgactgccctactgcaaaggaaaaacaggaaacagctgagcagcaaattttgtatagggttaggggtggggcatgagagatttggtgatagagaggcaaactgctatcagtcagtattcagccatgagctttaagaatcctcatatctctttatcctccccatccaaactgatattctaactgtgcttacttaagtctacaagccgttttctttgccttagctcagtttaccaagcctaaccggtgatatgtccaggaaggatactgtggtccaggttagagcagtgtctcaggaaagcaatccagaatgaaagcctgctattggatttcacagataatatgagggatgaaattgaaaaacagataaggtctggattatccagaagacttcagtggccctctccaaccaccagatgaatggtatttggcagggatgaatggcgtttccttggtaggcttgagggaaatcagtgtcatcctcagtcctccattgtgcactctaaaaaccatcaaatgtgctgctttgagacctagcttatctattgctgcccgagaacttgcctacatgcttcctggccttttaaactggaagtagtgctcttgccctgctaaggctgattcctccgcccccttctctagttcccatgattttagcttaagttccctgaaggtttttttcatacatccacgagcctgaggtcacagcttaaacctctgttaacttcttaataattagccaactgtgtagttcttgtctttgccaccatatactgtagcttaatctgatttggggctcatcttcctgctctgtaaaactgagtatcaattatctaatgtgataagtattcaaaggacatcgaggatacactgtcagggatccaaacagctggctgtggatcaacttttttcagagaccttggcgataaaggtaattttggtttgcccttttcatgataaactccaggaatagaaaaaagcctagagagaaagctgccagtgaggttgaaaacagatgtagtggatgtccaagagcagaaaacaatggtagtgcctcaagtgctgcctatgacctttatatggaattaccatactcctgctccctgctaaacagcttccacttctcagctaccctactctatgaacttagacttttctatacttagacttctctgtacttttatcccacgctgattggggtctcaaccctgatccttccccttctccatccctccaacacatctataaagagaagaaaagcaaggcctccttctggcatcaagcccagtggagtctagaggtttggccatttttttcacattgccgtattcctttgaaaatgagaggcaattaaagattggctacattctcgtgctctaaatactggcttctggcagttccaggcactctctccattgcctcacattcctgagaataacattctcgaaggaaatgtgtttgcacaagtgggacctgaaataccagtgccataagtcacaaaataccatccagctacttcactttacctcagggacaagaaattgaaggcaatagaaggcatatcgttgttgttgttttcacagagctacactgatgcgccttttaccatacagtccttattgtacctacagcctctactcctgaagtcccaccaagctccagagaggagcttgctgaagagtttgccatgacttgtctcagggttgtcttctcaccctgttaatctacttattgcagaattgaagcctgaagaaggccacgtgaagggtatttggtcagatgtgcccatcactctggcagcagagtaaaggataggttttatgagagagaagaccattcagatatctacgtattgttctgggggaaagcattgactcttgggctactcaggcagctcctgatggcatttacccgccctgcatcagtgccttctgattttgttgtgataggatatttctggatggagacacaaggaattggtaactcatgtctggggaatggaactgggaatcagagtggaagaactcttcatcatttatccttcagaatgaatgaatcctccaccattttctctgtcaaccacaaaaatgctgactcctcacctgtctttccagatgcccttatgggtcaagattaggggcacaattctgatacggttagaaagcagaacaggttctgggaatggaaacaaattgaaatgtcctcccaaagtttggaaacaaggttaccctccaggtgttaaatctgaggacctgaagccctcggaactctcaccaggcttttctctacttcgatctgggtagcaatttcattctcttttactgcatgtacaccagagaaattctacagtggattgttactctaagctgtgccattctgtaggctaagaccagcttaggagactccaggggaccgcagggcaaattacactcaacactagaggatgagggagtacaagacagacttcaaagtcagagttccatgcagataagcacacagatccgtagacagaggcggtagaggaaaagtgagtcactaagttttc

SEQ ID NO: 21 - DNA, 5100 bp, Artificial Sequence
  Nucleic acid probe
aggttataactttctgagttatttaccagccctaggattacagcatagtcccagtgtttgttagcaaaacccatcttagagtggatatttcctgaggctgagtattgaaaaactatagtggcacaaagaattgaaaaggcaaatacagcgccatcctgtatcaattgcatatgccttgttccttaatgtttcttacaatttgatcatgaaatttgggtgggttccccgtgagtcatgagtacttgaagttcaaatggcagtcctgtgtacagacaaattttcacacaccatatgtcccaagccaggtaaaaagtggagtataacatgaaagaataggcaaaacggcctggctgaaagaggggatttcatgataggaaacaggaaaagagaaggaaagaaaaagtcaagaagtttccatttccttctatgaaacagtggacagtcattaagaggctttttgagtagtagagttctaagaaggttgctcatgtagaataagtttagaaggtaattattacagcaccacaagtggtaacagggtccactttagtagtgatgatagcagtaccaaagacgggaccaaaacaaaggaaatagctagtttgattttcaagcatgttgtagttcaaggagcagctgagctttcagggagtcaagtctgggcctgttacatggaaaagacaatagcagagagaaagatgagaagaacagaaccaaacctagaaaataccctatttacaggatgcaagaggaagagaggctattgtggaggccaaaacacttaatggatctcagtgcaggacagccaaggagatagaaaggacattacaggcaaaaaggagaggaacttcaaacacatgggatatggctggctcacagagggcacatggagatgcagtgatagatttaacagcttgcaccagtggacataatcatgcaactaatccacaacagatcctggtggtgaatgctaggctgactccaaatctcagactccatttaggcatattcttccaaaaccacaagtcaaatgtctgtatgccttccaccagtcatcatttggagaaggtgtttttctcaaaagcaaaatcctaagtgagtttttggaaggtctgtggaggagaaagagcattcatcacaggtttcccagagagaagagataagtcagtgtactgctgacaacatgagccagggttaaggaagctttagaggacaagaggagctagctgcaagggtgaccacacaagacttcaaagaactagtggccagataccatcatttgaagagacctgaagaaagatcaagccatcccattacatagatgagactagcttatcagttcaaatcacaactttggttccagtttagtcagccccggattgtgagaatagagggattaagaaaaatatcactgaggtaaaagcagcaatgtttagtgattggctgaatacggcaaaggagaaaaaggagtccaagatggatgaagctcttattctacctatgtggtgtcaaaactatcttggtgtcctcattagattgttcttgaggccagggaccttataaaaattcccatgccttttcccctggctaaatttaaaacgctatattccgctccttctacccagtccctttaagaggtttcagttgacctgccagctttccattccgttttattagttttgccttggatgctgtctatgtccatctctctgctatacacaacaaaaacccccaccctccagctcagattcagtacaagccctcacttgtctagggagctagcatagatactaaaagccaaaaatagattgacctactgcatatgcctgtcccactgtatctctttggtacaaacacctgttccaaactgagacaactaggttgtctttacagagaagtgtaacaggaaactgcagcagctagtcttccacatggactaagcagcagagaaagtgggcctgcagaaagtcaagaattagcagatgtcagaaggaagtggaatgtccctgacagccttatcaaaagcagtggtatattaggcacagagagtatgttaggggtatataagcctccttgaagactaactggtagatgagaaatagttctgctttgtttacttacatcattaatcagacatgagctaagagaccacctgttcgcttttttctgacttaaacaatggagaccaaggagagctgaatgctagaagcacccagaactggctgtgcaaatttatttcaatcctgcccaaaactccttcaccttaaaatgccttggaggtggctctcatcaagacaagagctctggcccttccctctcgaaagtgcaaacactccattctcgcttcccctttgcccctcctcttccagggacgacttcctcctccgcaatttctcacttttgggcacctcacagcactgaatgtggtcatcagaaatactgggtcatgacccgtgggaatgggtcttgtaggttaactagatgcaacatcagggacactcgttccagggtgataatatttctaaagacagaaggagggatccaaatgaatagtctcctcctaaatatccgctggggacatgaagggtttcacaaaggcgttagtcagcaagtactctgcttgtgaaatgcagtgttccatgaggggacttcccaaacatggcttcccctcgcccccagccagcatcactgcgaatgagatgttaccatcatctacatgagcaaagctgggttataagcacactgtgtgtcagttcaggggaaggagaaagagcagctctataccaagcaaagtttcttttagttacaaggagacacttagactgggccattatgtgcctaggatgtaggaaagatgcctttggcactgtaggtgttaggggaagctagtaatcacttttgccaggtttggccttttatctgacgttacctggcctttggggtattaatgtcctcctcgtgcagtgctgtcaaggagtgctctgcagaacactctttccttagctcccttgataagttgccttagcatattctttgagcagaaccactaaatacttgaggtttgtagcaatggaagacctgcagacaaacttaaccaattctcttccatttccttttagcttatcttcccttatcccactggcttatcttctcttctcccactggctaccgcatgtgtctgttttctcttctgcctcttcggttttgtttctttttaaaaagtccctcatttgattctcagctccctcactgtggttcatggccttcaaaagaaaacgattttttgttccttcagtagaatatctgtaaaggtccacttttttggaaagagaaattcacagagaagggaaatgagcagtcacccccaaagtgtctattttatggtgtttgagagtaaaatgtgttggaattttctctcgtcatgccacagcagtgaccacctattctgactcactggcttcatgttgagctttcaaaacaagggctagaaaaagttaaaggtccaagtgaagaagaaaggacacatgagaaaactacagaggtcaaccacgctgtggtcaacattttggccattttgataggtaagtaactacagccattaatttctgtcctgaaaagtattttggtttgcatacaactcaacttcccatccctgtagacccaagttcattattatgttcacccaaaccactattttattctgaagtggagaaacttctgtgaaatggttagccttgttagataagaagaagtgttgggtattgggcaaagctgggcacatactgaatgtgatgttctaaaaaagatggtaagtacttaaataagtttcagtgacagaacaggaggaggaattgtcatctgtgcctagggtgatgaccagtttcaggggatgtatcccaatattgagaagctggtcattgtctcattattgttctgcaaaaggataccagaaagcactggaataagtgccgaagtcttttatacactttctggtaccataagcatcaacacaatgactgttcccagaagggcagtaggaatgtaatgtcatgctgaaaaacaggcacccacaaggctaatgcattcataggctacttgataagtacttttgtcctctaaatgcctattttacagttggcaaagccacaagaagggcatttccagggtgggaacatctggttcattttacctaaggaagtctctgaatatataatttatccaccccctccaacactgataatgctcatagcaactcaatgacctcagtgtgagaacataagaccacactagtaatagatatacctttctttttttgcactgctagtatctctttccctcaaacttcctcactctctctgatatttctcctattttcacaacagctatgtaagtggcttagaagggaaaaggacgctatacaagagtactgaccttgaacacacaatgatcctgcccacagcctaagaaacatactctagccaaacagagcagccatttgtcgactgctaggagcttataaccccgaaccctctcctccagaattcaccttccctatcatcacatcaccctcacactgtatttaggggttttgtatctgcttgtgtctcaacttatgggaggaaggtcaagagaaagatagctgctccctcaacacctctgtaagacctgcctttcagttcaagttttccatctgaagctcttcagcaatgaggacaagtaggaaagtatgacggtaacaaataacccactggccattcacaaagatcttggtgcctcaggcaagtagtcagatgagtttgcaggacgtaaatagcagttccttatataaagtaccaggttttttccccattctctggaaatgtttgtcctcccttaattttgcctcaacacaagaaaataataccctgagctattgttcctgcaaagtggaaataatagagagggacgttcctgcagacttaacccaatcaaaagtcacgtttgtgctttttgtgcatctttattatatgacaatttcccatcagtccaaaatggcataaaccccaacactgaaaatttagcagttgaaagtacagtcacctgtgcctacttctaaagtta

SEQ ID NO: 22 - DNA, 5100 bp, Artificial Sequence
  Nucleic acid probe
ttcaaggcatgaacagaaggaagcctagaactcgtctcctagcaccttgttcaccacactgtgcttggtgtgtgtatgttgggtggagggaggggaagggacagtccaattcttatcgtaaacttgtccctgatggaaactgctcatcttgagctgcattcacactcccagttcatgtagtgatgtgatagtaagtataactgctgcatgacctggtgtttctatactgctaatgactgatgcaagtagacacatgagtgatgagctgtgacaatctatatcaatcattacacaatctagagtgtcccaacagcagtactgcaaattcaccagcaggaagagttagtgcatgtacccagtgtggtaaggactgtgggtcagagaactaggcttttagtttaaacacatccagtaaaaacatcagaaaacctagaaccatctcaggttaaccacctgccccatgtgaagagtcagggtacattgtgggagcgagtccacagcacaggaaaggtgaagtgaaggttcagaagtgaagggtccacctcacaggacaagagaagcatgccctgagcattttggtcgctgttttctttgattttattacatccatcaacaacactagctaatacagtggaagataaaccggccctaagcaaatccacagggtgcaagttggtcagcccatcgggttggttaaaataattgaggcataagctttaggcactggttaacgattggttaacaaacgaagactcttgaaagggtaagtgactatacaagagttagagaagaatagctccttccccagaattctcagtaccccagataaacccattctctccctgggttccctgcctcttctagcaaggattaggagcattcccaaaaatcctgtggactgcccatccctcttctcttagtccagagctcttacttatttctacgcattatggccgtatgaaggctgtttgtaaacctgcaaaatacaataccaggtatcatgcacgagttattgaagacagctgttgtaagcatttcaggacaatgcacctcccttaacaccgagtccgggatttggggcggggtgccggaaaacgccatggagtcgatgcttcctcttccaagtcaggtcggctcccgttaccttctcagcattcgccgttccggtcttcctgagcgcgtgcatgaggtctttcgcgtcgacgtctaatctgttaaaggtaagaccctcactccagcttgggttttactgtgtggccgccgggttcggctctttgggaaaggcacggctcaggttcagcaaaggaaaaaccaagaaaatgctgcctaacaggtttcagtgactcgaccccccacttcttccatttctgttctcgttctgtctgcacgaaattccgagttgagttcatctttttccttaggaaactgctttctaacccccacccagttacaggacacaaccagatgacaactgatgaagcgataccatcgttttagggcactactccccctctttctttaagaaaactaattcggggaattgagtaacccatcccactctgcttttctcttttataatggtatagcaaatttctcatgcagtttattgttccaccccaaacagcaattgtttaaaccttttcttgctgagcctcacccctcttaattcttagtacattactttacccagctatttgaaggttcttctctgttccatgctacctcccaaattgtctcattctgcttgttctttttcagtcctcatcttccccccaggacttggtggtatgtcccctcttaacattccctccttggtcttctctctctcaaatcacgtttgtttttcactgacttccctcccctgtccccaatagaattatttctcaggattttatcttcaacttagctggttacttgttggctcactagcatgccagaggtcgtcatctccctaataactaactcttaactttcattctctctccttctgattggatctatgatttatgataaggcaaaagtttcagatgtgttaccaactgctctatgctccctcacagattgaggaaataatttctggtgggatgatcagtgaaaatttattctatatgaatagtgttccttgggattgttgcagcatagaaacccagaatacagggaaacaacaagagggataggagtgaaagggaagtcgggtcagagagcggccttcccctccttcagtgtactgtcctagagagatggttgcattcgacacagcctagggaggaaacaaatttcaagggtgtctgtgttggtcataatttgatgtggtatgttttgtcccaggcgttatgtagggatttctgggtgttttcggggcgaaccagtaggtcaacatttgtattattttgttgtaaagaaagatctatgtaacatcttatatttctggctgataacacttacccccagcccaaacacattttctttttcctaagccaattaacacaggacccaaacgtaatgaaagaattcaagaggctttgtagcaagttaatagagatgaggcagccttcagtcagtcagtgaaaacgtacataaatcaaggttcatagtctgaatctattaggagatttcagagcctaaccagtgtttaatttatctccagtacctgtctactctggtactctgcaccttttatttttaattgcaggacttacagttttgaagcttttacgccaggaatctgttacaaaacagcagtggtttgatctgttttcgtaagtaccccactaattctgtttgctagcataaaggaaattgagaatagtgcttgttctctgagtactcctgacttagaaaaggagcatagccctactaaaggggacttcaaagtagaaatcgtcaataaccttttacttaggaaatcaaaacctctggaatagtttttacttcttgctgcttttcctgtcgcttctgtctgcctccttaagacttttgagaagcttctatttcctcccttgtttgtttacctactttatattcacagggatgtgcatgcagtctgtctttgggatgataaaggcccagcaaaaattcatcaggctttaaaagaagatatctgcatttatttcaaattggtacacctctttttttcagcgagtactgagccatcaagatgatacggctttgctaaaagcatatattgttgaatggcgaaagatggttacagtaaaacactcaggcattacaatagtgtaacctgggcattgttgccccatcccagccttaaaatttgatctgatgttgacagggatactgttcaaacataaaaaacagactccaagatagtgcaatgaagctggtacatgctgagagattgggagaagcttttgattctcagctggttattggagtaagaagttctttttgcctatgagccatagcacaactttgtaggaggtatcttgtttagaacacattgtcctttggagctggtatgcaagatcaaatctatttgcacagtaccatggtaggtgttttcttttacgtggaggactgaccttagggtgcttcagtataaggaaagttagaatctagttgggacagtcttggaattgttgggccacagagttgtcccttaggaagattaatctggtgacaggaaagatggaagactggaatggggaggagctgggaataagaagtagaaagcatttgcaatggaaaagacaagggaagagagaaacttcttagaggtggaacattgtatcacatggatggggctagatatcacagagaatcaagccaataatgttctgagattgctatggatttagaagttgtgctcatctcaggcaaacatacaggagttgatggcatatggcaaaaagtttttttcttcacggaatctagcaaaagttgttagatttacctatgctcaatctggtttcactgcttaaggtggaagtgtttagttgggcctattccagtgatgttatagaataagattactcagcagtgactaatttttgatatatttcagctcatggaatgctgtgtaaatgccctggtgacatcatttaaagagactatcttagctgagtgccaaggcatgatcaaagttcctaatggtatagagccaatgttgaaagacttggaggaacatatcattagtgctggcctggcagatatggtagcagctgctgaaactattactaccctcttgataggttgaatgaaggagggaggtagatcactttgattgttacagtagttcacacctgagccaggcattgcaaggagagatggatggattctcgttatagtctcagttttgctaacctttaaagtagtgtatcaggactgggtatttctcagagatggataattcagtaggcctctatctgtatatgctcatagacccagagagaaaatcagctttgaaaatcttaagcttgcaactgaactccctgatgctgaacttaggaggactttatgggttggtttatgtttttttgttctaaaaactttggtctgtttgaggacttgtagattgtactcattagccccctgctcacaactacattttattactactccatagacctcttaaaagttcagggatttgcttgactggtaatgacagtaaagggagtgtcatatctatgcagttatgacagtgtcctggattcttgcttttggcagctatgaagtagttggcctcttccctgctctctttcccctgctcttactgagctcttgaagcctctgagactccactacatcttctgtgtggctccttggcctacatggcacactagtgtccctctgctctcttttcctcttaggttctctaactaccaccttcacctctatctaaggagaaactgggatgtttacaaatgcctctgcttttcctcttcttcctctctgaacccctgacataaaccccaatttatgatgatcaggagcctggtatctgaataccatcaagggtgagaggaattcacttgaccttccattagttagcatgatggcattcccttcatgttgcacactctttgacagcatgctgttttgtggagaaacttgcattcatgaagagcccattatcaactt

SEQ ID NO: 23 - DNA, 5100 bp, Artificial Sequence
  Nucleic acid probe
aaaactagaaatagcactcttggtttcttttgatgaaaagagtgagctggtcaatgcaacattgggaaacggcagatgtaggtgagaagttaagtatttaggtattttaaggtctcatgattagggatttgtagcctcaggtttgaatcagtcacctaggggtaagagaaaaaccacaacataagacctgaaaaacagaaaatcacagttggcatcctttattcaggtctcttctctccagcgtacacttaaaattggtgccgagcagggatataacctgcagttaagtgaaaagaaaatagacttcaaattctaattcaaaatgagagcagtcaggaatgtagcttttccactgatgatagcatgtggctatatgcctggaagaggtaataaaaaaaaaagaatatggactttttatatcacggaactcctgttttgacagtgtacaacccagagcagggtgacaatttagattaacaattgatggtgagagttgagttaccacacttgattccacttctgtcttttctccagaagctggcctttgtttacctctgcttctcctgtgttcactgaacacacacactctgtaacatgcttctgggctggcctacatttttgaagtggattacctaaatgtacagagtggttaagccgtggtcagtaagattatgtttgcttttggcatttttgaaccattaacatttctgtgtgcccactgaagaagctctgttgacactttgtacctgtctcaggatttcagtgaaggaagtgtcttagctatgtgcatccctccaggaaaaccactttttgagtagctcttcattaatacatctgtccagacactattgcctgctatgacaggaatgataatccaaagccacaaattcagatatgcaaagaaataaatgtactattcttctcccctcaatgctcctaaatgtctctgaacttctctagaagctgtcctgatgaacatagtggcactttttcctggtagatcttttactgtgtgataccctagtcatcagctttggcctctcacaactggtaatctctatgaggccagacctttagttttctgagtcatttctttaacataggccttggtgagctgttgaatagagaggttcttcggtgcaggaatcacaggggactttaagggacaaacctggagtgatggggaaattaaagacagggccagcagcgtgtcaggtgctcgttgaccccagcgccagtgtctccatccacgtccttcactcgtcaacctttcctggcccaggctgcggtgtcccagcgcccaaaaaccgcctaagtgctccgataacacccagggactcgcctatttttacagcgcgtttctacttccaaggtctcggcgtaatcatcttaatgagcgtaggggcagagtctcaaagaatttattctccacatgcgtaagcaagggagtacgcaccacagattgtcaggcaaagcctcaggaataggtagagaccttcaatgtttgtttttgttgcaatggtaggtcactctagttcttacgtctgggcgtagaataacattacttgcacaagggtgctggcggtaggagctttgcaagagtctaaaattgatcctgggtgactgagataatagaaaagtcacaaggcgtaggtttccagggaagatgagtttttagcctatttgttagtctcatgggacattcttttgcttctcagtgtattcatggcttttctctttgttctaaccgctgcttttctgtggcattggctggtcagctccagccccttttgacttttcatcctagaagagagggagtggaccagttgggaattagacaaggagggccattacagcatctcctaggaccttctgaagagtctctgttttgtaagggtcaaagtggtattgccccacctaggatacaggtcttgattcctagccagagttcctttcagtaagggaccctcttagagttgtttattatgcagttaacaagagctgccagccactaagcttggttccattgcaattcagggagccattgaaaaggcaggtcagtagttacttggctttttgtgttaagggagcaaaaagatttgagattctttctcataagttagtatgttttctcacatgctcaagagtgtctggatttgctaagtccatttgaagtatggtcaaaatgatagcgtaggctttttcattataaaggagaacacggtgcttaaccctgggaatcaaatcattgcttgagcccaggcttgatgactaatttgtagtcattaagttaagttctagagagcatgtccaatgttccatatgtaatgaacagaggatcaacaccatatggtggggtaaagcttgaagatttgattgtaaaagacgggctaactgatgaaaatatttcaactttttatcagggcagctgtgctgagaatacagcaaagaagctgaatattgcacgaaatgaacaggacgcttatgctattaattcttattttaaattgaaaagggatttactagaaggttaccgggagtgcctccaagaactgaaggatgaacagtagcaaccattcagctctgggaacttcagggattgttcactactactaagacatgctctcttggcttctcacacctgcaggcttttgtgctttattctctcagcctttctaaagttgctgcctctacctctagccaatgccagtacactgaatgatggagcagctgctctggttctcatgacggcagatgcagcgaagaggctcaatgttacaccactggcaagaatagtaggcccccccctttttttaaacagcatttgctgacgctgctgtagaacctattgattttccaattgctcctgtatatgctgcatctatggtgagaacaaagtgctggctgatggttccagatctagaatggaaagcaactagtcactccccaggagaggactccattctgaaagggaacagctggcagcaggtcttcagagtatattccaaattgtcagtaagggctatccaagtctcagtgggaaattgctaagcagtcacaggaaaagaaagttttaggggttcagggttcttagaaatctttggaacagtggaagctgtttgagctgtataggctagaaagctacccttagtccactagtagaagagaggaagatggaggaagagttccacgttaattttcagtaagagttggctattctggacaaactagcagtgcttcatataatcactcaaaccacagtgtgtgcagcagtactagaaacaagacagaagcccatgttataaaaaagggaacagatcaccacttccaatacacgacaatgcctgttcttaagcaggacagactgtaacagaagtatctcgcattgcattttatctggctttcaataattatctgccccgacttacaaaaacttctaactgaaccaggagctggataagatataggagaaaaggaaacccaaaacatgagaatacttccttgataatatctataggttcttctttatgtttttcccctgcccctgagccaggtgtccggggcacaggtaaatcactactatcagcaagcctacttactcttgttttttggtcatttcttgcagtgaagctatagatttctcacttcgcaaacccccattttgctgcccaatagatagttttgaattttctggatttttcttttctgattgagtttctcttatggtggtatgccggctaatggcactttccgatttagataaaatcttaggcagaggaggcttctctttctctcttttgtgggtgagtcaagattaggaaaagagactgcattcctttctttgttttgggacaccatcttatggtttggcccctgcgtatttgccacaggagcagtagtcggtacctatcatgtttacaagcctatgtcactcagagcacatgctctggagttctaatagccatattttaatttccacgagcttcagcatttatgtgggggtgagaaatatgtagtttgaaggccttgagttacacaaaattagcaagggtggagaatagtttagttatggctccagaagccaccatagtatcaacagtagtggtagggaatggtagcacttctaaggcaatgctaagaagcccaggtgtaaaagttgagatggctggctggacagatcaagggctatagagtagctaggtgggctctaggctgtttagtaaaggaagtgaatccggaaagaagacacaagttcaggtaaaaggtgaggattcaagtaagaaaagctatcactggttgacaccattttcagttcataaatcttacctgccgaggaggagtagaaaagttatttccattctgtccaaccacagatactgggatcatccctaccattccttatattaaggtggtcttaatgttattcagtcagtgaaccatgctgatcaagcataagactctacagccaagacatcatattacaatcatatctggattccttaagcacgtaactggtcaaaactatggagggaaaattacaatgtctacatggtcctcagaagtcaacaacatagtttgtggctactgtaatttagtcctgtaatacactactgtcaggttgatcccctcattttcatgcaacctactacggtctctctgtctatgctggtgctgcttctctttataacttacagattttagctttaactccaaatcctaccttcattctttaagctttagctcaagccccacattctctattaattctgacctctcctttatgtaatttgtaacagcacgaagttaccttgtgaaaagtttggtgacacagcttggttgacagcaaatacactgtttgaccttggtggtgtctgtaactgaggtggtagaggttgagcagctgacttacagatgttcctaacgctgttggatcagtcacacaggtagctatcagaatgttatttgaattgccaaaagctgtgcttgtggctggcatcaactgaatgttaaaagactctgttccattgaggctgaatcccctacttcggcatatacaacagcacctacagtttcttctgaagataggcatttaactagttc

SEQ ID NO: 24 - DNA, 5100 bp, Artificial Sequence
  Nucleic acid probe
agacaagattatagttggcaggttttctccattaatactagaaacagcactggtaagttcagtatctgaggaaacaaatggatcatcactaatgataactgttaaagtcattcaaattaggcacggactcaaaggtaatgtcaatgtcacacttctgttcagtgggtacagctgttttaaaggcctttttctgtatgctgtactgcaaaaaacacaacccactataattcagcctaagaagaaatggagaattggacttgattgttggcagaggaattaaaggaggcacacagtctgagagctcactctcttttgtatttactaccaatagcaacagtgaccattgtccccatctttcttctcttagtattaagcctcacaggcaagttgtcatttggttttgctgtttgttattttatatggtgaagtgagaaaggctccaataaaggaggctaactaaggtcagtcttgaatgaaagacaaccaaaagtaaaaactcttaggatttctaacatgtaaagatagagaagtagaaaggcattcctcattccaggaggacagactgacatcaacaaaggcaaggaagtagaaaatataatgtggttgaccactacaaaatatgacctcgatggatctgaaatttggccacttggtcgagtaacctgtgtacctgtggaaggaggagtggtaaactgtcctggaagtaattgtgaacattgtttaaagttggataccaacctctctgactgccagcaaaccttggggaactttgcatgctcctaacaaagaaaacaaagtaggcatttggagccattttagcaggttaagcagaggaagaaaatgaaacccatgctgaagttaggtggggtagtcacttatagctctgaaagaaaggttaaggtcaggaagaaaaaaaaaggcttggagacagcagcagcgaaactaatcagaaacgagggtgtcattttgaaactaactctcggagtcaaagttgtaattctattgaaaatgctgtagcatgtactgatgtcacaattgaaaccttaggtggaatgctctagtgtttggcagccttgaacagaaacaccaggttgtgctgcactttataacgttctactctcttcacctggaactgtatcgttaaccaggtccaacaggtgaaaaacctaataaggttgcctacaaagtcattatacactagtcatctcgttacaggcaatgccctagctgtgatttgggaatcaactcaccactctctacttgttgatgaatctgttttccaacctcttgctaactaatatacttccttttcatggtggagtttcaagtacttatcaccctagatgccttcagtcacttggggacttctgatagacggtggtattaaaatctactatagaagctgacagaaagtatatatgcaatcatatttggggaacatgtccagtttggatgcagcacaaggtttatgaagggtaacagttggagatacaacagaaaacttcaattcgtttggcatgtctgcagcataaaacagatgttaggaagttgagatcaagttggagaggtaggtaggatcagttccacagagcaccttgaagcaataagggctacttaagtggagtagtgagatgcaaaagtcagagtgcagtatcctgaaggataaaaggtaggtgaagaacagcaactaagtggaattcggagatttgtaacaacagtaaataaaatgaattgcgaggacaactgtctgggaagccatgacagaagtccaaaagagaagtaatggcaggcacattgcgggaaaaccatggctggcgttttttgtccattaaatgaaaagcaggaagtcagcagagtagctggtttgtggagaaagaatactcacacatcgatgtgaatttggaaccatttccgtttaacgtttcctgcaaaatgtgcttatcaagagccctgcgcaaataacatttttgtggacagcgctgggcatttaaaagaggcacaggatggagaaggaagatcacccctactcccaaattcgtagttataagggcgggttattagaagggcgccaggaaggtctctctacaaagatggggggcctattttggtcccaacactgaaccaaggaaattgcgcccaaggttgaagcacacgcaatccgtccgtcatgcagacaaatgctctttcaggggtcctaattgcagcctctgaagagaggagcatctacatacaaagaggcttaaactgcccagaacctccgaatgacgaagaatcaccgccagtctcaactcgtaagctggggaaaacctttggcctcaaaggtccttctgtccagcatagccgggtccaataaccctccatcccgcgtccgcgcttacccaatacaagccgggctacgtcctcctgcgccgcatctcctggttccagtggcggcactgaactcgcggcaatttgtcccgcctctttcgcttcacggcagccaatcgcttccgccagagaagcggacttggccaataacctcctccttttaaacgccctgaattgaaccctgcctcctgcgcatcctctttttgtgtcaccttagggttcagatttaactagacctgtggtgagcactcgtgagcgttagctgctgtatttaccaggtacagataagacaactacagtggatgataatgtatgtggtgataggggagtactaaaatgcgtgtaaaggcacaaagtgtagaaggaccctgaagctccagcgtgaggcctggcattgaatgaaatatattttgtgggttttcagctgctgaagagaatggcatgccaagggcagagttatttctaggaagatgggatataagcgtcattgtcaagttgtgcaaaggggtcaacttggttgagatctaaaaaggtcattaagctgaaggtaagaaaaacaagttcaaggaagtcacaccatggggaaaaaaaagtcaagtttagggctgcctcttccgggaagtgtcaaatcttgtgaatgagagaaagataacatgtcaattttgtagcccaccagaaaagtttgcaatgtgtacagtagttttgccttgcagtgaacaaaccttttaattgcatttttcctcatgaaatgaaacttcaccaaagaaagcactctgtctgtatctgtctatatcccccaagtgacctgacagtttaacagtactttagtaaaactagggaggaatctggtgttgtttcctgtttatagctgggaggtactgagagaccacaaaggaaggaagttatggtcagagattgaaatacttcagtttgcttaggagtcatttgtagcctttacgttactctgaacacaagataggctgtgtctgtaacagggccctcaacaagcctagagacagcctttgggaataggcattttacattatcacttacaaccatggctgtcactgaaacaaaggcagtctttcccctagtctgcttactatattctgtcatctagtttgttctctgaatgggaaatacaaaaaaatctgagacattgttccctctaggagctacttatgcatcaatacttagatactaaggggtctgacacagactgtagggcaaaagaagagagtattgcccagaaggtatagtgcttttctgtagttaggaactttattggctggaactggagtttcctcttgctctgtctatgattgcctttgggatattgtcatagcaagttaagaaggaggactggctctctgataggcaattagaagtgtgcacctcatggaattcactgaattgtacgcttcaacaatagaaacgaggaagacaaaacaggatgtaagcactaggataaaggaatggagtccctcgtccacttaacctttccacagattggtaattggaaccaaatgattgattgtattgctgtgactgaaaatgactcactggcacaaacattccttatctccctcaagagggcaaaaggatgcgtaactgcttacctataccagatgttaatgattaaataaagcctacaatccattcaaaggagtgaattcggtagccagggaaaatgacttgaggacatgagaggggacagtgtgcctctttatgatatctaaccatgtgggccattttcttagccctagccccagtgtatgtggaggtagagcctctggcctagcgagtagcaagttgggtcctaggtcatgtcccacatgcctggtgagtccctaattagaacctctgatcacagtaaattgtaggctgagtcattggtggaatccctggtcatgtttcccacccttggtgaaaacagagtgattaaatgttagagcaggttgacattggaatctgtggactcaactaattggtgttcggccagagaacagtttatcttttctggctagtttgagtcattgtactaacaagagtgaatgacttgtccaccagactgaccaggcttttggaaatagtatttattgagggaaacacatggagacaagagcagcatagcattgggatccagcagggccagaagtcaaattttagagctatcaggcttttaagttcagttatcaggggagaaggactagggtccaagccttataattactctaccagcatagtttccatggtgctttggagtaagccagcctataatcagttagccttttacctctcagtataagacttgttcggtgtttcttaaaagtaccttcactccctcttcaatctcaccaagctttccacttttcaagaaaaacaaccgtccctcattcctatagttctcataccctgctgtttgtttttttgtggaacagaaataggcactccagaggttatggaatttgagagagatactcagcctctagccactcccattcaatctcccagcttagtcttctgagcattccactcaagtagaagtctgggaagaagttacgcagggaacaaagatgacattgttcgcagtccctgggaattcttaacagtaaagttctgtaattgagacaataaagtcaagagtaaaattcagatagaggagcagttatacatcagtgggaaatttggttggcagaaaaattaccaggtcatacaacttaatgatagacc

SEQ ID NO: 25 - DNA, 5100 bp, Artificial Sequence
  Nucleic acid probe
aggtaattacctatcctcttttaccatagtctccccacagtaattactctttctaaattgaccacttggttggccaacccaataaccttttcttagtcttgtctaaactctaaatgttagatgttaaaaagccaagcaggctcccaaagtcccttctagctttagtgttgaatgattcttgtgcttctgtttgtgattttatctctagtttaggtgtttagatttagtgaccacgggatggagaaatcacctggttaaagtcagcttgaagctctcgtgatggtttttgtttctaattagcaacatacttacctccttgtccatagtggcactgcttttatggacagacctgatttacaacagaagccctaagtggctttcctgtgtagcataaaattaaacagaccttgtttttttagaaatagaaagcgctactttgctcccatgaaagctgttagctctgggtatggtttatagttattgtgatccacaagtgtttagacagtgaataaaacatcaccttctctgcaatactacaccgcactcataccaattcagcagagtggtaccctgaacacacatacagcttatttctcattagagatttttaacagtgcatacagaaattgctagaatggatggtctgactgatgagtactgcttgagagttgaccagaactctgaagaaatagtgtagtagagctattctgtgtatcctattcttagtctgtggtacatggatttcttggtagagagcagatatgaaccccaattttgtatcaggtgatagtctacaggttgcggttatacccatttgtgtacagaaatgtagttgataaggaagtggatcggtaggaagccttatatttgaagaagggtgccaaagtataactgttcctagaaaagggttttcgaaatgagtatcatctcgcatagtttgttggactgaggcatgccaagaaaagtataggcattcctctttctacttcctgctacaaaccctccttcaaaacaaattgtttctgggattccaggttcattctttaccctgacccttcaagaaagttttggtgattccctctttgttctcttcctatacaattacctgttggtttttgcaaacttatgttgctacttcagctgtaaacccagcatacacactggataaagtagaggcattggtgtccccaaggtttgtctctttttttttcccacccaccaaggaaaaacatttttaacctgcttttttccccgtacatgaagggcagttgggtacagtcatggtaatgcattatattttaatggggcattagattggaggatttagaaagcagttaaagatgatgtgatcacaggtgggttttccccctgctgcagaaatatgggatttttacaataaactcacatcacataagttactcattttctctctctaattcctcataggcctctgcctttttctcacacatgcaggcatacacgctctacccactgcagtataccactgttgagctttgactctgagctgcatagtggccaaagcccagagtcttcatttctcaatcagagcctgaaccacagattagcaacaagttggggctccttgaatagtgcaagttgacgtcctttgcatcagtataccgggtctcgtactgtgccaagtgctatttaatgtaccaaaaagggagagcattcttcccagaatggcagtaggtatttaattatttgggagactgtcaagaggtgcacagatgctcagattggtttgagtgccctttgctattctcagatgactctgtgattcctttctgtatcaatctctcaacaaaaaataagggtttgattccccctccccgccccagagctagatattgatagagagtaatcaatgtaaaagagaatcaagcatcaggggtcacagctaactcaaagacagcaggggagagggcatgtagaatacttattaggcaggaagggaaatcagaaaggccatgagatcagaggaagtgcttagttctgcctggggtggggagaatagacagaagtagatccaagaaatgcttcacagaggaaaaggtgtgtgagccgagttttgtaaatagatggagtgttgctttatgaaatgcaaaggagtttgcaccttatgctataggcctcataagaccctaaaggatagtaatatcagacttgcattttagaactaaggaaaagaccctgggtaaaagcaataatagttcctcttccttccacccacctgggacctaagcccatgtaacatacatactgttattccaaacctcttccttgtagttttcccagcataggtctgtgtactcaacttggattggggcagattgcagtcagtgtaaactaataattgccacaataatcaaagactgattttttttcttaccaggtagactgtgtatctcatcaggaagtcactgatgtgaagagcactgcttcattttaacatagggggatgtggctgggcagcagacttaggaatgagggccagactagtttactgctggtgctacctcagtacctctctgctgtcttaactttgggacagctcacctgaatagggtttgggcctgccagtcattcaccaatctggtaagggtatgtgagacaagaaatcaggagtgtggcctccccagggaagcatggcaggtagagtgcagtatgggcttgccaacctagtttcagtcagaagggatttttatgctaaggtggatttgggagtttcttccagtacttgtatcctacctaacactcaccagaagaaggtagttctaccctccaatgctttgtgtattttacactcatctacctaatcttccctccgtaaatctgtccgaatttccccaaatcatttggtgtgattgttccaatctgatcttaccacagtaagaccctttctgccttcagctttaggagggtgcttctcaattagcatcctggttcccagtcctctgggacatcatttctttcctctttcattgaggcacagtcactatttctgaactgatctccccgcttcctaagcccctccttcagagcactttaacctgggtgtatttcccttttcttttcagcttggagcattatgctaggatagtttcctcaaggaaacatgaagtgtgcatgatgtttgttcccctcccccatcaactaccatgtgactggcttatttggcacaaccagtctgataatgctgcatctcagtctaaatatcctagtagtgtttcttagtcgcctcctgacgaaaaggcaaatatgaatgttctttctttgatgggaatccaaagagttgtattgcatgcagagtagtgaaggcaaatcgtaggccccaaaaaaactgagattaggggaatgatctaggaaggaaaatgaagcaaggcacactaggatatgggataaagggaccgagagctctgccttccatgaatgcactaaatgcctcaagtaaacatgcaaaataatcagtctgaagccagtgttctatttctgctcaatcttccttacttaactgagctagattctgtgcccagaatatatacacaagttattccagtcagaatacactatctcttgaccattcttaaagcaaatatcttcacctgaaaccagaaacaccccaaatttctgagttcatcccactttcactcccacatatcactgtccatccctacaaaccttctcttgcttcttgaagaagtgacacacattatcctcttctccaatgccaacatcttcaatgtatcaccttcactggcttcagttatgcaaatctcttctggggtctgatgacactcttcttcaacatgctggcttttacttttagcatttcagtactgctaactactggcagaacaatagtggcctagaacctaactgctaaaaatcacctacctttgaagttagccgaagagttgcttgtagcaatttccttccagctggcaatgtacctaagcacattatatgaggattcattgctaagaaccttcaggacaaggagttgaaggaggtataaaaatggtgcccaccatctgccatacttagtgtcctttcacctccctctagcatgccccctccagcgctgtctttgcagtgtccagaacacgttaagagatacatttaggtatggcaaatgcagctgtcacctcagctcctgaacagagactgaaatagagagtctgaatttggctccttcttactactgctaccccttggtgctctgcgaactttcttcaatggccttcaataggatactgctgggtgacttaaatgaaagcaccttcaagaaaccaagataaaagaccttgatcgatagtgtgctgagccctgacaatctggggcacaaacacttaccatagctaggccccatcactgtctgatcctggaagaggaacatctgaatatccattaggatgaatgccagcaaaagccacggtagctgtcctgtggcattgtgcataactccgtgatggtgatgctctggatacaggaatttgcaacagtactcattagtgtgtttgagtccactagtgaaactggctctatccattcttccaaatgataataacaatgttcgcactaaatatcagtgttcaaggcatttatgccattctaactgtccaactgtagaaaaatgtccatagacgttgaaggatactttccaaggaattaggggttctgcagttagctaggggaaggcctttgcaaagtttggtcttactgagataatcagtgcttgtggccaagtagtgctactgaaatatacccagataatcataagacttcacctactatcacttagcggatgatggcggggaaagtcttatgtggaccgagatgcctattttttcttccctttgaactcaatggttttatttatcaattttttgtacttgtttgccggcccttagcttgaatcggccaatggtacaagggaaaggcagagcaggatttcaaaagctcacctgagaaaattccatcattattaccatctgcatttgttcagtgtcccaggaactacagacatgctgtctatagggaatctcaagactgaggtctgcacaaaatattggggtt

SEQ ID NO: 26 - DNA, 5100 bp, Artificial Sequence
  Nucleic acid probe
agcaacatgtacttactcatcaaagttcagtgtatttgtccagttcagcacttcatccacttcccattccatcacagaatctatccccccatcttcaaaacaacaaagacctaggaagaatcaaagcttaagttgcaaaggaggttgaggaaagctaagatctataggacacaggaagggactagatttaaaagccagaaaggcattgtaaccctgtgacccttgcaatggaacccagggttgccctcaacccatgctgtgactatgagataaaggaccacttcagagctgccagggaggctaattaagtgatgggtgagtagtacactcagccttcagaagcacatgctacagatagatcaacaaagtactagtgtactgaggtgcttaataagtgttgaacccatagtaccaatataacaatctaaagccccactacaattgcctatggaagatggtacagcccatgtctatactaatagtattctttctgaaaggagtttctaaatccagcctctgtcatactttctattgcatggggtaccattttcctcccatatataaccctcttagaggcactcaatatcctgatctaatttagtctgtgtcagtttttctaggcgctagtacgagtatcttttgttagtattctgtctacccagtgttctcactcaggagtgggaaaattgcctgggggctttaattaccaggctttctctaatgggtatcgtcaagtagccagctattaagcagggtgtctctcaaataccaaaattaagaggcaacacaacaaaattaagctggagacaactggcacagctgccccatatacagaccttcaattaacccctgtcttcagtccttctccttccagctcttttaaattgttaactccaaatttatctgctcatgaacccccttctctttgttatggattaaccttctacacagtgtcgtttcaatggggttctggagagatagaaggtgaaaatgtgtcctctctttatttacatggcttctctgctgaagctccttcaagggcaaggatgtattctgaaatgcctagcacaatgtcaggaacacaacagctagtcaaaggtgaaaagagtgaggtcattttacctcttggctctcacccaaccagataattcaacttggaatattccttacacctataagaccagctctacccttataaaatcaaataaacctttcttaacgtgcatagagacagagttgcatccacaaaattgcgcaggtccctaacctaaaattagaccttatgtggtcaaatataaaaatgattcgcgtggcctttataagtttcgtaatttcgcttttcctttttacctgtggtccttgatgctaggggcaggagaagaaaaactaacctattctttaagaggggcggatggggaatggtatcaagggtatgggttctttagggccttgaaacaacgaaggctcaaaccagtgggcacaaaggctaccggaacagtacctcctagccccgccttctaggccccgcctttcctctattcgcgctctgacaaagtgcaggggatatagaccaaccgcttgtgaaggctgctggttctgtgcacaggtcctgcagtggtcctaaaggaggccagagggacctccaaccaggcatcaatcctcttgttgaggcttagcagtttgaagaggctagaggaaggtttcaaaccagaaagccattaacactaactctttaaaggctgcggctgaattggcgagatgaagaattatgtagattaaggctgctgtctccctatagaattttttaaagcacatccttcctgttccacacactgaatctgcctggtttgtctcaacttgcttttaactattaggcctgtttggcagaggcaatataaataatgtacaatatttaagacacaaattcattaggtatgggctcaggacagcttgctcttatttaatacttgcgtgcttgtggttttgcacaatggatgggtttgcctttgttcttggcctattattagtgaatcccaagatagcaggcttcctgaagattgttgtagaggttttcagtgttttcaaggatccccttttcagcatatataaagccaccgggagaactagtccacttggtgcagtcttctatactgtccttcacagcttagattcaatctttccttaaagtgtagccgggatactgactcaaagttcttctcttgaaggctttttcctgtggcactggcagatggctgtgctatcttctggctgaggaacaagttccattccatcacgtactttaaccatctgaaaagttggcagtctcacacattctttcttttaggcctatagtcacacgcatttttgaatggcaacaggtctgtgtaatgttcctccactaataaaagagagcactgagaagatgggaggagttgctaatctatatttgtgagggctcccagcgtcgttttacataatatggaataaagatgaaaatatgtgaggacacccgacccagcagtgtggacggacactctgaactgacctgcagtacttggtaatagtagcagtaaagcctgtgtggctgtagtaggtccctagaggctacaggaagagacagagtattttcctagacatctccgttaaagagaaacgatcacatgtttggcccctcttccacagtactaggaagccactcctgtaccttcctggccacccaggaagtactcttcagcttccgcatctgtcccaccagcctatttcctctctgcataacaggagaatgaatccgttttcacgtagttaatcttctacaatgctgcccgcaggtaagccatttgcaagcaactgaggctcttatgaagggtcacaaaagtgattctgcctcaggcgcccaattaaacataatgcttccaaggttctagtgccatgtagaaatgttcataatatggcgagtcctgctttaaaaccagactgtcgcccagcatgagatatggatattataaacctcattctctgagtccaaatcccctgttctactcattgtatcactctgggatgaagaggacacactcccagatttggtaaataaatttttcttcatcaggttttgtcacattgttcacatctctcctctgggcctcgtgataatgctctgaaggagtaggcaggcatcacacgggtttaaagaccaaggcaccaaatacctgaaataatgtcggaccgtgttcaaagcaaaaaagagattcagtgactgggtccactcaccaaggcattagcttcttccattttctgtgtttccgttggaaggacaacatctcttgaatccagagagccacaccatgaaatgataggtatggggctaggggttccattgacttttcgatgctccaagggccaaatctccggtaaacatggttattgagaatcgtgtaatgaacaatggcacctctctcatccccaaaccttttggggacttcttttagcatttgctggagattttactttccttttcacaccaccttggataaacagtttagtatatgccctctcccaatctaacctactctcggtcacttagctcataaattcatctctaatttactgcacatgactttgaggtcatcaggtttaggccattctgaaaggcatgggacctacacacaaggctgactctggagtatataataatcacttacctctcatcaaaaatgttccatcattcctgtccaaaggtttagggacatgtatccggaccaactctttaggtgaaagagatttgactacgactttgaatggtgcagaaaggcaacatggcacagcagatcgaaatctgaagaggaaagtctgggaattggaaagccattttcattgctccctccccaaactggaaatgtacaacagttggaggaatacttctggcacttcggatacaatggagtcatctcctcctctgttgtttaggaagctcattaaggccctctacagctgcagcaaaagtgtaccatgaggctaaatggaaatcctccaaaaatatctggctagctgccaacgacgaacaccctccaagatcttattgtaaactgacgtacgacaatcatattctacactgtcgaatgggagtgataataagggaaactttctaagttgtgtacaacaggctacagcttgagaagatttttcctcttttccaattagttcagctattcagttggttcttgaagagggaaagacttcaggacttggaagtgaatgttggtggcagaaatgtaagaaactagttgggtgagttttcagaactacttcaggagtggtgttctgaatcaaatgtagaaaacaagccactgaggtagttaagggagaggcagattcttgcctcagctcaaatttctccagtggctctaaaagtgaattatgtttggttcctaaattctctgctttgcccagctgtcaagacccccttacccctttcccaagttgggacacacttctggcagctattctaattgtctcttcgctgatacaacaaggtgctagttggccagagccccaaattatacattagcaaatcctgactgacaacagcgtgaaaccagcaggacgacgcgatccgtcggcttgatgactcaccactggaaagcgctccctgcaatccctcacagttcagacaactaagtccgagatggaaggcaacgtgtgactaaaggacgtagacccggtggtgattgttgtgggacgatgaaagggtccggccccaagatcgatgtagtttagggaaatatcttttcccaaggaatgtctctcttgggggaacttaatgcccaaaacatcagagccaggcactggacaagcctttgtccgcctcacggctttaaccgtgctactcagcattcttgctgtggcgtttagcaatactaagcctgcaccaacctgttttctttctcatcttaacctgtctccaaagagtatatctcttcagagcaaccagtgctttgaccagatcagttcataacatttccaccttttcggtaactcttctttttttactccacagtccgcctggttaacagtgaggctttgtaacagcataactttaactat

SEQ ID NO: 27 - DNA, 5100 bp, Artificial Sequence
  Nucleic acid probe
tattttggaaactgactcatgctgaactaatcccctttccctgggagtttaaaatcaaggttaggagatttttaacgtaccgactgctctccaacagcagggcatgcattatgcaaggtattgggtatataaactcagtccctgcccttccatgtattacagtctatagggtacattttagactgtgagactattggtttttgtgcccagattcttagggttgtataagaacattgattcttccacagtcaacatatggcctgaagggaggggatgtcagtttccagagcaaagaaaaagccagactggaaagatgacataatctagaaaggatgattggggaaaagaacatgggcctagagggaaacttagtgctggcatttatattattaaaacacaattataaatgtgtgaatctccagaaagaaatgtcagaccttcttcattcctctcctattatgcaacaaagcacattgatttcctgatgttggttcgttgcttaaaaaccacctatatggcagatctcattcaacagccataagagagactggtttgaaaacttatgcctcccctgagccccctacattttatatttttcctaaaagtgcttacagcttatttgtttcagatatacagaactgccagtcacaaagagccactaagtggcaatacagccacaaacttgcctgggaagtaaaattaacctaaatggatttttaccccctccagtacacatatttacctcctacacaaagtagctgttattagcttatcaccaaccaaacctttgttagtagcagctattttcatgaattttgtgttttcctgatacttcagccagaccaacatgcaaacgttacaaatagagcaactgaacactggccatcaattacctttgaattagttaccgcatgttcattagctttgagttgcatgggaaccatgtaaagtaacccacgcattttgcgtctatgtaagggaatgattagaaggataacaacactgccatctattggtctttatcctgaattaggagagaaatcccccgaggttctgcatggcaaataaagagttaggaaagccaaaatatcagtgggtatatcctgcagcaaagaatgaattccagggtgtcagatctgacttgatggcattttagggacttggccaaggttcccacttgatatttgggactaaaatgcctttcagaggaatttctaggactgctaaccttctattggcttactagggattcaggtcctaatctataacctcaacatgtgctttgaccatgtgcctcccaaagatgaaaaatgttaggagggaagtaaagcagaaagcccaaatcagacagtgaatcagggattaagtgggagtggtggaaaggcaggctaagaacattttgggacatttcagagcagacactgcccagactagatcttttatccttccatgcacatgcacatgtacaccttagttccattataagcttttggtgaaaaagtgtctgtgtcacaatttgagtccactgggtcatcctcatagatattaagcggttcacttggagataagccatcttctggcacctgactactgggagaatgagttcagaaaaagatctgaatcttcgactgtacccattgttgacagagaattcattcccaagggtcagtcgactcagggcattgtcaatagaagtgcttgatgaagcactcctttgttgctgcagaaaactggccagtggactgcttactctagatttctgtgcaggctccagtggaaaaggagggctcagcctcctcttgtagaaggttctctgttgggtagtacagtaatggggagaaggttctcggatttcttcacatgctttccgttctcactcaacctgctttcgccaatggacgccaagagataaatggtcaaagccagatctgcttccactttgggggaagatagtagctacatgtcttctggggcttacatctttagctgggaattttctgccttcaggaagtgaaggttgatcccaagctgacttctgcatttcatcttcagccttcctgctctctgtcatatttgcctcctgacttctttgaggtaattactctctgagtgagaaagtgttttattagtcattttttggcagttggttccaatgtcccctctgccttccctaggctgtgatccactggagccatcacctaaggcagtcatattttcagtggtgagcgtctgatcagagttgacggacatctttagattttcacatgaaggtgttcctgactgctctcgtgtagaataattgttttcccacgttcttttcctgacaagtctgagcaaaaaggtaaagggtcttcatttgaaagagcaaatacacttctcctggaggcagccattttaccctccagttgaggcatgtggctcccctgagggacatgacatagccctgttgatgaggaatggaagtggtccttttctaacggaagtagatccactgctttttttctctctgtggcttcaggtgcttccagggctgagtctgaaaggactttggaacattcattcgctgactcaggagaactgggactaaatttgtttaacatatgtcattggatttatctgtgtgcggtatatacattttggaggtatctttcctggaaaagactctgggtgacttttttgaacttattatggtagatgcataaatcaggtaaagagtttgagatccctgtctgtgaaacaggttggggtatttcacctgccttgtttgtctggtccatcttgcttagctgtttgtcttcttgattctgcaagttgggactctgaggattccttctgtcatcagaaatctgggaaaaggaagttttgaatgaggatgctagagtctgagcaattccaaatgactatccagtgtggatctctgaaaatcaaactgccaaggatgtggctcttcttggcctctggaaacatctgttccataaccagaagaaaatgattcccagttgttctccgcttcgtccaaaggtattgctgaagaaagatctggtaaatgtgttgctttgatggtagaatggcatgttctctgagttctcaaaagtgtcggttctcctgaaaacccctgggatacacatacttgtcagcgggatcaatttccattggtgatggtgccctcaggaactcttcctggttctccctgtctctagaagcttcttgctgtgaaatgccctgtggctggtaaggccgaccgttgcctgctgtcaaaacacagcgaagtactgccaaaagtattcttttgcacataatgatattaccatgtctgttaccactggaataaaagtgaccgaactgtgttcttgatccatagttgaggggtgttctggtattcactgagcttgcagactgttcaaagtcagtgcccaggggcccccttacgataactgaagacctagaccaagcccagacccagggctcatgctctctaggaccacagcctctgtgatctttaaagccacttagaatcccagattaacaatgctgtttactcatcactcctgacatcagaaaaattcctgttctgcttctctacattagtttcagaaaggcattgcctttgcaaactaaatgaagttcctcaaagtgaagctggtggcgacttcagagttaacttttcaaatggccgggcttatatagaataacctttgtaagtgggggttccttgcagagaggaaagaaatcgacccctcttgagtttggagtgctgtctccatggctccaatggcttggattttggatttcctaacgtttcataaccttgcaaacattatccaccccatgccttcactttcttgtgccaaaactccatttgcgtttcctctgtcaaactggctctgtcactcatgactctcatccacttccctttccagtctttattttcagctcactctgctacccagtccttgcctcttgaatccctctccccacctcaaaatttccctttctatttacttcttaatttctccataacaggtctgtctgattccacggcgctggttctcttaacccctacattccttacgaccttgtgttttcactcctgcttgtttgttctatgagcaagccacaattgactactcacaaaggagatttattaatacggaaggagtgctcccatgataacttattggtcactgaatatgatttagcacaccaaacaatcttttctctatgtatgtagaccagtacatttcttgtggcccccaggatcacattcctttgtaatgataatatacagtgaaatgaaaagcctctagagaccctgagtataatttcctctggttttatttctgcccagaccctctacttcaagacctatcctactcaacgtgttcaggatgctttcaacaagacactgactgatcctaattagggctcagttttggactcagaagcccctcaaaataatgctccgtgctttggagttgccataaaaggtgaagcctattttgttgctgctactattcttttgtggttatagagaatttcctggaagactcctgttgatgctggtagccaaatgaggtagaaaagtatcccacaggatgagcaactccaattcactccactgatactgtctgtgattctgacctgactagacaaacaaacctctgtcatagagtagccttagagtacttgagaacttcctacaatctggacaaaaagcccagtgtaagaagtgccacattggaggtgtgttcctaagggtacagagctggcagtatcactggacagtagccagagtttgtttaatatagtacaacttcccatgaaatagtggaatctgcttgtaccattcctgcctggctgccaaccaagtgatgagaagttcatcacctcaaatcttgggcttttcataatagctattcatggggagcaacagtcactgggttccatttcccttatttgcccagctcttgccttttagactgacatgctccaagcagtgatctttcaggcatttgcctttctttttacattccttttctgcctcagaccccgtggctctcaaacggcagaggatggtcagtctgaatacttcccctagga

SEQ ID NO: 28 - DNA, 5100 bp, Artificial Sequence
  Nucleic acid probe
ctctcagagggcaaagctggatggggaggtcatgccacttcagtccactgcttagttaaagcagagtctccgcaggtggcgagcaactggggtccatgtgctatgagaggcttcctctttgagtacaaatgataaccacactcagaaaggatctggtgcaaacccatcctggctggcaaacgtggctgtggctgcctctgctgcccaacacctcagggctgtacaggggcaacacgtggcaaaatatcagagatgccactgatggcataagtggaaacatgatggcctcactccttcctggagctaaagaaactgggtctctagatcagatcaattaaaagtggcaaggcacagttgtccattcaaacttccagcagcttcatggttcataaaagttgaacttgggcacagcaagggaggccggggactggcaagtaatccaatatgcctggagccaggactacatgccagactgtgaaggttctttctggaatcctgctgaaaactatgctggtaaaaggcccctggacttggtcatttcaagggatactaagttacctcatcttttgcatttacccacaagttctattgatgctgtatccacctccagtcattctgcctgacagtcaactttcacaatttctcttctggactcctctccatccttcacatcaccaatagaaagatatgtctaaaatgcaaacttccctggataatccagaaccctttaagtctcatctgctcacattccactcttccacaggtactctagacttgtcaaattaattccacctccattatgtacacacatggaaatatgccaaagggtagtgtttgtccagatgaactggctttgggtgattttatctgtcatgtgtataaaacaaggagaagagtggtcctggcttttatcccccatgccaaaaattctgacagcccttaccagagtgggaggaagctaagctgcagcagttccgaaatttgaatgagcacccaaataaagccacccaggataaaacttaaaaccactcccctcgtcttgtttctccctcttgccaccacaatgctgccttttatctgtggacaccatgcctggaatccaatctaggtgtcttagattcccatgtcttccaactcagacataggcactcaactagcaacgagcaagagtacacagtggggcatttggaaggaggaggggcatctgaagggcaacgcatatcacttattcatagtagggagggcatcactggaataactgggggtctggaacaagaaaggaacaactatagattctgcttgtataagctgatgacgtacaccattggatgccctgtggaaggcatttgtgagattacgaagttactgaaattctatgttaggggctgagcacatgttacagtgtgacagtcaagcccttgacggcttcagcaagtagaagcctgactgttgcctgcagaggatgactgtgtgtccctgggccattagtcagtccctcccaaagaaagttaggttctacctgagattatagagcccttcagaatgtatgacctgatttatatcacctgtcgtaatacaatgccccctttaataaacactttacagtcacataaatctacattgtggtctgctctttctttcatcagacaaaaacagcctcgggtgaaagctggaagtgaaaaaccctccattcacccaagtagtttttatttaaatatatcttcctggacaggacataggccagactcttggagcagtggaaaaactggcaatcattatttcccaatatttaacccatcaatgggccgtaacatgacctgagtttaactcaccctttctgtcccagtgatggaagctttgaagtctcctttccagatttcctgaatgagaacagggaagcttaaggcagtcagagcaacctctctcctgcgcctctgatggttctgctgcatagagaaaactcaaattagtagggaatctttccatgtgtactgtgaatcgttaatcaatttagtgtttatgagggcccaggtaggcaatggattcaacagtgaatgtaaaaaataggatacttgcttgtccatccagtggtcaaagataactagacagcaatcaacatgttcaacttacagaatttgcattggtgcctccttttttgccatttccaccctccgatgcgtggtactcctgtctttgttgccacaacctccacttccttggagccctcgctaattgtcagttccccaaaagcaggcaccacccttttctgtttctgaaaacatcggtctcatttcaggagaaaaataatcacagttcacttaatcgtacctcaagctttccttccacggttgtgtacatatccaccactgcctgcttcttttctgtcttctggccaatgaaaaccaacttcataaccagcctgcacaacgctcttcctcttccatgtgggcttgttatatgttcagcaccaactgggatgtaccatccactttatgccaatgaatccgtctgtgaaatgctgccttggatttcatcctagcacctctggaacacaccactcctcttatgttccgattcctgagttaaacttatttttcctttccccctcctcttctttgtctttcccaacttagtccttgctgacctttgcttgtcacttttgtttgactttattctgagcaaggctttgtgcccatctttaaagtggatgaacaggtacagtcaacagctggaaaattcaaggttcgggtagtcagtgatggcattgtgtttactattaccaaggaagcaaaacagaaatcattcctacatcacatccaaatcattgggaaatcttataaagccgaaagaggtctccagatataatctggccaaacaccctgtcttcgggtgaaatcatactgagctcttgccatctgccggattttgcagttcttattgccaatcctcaccctaacactgctccagcatcgtcctcatttacagagaggacatggaagtgaatagttccctttatcagctcctcttgccaaaggcatccattcctctttttgatgttctcacttctctgtttatctcctctctgacatctctgacatttgtcttgttacacctcgttctccttccaggcagctaagcaccccaatctctgtcaaccctccactccttaaggacatcttgccaaatagtcccttgatctctctggggaaagtgtggccttttgttctcctactttagaaaggcgatacttccaggtaataggtttcttatcactcaccctccagggaaagctttaaaacacctgtggttcagtggctgcaccagtagctttatcatccggggcctggtctctctccctgttgatcttctgaaaagattgttatttgagagcctttgtcgccctgtaaagaaactcaaaagcatcagctatttggcaaacgcccaacagatccactcccagatttctatgtcgtacttaaatcacagcatgatcatgagaattcacaaacacagcttagatggcaagctcaagacaagaggagtgcactgggcaaggctccgcctgtcatcacccggctagctctcacactgcactgaatgatatctcttgtagcccattaaaacataaactacaagcatgaggacgttgcctgtgttgttttctggaaagatgtctgacttagggtatggactcagttagggagcctgatttattcaacccatgcctagaataaacctcctctcttgagtctctgtactttttttgcatgctggttgtctcatcactggattgtcagctcaactagattttagcttttgcgtccctcttattcccctttatacagatgctcagacatcttccactgaaacttgtaggacaagggttaatggcaagttgagctcggagctgcagaatggggacataaatgcctcttgcaagtctggtatactagcacagctgcaaagcaggttagaaccatgaaggttttcatccatagactgttcatggcttctttccaaagacaactacatccttaattctgaccacatcctctgcactgctagttatgtagagttaggtaacaagatagaatggtacaaaatagctattcaatctttgtgttaggatgcagaaagctgaggaaatgcctggaggttttctaaatgtcagcaccatgaatctactgctcttggttcccagagagtccatgactctcaaaccacatctgcttcaggattccctcactgggtcaaaggaagcttgttgttgacatcagttaaagctcattggtttggattacttttccacatgattgctgccttttccaattcctctctggcaaacaaacccgccttacaaagatgagtgtggcattgaaaccctttttatccatctactgtgtcctactcatagtaaagtggtcatgggtagttcctgtgctttgcctcgtgggatccaacattttttgcattcctaccctaatcacccaaaagctaaacaggtattgagcagtaccatgaagagaagccatcaggcaccagagtcttgaacaggaggaagctatggggcagagaggaaacttgcaagaagaaaatcagcgaacagagaagctggaccacaagcctgtggttactagcctggttgattgattggcaccagaatgctgcagtattctgatcaacattccggttcctgaggattgaggaaactgctctatgttccttgtcacctgaaagaacataatgaaaacatccagccataagcagcatgtaggaatttgttcacagaggaattctccaaaggtaaagagccaagcaaatggcaaaattggcaactcttggcatttgcttcagagtgtaattcacaggctagtctatttcacactaactggggactcaggctgaatgttgtcttcaacacacctaagcttcttggttgtttgcttcctattgctcttgcctttcttcaagttgcacgatcactacaaagagaggaataatagacactctctgtcactctcttcatcttttcccagccaattgccctgggaattggcatggtagcataaaatcactgtccgagcttacgtgagggtgctg

SEQ ID NO: 29 - DNA, 5100 bp, Artificial Sequence
  Nucleic acid probe
cagttctttgtcaccagccccatgcttacctatctggacttctttcctatgactccgtcatttaaacccagggtgccaaccattccaagcactttgccatttgtttacaatgccctttctacatattctttgtcacacgatctaacttgtgctggcacttagctaaagggttgaaaaacgccttttcaagtgaggaaaaaaaggctaggaaagaattcatgaagacaggccttaaaagacaatcggaactagacttgctggggaataaactccactaaggctgggtctgaatccattattttcctatgctctcatgtaaagaacaagacttggaggggaaactaaagatttccttttgtggtcttttgccggtcactcaacacagatagcaccaacacactgggagtcattctcctttacctacctcccacttatccacctgccacttcaatgccagcctctatgaagttttgcctaataattttagctccaatccatctgtatctctttcctttacccttgtgcctgctgaagatcttattctcagccttgctgttttaaactcaggggtgtgtgtacaacatatttaagcaaattctggtagaatggccgctttgtctctcttgagtgactctggacaagcttcaaaaccaaaatccacagtgccctatcctggtgggcagaatacactgaagacaggacaaaacacccttctgctgaccccgtaccaaaggggagggtgccggtgaaagccagcttgttactgcttcagggaggagaagcagttactgaaataaagtgctgctctcagcttctctcccagaagtccttcaccaggccatctggagccgaggaacaagttccaagttctcttggccagaattctgctttgaaatctgtttgccttctcttcctccctaatcgaggcccctaaacacaattcttccctaatccaggcttcactcaccacccatggccatccacctaatttgacctggtcccaatgcttaatgttgatggttgacctgcaatcaaaaactccaaatgactgtctaccttccagaaaggaaagcagatctgagccaagagagtaactagggaaaggaagtctaaggaagaagctggagcctcagctcggtggaactgggaatacaaagagctgggtagagctgtctgagggtgaggcctggtgtggctttgtggatggtagaggcaaagcaattacacaaccgtggatagacatgacctaatttattggccttcccagttacctactgtggagccagtgctataccataacagaggtggacagactctccttgatgcactccccatggagaaggccattgtcagaattccgcctgctttttctactctgaggagtgaagagctttttttacgctcccaagggcaaactcgagtgcaaatgtagtaattcagccagcaactgagacctcccctcaaatatgtacaatcccaaaagtcattttcagacaccttgagttcaaagaagccagttcacaaggccaggatctaaccagcttgtgggcacgacagcatttgtgtttatgcataacctacaccaagtgcgcagtgaaacaaacaaccagatctaaatgacaatcagtccacttgatggagacctcatgatgaatggtctacccttggttgggtttgccacctgacgtagaaaatgggaatgtatgaaagctgtgttttcccctgtgatttccttctgagattttatacaaactggtgctatagcatttcgcctcatcaactctttgtggttatttggtttccctcaactcccctcctttctgattttgttgtcaacatcagagtgttcaatctcaggggatggatcatgtgtggtcaaagtctgatatttcaatcccatcatttcctctttcatctagaaagcctaacacaccatggaatgaggaagctggctcctgtgatctgttaatatttgaagtatggccttttaatagggccatttggggaataactcactacagaaaaaccgacctgaatgaaggtgaaggagagaatccacccacgtttacgaaagaccatgccttagctagcacttcttagaaagtattttctgactaaccttcacctgactttctttcgcccttcacctggtttcctcagatctttctttcccttagtttgtattgcctatagcagtaagcattggaaagtgcagacagctcagagatccgtggtgtgttaatctggtactttgttatgtttcagaacttgctcaagcctcctgagcacacttgtgacttaggtctggtacagatgaggggagatcaggagaaagttctatgtgttcaggaacttttctgtgggtggggctgctgccttctgcatgatgaactcaactactgcatgccaatatctgatactgaaattaatacatgtgagcctgaagtgggtaagcagaacaatcccatggagcctgcaagccagacatgggtatttgataacacacactgagaataaccgcagtttcttagtgcctttggaatatgcactgaaattccgtgcagttctgaaattcttcttctgaaagttgggtgtgccttataaagccactttttctcaaggcagatggtatgaaatccatattccccaacctcaaatattccttcatccctcaaagagtcatgtttaggggagaagggtacaaaatgacttgctaaattccaatcaatggcacacagtgtggtctgagagtgatgtcctgaattcccttccacagttatctagagaaggacttagatggatattggcgagagagtccctttaattcattccagcacttatcccacacttccccagcagaaaaactggttaatgtacatcctgggtaattatctgtgtctctgcgggacccaggcccaggacaaaagaaatgaatttgctctgtacctgatcctgtccttctcggccctctgtaactcctcattcctttccagcacctgagtcatttaagaaactgaaatcaaacgccggaggaactttcgtcattttctttactgtgtgtgagttacacttaagctccttggcgcctcctgttaggaagaataataagtccgcccctttgaaagtctaaaaccacaaacctagggaacgcccacacctgaagggctcatattgacaataccttaatgacatgtttctctgctaagtgttggcgccaaagtatttgcttttgctgttgctgtttgctcgcagaacttgagcttgattagtaaaggagtgaaaaaggagattcatgactaattacttatctgaaagaccaggaagaactaaactcaattacactactgactgtggatgaacctggctgaagggcggaaattagtgtatccttggaaacgaaggctagtgctcagagcacagtgagctgaaaggtaggctgagctctgactatgaatgccatgtgatgaaatctagacttcattgtgtaagcactgaggagcgagcagaagctgagttgagaactttgagtgtccaacctcatcagacttcagcactgaggcatgcttcagccctgggttgtggcacaatggggcatactatctttagaaatctctcatagtcccctaccctctctagtctgttggccaaatccccaccacagggcttagttttttccatccttgacaatcaccaagccaagcagctaggtggtcaatgggattctgcccagtttcctcctcacacctggctggcttctgggataataaaatgaatttccagatgttacctcttctctcccttgcctttctgtttagaagaagtgccctttcaataacaatccttgtgcagacatcaagtaacacctggaaaagaaacctttttgtttgttgctgggcagcctgaaaggagtgagattgaaagttaagttgtcctttctccatcaacagcctaaatactggtatgctgcagtctctttgcaaccttttattagatggatcatttttaaattcattatcaggacctactagtgtctgggtggaagactttaagcagagctaactgagttaactcaccaatgaaggccttcagaacttaacctctaatacattaaccatacacattttaggattctagccatccctttttccaccacacacaattgttttcctggttatctttgttgtttcacatggtcaggtgacacaaagaagttgttgccatgccttaagaagcatgctaagtcctgcagcagagcccatggatcggcaagaactgttcaccatacaggggcttcctgcagtgtcatatatgcttgtggtcctgagttagaatgtctaacttgagtactaattgagcagaaaatgtcaaccctcttggaatagagtaacatctgggtcttgtgggaaccatttaatgtgcttcccctcatagcttatcaaattgctcccttgaaacactactgtatcctgcatccatttctaatgcagccatccattcattatgtagatttatgttatccaccgattttccagaaaggcaaataacctgggccagaggggtttctcccatgtaactcactgtccacctgggacttccactaagcattaagccaaagaggctgtaaacactggcaagatacaaccactctgttctgatgaataggccatttgctccacatgactgaatgctggaacaaatatacagaaaaaaaattctgggacaagagtgtcaaaccaatgatccccaaagttccaggtggaaacgggttgtgatggtgtagcaccttgaaagggtgcctcatttccataaagagttgatcacaaaagcagtgacccttctgctggggctcttctcatcctccaaagacttagtgtctgagcagtgctggcttggggtcacaaccttctacaccaaatccccaaactgcctagctctaaagccagtcatgatgtcaccctgttaaatcccccacaccagtccctgtcaggcatgcctgcccaaggtctaaaaatctctgtctcaggaagttctttgaaataggaatccgagaacaaacagaagtacatcagctgttccaggcaatagactgatacaacataa

SEQ ID NO: 30 - DNA, 5000 bp, Artificial Sequence
  Nucleic acid probe
agcatcaaacaattggaaaccttgtcattgctggtacttacatctgggcaattggaaatgatgatgtgactggcatggtggctctacgatgtactaaccaacactgatttatacatgcaggaatccctccccttcactaatctttcaaacctccacccagagttgcatcaaacaagggcccaagtacctgccaaatgcctatgtgttgttgaatgtgtatttgctgagtataggagaaaggaagttttccatgcttctctgcagctccttcccagaagctaagagaattgggctaataccctgaaggctgattccagcaaccattgtccttgagaattattcctgcctgaattagtcctctatactcaaactccttttgggatcttggtatctcacagtgtgtatgtgggctcatgagcagctaggctataaaagaggctcagccttgcactgttgctcccctgcagaaaagctcggatgttatttctctccattttcttattaatgctgcattaggtttctcaacatccctgcttccattgacaaaggagcaatttcacgctgtgctctgattagaagcactggagatgtccaacgagctatgctctattcttactctacccagatcattacccaatagtctagtctgggccacacttacagggatatatatacaagtattggttatttagtaactctcaagtattaaggatagtgtcaagtcagttcaagaagcttatgtgaagggcctaccattagcccagcatgtcaaagttgagaagtgcattaattcattcacttgtgctgctgtctacacaaaaaggttgaaaagggctgaaaaagacaagtggcctatttctttcttacccaagtccaggccaaatgctttgcttatcaacaatcaccttgtcttccgtgcgttgactaactagaaagaacctcctggtttctcctgagagattgctttctttttgttgtggtcaccatcaccatgacactgattccatcctattccttaaatattaggtgagcagctactcagtgtaatgctcagtatagtactccatggtcttttaaaactttgcaacctggcagaggagctgtgaatgagaaacaaggcaaagtcttgttctccttctccagtgtgttcccctcaatgctctaagagcattatttgttaatggaacatcatgtttgggtaaggaggatgactaaaggtacttcagtggttcagaaagaggtagaagatttacctgttggctactgccttgttagcctactagatgcagatgctgtttgttttttccctatgtcctttctgtattttctgttaacaccagatgcacaccagagatgttctaagcaggttaaaagtggcttgggattatatctgattagcattccacttagcacttgaaatacacaaatcctgaacagaaaaggttcacattgcgtagtcccctgcctgaaaaataatcatatccagttttcactggagttgccactaatataaatgtgtactgatgctgtccgaaaatggatggtctacctcacatggctttcatgatctgtaagataatccttactgttcctatcattggttttgctcttcttgtaccttgtaacatgataccatctggcttttactagagtgtcgtttatggccgtggtggaagctcttagtctaacattgggtcaagttctactcttcttgggaaaattctttcagctagttctcctctcccctcttgccagtggttgggggaagcaggacacctcttctccccaaggggaaatggaatagagaaaggaaactccccataccagaaatggaaaaaagatgtgaaagccttgtggtaagcacacagactgactctgcaattttacacttagaaattaatcttcattctgtggaacttccttctggttagtatcttcttccctgggcaccatcaggctcagcatgaattatagtagggtactgtgaaacataataaatgtggtgcaattggcattattgttaaaggcagacttctccaagaatcaacataactccgtctagcatgctctctgggtgcagcattgatattggcatgcacacaggtcctccctgttcaatcatgttgtaaacaccagtgcttaagcagtagtgaagttattgcctggctaaacagaaactctcactcttcaataattgatttaagtcatcatctgtattataggtaatcagaaagaccccaggcccacaatttcttctccaatggccctgcttaggtggtaaatggggtaaagtcaagagagtccctgtactaatagctctctcttctcctgttatagttttctgccaatgtgttccagcccccaggaaagcctcacgggattgcctggagagggtgatgctattctcctgtctggtattgctttctgttctgtctttctttcacccattagacctgttttagagcctctcatttctattgttttgtccaagaagcttgtttttgtttcttggatacctctcctaaattaaaccatctcatagttctcaagcacattctattggtgttgctcagagttttgccaaacatagggatgagtgttggtttccactccctcctacttccttgtccacctggagaacagagttggaaatgaggattcttgaccacagtggccctgggagatctaaccactggtatcaatttagttgccagtcaggatagccagtgtaaataaatcgcgttgtttgtgtctcagaaacagaaacgtgtattgctgcatttggttcattacattcactaatatgcctttgagtagggccatctctgctcccaggaatcctcccaatgtcactgaggcaactctaataaccaaatatgatacccctaagattgggcaaaaaaagattgtgataaaccctatagaaatcctgaaactttcttcctgggttgaccaagagaagtttaatagagaagtcatcatttgagttcgcagtaaagttgaaaggtcgaaagagatttccaactgaatttaaccccatagggctttggcatcctagtaaaacttgaaacacagactttgtgaatgcttcttctctcaggtctctgctctggtctttgcctggcagcagaactctgggtctaggctgcactgacagctgggcactcagaaccaaagtgaggcatgtgagaaactttggaatgacccatttcagaggttttttcaaagcataagatgaatagcccaccagaggtcccacgagaaaattcaaaggacaagatgtcattgtatctgattatagtagttacactcatggatggccaaagctgtaattattaaactgaagagtgccaggggacttggcttttcctccactccttatgttccacagaggggagggagaatcccataacaatgccaaacagcatgagaactctatgcagaaagaattgctatgagctcgatggaggaaacacttgcagtgtgttttaggtgatggctataaatacctaaggggtatgttgatgcttgtgtctacatgttaaagacgaccataatcacccaaatggtatcatgaacaagatgcttggaaaatcccaggaatgatttgtgtttgtgctgaacaagactttgccagctaaatggtctggcagaagcactacgcatatatatagataaccaagcaagaaatgctttcaatttcagtccactcccaagggaaaactagcacttaatcctaccctgccacctgctggcccatgatagaccaaaaataactatctggaagttccaatttctcaaaatagttatttgccatttttacgtaacaagaggacatggatgggttcaccttttagcatctttaggccaagttattctgatcttgtggactctcttaaaggagatcttttgatctgagaccatgtagttctgttcttgtaacgtcttgaaagcatgtcaccaccatcttggttcagttacttctccacaaaagtaagagttcctgagtgtggaaattctcttgttcacctcctgtcctcactcccttccaataagccatgtcttgtttggaggatgggcatagagttgagagtagtgatactcaattttccaatctcctttgctacagctaagctctcccacccacatccggtgttttcctggaggctgtaggtaaatgtgtcaaaaaattcaattcatttttaaggcaaattcaatttgattccgttccagtttccttgctgttgcagctagagtctgaggggcccagaggaaaaggaaaacccagaggggcataagattattcctgaggaagtacaaccaccatggatggaaatcagaagtcactaactagctgtttacttctgggcaaatcttttggctcgttggcccgaaaagtgtttacacacatactcacgaacgcttagcaaagttgaacctggagcaaatgtgttaatagcccttaaagggtcagtgtatgacgtcaccatagtaacaacacaaacctctgcaccgttctggccactcttctgcttgccacactgttcctaaaaaaataatagaaatgcggccgtacctaagggtagggagactaatgctgtcatctatgctccttcctgtggccacctcattttctccacaggcttcatgtccccatccaagaattggcctagaaaagctggaccgctggtctgtttctgttcccagatgagtatacctgtaatgatcactttgacctcctgacttcttcccccaacctccagactccacaggtctgtcaattcctcttttccctttcccttgtacataaaacttactacatagccatggtacacaggtggtgctcagtagcctcactggtgaactactgaacaatttgattccgaaaccttttaaaacaaaactggggattgaggctcactgagaaatgttgagctagctgtgttagttcagcagctgtttttactattgctgaatgactctttgcatctgggattcat

SEQ ID NO: 31 - DNA, 5000 bp, Artificial Sequence
  Nucleic acid probe
taaagggtgtcagagacaatctgagaggtaaatgctacgctatggactaacagtaaggggaaaagaagatggtaaatttgcagagaagatcaagtttgataggagcagttgactggctgcctcttgctgtggttcctgtgaaaggcggctttagtttggatcttcagtggggtaaggggctcttgttcagaatagaggctcaaaaaacgatgatctctgttacagcagctaaagtggattttccccaccccagtaactcaccacatttccctattttatcttcttagtagtacttgccagcaggaaccctattgtgttttcagatcctgtaacccatgtctgcctagagagagcctttggtatgtgcctgcagagctgaatgatagacaattttacatttcctcagcattgtctgacagccccaactgctcgagacgtgcccaaaggtgaggaactccctagtacttccaggttttcacacaaatctgaatggctcaatgaaatctattgaagcccctgactggctttacttgcagaaattccctttgcagaaaagagaggcttccagacactttgcctaactaaataagaaaatggacatgaaattaaactctatgacaagaaggctttttgcccttctaagtaccagaaacccaactacatgatgtccaactacagaaagagtacagacttaacttaactaaccttcccccttcatctccacgtgcacagtcatcgctttggatcctcaaagcggttgtgttttggttttcttttttcactcagagtttctcgttaagcggctgttttagccctttaaagattccaaatctggccttgttccccatcccaaatggaagctgctgacgctggagactgtggagagaaaagggtggggccgaagaaatggacaggaggactgaatgcaagatcaggagagagtctttcaagacaggactagaacaagatgggactagaacgtgtctatagccaaggagcagccgaggggcagggcaagatcaagagtttggttctgtccgcagcatggggcacatgtgttccagagaagctggctggaaagtaacgctgtgggcaagaacagctgcccacctgctatcatgtgatggcagtagaaagacgaccaatctctttaaacacactttccttcctggttatggccttcaattcactgtatgaggtgaaaagggagcttagctggacaagggaaggatgccaaaggctggggcttaaggatcagattcagttgaccctacccaaggcacaggaattgttctatggactttctgctctgctcttatgacttttattcacaagcagctttctaatgtctcaaccttccatggcacccaatactatttgcttagatatttggtggtgaacatgtgacatgagagatcggccgagccaggaaggagcccatgttccttatctcatatacacagaaaccaagggccttgtccccaggcccaggattgtttatttctgatcgtgctcttggtaccagttctcctgagagtgagcttcactgtgctggatcacaaatgtccggtggaaagacatctagctggggccatctctcgagagaagggagggtaacagaggctcttctgatcctccttgcttctggggtctcttgctgggcctctgagtaggtctgcaagtagggctgggcatcggggttgggagccatagaggggatccggttgcactagcctgcatcctcaggctgctgaactgaccacggtgggctaaactggaaaagtttctttgggcaggaaagtgaagcactctcaggaaagttattgcacacagtggaatcatggctacgtggctacctcaggatttgtattagtacacttagggctgctgtaatgacctattcatggtacccactgggggagaatattagtgcaaatagtgcccaatgatggataatcatccagggatttgccttccagtgataccacataagggcatcacaacaaggtctcctccgaatgctgagtgccctggaaggcagagtgaagctctgaccccattgtgttgtgaggtggagagtaaatgacagtggtttccttgccttgggcttcatccattctcaccctcagagctaactggggctgactgggaatgagctaattaggactggcctttccctatatgtgttccccagaaaaggttttcacaagggtctggaaattggaatattttttccacgctaactctatgacctgcagtgtggctaaccaatctagaccaaacatgcagctctggattgttataaaaaaggacagcatagttgctagaaccctctagagatgtacagatattagatagttttggcaggaagtggtggccaaaatacatgaaacaacaaccaaacaatatatttcaggcatctcaagtaatccacaatctggaggcaaaagtcaagactgagttgacagagcacaaatctgtttcatttgactacattaaaggcctgggattgttgagggatgcttatgcacagagaggaaggtttgctaatccaggagcactgaatcatcatgatgtaaacttgcaggacttcatggcttcatatcaccttttctcattttccagaagtatgctgagttccttggtcagccaaaataaccaccctctcgcccatatgagaggatcttccttgtccattttaatgcagttccttagatgctcgtctggacgtgatgacgtgcccgccatataagaaggtgcaatcataggtatcacaggtagccagatagaagacatttttgcaaccagacccgacgacgtgtgtcctggcaatttctctccaagaaaccactagtgatgaagcagtgctttaggtgcttgggggttgagcaccttcctgaaaaagaaggtgtccaggggtctggccttgctggaggacttcgggggatggtagcagcagcataaataacatagaagcatcaaataggagactaagacttccaagaatccttggagaaaccctatcaagtcctggcctacttgaagtcagtattgaggctttccctggcagtgaaactactgaggccaagattgctttcccaaacggcatcccagatgaccaccctgttcctgcagacacacatctgggcttctagccactgggggcaccagaggctcaccgttttaactttttctgagcatattccttttcctgatatcctgagcactaagatctagctcaaagttcagccttcactttgacagccattagagaaagtaaatgcacttcataaactagggctctctccctactccaaaccattgcctacccttccgctggagttactaaatctgcctatgtaaaactctcctttggttcctagaggataaaggattacttcagatttggtgccagacctctagtgaggaggccactagagtaacctgggggagaaatgggtggctcagccactagaaccaactaagaagaccacaattataaaagaccacaacgttttagagctatcttctagtggtacatctgcagttttacctttagtaacagtgccacattcttcttcattctagaccactgcattctgagccatcagtataaacatttatttccctgaatgagatgaagccaaagacctactgccttaataatggacagacagtggaacagagcatgaaatagctgatcatttcataaagccaattgcagcatctctgtccatggggcttcaggggaagagatgaaacctttgttgagattctaccatgtgggtcaggcagggacaaatgactggctgtctccatcgcaaacctgaataattgtagggctagaaatgagctatccctttggctgggctgcggcttggggaagtaattacctcagaaagataagtatttgacaaagtgttgcacagccccttgttctaagaaaaagagttcttggattatgtgttgtggtgatgttgcacgtatacactctctcttgaaaaaacaaatgttttctcactatctgcccttagggccacacctccttgtctccatgtaaccagggatgatggtgggtgaataattaactgctgtttatttctactattgtttccactcatccatatgaacagcccagtccgctcccaacagtgtctccctgccaggttgcattaataatgcaaattaatataagtagtaactaccctagtatggacaaatggacttttgcaataagcagaatcaagacagtgaacaatgcctcgttgagccaggaaaaaatgcaagtctttgcatgttagagtgattggttcaggtcacagggcgttaactaaaggacagtgattcttaagaaccaggcaaagactcagggtcatgaagaggattcacttatctctgaacattactaatcaaccactacttactctgaaattgaggtaacacagcctgaggcaacaaaatgctctgacaattactgaaacgtttatctcctttcacaaaaagaagggcgtttgggatggggcttagaatgtttataagcactgaagagggaggcccatgacatgagatatcgatgaaataactgacaaagtgacgttccagtttggaatccagactacagatagagtgtttgcaacatgcagagtaccacttctgggatgagatggaaagaaggcaggattggacctgaagagagggaatcataagtaataccctaggaaggaaaaaacagaaacgttccataaaagctgagccgtcagaaagcattgactgggaagtatggaaacacaaaaagctaagtatgttactctcattatgggactaccagaaatctagcagttcagatcagtgaggttgtaatccaggcactttcatcccccagtggccgattgcaaacaaaaacaagcaagttttaaacccaagcctgcagtagcaaaatcccagtaccaaagaagtgaggaaagatgtgagaagtcccaccctcagtatagtttagttca

SEQ ID NO: 32 - DNA, 4900 bp, Artificial Sequence
  Nucleic acid probe
agtgtacaagggcacctcggctgaaggagactctaggactctgttctaaatcaatggcctgacctctggatagggaggggttttggataatgcagtgtggcattaattctccagagtagaaataagggttcacactgattttcccagccagtggcagctctaaaaaggagggggcagttgagtagggtgcaacatctacatcctggaaaatgcaccatctcattagatccaatcccactgctttggaattcaaaccacaaactcccagacaaagctgtagatactaaaagcagttcaaataccaccacatctccttcctgcggaagactgtttgctggcagcctatttttctcttcattgttgtggtaaagggctccatcatttctcatcaccagactgtaaaagctacacattgaatttaaatggcctggagttcaatttcaagctctccctttgaaaagtgggaatgtactgtcttcagtttttacatctacaatgaggttgaattaatatgctgtgctaagcactttgtagtggacatgcagtatctgttctccctctcccgacagacttccctctggagatcaaaacagttccaagtacacagacggttttagaatctgtacttttaatgattggtgctgcttagacggtcaaggcaaatttgagtacagccagaatgccacataaacatagccacttcatccagttagtagctgacagttgctggacagctttggagaaataaaacacagtggaatgagtatatctgaccagctcttcctagaggaaacagatgaaattatattgggaaatctctccttctagaaccacattccagaagcatgaaactacctgggaaaagtctaagaaataataaggtggtgaaagaggctatgatggaaatgtctgggacgacctctagaattaacctagctcaaacctctgttcccagatcagtttcatcctgaaggcttccctagggatggatcatcaagtcttgaaatggcaagttctgacctgaggtgcgacttcagacttctgcagtctgccttgtttcgactttaggaggtgagaggaaaattacttccaataatatactccggaggaagcctccgagattcctgtgaaaatgggaggtccacacgcccattcgcgatgacagacacagagagggctgcgcagccttgaacacagaacaggggttttcctatgcagaatttttaaaacgcagcaccccttggtcccggcctcgacattatttctagggatggattgacagaagcgaatcagtttggggacggagagccccagtctccctgccgtggctggtttccctcacatgctaaatagactcacgtgggccccaggagtaatgcctgttagtgactgcactgtggggctcctaccgtccgctactttctgcttccacacgggaggctgtcacctgtcatctccttccccacttagatcatccttccgtggtagattttacatctttgcaaaggagcaaagtgctattttatttttcaacagttagacctgttgggaagaggccagtcagatgaaattacagtacctctcacaatgactaaggagtaagtcagtcacatcttcaatgaaaagccagacactaaatggccgtgaattggacattactatagagaggcagaagggccatagtctttcctagctggaaaaaactggttagctcctgaaacagacttccctgtcttggcagattaacaccatccatgagctcacttttccttctaatatctgggattctaatatccattcaagaaagaatgactacttcaaaaaaagagtctgtctgcttccaagacatgccaggaactcgagaactgagaacaataatgtaaagcttgtgtttcagagtaaagcagactgtcataaaccaaggggatttctgtgatccctgcccaacctcaagttcccttactcgaaatgcaagtaccctagtgtatttctaagtacttgatgaatatccccgggactgtcagaaacctgccttttgtgttgagaaggaactgctatacttctgttaaagcaagtgtgtctatggttgtagatatggtggaaataactttgaaataccaagagttagtgagaagaagctccagggatatgtttatggccaagagaactttagtgtagcagaatagatttgcagagaattgaaatgacccaaggaggcttcgatggagccaaaatcatcagagggagagtctcgggagaaagatgaatctccagggcaggggccagggtttgttggtctcgttttaagaactgaatttgcctaggcattagaaaaagtacagaaagctccccagagcagtctagagcccagcagtcgcaggcccctaggccatttgagttactattccttctgagtgtctgtcaatggaggttgcatgtgaaacaggaaagaggtttgctgaggctctatgtttatgtttgccttttcttcaactggagaatttcttaagatgatgtataatacctcccgcaatgatagcttccccgatgtaacacctcctgcaagttgtcctgagctattttaagctgtaatcagaatatttttttacagctctgaataccataggactgccagcatttcgggccccagtagcaaatctagccctaatatagatgctgtatgggaataagacactccaattcaggccttgtgaagccttgttttatcttattctcagcatctaggttctaacccctactgttctcccattattgactgtcccttgccaggcctctcttacccaggttttttgggccataatgatgcccctgaggtttgtgctcccggaatgccccagagctggaaactaggcagcttttgcacataaaattactttattatgaacttcttaatttcatatcaaaatgcgtagatacctcctgacccccattcttgccctcctccatttccctgttaaatcctgacctttctctgtagacaaaatgctagggtgagtcaccagtccttctctcctaacaaaaaagcattgtactgtacagtttgatttcggctcttgtgtgcaacttgtgattgcaaacttgtactctacacaaagagcggaaaagctaccctttaagaaagagactcatcttcgcatggcctgcagagggcgctcttgcatcatagctataccatgcatagacactattttaatggtgaatctggacagcactccaagggtacattttaacaggttggaagtaggcttttaacatattatgggggggatcaactgagtatagccaaactgaccatcaaattgagagccaaaaagccattaagttatgattctggacaaggctctgattgccacagttcagaaaagttatttgacatcttttcttgtttcagagaaaccatgatcctcctccaggctggttcttaatcagcaccccctcccttccctgggtgcctcccatttgtaggcagccattattggacggcagaaaagcagccctcaaagaaatgctaccatgggatatggcctcaagagaggagcctcagcctgagatgagttggggggtggccttgaagaaaagtccattaggtagggggcccagatcactaataatcagggcgatctggtgaggcagaagatgccagcaagaaatgccctggagggcctgaagcagctgctgaaagatactaagtgactccgcactagcacttgccacactagataacctggtgagagaagctgctaattgtaatagaggcagaatgttatctgtactgtttaggctttgcagaaagctgttaggaacagggttgccaggtgtcctgttttgaactggacagcatggtattttagcttctagtgtacgaaaatgcagtaaatgtacactctacaagaagtttttttccccagtctctatggggtggttatttctgtaatcctatttatggatgaagcaggggaaatacagtcatcactggggccaatgatggagtcaaaatgcacatgcagcaatctgacttgagctgccttgctcttagtaagggtgaacaaaaactaggagagaggggctgctgcagcccccgcctataatggaagctttattggaaaaggcagaaggtagaaaaacatctggaaggacaaaaagcccaccaggcaaccaaccagtcaaccagtaacaatggttattttccatttgctaatcagttggatccatttgagtcaaactgtgcctttgtagacttggttgcttcttttggaagcttctttcaaagtttctttctcagcatgatttccttgatccatggaagttattaagttgttggcagtccacttcttgcttttgtggagttgtatttttgttctagctcttttgttcacagatagtcagactgagcagcctgtgggagaaagctggctgccatcattctgccactcactgctggagggacaacaaggcagaagcagtctggcgggccaagcttttcccaggctcccactgcagaccttcaaaatccatgtcccccaagattctgtatatttaaactggtgtccagagcagtttctatgacctcaaaaggttgttttttaatgatatcacaatgtccaggtactgcacagatgagactgaagttccacgttctaaagccagaagctgcatctgaaatgttgcatagaaaactgttcttttcctatatttatgccactttttctagcttttccctcctggttccaaaaacatcatctctagcctgcctagtatcagaggcactccctgcccagtactttgctacaaatactccagtaaactgggcctgagcactgctttgggcttccagactgtaggcttcctctatctcccaggcataaatagactcttacct

SEQ ID NO: 33 - DNA, 5000 bp, Artificial Sequence
  Nucleic acid probe
ataataataaaaaagttccattgtatctagggaggcttaccagaccccttcccctatgcacagttgctccaggctttgttattagatgttcctcactctgaaggaaatttcagtgtgacaagaatgttgacaatccctgtgctatctgtactaggatctagttccactgtgctcttccagggaaggatgtagctgtgtatcatctttttctagtggcagtttcccagtcgctgttctgtgtattaggaccacgggaggagagaaaaggttattcttccttctccatttttccacacttaaatttatggtaggttttgaataccgtttctccaaaataacctatttcctccccaaacccaaacttcctcaactgggatcgaactcaatcctacagcgtgttctgagattctatactcgcctcacttctcccaagatcgtaaattctggaggagccattccttcatcatccatgtttgcatccttcacaccgttcactggttcgggggagggcgccctaaccgagaagctgcttaatacaaagagctccaggctcctggcggttcaccaggtctaaacagccgggctttatttgtgggggcgaatactgttctttttcttctttctttgcagtagcctctagttcgttagtcaaaacgtgaaaaaaaagacctgctttgccctgggaaatagtaaccctgccaaaagcagaatttcatttacataggcactccttctagtagaacttgatggagctgttaaccggctatacttccttcttgcacaatgggagcgtccctatcaatacgttttttttcctttctcccagctggcttactaaacctccctgggaagtcctttcctaaaaccgggctgttctgttcattctatacatttatgatactaattttatgaaggataaaactggtgcttgaacacaggtactcaaaagcaggttctgtgctttccccttcactagagagcctgttaaacctagtttgtaggaaaaggaaaaagtttttcttagtgctctgaactagcgaggtaatgagtgacctggtttatatttctgagaatttcaccttagtctgacaaccctttgttccttcaaaactttggaggcaacaaagatgtctaccatggtttcttggatcaggtattagtcacttgtggaagtggtcacatagttttgtcttagagaaatttgcctgaaaattcttcttggcccctgtcgttgtatcctcaattttcttcatagtttctctttgcctttggtgccctttttctcctcctcatcccacccttatataacataccatttcatatttgtgggcccagagtaaagagctcattctgttacagacactgaatagccttgtttttgtcagtagtcagctctgtttggaaagcccacacaaaggccttgtgtataggatgccctactattaaaggagcatgttctagtctcagtggattaaatttgctttggtgtgcattcccttaaagatctcacatgccctatttgttgtagtctgtttgatgatccacgggttttgccttgctcccacaacttctgcaaaaaatgcttagaaggtatcttagacggaattccttgtggagaccagctccattcaagtgtcctacatgccgtaaggaaacttcagctactggaattaatagcctgcaggttaattactccctgaggaaaagtataacaagatcaagatctctcccaaaatgccagtatgcaaaggacacttggggcagcctctcaacattttctgcctgactgatatgcagctgtctgtgctactcgtggggagcacaccaaacatgtcttctgttctattgaagatgcctatgctcaggaaagggatgcctttgagtccctcttccagagcttgactttgagaccatgaaacttgctgttatgcaagcatatgacccagagatcaacaaactcaacaccatcttgcaggagcaacggatggcctttaacattggctaatccttctgcttggccttgtcattgtctttggtcctaccatgttcctagaatggtcattatttgatgacctggcaacttggaaaggctgtctttcagaaatgggaagactaccttttttctggcagctatgtgttgttttgttcgaaagatggcaagtgtacaaccaaaccaatccacttgcttacagaaagaatgttgctttttcattttgtatactgcaagttcccaggcaactcgaatttgcaaacacagccatggatacactatttaccttacagtagtttcctgggaatctaaggttttctacgataaagcagtttcctgcttctcgtttggcacgcatgttagatggcagagaccaagaattcaagatggttggtggccagatttttgtatcttgtggaggtacatttcctaagccggaacactcaagcttttttcagggtgtttggctcttgcagcaaaacaattgagatgctaacagggaggattacaagcgccgacctggaatggtaactttttccctcctcagatgaccttacttccactgcctccacaaagaccttcttaccatgacctggttttccagtgtggtattgccttatttttaagagattgtttcagtggttcacataaaggctcgctcactggtttctcttgagttccttacacactatataagttgttctttcagtacaactcaaaatattgggataggctgtcagtatgttaaggatagttgctcctgagtcaattcttcacttactccctctgttgttcttggctggatcctaaacaaatgtcctcggcttactgattgacactttattaaaggaaggctttcatttggtcagcactagaacagtatcttctgaagacaaaactgaatgctatagctttgaaaggataaaaagccctgaagtgctcatcacgaatgaaacaccaaaaccagagactatcatcataccagagcaatctcagataaagaaatgaagttcatctgctgccatgccgtctctgggcaaccaggccccaactgtgcttaagccataatgcctgctgctctctagacaactccatgtacttggtgctttgctcaatctcttatattctactgctatttttcctctgtgctagggaagttgactgtaatggcaagtttagcaacgtagaagctaccgtgaggaaatcttcattaagacttgcctaaccacacaaggcttttaggtaggtagtcttctgtaccttcagatttcacaccaacacctcaaaatccagaaatcattaaagcaaattgtttaaggttccatgagttcttttaagatgaggactgagactgctatagtggtctatgtcatactcccccatagtgctgggcatctaacaagctcaagttatttttggccatcttccggaatttcttgttaaatctcctttgaaatgccacagttgcactcttggaaaacttgtcttcctggttttcaaatttctgtttagaatgccaggtctttgtgctgtactgtttcttgaaactggagactagcacacttcccccaattcaattacttgctttccatttgagaaattaaagccagtccaaacaaatcaataggctataatgcctccctattccctgctatatcaagaccaaaatcctctaattggttttctaggcaaaggctcaatagcctagttaatacaaattcatgtaacctttgccttaggttttatggcacagatcccaggttacttctgggggaatgttgcattcaattttggcttaaattcctacctaaaatgagctattatctgacactctgctctgtacagacgtgtttctgtttccattttcagttccttctccacatggtctaagtaccagtagtttagtcagtcatttagatcactaatgaccccccaaaagataacaaaatttcacaggcctgattattcatacccctacccacactttgctggttttgctgaaaaaaaaaattttaaggtacctgagacctgaggaatgaatgttaatagctcacacagaatgagaagagctctgatcagtgaagtagtaaaaggtagagggcactcctaatccaactctatcacttatacacaacacagccatgaacaaaagcagcagccattcttgataatccaatcccctctgaaacaaacaatttctggcagtaggatttcatttgcattgcctttcctttaacaacttccagtgggggtagttttcctttttgttaatcacgtctcacacagtcaaggtgtcgatctgacaaagagcacaattaacttctctgggacaggaacagtttaagcttctgtttacactgtcatttctcgggcaaagttaccaagctacctaaatacttctttagcataattttaaaaaggagttctctctatggaaggcctaatttggaaatgtacttgggtccctatacaaagtcgtccagaagccagatgatttcccagtgtagtccgccttggattttgaagttttcttaaggaataaactgtagagaaaccatagggccaaccagatggatgatgaaatggaaaaaacagcaattaaatactttttactgaatgtatatgaggttaccagcctatttactagtcaccaaatgacctgagttcaataagacagcctctgggctcaagcttattagccttacaggctaaatgctgacaaaagtggtcataacagagggtttgacttcaaatctgaaccataggcctgttttggcagcaaacatccttcttccaaaatagccagacttcacatgagaaattctgccttactaaaaaaaatgtacaggcattagttgggggagaactggagtgatgaaatggcataaataaaggcaaagagaaagaaaacagaaaacactttcaaagccatcaatgcaaaatttgcaaagtcctgatcatcacacaatacctattaaccccccacttaaacactgtttgtattctaagt

SEQ ID NO: 34 - DNA, 4900 bp, Artificial Sequence
  Nucleic acid probe
taataatctcctcccagtgtggtatggtttgatttttcctttccacttatcttcgccttaggggctgccaaagtaggttttatactatgccttcaaaaagcttacttctattctgcacttgaagctctaaggaactgtgtatatacagtgcctcggccacagaatgactatccttgctcaatgacagtggaataatacacaattattctcattattaggtactgtgttgggcagataactggttccctattccatacttatctctccattcaaaatggactagctgtgaggaaatctttatcttcaatcctttatcttagtccactggaaatatcatcatgactccatcccttaaatctctgcttttacgtttgctggctgaaagaaaagccactttgcctctgaagacagaatcattcagtccttatcctttacacaacaaccatagttccaagtctggtggtttatattctgggctgtgggcctttgatataccagaactactcctgaccttcaactaaacagactaccttcctgtctaccctgaaggtatcactttggtgttgctatccactgattcacttctgcaaacttccctgattgttaattcctttgaaggaaaagggctcaaaattgtgattctcagacttggagattatagggcctgggtataaaatacttccctaaataacttaccaaggaggttgccaccgctgtattctgatccttatacagcacagaacaatcagattataaacacccagcagcatttgtatgtttcagcccagtgcagcattcttagaaaaatcaagggaataccaaaatgaacctccatatcctatctctggactcccaatccagctgtactgaaatctaaaactatgccatacttacttccctcaaacaaagagaatagactgaaatttggcccaaggcacttacatgaaaacaaagctctgataacacagattttcctcatgtgactgaggaaataccaagagcttagatccaagatgaccaaagaggggaaaaaaagatgcaggaaaagtagatagtttgtgggtatgaacaatccctcctccaagttcattttgggcagtaacctttctgctaaagaatgtattctctatactgtgtatgtggtacagtgacagttgggctcttcccacctgggtttgtaaaaacggatgggtgcaggctctccaatcaagaaatgacactttattttgcttacaaaatgcctctagctcccaagtgccccatatctaatcaccaccatatgtttgactttagacactatcctttggccaccattattgcatattgatgttattctgcacaacagctgacaagcagcatcgatctcccagcctctgctcccagctgtaaggggtaagaacagatttccatctcctgcagttccacagttgatccctgaacacagaggtatggtcttcaaaatgaccctaatcttgccaatgttcagagaagctctaacttaatgaaaatcaatgctaccaggaagactgataccttaccagtcccctttcccccacaataggtaaacataactttaaaaaggggtgacctattatagcaatcgtaaagtcgagaattatagtttagcgttcaacattcaaaccccccaggcccgcttccttccagccaatcagggggtaacaagtccagagtttagggagctccgttagacacaatttgaaaaacatcacattgcactcttctattctcctccgggaaacattctaagcgagggcgggtcccccagtcttcatagaatataagaaataaatcaatcgttacatcaattccacggggaaaaactctgtaggcaggcagtcctaaagaagcgtgtcaccgcggttcccaggaagcgttattcacaactgaacagaatagctgctgcagaaagcatctccggaatatttgcaacccagagccgtgcgccccacacgcaatacaccggcaaagggttaatctaggctaagggggaaaaaaaaaatcaccgaagggaaagacaaataacaactctcgtccctcctcctcagcgtcaagaaaaggtggggatcgcaaaattaccgttcggaggaaatagccacgagaaggaaatcgttagctcgaagccccatctttcatctcacccgtgaacactcctcccctttgagaaaggacaacgctgcgacaaaaatccccagaaagaagctaaagtccagcatctttcctaagagcccgctacaacctcaaaagcctgcccctgccccatcttggaagcgcgagccgcgggacccatgactccgggtcctgaattttctgtcgaccttaagtttcttgtcacgagatgacggccatcaccgggtcgagaaaacggcgagaaaaggcggcagaagaaagaactcaattacttaggtgctcaattagggcagaagtgtcagcttgctttcttcaatacagtcaagagttaatcatccctcccccaccccttaatttaaaatctggggttttgctcttggaaactgaaatgcaatcaccagctaatggatgctaaatatgggtagtgtgaagaacagaggtcttgtaatttcatctttcagtcatcagagagcaaagtaggaaagacttagagaggcaaagtccctctgcacctaaggatacactatttttactctaagggggccatgagggaactgagggagcataaataggttaagtattatccatgacagtagggtgggagttaatggaagctcatttatagcagttctacttcctgatttatgggtatcaaagtcactctacctcaaacttttccacaaaagtatggttagtagcagagaggagtgaacaattgccttaaaaacttggtcccaagctagaattctttttttacggtttagccaaggctatttatagtagtgtatgtcaaggagtagactgattgatgttcaccctcttcaaatgtgagtgtcacagaaactgaaattctttcaagtaagatttgttcaagaataatgggcttatgttgtcagttgtgggttcaagcagtaattcactttggactgcttctattcaacagtatgctgtcgactcctgccacaacaatgactgaaaggttcaacaccaggaacccaagagccaggacatctactgaaggctgttggttaagctgtatatgtttctggccagaagaaactctaccttcaaccaagttgtggtgaagaggttatttatgagttgcaccaaatggccatctctgtctctttcctggatgatctggaagatgaaaaactgaaggtgtgtgcactgcacatgagcagttgggcctgcagccaaatccccaaggctggggacaagattctcacctttgaccagctgaccctggacactgtggcaccatcctgctctctgggcctcacaagggccaagaagtgtactggcatttcagcaaggccctgggaacccagcatagccacactaagccctgttataggtaaatgttcctcccaggagtggtaaaatgctgtggctttgccaactaactccagcttctctcatgggttttattgtggatgattattgatttgtcttgggtttgtccaatagtgatggctacatagtcttcctattaccttttatctacaggcaggcagtaggccataatagtattagaagcccctccctttctaatagcacattcaggatcatggcaaaatcagctaagtcatagttaacacacactccttgtctctacgtatgtggtgctaagtcatatttttggtatagctgtataacattgtccagagtggagttttcatgttgcgagaaagccatgtataggccggagtacatgagtaaaaggaataacaactcccccatcccctgccatactttaaaataatttcctcaaccctgccttcattcaaaatgtctggagacagactggcccacttccaattttctttcacgttatcattactgtgaagtccacagtttggttggggttgttgctccttcagaaccgcattatgtaattttagtgtttacttaagctcctttgggctgcctccgtggtagctatgagctcaaagaaaaaagttttcatcaccgtggaggtacagctactgtgaatgtcctacctagtttgggagagaagggtatgtgtgcacaggaggcctgacctggagggttggagattaagtgtgcttttgaagtgcaaaaaacaccatcatggggactcagtctatcccaacacctttccttgagggcttagagactgagcaggggaagtccacaccctaaaccagaaatctgggtatctcttgctcttgccctttacagccaaccagttttcccattccaccaacagaattccaccaatttaattaacttaggtcacaggctgttttacattccatcttcaagtctccaggacttcaaccagtgtacacctcaatatgacatttccaaaactcctttcatggctcttatgtattttacaaatcctgcaactaacaacgcagttcttgatagactgcgatctactgggaggactgctcggttttcacctgaattattgctcgtggttaatcctccatctactccagaaaacgttctaactttttgagacgaactgttgtaactagtctcctggcgaggcctgctatgtcttataccacaggctctaaaagcttatttcccttctcctaacgcatctacctataaatagcgctcaccgcgctgagtcaccaaaaccccgagtttccccaaatagcgcgtaacgactgcgttcccttctaaaagtggaggctcgggggaggaaaaagaaagcccacagtgttgaaaggggcggtacggactgacctgtcaacggcggaggaggaagacc

SEQ ID NO: 35 - DNA, 5000 bp, Artificial Sequence
  Nucleic acid probe
tttcagcaaagctccgaggaagttggtccttttgttccactatttatagattgcgtcaacattgcgaaaaggagaaggcggagcggtggcccctccccctaagccgctgttcctgagcttcatgtaaacaaggagggcggtaaacaccagaggaaggccttacaacaataccaagtgcaagtagctcatttgagtgttatgtttaactttacagggtgggaataaaataatttggcttctcagctcactggcggaagaaactagagctgagctgggacgagggcaagaagcgcgtcgcacagtgctgagagcgctttcgctgggccagtcaagacagcaataagtttgcctctatttgggaaacctttccaggcttgcctgccctcttttaagagggagacctagcttcgcgccaagctgcagtgtccccgacacactgtcaggacacgcgcttttcgcccctactttgttcttgcgcagtcttcccagctgcggagaggtgctagaagttgtccggagcccctcaacccccttggaaaagcgaacgcggatgttaatataccaactccgcagggaacttttggcccgggtcttgagctggcagaggcctcctgggattctaggcccacagaatttttgctccagtgaagttggataagcactccgcctccccgcaaaaaccagagttctcggcttataattcccagcgaaccaacactggcagattggcctgcgagccgctcgctctgttgctctagcacatttatgtagccaaacggtcgaagagccccatctttaagtgtgcttcctctcactggcacctgacgcaaggcgcagccaaggactattacacctctggctgctcggacgcatcttcgcactgcgccccctcgctctttatcaaaaacagctgtaattttcaaagatgcacgatggagttcgcttaggcgaacctcgggaaggcagtcttggtttcctgctaatgttttgccggtaacagctccgcccaaatttctctatacgatgctgagaggcgagctacaggtgaaatagactcctaggcacttctcttagaaaaagccttctatttcctctttgaacctaaaacgttatgcgtctgggcagtccccaagagctatcgaaaggccagccttgctcaccccattggaaccagcatcagaaacctcgatttagagtccactttctcggtgaaatgggaagttgcctaaacgatgcatctggctttagattttatggggaatatgatctggaaaagggaacgcctgtttcagctggggcactcgcctggggtctctggcagtggctgacgaggctttttgaccaaaggagaaagtttgaggctgcagatcacatgtccttaaaggatctggggagagggacgaggcgaaaagaactatcctgggggagcacaagtgttcccgagactttgccaaagtttccagagtccggaggtagcttgcctcccacccccgcccaccagcgcttctccagtcgacgtggaaaaaaaaatcgtctcaaatagcacccctcggtctgcagccctttaacactggtaaagtagataacaaggaactctgctaactcagtttagaacgtcccagagtttccaagtttgtcaagcgactgggaatcttggtcgccgcgtggagagctgcgcccatttccccttccagaagaaatttttgatagtccggaaaagaaacatgacaaatagactcaccatcaggcttgcgtcagagaaccattttccccagactatagtgagattgtttttttcattttatgatttacctacctgctgcagttgctgaatgtattcttctcccctccttttccccacttcatctggttgtcaactcctttccctccccgcccccatcgctagtctagcccagcgtttattaatggtcaatataagccacagtgtttgcatagccctcacaaaatgggtcatgttttcggttcctttgatctgcagccgtttgcttctagcatttctgctacagcaggcttcgccggcagactgatctttctagaccttgtctctttcagagcgccaattttatattgaggctattcctttaaaaatcaaatgagaaaatggagaaagagtttgtagcactcacgggtggagaaaacccatctgcctgatccttttgtcccggtgcggagtcgctggcactactctttaaatagaagagaattcctccagtttgcctccccttataggctttcccgcctcaaattgttttaaatgctcctcacagtctttttgccccctcttggagcccagctgccctgctgagagagcgggaggatagtcgcacctaattagtacgcggctccggcggagatgagtgcgtgggcactgtgcgtgcattcaaagcgccctgcttatgaaaacttggggcatcaatatcgtctcatcactcaggcgcccttagcgaatctggacagatcgaggggcgcagtcttggctgtttagaaaaatacaccttatgcgaacatatacagctattttctcgccctccactccagtggtctgcaagcccctggtgctagtgtgaatattcctgagtttcccgcccgtgcagagccaagaacactgagcggaggtcactgtaagttcagagccgtgcatcctggttagaattgggagaaaaaattctaagcccgcccccttctgtggggtcctggcatcagcgtgaccaggacatgctattattattggaaaccgagtcttgaagtggacccccagcctccacccaagccctgcggcgatcaggggcggagggaattctgtattctagttgtggatctttcgggtctctgtcagttaacagaagcctttggggtgtgggtccgggacgccgctcggacgcccatggtccagtggcagggaagcctcaggctacggcctagatggagcattcacaagggcgtttccaggccccagtggcctctgagcaggggttaactgtgggttacaggcctggaaaagattcatcccctgctcacacatgtgcctctttcttgggacgttgtgtgttctccacactcctgggccggtcaggaggggttctaagaagacaggatgctcagtctcaaggtgggacaaggccagagttccttgcgaccaaactgggatcggaaatctgtattttcctggctaaacttctccctaggtttccttctggtgtgaagatttgaaggattcacttttatcaagggagctgatgtttccttgccaggcaccacatatatccctgtgctattacatatatgagacattcgaattcactggagaatagagagcaggctttatgctctcaggaagttgggttatagggttggggcatttagttcaaggtagcttgggtgtattgactgagcccttattcttatatggcattcaaaatcctcctgcaaacacagtatttctggggctcagaattgccctgaattaataactgtgctagactttccctgggttgtgactaaaggcagctgtcattatcatgtaggccatgtgaggtttgtgactggagaagggattatggtcatgttataatgttataatctgcttacacaggcattggggaaagagagctagaccacttgattaaagtgaggtggcctagagaaaaacagctttacagcctctgacaatgttgacaagtaaagaactgcagtagctgaagaagatggatgaggaggagggacgccctgacaaccaccagaacagccatgcattcagtagactgaggggcctggcatcatttcctctaggactgtttccatttgacataatcaggaaaagtacccacctgagttcaggaaacttggtgaaattcaggctgcattttagggggtgagtggagacattgtggacaatgcatttgggttcttgtttcacacaccagagcttcacttttcatctgcaacggcattctgaccatttttaatgagcattagttattctcaatgagacattaccctagaacctctaccaccatgactaccagcacttctgaattgaaactgtctttggctgtcaatttgctttggggaccttttctcgcgtttgttcattggcactgccagagctagttacaacttcccggtgcaagcatgtgatggccaggggaaggtgtggggattcgtggctgtcctccccatgtggtgcctgagacacagcgggaatccgacgactctgagaacggactttcccttttaaatgctggaggctttgaaatcgtcttcacaatgagggaggattttgctttgtatttaaggacacaggttaaggccgtattatgaaaggtatgatttgggtttgggggagcaggatgttgttatgcgtgaggggcaaagtgattatgaatgacaattgttactctggagcatcaggcatgtagaaagtatagggattgggcttaatacaaatttagtggcataacctggcagtcgcctatttgagttctctgaatctgtcagtcaattatgtgcatagttcgcaactaggacgggatgtcctgtgaagaaaagataggcattaagcccctgtaagtatgaggataattgacccctctgccatgtggtcccctccttccctccatttggccttttatccttggatttgggtcacaacctttggggaagggtggaaacaaagcagaaagcagaaaattaaactttctctctccctgcattctgccaacccactctctctttctcagtcacattccctgtagggaagtccatttatagtttgaggtataaatcaggccattccattcacactactctgcactctgtgatctttcatgctgcattactgggagtgaggaggtattttctagttaatatctactgccctgatttgtattccaggaactagagtctgttttgattgagcgtctaaaaatccttgttagggagggaggagaaaagtgcccaagatcagtgggtagggcccc

SEQ ID NO: 36 - DNA, 4900 bp, Artificial Sequence
  Nucleic acid probe
accattttaatatagcactccactgtgggggttcagtttatgacaatggaatacagccataactagtcacaaaggaaccgagatcatgatataatggggatagatcagatgatcacaatacaactggataaaaagatcaccttgtcactgcattcagtttgtgctgtacagataggccttttattcctctgtcttaatttaaagacacattttaaatacacacacaactcacagatgtatgcagggacatgcatacaactgttaaggcagcctgctttggtcctgaagaatataatttgacactcagatctatacagcctaatttgtttttacgaacactagcagagtacttcaggttggagtttttgcctaaagaatataacaaatggggagtaagttgagccagtatgacctattttattgcagtctaaagttgtactgaagggtgccttggaaaacatgttctccaaatgttggtcaagactgacattcacagaatttccaatgacagcttcggaaatatgatgactctgcaagaaatcaggggaaattcagaatggctgttaagaccactcctaagactatcttgactatgtgaatgttttttctgtgtcatcccagcttgccatctctagctcctgcttcacaggcctcgaactacctctccagtccacttcagtgccttaacagctcagatttctcatttaatgcttcattcacaaagattcttaaagaagcaaccacagtgaggacgtgctttggcaggcaaggagaggacttgcactctgtctcccactccagcgtgtcagaccatctcagaacaaggaatgtttgtctcagacattattatgccctatctcttgatgaaagactgggatagagtcaaatcatgtgtcttggatcaggtaaagccaagtgtattttagcctttaaactgggatatgcattccccacctgctcagactgaaaactgtcttgtaggattaagaggatggggaggtttgctggcctggatttcgagtttagtagagtacattcagctgcattgtgcatttcagctctatggaaccgttgtgcttcgtccctgtttacctcatcctgtggtgggaccatcccttatttagtgaggctgttttttttccccccagacccgagttgttgttgcccatgctgctgaattcccaatatgaataaatcatactggcttccagcagagagattttgtggcttctgtatcctgccgctgcacttctcccttctccagcctcctttctatttggtggtgcagaatcaccttggtcttgatcctgctgcccttgcatacacaacactcccattgacgctagtgaagacttaagcgcatgaaaggattccaggatcaaaaggttagctctatggggcttgtgaaaaagtgagaaggcatctaattgaactatatccagaggagatcatggcagagaccaacaggaagaaaaacatttccaaaccacagcgatattgaaacatttctcttgcattttcacataattgggcttgacagtttcctgccattgtataggataacttcagttgatggtgatcatgtttttgcttaagggtcggtgaagggagcaaacttttccacttaacatgtgcccatggaaatcaggtacttcttttttcagctaccttagcttctgttgggaatttgagggtgttttgtggttaagtaggcttgctgaaatggatctagaaaagacccaggtcagtcactggttgaaaaagggttgacagaaaattaaggagatatgactgagagcagtttctctttcaggtccccaaatttctcctctagccaattcacattaaggtctgaaatttcccccattggtgtcattccggccattgtttccagaagggactttcaaaaatgcaaatatgttcctgtggagcatttgcacatttggtgagtttgtcagcctaaatgagcagttatctttggaactaagaaagaagggtctcggcaatggcagtgacagctcagtccagttctcacagtgtgagaagaacttgggacatccagctaatggatctacaagagttccaggtggccctgaagaaattattagtactgctcacatcatgaggtatcaggatgttttttggcgtggtagaccatctattcagatgaggaagtagagtgcatccatttatgaatttagctgccacagaagacagaaagtcatatatagattctccctttcctcctcattgcttcttcccttctctcacccccatgccccattccttatctttttactcaaaaaggttaaatgacagcagtagcagtcacccctcttatgcaagacattctgggcttcatccaagataggtggagcattgctgtatcactctcatgcaccttactgggtcagatgtaatcctggcccactagccactcatgcctcttgcagggtcatgaagctgcttaaaatgtttgttcggtaacttaattactgttggacagagaggcattctcaagctcctggcccatcctacccaattcaggtagcaacaggaggggaaagaggcagttgtttgtataaggcatgtaaatgaagtcctttggagctgaaattaatggatattttcctttgaagggtcgactctccaggctatatccaatacaatacagggacctcacactgtctttttgtgagggcctttcctgaaactagacatttaagctttgatgagctgacaaaggggggatgggcagacaacaataaccttgcagtctattttaggctcttaaaaataacacaaagtgcccctttataagcatgtgtacaagataggaaactaccaacatgaggggaaaaaaggccaaaaggctgatcttaattattgaactgtgctgtccgagtctttgatatatagagaccctattagacccatcaaatgtcctctcagcccttggattatttctcttccaactctgttttcctataatatgatctatgaggaaatgcgttgtcctttcaaaatgaattttgatctgagggtagtaggtagagagttggggtttcccctgggattcttgtctttaataaacccgagacacaatggtgttttctctagatgtaaaacaaagtatgcagtgccttcatcagctgtgtagagtattgggctgaaagtattccaatttagtgtgtctgccaattcttcaatcaaaaatattgatgaacaaagccctgggccaggtgctggattcccttgaaatgtaggagtctgggaactaggatttggcattcacgggtgaaagaaaattgcagcttttgatgaatagcctgataattatgtcccttagcctgctgttgtggaggaataggagtcattttgaaatggggaatcagaagcaaaaacagccacaagtcaactgtcagctgagtgagtgcctgtctggacgttatcctgcaggcagttaggaagaaacattgtttgaagacgcctgtcaccagttctaaactattgagtcaaataagctcactcaggtattgtcagaatgaacccacctaatctggttaacattttggtgtctgggcaaggtctacccaggatttatttgggtattgcgggagacctggcatagtcagcatccagcagtgtttccagagtggctggccttgctgcttctcctcacttgctcctcaagggcaagagactggacaatggctggttataagtagttgaataaaatccctgcagttcatccattcttctcctaccagcaggtgtcattatggaaatatgaaaatacttagaattactcaaatatcagtaccaagtccacacccttaatcagagttgctagggcccttcatgacttgctgatgatcactctaactcaactcttgtacccttggaagctggctacttcacccaacatgaatataccaaagaccatttggtaaatgaccagctcaccaaagctaattctgcaaaaatcacttgaccaaatcttaattacttttgcctctgtccctattgctcctgcttctgcaccttcctgtgccctggtgcaatggctatgagaaattgctctaaaccctgtgtaaaggtacatttgatgtaaatttgcatcacttcatcagaagtccagaacctgtgggaatgggggtgcagagagtgaagaagcagttttagactgacatcgttggctggggcagacggaccatctctgccaggaactctactcctctgcctaaatgaccatgcggccccccttccaagggaagtctctaggtggacatcatgtgaccagaaattcctcaacatttttcacagtgtatatgccctctctagtttccagtactgataaaagcttttccctgtttttgtattccgcttgtcacttccctaacattacctgagatgagagaggcattatcgtctgctttcagcaggacactctgcttggagccctggtctgggtactcttggattttctgctctcagcatgacagaccctagcttcagtcttgaggatggctttcagaaaatcacggctggatttccttgcctgtgatcttgtttttgaactgcctaaacctgcactgtcacagaagacgaaatggatcctccatctgtctcaccatacaacccttgctgtatgtgtcaacttgggttgaaatcttatagatgctgttgtcgatctctgatagcatgactctccataaagaaaagcccatgcttactcccaagatgaggcagtgagccttaataaaaaaggtgctggaagtaatccccctttaagacagtgcttctgaaccagattcctatataacttctctcaaatgcagacatagatacctgcctctgccggtcttgatgctaggatagggctgatggtcataggttg

SEQ ID NO: 37 - DNA, 5000 bp, Artificial Sequence
  Nucleic acid probe
actaaggtgtttaaagtatgctcatgtcacatctccttgttctagcattgttaggatgaattgcgaactcttgactctgaaactgggaagtgctctcttttttcttaaattaacttctggggcaacatcaactctggaggcactgtctcctttctctaccttaaggataattctgagactctaaggacagaatggtttcttcagccaggtgcatttgctgtgaggctaaacatccaaacagatttagagtgagcgacagtgggtattcaggtcaagtccaccagtttgatgttccttcttacactgaccttccctggtgtatgcagtcctctggaacatgttctgtctagctctggtgggttttcttgtgagaataagccacattgctccagaattttggtagtaaggtcactatgaggagaggtatacagttactgttacagtatttaccaaacagtgggcccacagccacactcctcacttctccttttggtctctcctgctgtgcctcattgctttccagtctgaggatgacttctaccccaaattattctttgagttactcctagcaggatgagagcaggacacagggctctagggcgggatgctaacatgttgtactgatgttacttctgtggcaaaacttctcagagatcttgggcccagtaactggtccaaaagaaagacttgagaggaagaacctgccttgtactcacccctttgggctaatgagattctcagctttttccaaacgctgactcttctctcatctctaggtcctttgggtaaacatctcttcccctcctgaacagctctgacttgctttttttctgaatccagcatcagtcctacaattagtccattggccaggtgatggtcatgtgcctggttagagtagggtctcacttttcttctcagtcctactgctcgccccagctttatgaaacatgagctatgtcctaagggaagagcaagtgggcctcaggtcctcagaaagcacggtagcattctgccttagatatgagagacacttcccaggctcttcctgcatttccatcttatgctctggcaagcaaatgccaatcttggctcttttccactattgtttgacatgtgaatgcaataaacctctacccacataagctacctccattgaaggattccatacttggcacagaatttctaactgacacacactaatctcagtgcctctggcatctccttcttttgctatgtaccagccctgtcacctagtccctcccactattaatcattaatgacttaagatgttaccacctcctttacctgcccaacctcctttaaacttgcttctcatagtattcatcccttcccctgaatccttgatcacttgtactgctcatctagcactcatttgtacattaaaaatgagacccagagatcttgtgattacacaagattctgtagctaagattacatccctgttactctgactgtccccctgccggcccccaccaatcccatcctagaagaggaaggccaggatcaacatgctgatggattgactggccctggatagaagtggggtactttctttttgaacaagaagtggggaaggtaaggaacttgagcaacatggaacaaatttggaaatgctgctgtaaggaaacacaatacaggaatatgcagggcagaactgaggtttgagctttaaaacgtaaaagagaatggagatgttgagagtgttgttgaagcgattgactaggataggctctgaattattgatgaccaagagttcagaacttgttacatataggtctagttgatggatggtaaagggagtgagaattttttccagccacttgtggaaaccctagggtgttgagtcaccgtcttcaagatggagggagagagcatgtctgccagcagtgacatcacggtcagaatgcactcttgctggatcaaacagccctgagaggaagcatggggcctcatttttatgagaccaactgggaatagcctgcatgggcactgagccaaagaaacagtcttatgaggattttctcagcatttcctctggccacaggtatggaaggaagaacatcaggaaggattgataaaagttcaaaagaagagactactgaggaaggcaagaacagcaatattttggaaaagacaatgaagagtgagagggatggccttgtgatggggcaggcctggagcctctatgtctctacatcactcaaaggtctatctactccaaactccaggaccgggccagttcagatgctcatcctgcctctcaaatgttttggtttttgtttaacagttgccaatactccttcttgcccttaggtttctggtctcctcaaaattagctgtagagtgggacacatttacctggttacccttcacttttctatacagtgatacagaatttcaggaaatgtagacttgtctgttattcagggacaggttggggatgtcttggctgccgcaggactgtatcgaccccgccaccgcctcctgccttgaaagtcttccttaggcagaacccacataggatcatgatggatttgggtggttgatgtacctgcttggtatagaaggatcaagggttatctttgggagaaaacatgctcagatatacttctttgggagaagacatgctgtccctcttggctggagctggagctcccccatcccgtgcctctctttcttagccccctctttcaagctccctgtcctgctgacctatgctttctcttgggaaggactgtccccatcctctggagggacatcctgctgctgtatcccttcttatgtcttgcacacttgagctcatcaggctttctggcagctaatggaatacccttccttgggggatggggtgacctctgggccttaatgcttggaagggggcttgagctggactctgaggagcactggcggcagctctcatgtcgtctctgaaaggctgagctagccctgtctggcagaggccttcacttcacagcactctcctgttctgtttggaattcatttcccttagtgtgacagttgagataagctatttggactgcctccagggagtgctccagaggtgggcagggaggaggccttgggagctcggctcagccatccctagtgaaatacagtggaaagcggcagctggaatcagatccacaaggaatagcctgagttctccagaccaaagttataagtcacaggcagggctatggtagtcattcctaggtatcatcccagtgggacttgtgtacctgggatccgatggtgggactgctggctctggagggctaggacagcctaatggcttcacaggtccctgactatgctttagatccctgagagtggggacagtcactcagagccatcaagtaacaccaaaatgcactttactttctgaaaatctagcaacgatcccattctgaaggcccaggagttttgtttgtggatcaaggagaaatgaagtggcagagggtgcattttgtttggaacatgaggcaaagggaaatggagctggagtgcaaagggaggggaaggcctgtcaaaggcaacacatagtagagtgactgtctttgaacagcttcgtgtcagacctcagggcttatacaacaagaaccagttttctccagattctgttccttagttggaggccaaatcaacagatgtcatatgtttccatccttccccctaaggttgcgtgctaaggttttatctgatgatgactgcaagaattatttccccatgggtcagtccccttggggtagttgtataattacttgcctaattaatgtataagactatattgagtgaatctaacaccatccctggtgctccaaaagcagccatttcttctcacttgtacttttgacccaggggttcttgggctcattcacataagcagactgcagagaaaggaagggcttttcagtttactcgtcgcttctagttggctaagggcatcttatttgagtaaatatgggtgtggtcattaggctttggaatctcttgatggacacttgacatctggaagatttggttttcccaattccatagttcttgaacagttggccccattttagactctgacataataacttaaggatgaccttttgctaccaaaacagtttaatgcccattctacccagtcagccctgttcttccctaggttgctgactttgattaaattcatgggtcggtttattaaaaggccacaatggtgttatctcacagaactctctggaagaaagtccctaggcctcaaggagaccaggaagaggacccgaaggtcagaaggttgcagagccagccttctgagcatccctgatcctgtaaccacatgtccacaagtagaacaaacaaggtggctagggtaccctctttcccaagattggccaaatctaatctagaggcgttagtaatgcagaaatgtcatatagggatagacacaggggagatgtaagggaactgtttttactgccctgcttctaacatgatgagcaatcaaaaacacacaagtttataggcttcaggccaacatattttagtcagctgaaatcagggagacactgcctggttggctggctgcctccggaccttccccgtggcagctgtgaacatgtggatcccttgtgcctgaggttctgctggtagctcctgcagcccgccagactccaggctcaaggcttaggggtgttgaacatggagttgcttttctttgcagttggttgtagcttaaaccatagtgcattgctgagtcgtgtgatgtgtgattcatcaggcctcaggtgacaacgggagatccacgggtgctctagatatagccatatttcagcccctggatcctttgttagtgccatattaggagaatgcctgtggaaatggcttcttttctccaagtttttttggccccagtgtagaactagtgtctctgttctctgctgtgacctcacctggaaatgtctattagtttatcatgccatgctggattaaagaaa

SEQ ID NO: 38 - DNA, 4900 bp, Artificial Sequence
  Nucleic acid probe
gttgagttttgtactgtaagttccaccggtcagcttctcagccagacttgggtaatttatgtccagggtgacaggaagcactgaggaaagagcgttctcagcagggggaatggcattgggatgttcccatgtctgaaggggacaaggagttgggaggaactgacaaaagaccactgaggcctgaggtgtggaggcaggtgcggagaaactggccctagctcagctgccagtgaccagtgagatggattcagaatgttgtcctagtgagggtgctcagtgacaacgcaggtcagaggcaggctacaatacacacagctgctgcctggctgcgtgacaaaggtgtcttggcagaagcattttggctgtctgaaatacagcctttgcacaatcctgagactatttcattttctaacatccaggcacgcttttatggcatctggagcattttgctgaatgacttggtagtgcactgctgaccagacagcacatttccagatttatgggctactttatcccatggggaccaatcctgtaaagtgtattcaacagagatggccctaggtgtttgcttttatggttgttagccacattcacactgggtcttaatatcagaaagaaaactggggagagtgtcaagaattggcatgcacaaactgatagaattgccagtggacgttcttattttctgagcatttatgagacaaacatttttatacatttgtactagagatgcccagtattgcttgctcaccctcagtccctgttgtctttaataatcgtgttatatacaacacaatttctggtcccttgacttcacatccagaaatgtctacagctcagactgaccagttaccaggttctgtgtgcactggcctacacagatattatgagtccttcagaaaaggtcataggtagactgatagctaaggcagccattttggtcatgtgcaaggtatcagttggtaaagggaaaaaaatgaaactcacatgtagagagagaggctttcataaaacaaaacaacaactatgcaaaacctacctgtgtcctctgtggttggattgaaaaaaaagggggtaggttgtgaaattaagctgaggacttgcctgcaaacccccacgctttttagcaaatattattttgaatgtcagagtgcacagagccgttctctagggttgggctaaatttgactctgtgggagaataaacctagaattccaaatgtggtgatcccaacagagtatgtgatgcaagaccaattttccacttgtgccgggtgggtttgttcaaatctcacacagaaattctttgtgcaaagcctggcggttgcatgaagaattttctcctgaaatgctcctctatgcaatttactcttggttcaagtcgatgcttgtgatgctgggaaaaaccaagagacccagaaaccctcatttaagcaaacagcctttgaaacagaagcctgactttagctatcacattcatattcttatgccaagagagatcatattaaaactttggtttcttttagggagggagatgttacccttgtcccagtggaagacttgtcatgtgaatttgctggtagcctccctggggttgtgagctttccttctggttgattcaatcattcatatcctcaagaaaatgtgccaatctggagctcaggagatcctgagaatattaacaataacagcaggctcaagactcgaatcttctgttccctctgtcgagatgcctcgtctctctaggtcttgctgacttgaatgtgtgtggagagttcaaagccaggaaaggatatgtttgcctcactacaggccaccctcaaagtgggccagttcttagggctcataggggaactatgacccagtgtacccctacagaaaagccactgtctccatggctttgtctcttttcattatgtccatcctctctactcctctggattacaagtgtcgtgagaaggggggttgaggctcctatttcctgtagggcttagattactggaagcccttaaattcttgatgtaggaactctccctccaacccagagtgccagggttttgcttgccacctcttctgatctgtccttaccttctccgtgtttctcaagccctgctcccattgcattggaacagtgtgtagctgtgcacactgtccacacttaagaaagggtgatgccagactcaaaaagccttggggaagagtgccatttctggcctcttggaagcacctctgtaaatagttctgcttaggtgttcagacctgggtgggcaagaggggtattcagtgatttgcatggacccaatcaccttccagtgccactaaatcatgttttctgagcagagcagagtcacagctccctactttaccctgtggtattcagcaggccaaagatttaaggaagtaccattcactgggcactattaggtactaggtgtccggggatatttctattgctgttcttagctctattgttggtttacttggccagtggttgctagcttgtgggtgtaaaaaccagacttctctctctgtgaaagttgctgacattttataccacatgctggaccgttaaatcacaatttacgtggtcacttgtaaatagcacactttgacatgcttccctgttgtgtgcctctcccctttttaaattgcactggatacttttgttctggaatccttggctgtctaacctgaaagcagtgttggaggattacagaattgatgctcaaagagaaggctttgctttcattgcttaagagggtttcttggagaggttcagcaagagaaacacaaaatgagatccttacaaaatatcacatatgagaaaggttgtagtctcttggtccaaaggactactttcacctggagatggttggagttgtagtatctgagttagataagtgggccaggaagaatcactgaccaggaaggtcacacaaggcctgagtcctttctaacaccatctgtgcactgtatttcagggcctagtggctgcattgaaaggaatggaaagtagttgctggttatgttgagaaatatcaggaaagtgtgatgcctttctcctgtattccctcttccctccctcattttttcactttattgataaaaataatcccaaaagatcagagaggggttctataggtgttaaaccactgtgttatgacttgaactacaaagcccttgggggcattcaatttcctcctctctccctccaggccagaggccatgatctacatccaacttctctgcaaattcctgaactctgactcgattgtccctgtcaatcctgcctttgtgtcttacctggcctcagtcctaatgaggctccttcaggatgcagcctctgtgagctgcttttggcttgtcatgtaacagctctggctaggactgtgctgtgtaggagagaactccacagtcaggatctttgtgcactcaggacgggaagcatggacaccctggagagcatttgtattaacatgggcatttaaaaaggtcatacagcagaataggaaggcacactgaactctgcagctggagtttcctcagatgtatttctgtttccctagtctcgagagctgggcttttgcaactcactgttataccaaagcagattgactttttgagttttgtattgcttccaactctctcaatgtcagaggcctcaaattaattccaacatccccttgctgttttcccttaagaagctgtgatggtgttgcattcactttggattcagtccatttgtgtttattggaaggccccttgaatcagcatttccactgctctcccacatttgtcatgtggtgacacgtggacagaaatccccctgcacgtagacacacatatgcctgccttgacttctgattgaccttatcccttgaacatcaggcttaggccacttaatggtggaagaggaactttgctccatgagttgtgagtgtatgcacagcacaggtgttcagagtgattttccctcccccacgttcactacaaaaagtaccggaaatgccaccacaaacattccgagagcagcattgctgtgaactgcaccactctccttttgttccattgaagatttcagaacacatgcttctttgctctttgaaatggctttctgagtagtaatgggattggaagagttcggagaattccatgtatcctatggtgatgtctggagtgaaacatggtatgaatcaaattgtcacagctcactattatggatgtcagcctcaacaaaaccaaaagagcaaggcacataatcgatcacaatgccaagaccagtgactttcagatcagttattttgttacacatcattgctcacaaggattttgctcagtgtgacaaggaagtggcagtttgcttgctccttccacactcttgaaggcctatggtgctctaaaggtgactgttgacctagttagagcagcctcccaagaatgcaaaatgagacctgggggacacacatcctactctgtattttgcagctcatcagtaattttatgaggcaattcagctcagatggaagtggtaatcttgcaagaaacccaatttggagttgtcgggcttagaagtgattagggctctgagaaatagatttttctcaaatatgacattgattagaatgccagtgtttgaggtgatgacaagagagtgtttttggaatagggactggaatttccagcacacacattgagttgattttatatgattatattccccagacccttgcatttaaatacaccctactcttcttgcccagtctgtccattggcctttaaggaaataccaatcttgtaatcaatgatgtttctcggcctcttcaatggcatgtgggaattagtgtttcctgggtgaaggttaatttgggggaagagtctagataactcagtggctttaggac

SEQ ID NO: 39 - DNA, 5000 bp, Artificial Sequence
  Nucleic acid probe
tgttgtgggactgccatccaagaacctgttctctgtatctatagcatggagctgtcctccagagtgctctttagaaagagctccaataattgggaattattgctatcaacaggatttccaagcaaaaagcctggcactaattattgccctctctttagtgtctttaactgccagatctaacatgtgttaaccttatacagtttggaaaagctgtggggtggaacaccttcttactctttgggagcaactgggaaactaaagagaaagagatatgtcttgtcttgaaggaaacttgttgacctaaatctgtgaactattgcaggtcactgctgctgtgatactttgggaatgaagtcccaatgatacctctaaacagtcacagctgtttgagctctctcagatggagcgtttggatactatgtttttgatgttgtgcatgtgttcttggggacttgacttcatgtcaataaaggacagcagcatctcataatgccactgccaggcttgaagtggcaaatgtgagccagtggtcatagcttgagctatctgttttctgtatgcactgcatccaaggctggctttcaactcactaaagaaaataaaattatgatgatcaactctagtacttccagcctgcagaaatcttggtcctttgtatgacatgaagttgccaaaaagatacttagagtggtttcatctttgaatttcacagctttgcatcccgagtcaggaagctgtcatggcgttgtgctggtacaccagtgcggatgccagcatgtgcatatgctgacacatatgcttgaatgttagacatctctcatctaaattcagagccaaggaggtccgtgtaggcaggctttccctaagcttcccttctggctatgatttgaaattaatcatatgagacttgacaaattttcatagcctaaggctacagtctttattgccagagcagtaagccacggaacatagtcattgtggtaggaaagatttttgtctgtgtagacacctctaaacaagtaataacaaactgatattcaagcgcaagtggtccactctgactaattgtccacctctcagggtttagagacatcccttgtataaacatattgtctccttattcgagtcgactgtcttgtgggttatagcagtttttgcaagtagattctgtcactaaaatgtgctggtgtcaccagggcccttcccctgggaagaggaatgtgagatggggtacggcccaggcatgtggcatcccagcctgcaactgtgctgctgattgggtgtccatctgatcggctaacatttttcccagcatgggcaaagccaccaagctcattcattaccaggccaatcagccgtatccacatgtagcagcacacccaccatcaacttgtactaagagtcagaaactgcctcgacgttttgtggtgctcctgacgtctgcacagccctacttgcacagtctttcctgatgtcatcacgtcttgcctctgctactcaagtcctcagagaactccccactcctctcttcactacagaggcataagacgcaggccctgagggattgaatgtctcggcaccacccagccgatcctcccactcccagtctccagggaagtgaaagtttgggagtatcttcatccatgtgtcaaaaaatagtgaaagcctgcagaagcacaaattcagcaaaagctcacttctacattttcagaaatctccaacgtttggatcatgccaggatgttaactttttacaggccgaagctgaacggcctgttatgagggagccccaaaatatgtatggttttgtttggttgccctgttgggaccttcactgagtcttacccaggggttcttatagtgtgtctgaggtgtgttccccaactggccctgggtgcagactgaaaagcatatttaagcctgatggtttatcagcttggttgactcccacttctgctctcaagtgttagcagaggaaacttggtggacatttttttggagatttctttttccaaagaaagcggccattctgcacatctggagagggtgccgtcatttgtttggttcccagatgctttgcagtccacacttctgggaagaccaagtcatcattgccttcaaagggatttctttctcagcaaggtgataacgttcactgccatagcctcccctgattaatgctttaagattgattgctggtcatggcagtaaataaaacacatgcctttcaacatccctttttcccctcagctcacagcttgggagtttaggttaactttttgttataacaggcgtttgagactccaactgctgctaatattacagcttaagaacaggctgatagatcttgcatacgaaacatctcactctgggaaaatcttcacccttctcattcttagttagtggggacccttccagctgaggcccaggacacctttgtttgtcctagaatactcttctttgattctgccctgaggctacatctggggcactctaactgccacctgagcacttggggaaagaacaaagtaaaaagctagcatgaaggcagtttacatcaaggtaaagcttccagaatatatttcaagtcagcctgagcaatagtgtgccggatgattctctggacacattgtaaaacaccacaagcagttagtgcaagggtgtgaaacacagcaccttttttgagttatatctggctgtttaaaagcatgattttgggaggaaagagagaagaggggatcaaaatgttttccctccttttttcagtgggagagcagagaagaaatgttggtcaagatgcccccataagcctacccatttcccgaagtgatttgagatggattgttttcatccaggccagtcttggaatagggatgcccagcagggcccagggtctggcactggagaaagactcacctgtatttgttcttcctccactgaggaacacctctgccttcggtttattgctgttttctctggttacaattttggttccattgaaaagatcccagctgtttcccagcttaaatttagcagggaggtaaatggaccctctgtgggtttagataaggtgtatggtcaagcagatgttgtttttccttttatggtggaaaagttaggctaggtggagagcattcagtgtccttcagtcatacatagtagttatcacttgctgatcccaggaaaccggtaatgctttagggctgcacggatgcagggacccatgctctttgtgacttgtgagactgataacaaatatacacgcgccatgtttagtggagggttacgtggtctttgtaaggagaatctgagagtgaggacccctctcagtctcaggctttctggtctaacactgagtattagggagatctctggtcattctccaagcgcagcctctaacttgatactctcaaacaatgaaaatgatacttcatggttccttaagcatggcagagagaaccaccagatcacataatgcaactcatctaatattcctattgaaactgaccaaagccgaccatttttgtgaagtttattctggtggagacagtgcttgcaggccacatccactctgttcttgaagggagaaagaacaccagtgcatttttcccggcatgcacaatgtcgccagtgcatatgttgaggtctttgcgtttgaagcgtggatgctgtttagtccttactgcatatatttattatgttctcttgttcattagttccagcacccagcctgctgatagacacatagcatgagctccatgattcctcataggaagattggagtactgtagaaattatagcccaaacatagcttggaggagctgtcatctagtgggaaaaattaatggtctacataccactgggtcctagtcctggcattgtactacaatccttgcagctgtactggactagctatcagtcaatcatatgcttataaagattccaatcctacctggctcaagaatttcagaggggacttactgagttggactggttttaatggacccctttgtcctccagtacaggttctatgagtttgcaggatcttaaaatttgtgtatgtaaggcctcgaagagctcggctgcctggttctgtcctgtagattcaaattatttcattctagtttgatggcagctttcccttcttctgtgacacatcttcttgctattggaggtggctggggtcttcctggtttcttccacatccatttaaatgatgttcaaagaggaatggtgggacatctcaagtcaaacaaacatcctgaactcagtggatgaagtctccaacaggtttcactccaagtttcagcgaaagtggaagcttcagaacctacccctccaatttgctgaagaaggtgggacagaaccaaggaaaggcaataaaaccgtgaaccacagaaactcccactctcactgtctccatttcagaacccagttccttttccctctttccgttcatttcgagaagacaatgtcagaaaacagaatgacgtatgtgtcccactttttattgactccctgccatgagtagaagcttaagctacttacacaacacccgtctgtcccctctcttccaaattttggtaatcattattttaattgctaaggttatgttttcttctctttatgtctagcatcacaagctcttgaccctcaagagacactatgctcactatacaggtcaaaaggataaatgcttttattcttgaatgaggagggccactggagtgtcattgtctcctaagcagccttaattcttcccaggaggttgactgcacttcttgagagaggagtcctctagctctggctttgttctcctctttgccatgcccatgccaggcttgccagcaaacagcctgcagggctagttatcttccttctgtgtggctcttctcacccttagcaggaggataatagagttgcagtgcgtatgctaaacaaaagacgactttcattccaaggaaatgagatagagacagcacgagtcttacttctgttgtt

SEQ ID NO: 40 - DNA, 5000 bp, Artificial Sequence
  Nucleic acid probe
agacgcatccttctcaaatacatcctcaccccctattctcattctggctgatctcctgtgtctctaggaactttgttcagatattctgcttcctcttatttggagcctatgcatttttccctatgggcattgatgacccacactgagtagaatgagcaatgtgggtgttggattgctgggcaattcttatgctccctggaaatgccagcagcaaacatcatttccaaagcattcaaaccacctatgactaatcacaggatgaccaatgttccccttgtgacgtggtaatcaagtgcgttgcttcactcttgtgtgcaaatactcttccttaaagacacatagagaaaagctggccaagacccaagaaatggctgtagaaagatattcattctcagggcatgaggaaaagtcaaagggggatggtcaggttcaagcatgaggcccttggagactgctggccaggggtgctgcagagtcaccgtttctattgtgtaaactttttcaagggcagttactacaagacaagttttcaggaccaacttttcatctttgagcggatgcacagtccggactggttttcttccgtaagagagaaagaggtatacttggaagtgaacagataactaaatttaatgagatggtctgggacacataggaaccctcaaaatgttactagaaggttatggtgagcctagaatatcttttatgaatagaacccctaccaatagaaccaagcacctgtgaaatccaccctctgtactgctcacccagccttctctacttgcaagttgtcccaaaatcagccattagagagtaaaattatgtggtttggagttggtttgccttttcaagccccaagggattataacccttagttatagattaccttcaggtgtcaggagtgtctgaggttgtcttttaaagcccccacataagtttctgatcatctccaagagttaacaagtggagtacgcctggagatattatgaagtagacaattatttgtttcccgtcccactaaggtaaatagctcagtgcgtttggtgcatttatgattgttgtgcagaggagtgcagctgatgaattacttcctttcattttctctcttgatcaaagacaggtttcttttatgagctggtttcttagagccttctcttgattttgcaggaaaatgggggtggctgcccccatccagtactctatgagcacattttcctctaaagctcttaagtacttgtaaatcaaggctggcattagggtgtttggatacagagattatggatagtaaatgtgaatttcttcacttggttattggctatctaattgttattgagtttcctgtgagctggtagtaccttgcttcatcagcttaacccactgccaaaagttctttatgcttcctgcgttattgttatcgatatgcccatggagtgacatctatgttatcttcaggagcaccgcactaataatgacttgtgtgcactctgcccattagaattttgaatagattacaggcatgaaatattggtctggattagttgccttgatggagaacacagtcgtaggtggccaccccgaatctctgccttcacacattgatgtttcaattgcaggaactagaatgtgtgtagtgaaggaaaaaccacagactgaactttggaaggagtcccatgcaggtttgtaatcttcatgtagggcagatcccatggtgattctcgtgtaatatgaagaaggaaagttgatgggtgtcttctctaacgagaactctgattagtggaaggttaagggggtgatatcaaagtcacagtaggtgggcaaaggacttcagaccctggagacacaaaggtaacagctcctctttacactgccaccgtctgctttttagtaggaaacctgggagtgccagtaatggtttttaaaggctccatggaaactcttaacctttcaccaccttaaggcaaagagggactgctctgtcctccttcatatgcagactctacattttggccctaatggagagaaaaccagcagtgcagaaattcctcaggtgcagcacggtccatgataggagcccactgagtaataaatgccagaggaagtggaaggcaagatactagccagctagcatggtgttttttccctgttctctgacagaaggaggacagccatgtgaggaagataatcatggcagcaaaagttgttacattacagtaagaagtacatttgtatggagttatacatgccacaggcctgctctaggacacttggcaatcagcaatcgttcagacctgctttccttctgatttcctttcaagtagttccactctgtgtaacaatgatgcaaagcaggcggagagctgacctagggctttcaagtggggcagaggtaagcaaagagataagcagtcgcttagactcctaaatgcagttcttctctctcgagaccaccttggattggagaacacccaaatgagcaggattgcttttgctttcaaaagagttgaaaagctgagttacatgagaaggtgacagcctcataatggcccgtgttccagccccagcattgttaccccctacaatgctgttgtaggagtgtaaaggcagtataaacaaccctgacagaaatcttgatttatcggttcttgggggattcagcaaaggctttagggcatgaaaggcataaagaaaacttggcaggaagccagatgtagcctttgctcttatcagccactctttcccacccttgttggtaccatgaagtttaggtagcatatgacgtctcctaatagaaatcactacaagtctggctgtttgcagagcttagataaataagaataagggcctacctagctggatctagcatttgcttgctctcagaagttgcagtgacaggagaagaacctgtcagaccgatctgggcatttctaaccactgtccttagcaacgttcacaaagggccccgtcttagactcagaaaggcagctcacagcccttaggggtacccagttggtaagaaaagtaattaaaccaacctgctgtgctgagcaggcctgcagcaatgtaacacttagttatagcaacagccatagataggaactcaaaagatgaataagaaagagctgtttctgattcaaccttccaattcctgggagaaatggatgaagtaggaagtagagagagctgtagattacagaaaggccctagtttagtggtcttgtctcctatgacctttagtcacctcctgaaatcaattatatcctcaatgatggctattactccgttcccccttgaatatattaccctggagcagtcgtaccactcagatggcccaacagcctcaccaatgtcttgcctgcatcctctctgtttaaaaataggactcccagagattctaccttgaaaaatatgtaacatcattacctctgttaacaccagtttgtcagcctctagacccactgttcacagtggcatcaatcctggagaagatccacaccttagaactgggtttcacgtgatttatgttttctctggtgagccccgctagtatgaagacagtagggctttgctagaactggtgtgttctcggggtagtaatggtctgaatatctcaccaaaatgaaagaactacaggtgaaaaaaataaacccagccctatggttttcctttgctcactgttatgtttcttgtaggaggcttgaagcatctctaaactgcttgagattggatttattctctcatgattctcggcacctagctcagtacctgagatttggtactttttttgggtgaattaacgagcaatttgtcatggtcctagaaatgcttatgattaggatggggatcttgatagtagtctccatcacctttacttcatcccatggttatgaacagttgtatccattcatatggaattctcatgggtctggaataagcatttaagcaaggccttctactgaatatggctatcaggtgtggagttcaaaatgtgtgaaaaaccaagagctgaggacgtgaccatgtgtctgatgactaagatggaacccatgtaggagttgagtcagtaataacatgcctcttgctgcagctcaagttgcctgcaagctaaaattgttatttctgtgttaggcaaaatgttgggacaggctaagcatgaccttgagtccttttggatgtgctgtcttgtgaccctcttcttggtggcctttgttttcctggacagggaaacctctttaacattggacttcattgtctgtaccccatttcctgttcttacagtggctgttcagtcagcatgatggccaagggtgtcctagttgttcgagaccctatcctggactgctctttgctcctatctatccaacttctcaaagttcagctttgcttttcctttcctgccttgttatcaatatataaacacagacttaggagtgggagatccagatcagacatgtttacaggcctgagctttttccgtgagtttctggaacattgtggtgcctgcaggctctaactgccaaatctttttcagaagtctttcagttgttgcctccaaacggagctctttgcagtcctcgggcggacgaatggacggattcgagcgtcagaccctggccagctctaagggctaaagcagctctgaggtgtagcacttcacttccatcggttttagaggccctacctggggagaaaaacggcagaaaaaaacaaacccaaactcaaatccccaaaccctcaaagcatcaagtgcttgaagcatttctaatataccttccgcatcaaccctcacctccttcttgtttccaatgtactgtttctagttgataaaggagggaaatgtatgcatccttgccatgctcatttgaaaacagacaacgtatctaaagatgtatagagtgattggaaagcgatgaagggagaaaaagacactattattggtcccatcgcctgttagcaaatacaggggtctctgtggccttgtcttcttggtagcctggtgtttc

SEQ ID NO: 41 - DNA, 5000 bp, Artificial Sequence
  Nucleic acid probe
tctcagctcacctagtcatgttcagaatggacaaatccctcagaggaagcagacacggtttctcgggacggtgatcctttagagccacatgcacatgcttttccagctactttgttgttatggagatccagaacccaattaaagcctgtctatcatgtttgaggaaagtgcagtttgagtcaaagcctagtccagtccaatgggctgtacttttctccagaattgtgtaattcctttggcccattgggggtcagaaaaagaatggctaaatgttactatcccaagacacttggattgattgaaagacctgccaaactcgatgagtgaacaggcagcccttcttcattcatgcatgcattcagtttttgaatcaggtgagactttagatctcacgtgaaatcagtttgaggaaacaggaatcttcctcttgcatgccctgctccttccacttacacggagaggcgctccatccacgcacagtctttccactcccatgggggcacagtggggtcctgtcatttgcatacagatattctcatctgcatagagaggttctctcctgcgcagaggggtcctgccatttgcatagagatactctcaagcgcagcgggatccccatcgtgacctctccctaccagatccacttcaccaagacacccaagtacttcaaaccaggaatgccctttgacctcatggtgaggaaagtggggttcagtctgatggggtaggcccagtctgagaggggaggccgagtatgaagatggattccagcctgatggggggaggcagggccagtataactcacgaggcctctttgtctctccccagttctctatcggatcttcaccgtcaaccacaagctgctacccgtgggccggacggtcatggtcaacattgaggaatgccctgcccacgcctagatcccagccaacagggagttcaagtcagaaaaggggcgcaacaagttcgtgaccgtgcaggccaccttcgggacccaagtcccctcgaagtctcctcttctgaactcctcccctcttagtctccatcactttctaagttccctcacctgagtccctccccctttctgagcccctcccatgcacacagcctgccaggcgctgactccagtgaggactggcgtctcacatccgtggaatgacaagcccactcccgtgccccactccgacaggtactctatcaattgtgggtctggaagtccagagacttggaggagttgctgacgctggttggaaggttcagggtttggtgggatgtgtggccccctcgttgcccaggctttttgggggtgtttggagactcggaggtttggactttacaagatttgggagtttgcagcttggggacttgcaattttcagtgtgggtttaaagattggctacactctacccacaaacagggctggttctggggtggaagcagacatttgggggtccaggtccctgtagaattcagggtgcatttgggtgtttgtggattcagcctcaggttactcaccccatggacatgttggccccagggactgaaaagcttaggaaatggtattgagaaatctggggcagccccaaaaggggagaggccagtccctcccccattctacttcccctccctcagcatggaagcctctggaagtggggctctgactcccagcctacagagagattcctaggaagtgttcgactaggatattgacattagacaggtacaaaagctctagaaatgaggactttcctcagtgatgacttttttcaccaccaaagtcactcaggcatcctgacaaggttacaggaaatcagagaaaactccccattaaccccttcagtggggtttcagaaaccgcctctccagggataagggggccccacccacagacccttctcctgcacatctcccagaaaaaagtcacgaggttctgaagaattcccggtctcatctccctccctccttccctcccagtaggctaccatctgctccagcctccaagtaatagggtggaagaaggggatggccttgctgtgggttctggagaaaaattctaggaaagcagccacctcagcctggaattagacgatgggataggggcccccaaatattgggcagccctgtccttaccccgggaccacccctcccttccagctatgtgtacaataatgaccaatctgtttggctacgccgttgtcttaaggcgccagcgactacgtgctgtggaaggagattttgttcctggtggacctcatctgctgtggtgccatcctgttccccgtagtctggtgaggaccctgacgccctcccctgctccttcaggctgaagggcgccctcctcttcatcaccatcgccctgattggctcaggctgggccttcatcaagtacgtcctgtcggaaactattcaccccttcagctgtgactgttccctccattttttcagccccgattaccagttttgagtcccacccaccgtcctcattattcaactttctgacctccctgaccctcccaccaggtacagcgtcttcaagatccactggctcatggcggccttggccttcaccaagagcatctctctcctcttccacagcgtgaggtgatgatccgggagaagaaccccgatggcttcctgtcggcagcggagatgccccttttcaagctctacatggtcatgtccgcctgcttcctggccgctccgcccagttccacgtggtgatcggctctcaggcggaagaaggccagtacagcctgaacttccacaactgcaacaattcagtgccaggaaaggagcatccgagaagcagagagagaaagccacttactttttagttttgccttcaagtcttctgattgttggaaggaaggagagagggactccgcgggtgttggtgtctcaaacagtagcagtttcctggtcctgttcctcatcaacaccaaggatctgcagtgagttggggaccgggggctcgctcagatccttcgtctgcgtttccctacccttcccctgcagggaggaagtccctctcaactttctcttgtcatcctctgggcctgtcctaaatagaggggtaccaggaggcccttctggaggtcctgagtggggtcctctgggcacttaatgggatttggggggttctgttgaaatattgctgggagctcaactggaatgtaaaagttcttcgggaaggtgaaggtgtccttggggatactgggaaaggtagtttatttaatgctttgtggcccttgtctgctcccgctgcattctgggtaatgtagtcccggacagttaagagtggaatctgggggtggggctccaattgcctcactctggaataaaaggcttagcctaaggctggggtctcagcctggggtcataggtcatcctagggtcaaacttaaccagaaggattagcaacccggtaaggggtcaggtccaatctggagtcagggctagtccagatcggtggcctaaaccagagattaagcctcagtgtgactgggactgatgtctctgctgagacctggccccacctgctggtggcttctgagctagaaaatgtctgattgcagttgggcaattgaacccagtgagttcgtcacctctcccataaaagtctgttcacaccttgtaaatccctttgaggtgctggaatgaaagaggagaggaggcactggggattcccactcccattctcttccagttcgcgcctgcatctgcccattttccagtttaacctggtttcattctcagtactgcccctgagattccgactccgcccctcccccagacatctgctgaatctccctgcttccagggatcccccgatgggtgactcaatttaccccactgaccccactacacatccaaaagaaatcgcagaattggaggagggggagttgtttggcgtaagcctgttccacttcggtgcgttctttcacgaactttacatatctgtccaacaggtccagcccccactgcgtgtggcgctcgagcaattcacctcctggagaatctgtacgaaggactgttgctggctgaatctggggttggatgagaatcggggaacagggagtcaggcccctcctcccgagcagcccccatgcctccgggccacagtcactgctacctctgcaaactcacattctccagctgtttaaagccattttccagctgctgctgggcccgccgcccttctaggtctgagaactgggagcactcacatcgaatatctggggcatctgtgaaaaatagaagtgggcttggtctcggttgaagcgctgcagttgagccgcatcatgcccagcaggaagaacagctgactaaaagcacagctcactgttactataggtttctgattccctttcaatctagggactgggacattgcagctggggacgttcttgggatccacagcatttgcagccaccttcatgccctccaagcacttggctattatgggcaccacctccagctcggcctccgacaggagcccatcacttcactgtccttctcccccaccatcccaagggtcccggcccccaccttgttcttcttgccaaaaggccagcgcttggtgcggctgcgacccgggcctgctcggggatcacaggtgttaagtcaaggagaggtatgagagtgtaagtgaagagggacccaaagtataaatcaaaggtataaaccaagggtgctaaggttgtccacttagctttctgggctgacagtctagaagacgcatgcaggaatcaaggtctcagacaggtaagaggccacagaggacaaaaataaataaacaaaacccgtctgagatatttcactcccttccccagccatggttcttgacagcgaagccatgcacagagcaaagcaggtctgggttgggcaaccagggaagaggcctctggtcaacctccttctgaagttcacgactgcgttcttccaactgctgttgaagccgttttcgctgctgctctgggggcaagtggctaaaatcctcg

SEQ ID NO: 42 - DNA, 5000 bp, Artificial Sequence
  Nucleic acid probe
tataatttggctgcctgtttgcaagtggcaaacatcattcctattatgtaacagaactgggaagaggattttgttgcctggaaagagtgagtaaagattttcagtttctcatccctgtctcccctatgtctatatatacaggtagtcacgactataatggacctttggaaaagaccagatctgagctcaacttccttccacgcccctccccagggtctttcctgacctcatacttctgcacttccaatttcagccgttcaatgttgctcagggtttcagcgatctggggctccaagctggcctcgaaatcctcatcaaactccgtgtaaatgggggtgtcctggctctcttctgagggaggctcttcagagctgtttggggataattcggcagataaagagctttaggcgaatttaaactgaacctagccagagacactgcctcccataccccgccaagcccactctggccgttctcaccttcaaagtggtagatggccagtatccttctccctacctgagtcacaagcagacacccactgacaatgacataggccaacttccagaaaggggcagggttgacccaaggtgtgcccaagatacccttcttcacacccgggacccccaggtttttgcgaaccccagaagagaccaaaggagtcgtcccaggctcctcgcgcctcaggtggaatcctgccctggctccactccaggaaggttctcaccccaagcaaacgctgcatccagcggtattgataggaacagggccgtccacctcaaaacgggctgtcctgcccccagctgcgaggtgagcgttccctctgcgctggactgcagacttcagagcaccctgcaggttctaaccggatcccccaaacccagcctgtcccaggaggggagcggtctggggcttagcatcccctgggggcccagaccaaatttaacaggactcaatctagtcctatgcccagtccctgcatctgttctaggcccggtccctttgctcctccccaggcccagggcaagacccaaccatgcctacataatcacgcttcacctgacttccccattcccttctgcaggccacaggcctcctgggagtgtgtattcccattgacccctcttgtatgacctctagaagcctccaccatgcccatatctgccttgccccgaacgagcagtgacccggtgttgctgaaggaagtgcagagctcagccctggcaccctctgttcccaagagctccatgcaggtgccacaggatggagaagaccttgctggccaaccttggtaccacggcctgcagctttagcgaggacaccctgatggatggcccagctcggatagagcctctcaggtactcacagtctccctgcctccttgagaacacccaacactcgcaagccctctttcaactggaggatgagcaattccccagcataccggctctggttcacagttatatgacaggcaggcgcccactgtcccaggccacaggggctcatctggtggggagcctctcccacccttgctgaggattcctgggtccctggatgagggacttagtgccaaggtctattgggacaggtggcagagggctgggctaaagttttctaaccatgtgtaagcatgtagcatttctaccgtcgaggtggttgaaattcaaaactgcacccggataggttgttataccccattattgcccctgactgggatgttttttcacccagagtcacctggcacactccccagaattccttccagactttactcaatgagacctttcctggtcacatgatcaagacctatggccaccctgtgtcctcaaactaaccagaaccttgccattgactcaacttcactctcactaagggagacacaagacaggaacttggggttgggcggacgtctctccacaaaacccgccacctaaatggggcttctttccggaaaatccgggagggcggtctggaggaagcgtacccctccttacgtcacagctctagaccccttccctgaacatgcccctaccctctgactgtaccaataccagaccggtgagggataaggaagaaactttcttcccctcttgagcctggagtgccattaccttatcattagctgaaatcatcttcatgagattaggtctctctgcccccatccctagcttccaccccagtagagtgtaagcaggccctcccctctcagattgtgaccaccccctgggccagccctggacctgtaagcatattccctctcctttgcaggcccagaggtggagcagcacgtcagcccctcgactcacagtagaggcgcctggggcttgagagagttaacccctagtttgccagttctcagcccagatgcgggccaactcagggcccgtgaacccacacagggagacagatggggcctaacagatcccatgactacttcctcatgccctagaaaccagaagattaagcaaatgtaggcaaagctgggatgggctgtggccagaggtagctttagctaagacatacacaagaatggaagttcatccttaagtctatccactttcccattagctgctattgaagcatcacagagagcttgactccctgttaggcacccagaaacccagagaggttgttcctgtggcagcttcctctggcttcttgtcaagacctcttaccttccctcacacgtccccactacatacctctcagtatagctgtggcttctcatgatggtatcattctctccctgggcctcagatctcaaggggaccagccgcttggagattcatccaacctgacctcccatcatcttggccccagcagctgcccacatcctaggtctaaccagctcccatgtcctggtggatctttccaaccatctgttctttcctctccagtctcttaagtgctgaccctcctagatgcccctcctgttggccctagcaggcaaggtaagaggaggttttccaatcagcccaacccattttgctgaagttgctatcagaatcatgttcaacgaaggataatgctaggaccttggcttcagtgaccagaaagccaacccccctattttccatcgaactcagagtgatgagaatataaaccaccccctccctcaagagggcatgaactcagaaaagtattaactgtgtccattatgtcctgtgctgtaaccccagcaatcagtgtagcccttatcactgtaattcttgccttcctggaatatgagacctgcctcctcttttcatccacctccacccttcctcctcaatgaaaatgtgagctggatccatgactcccaacaaaccagcaggaccagtagagctaagacgtgccaccctgtgccaggacatagtaaaccgttacgtatcatcagttctcagaccaatagcaatattgacaggattttgaagcctttgaagtatttctcccacctctctctcgagtacacactcaatagcaagtcaggctgaaggcagtagacccatatccttggagtgcttgtgcccatatgcaaatcagcacccaggcgcactgcggtgaaccctgaggttcattcttccccatttgccagccagcctcccatcaacaaaacccctgcccccctcctgacctcagggagatcagctgaacccggagacagcttccttccagtcatgtgatgcctcaaagaggggaaagggggcatgattcatcccctctcgtgtctgtctgatcctcagagacctcatggatctccattccagcttctcttgccgattcttacagtaattatatgcagggagatgaataaaggactgctgcacttaatgtctgttgtaaccctgtttctcacacttccatttgcagaatgtatcatcttctcaattgctgccacagtctatgcaacccattgcatatggattcagcactctctgaggttccccagatcctgaagaattctaaccagccttccatccaccagcttcccacagcttgtaggcttggcctctcccacaacccccctgacagtccttagcttgcccagtgtagacgtcaatagaaattaactgaaactcagtctttgggcttcaagtgtgcagctcaccttgccaaaatcctgctctgaggcgctagatcccatgcctcttggaagcctaattcaggattttctctacctcctattccccataccatcgttctctcaaatgagccccaccctttggctgtgtctccccagactctcaggcaaggttgtgtgccccagacaccagggtatgaccttatttcccggagggcaaggccatgtccccccacaccctccgcgcccacctgctggatggagcccagcgtgtcagtgtacttctcctccgtctgctggatctcccgcaggcagcagcagcgcttgtcatactctgtcatctagaaatggagaggtgaggatacagaaaaagactgagcctgggataaagagctacaaaggagagaatgacacactgaggaagaaagtagcgagaggagaccaggttatccgcactagacagacccaagagggctggggatcggccggctcacctcaatgttgataaagatgatctcaatgtcttgaggtttcaggaaccgtaagacagagtgaccccagagcttcagtctggccagctgggtctcacctctccttgtatttgatgaagacctggtagagattggctgcgccaggggtgcccgatttgaataaatacgtgagcagctctccaccccagagctgtgggaatttttctagaggccaaagcatcatatctgcccccaaggaaacaggaaaaggacgccccgtccccacttttatagacacaggagagtaggtacgtggagcccacgcctagagatgtgcctggcacctggagaaggaggtgatatttgagaactcccctgcggggagggccttccaaacaatgctagctcaccagctcctccatcgtactgctgaccctggaggctgagacatgaccccagtgacccatccccga

SEQ ID NO: 43 - DNA, 5000 bp, Artificial Sequence
  Nucleic acid probe
ctccaccgcctcaccaggttctcaatggacagctggaaattggtgatctgtcgcagtgtctcgttgtctcgcttgacctcgttcacgcactgagccaggtcgttccaccgaggtgatcttgagttccccgtcgatcttgggccggccatagtgagccagagactggtcctatgcagtaggcagggggtctagcaagggtctcatctaggagcctggcaccatccctgcccaggctcctcccagatctgtgttgactccacccatctgcctatctagaagcctggctctgcccctgcccagcgagcaggaaggcatacctggaaggatggataacgcattcctgtctggctcttcccaactgtccccatcagccctctgaccagttcctccacatcctggtggctaagctgccgactgcgtggaggaccccatagttccggcgtcaaagccccaccttcttgttgtctcggtctcctgaagagtcatcccgaacctggaagggcaagcccatgacagtctctatgcccctagcaccatgcatggatatccttgtaaaagggctttctaggatatccaaatacacaaccataaacccaaaactcctcccagattctcacctaagcagcatctgacaggccttgcaggatgtggtctcctcaaaggagaacatctggaagtcatgcccgttggcggtggcattacctttacagacagggccaaatattctgctaaaaacataaagggaccccttgggttctctttagatcaggagagtgacggagaagcagaagatcaaagtaatcctgcccagttccaggatgtcctgggtcctccccgcgctgttgcccattatttgtggtacacaggaaggaccacgggaaagtcttaccttcttcatagatgtggctgttaatatgattatggagatctgtccctgggtcaacatcaatagagatgaattttcctcctgcccccacacagcaacccctgccaactcaccactctgcttgtccatctatctcaggatggcctggagagcccagagaggtgataatgagtattagcttcctgggaagatggcccgctcagaaaagccatgctttctgccctaaacacccaggagacaaaaacaggaacagagaggttataggatccataaccccacctgccatctaaacccataaccctacacgtggatccgtgaagccttgagttagagaaagacctgccatgatgggcagccccctccccaactccgggggtgtctttccaagtctgtccatcctcccaggacccgcccacaggcaatggaggccacagtgctcccacagaactcaatttggtgtctcagaaggaagcaaggggcaggcccaccttctaggtccctgctccttcctcccgcccccagcccagagctagattcagaagtcggtgctcagggatcacagaatgcataactaattggctcggaggttcctcccatacccagcctggaaccccagacatggggagcgggcttccacttctacaggtcaccactaaggtttgacacatggattcggggggatggctcagccctttgggtctggctgggtgtctgattgccatatggaggttgcctttccccctgaacccctttgattttttaagttggtgacagcctggctctaacagcccttcagcaccaaggtaggccccgtagtctggggaccaggcccattccccaccaagccccagttatggaggaagcaaccttgaccccttcagggaggagtaactactggaaaccaaagacaagcttcctaggttctggaaagctgggattgagcggacattgacccttaccgtaagcccccggaaagcctttttctctgtgatccggtacagtccttctgctgtcttcccattgttcatcattaagagcataagtttaaggtcacttgcactgtagtgccactggaggggggtcaccctgaatgtgggaaaagggggtcccaaggacaaggcaccaaacagaagcaagaaccctcaaaaggcatttgagttgttgctgaaatgattatgcagcagattaggcaaagacaagctatgggctctgcctgagacctcctacctgctggcctgctgatggttctcttttcaggctccttgaaggggaactgcaaggtggtgtccagagacttgaagcaatcctttagagaagggctcctcagtgctcttttctgcagagtattagatgccctgtactgtaaggggacacaattgtactcccagcatcatcctcccgtctgaatttagaaagccttgctgtcccttcttgttaaggatcttgatgatgtcaccctccttgagcgacagctctgatcggtctcgggcgcagaagtcatagcgggctttggctggcagaaacaagtggacttgaagacatttgaagtctattccactccttggaatcggaattcaagcaggcaaagtgagcacagtggtttagtcccagctcctttacatcccaatccttcctcaatgctccaaaagcctctctcaaagcctaattgtcctccactgctcattaaactgaccctccaatgttttaacaaaacccccttgagccccgtctgcttgaggggacccaagaggaaatcggggtgttataaattaaccatgtcagggcacatcccgggagcaccgccagctggacgcctgtggattggtgagggggctttgtcagtgtggcacactctctccccatctctctcctagcaggtccttctgaacgacaccagtccttctatccatcttcctactttttcccaaattaatttatagcccatgatccttccccagccctgtcagcttaggccagcccatctttttgcctcctaagggttatgctagcagccggttgcagagagagctaggatataccaagatccccacccggcactagagtgacggttaagagcgtggttctgtgcttaggtgcataccgactctctgcctggtgagggccacccaggaactccaagaacaagtccatgacctcaacctcaccaacaccctgaactagacagcccaggtagccaccttaggccataggtagaatagttcaagttgagacccctagaaagcactagaaattaaagacgtgggaagaatcctcagaccacagaaaacagctgtaggtggagacccttccacctccttgcgtgaatggtacccatgaaatatgacagctccctatgtgacaatctgcctcgcgcctcacttcattgcctggaattccaccattagggagtcattccaaatcctgcagcttagagctcaaacatccaaacctgacttctgaggccccctaacagccttctctttggaaacagtctaatcagagagtttgagacacagggagatgcagattacataggaccttatggttatccccgtgcctgagctctctgatgttagttgtgcttttgctgggtggactggaggcaaggaaagtgttatccactccctttgcaagggacacctaacgtctgcaagcgttgctgtgcacagattctccctctgccaatttctgcaagggtttctggctgagctccatttcctaactgcaggctgctgataagaaatacatcttggaaacctttccccagtcctgtttagagctcggggcacaggctggtgaacttacacagtgggcattttatggtgcattctgcatgaggggaccacgttattcgtcagtgcatgttctacaaagcaaccaaattattttatgctgcctgctaaatggaggaatcgcggccctgcagagttgcgggtgggggtgtaagaacaggaaggggaaagatgtggaagaggaaatcaacaattctggccaagaagcttacaaaatctaactgtctagatgtagcaggggagtttgagacagaagaaggattcgacatttggaggctgggctaaagagttggaaggttttgcaatcaaacccagatcccttaggttggaattttatgtttgtgttttgtaggtgggtattggacaggatgggttcacaggtgggcagatagggaggaggctggggatacagccaggagagataacttgttggcttctgactcttggtgtcttttgacttctcatttcccttggttgagatggttggtattgttatcaggcatggatgatgtccacacgcccggcagccactgatagcagctgtcacaaggtacaaaggaggtgcatagaacatccccgaattcttagaaagaaaagctccacgtgtttttgccttccccttcatggtctctccctttttctttcccatcaacatctatctcccatctctcaacaccttctttcattgcacccaataaaggagcttccgcttcctgcctccagagttgctcaatgctgcctggtcatcatagccctttttgctgacttgctgcaactaaaagacagcctagaaaatcctaatttccccctttcttcacaatagaccatccatggccctcccatttctcctgaagtctatttccaatccaggcctcccctagccccccattcagcctcacactcaccccagaagaggagcaggagaaaaatctatcctaacagatggacttccctgagctatggaattttgtcaaaggttccctgaatgaaggtgtttgcagcatccagtctggacttatgataggactcagctcgaactgcccctcctcaaccccagacttctcccttcaacataactcccgaccacattctataacaacttctttcatagcacttatcactaaactttctctaccccttcaggcatctgcattctctctggccaacttaccctttgtgtttggtttccgtgtgggtcttatgtgcccttcccagctgtgcagtggtctcttaatgctgtaagtttcccatcattctcttccactagccctctccctcacaactgaaacccaccaaggtcagagtaacaacccggtcttgtg

SEQ ID NO: 44 - DNA, 5000 bp, Artificial Sequence
  Nucleic acid probe
caagccctgttgatcccccttcccaaatataatttgacaattacccatttaaccccaccaccttggctatgcctgatccagatcttcctctgaatttaaccctttgcatcgcactcctggatccttgaagtgattttgcccattgctggacaggaagccttgtttgcaagcgcaatagtaactgtccactgtattggtgcagggggatataatgagggcttgaccactccttgcctttctcaatggagtcaccatcccataaatgaatgtggagaataacaatgcactagacacaggaaggtttccaacagtgttattacaagttgcatgctctgggcaagctcttggatctgcacattcatccacgtctgaagagtggaagagaaaacccaggtcaaggattattggcttcagatttgcagatgaaagtctcaaactcaaccaagacccaccctaatcccctcactgctgtcttttcaaacagacttggacctgacttccattcatcaatatctgtggggtacagaatgggtgtggagccaagttggccaatgagccaacaaaatcatgcttccctttgcaaaatatggagtcatgtttctctctgctcacctctacattccactccttggtctgtgaaattcaactgtccattgcttggtgcaaagccagggtggcaggtgcaaaagtagctcccaggaaacagattccattgccaagacatacaaagagattattactttggcagctgaagttgccatctttctgggagccttctggattgggatgaaagcctgcaagacttaaaatccatctattaccctaatacacaagggggaaaatgaacaaaccacaactacacactacaacatgaaggacgtgtttactacaacacacaatgatctgcttattatcgggtatcacatcttccttacatttgaagagaacccctaggaagaaaacaacagcaaccaaaccaaacttcaacctcagcaaaataaatctacaaattagcccataaagcccatcatatgaggtggctctgaattcctgagtctctagaattctcagggaaactaaaactcattacttgatcagaacatgggtgtgggactcccctccccactgacatagcagaaacatctcaactaggaccatctccaggaaacatagtactcagttcgacaactggcaaaatcttcattaatgaaataacagctgtcccttttctgattaagaataggctgggagtccatccaaaggagaattaaaaggggcatccactcgggctcctttttctgatatttgctgagggtttccaaaaagatgccagtgtcatgctttccacactctccaggaagactgtggccagggaggacgtctcttccttggtgaatttacttggccgtctgggcatccattaggtaggaacttcgtaggtttatgtgaatgatactggaactactaccaggagtggggtgctaatgtaaaagatctacacagaaagtcagtcctgaatttggtttctgtccaaaaaaaggtacacctccatcatggcgagcaaaggaaaagaatagaaagttcaattcatcataggaaaggaaaggagaacttttgatgggaataagtgaccctctcagctgcacacgtccctggaaattccagatctcgcaggtctccttgctcctccttcagaacactcccctttaaatatggacggtctccagggatacgtcctatgccgcttcctgtcccaaccagacagtaccaacccccaggcttcatctgccccttggatgttagacaagaatctcagtagagacctcttcacaaacctgaatgttctccagagtgtagatgattggatctgagaagccgtctttcttctctccagtcattactcattcctctagcggtagcttctgttaattggcttgaattgctggaaattgtggaagcttaccggcctcaatagcttcccgtaaaaaatgagagttaagagatttgccatctgattacagctgcagatggtatatgtctcagaagcttccaggatcacacagccaaaggatgtccatcttccacccttcacatcagtgccacctgctccaatttctagcttcaggactagacgggccactttacccattctgtgaaatgggtctaaaaccacttaacttaaagagcccagcacactgtagcctgagaatgggatcaaaatccccccaggcgggaagaagagttatctaaaaccctggctcgggcaaaattagcttgggggaattttctccctctaacactggtggggacacagcccgagcgatgtagaccttgttgtcagtcttgtgtataccggcgaggaagagagtcttcgccaagaggagacacacgcagaggtgctaaccaaaggcacagatgtgcagcatcttgatgttgcgagagctgaagtaattcaccaccttcaggtttctgaccatcaagaacagtatcacagcctccaactgggaggtgagactcagatgatgctcaggatggtcccggttgcagttcagtttcctttagatcactgcgcttcctcttttagaccttcttcctgggcatgtgcaaactggccccaagaaactccagatgaaccctgtctctgtattcagccagcagctgtggagagaaataggaggagtcagcgcttgtctcagagtcgtacctgtggattcagattcctcaattgtggaacacccgatcttcatcttatcccccttggctaccaagtccaacgtcacattctcttcactgcattcttaagacataagggaatgagccaccatgagtgagagtcagaagaatcatcatctggcagggctgtcaagtctggatggaggttgggaggcaaagagatgggtcattcaatctccaaggttttagattcttgattttaggaaatacccttagggcagctacagcattctggttcattactgctgtctaatttatggcctctgaggatctgaggtgtcaggctcagggagtggacatcaccctgagtgcaatggagaagctatagaacagtttcacacagaagagcatcaggatcaactttgagaaagagggctttacctggtgtcttttagcgttgagacttcggcattaacactggaaagcctctgcctcaggatccacaaggtccaggtcaggagaagggaaggagcagcccaggatgaagagctgggcaaaggccttgaaggtcagtaacctggggagaagatgtacagcatgtaaggctctgggaggtggctattgtccagctgggcttcgtcttcccagtgatccacctcttgtattcttctcgtacctggagaaggagacacatcatccagatgcagggaaatggtccagtgacaggctgctaatatatacatataaatccccagtcttcaactccacacttcatccatgagctaactagggtgggcaccctcagctcccattcagtacccaacacgatccatgatatgggcaatttttccagtggggtgattcctgtggctcattctgaaatcactggacttaacctctatgacattcggtttcctcataatgaaacttcttgcaaggaaaatcatttccctatttaattaagccactggatctaaggtgcttgcatccaaaaatatgactcactgggatctgtatgtggtgggatgcaagaaaggactttaaccctgtgagagagaaaagaatttcctcagagtctgcagcagaattaatacaaacgtgattccaagggtcatgtctacagggacgatttctcatcagtgcatacaaccacaggaagaccgcccccaacattcttccccagagggttctggggctggtgggatcctcatttccatgttaagctgacaggcgacaaatcatttattgagaggttctctgtatcaggcgtatgataggctctggaggggcatgcttagaaccatgcaccaacaagggcaggagaaaacacaactatttttaagtgtttgtgccaaagtagcttatctgtctatgtgccgtcttggttccctgggactctcattacagttttactgaccatttctttattggttacctgaaggacctcaggaaaccaaagcactttccaaaggttgttgtcaccaagtgcttcaaccctccagggagtgctagaattctgctacatcaaagacatcaattgagttttcagggaagttattctaagagaagatgcaaagggtccaagtcaagaagaagacatttctctctggctttaaagacaagctctttatcatagatgaactcttgttcacaaacatgtgtcccacctcccaagttaggctgccaagaataacaaagacagagcctccatcaactgagtttaatatatatagagcttcttaaggtgactatatttcagaccaacatgtaacaccgttgtacagacagacaaagccttaggcaaatcttctctcaggaaaaaactttaaaccaagagtgatcacttcacgacacagaatatcagaatagtgttctgttgctgtgcagtagatagacacactctctttcctggatatattcatttaacctgggcagctgagaaacccttgtgccgaattcctagtcatgttgcgattgctctctggtccatggcagttgaagaaagacagaataaataagccatgtatgagtagtgtacatccactccacctgctggctttgaagttatgggattcctttggaaaagggaaaatgaagggatattcacatggttgcaaaatgtgtgcaaccaaaaacaaaaatgcattatcatcctagcttaggggtgtattgtcactgcgcaggctcttttgtccaagacactgacattttccccattttctttgccttcttcttgcagctactgccaccttgccagaaactcactaactggcatctggattcagctcatagttccctttctggcctctctgctgtattttatgctc

SEQ ID NO: 45 - DNA, 5000 bp, Artificial Sequence
  Nucleic acid probe
aactcaatttattcaggtctgtacaacatgacttacccttctacatggtgtgctgtttcacaaatgatgtcacgtgtgtttgtaaatcctgcctccctgaaagaacaagacatttgggagaattcttagatccagagtccagtagtgtggcacgtgcaatgaagtgtcggaaggatgcattttaaagatggcgggcgggagtctacagcgcccctgtggtcacacatagattggacaaatgccactatttctagctttcctgtgaaaagtctaggctcattcacctattttggctttttagttgtaatgtcagatattgagctttggacctaacagaaatcctagcttgtcatctctgggcaagttagaagttttagttaatttctctgtcaccttttcttccttctgagtgctttattggaatccgacatctcatgcctctagaagctttctggttccttgatctgaaagaaaagagaaattgcattaggttcctgtattgggcatggccaagattatatggaaaactagagagtagggcttggtgtctttatctttagcctcccattttaggatcctttctcccaaaccaagctctgggacaaattttcctggcattttccctttgctgttgaataaaactaccagcagttaagacgctagtggcaaaaggctatcttaacacgtcattaatttctcttcatcaacacccttcagggagtgttgctctttgtggtacactgtctccttaatcgccaggtaaggctaattattttgtctgtcatcagcctagtcccaaactggatgtaagaaaaatcaccaagctagatagcagttgtggaaaacctaggaaccaaatcaggaacaggaaggactaattagcaagtttatcacaaaccagaacagggcagagccacctcaaaggcgtctcacaggtccattctccaagcctcttacttcaaatgcttaagagacaaaatctggttggctggggggaaaattgagagacttgttccagatctgttagaagcccccatccacttgagtccctttgtctcctaagctccagaattaaccattgtgatatggaatcgcatatcttaagtttggaagcttttctgcctactgaatgttagagattcaggctctcagcttccctttaaaaataaggtatgctacctttgccatcataagccaagattatcagaccagtctgacatttcttaaatttggtttcctcaattcttgagggtcaaagtgccacccttcttggtgtttccccatccccccatttctcaatagatgctggatccttaccccaagagtgggtaaggatcttgcattggtgagtcacgtgcatgaaaattaaaggactgtctaatttcagaccctgtctcaagattccaagtcacttttttcatgcggttcccacagataaacctggtgttctacttccaagttctgtggattttgagaagcaaactttcctccctcaataaattcctgaaagtactgtctccacttttctttccttggattcctcctggctcacttgttgctttgatgtaataatatcacctctcctgggacttcttttcaaaacctcttcctcttgctggtgaatagacagttaccctgttgtgctacactaggttaatgtcacactttggggtagagattaactatctcgtctatttccttcaaggtggccaactacaccagcacgggcagattcaagaagaggttcatgtaccctgtaggctacgggatcccagctgtgattattgctgtgtcagcaatcacccctaactcttctgttccctccaggtgctgtgctccatcattgcagggctgctgcacttcctctacctggcttgcttcacctggatgctcctggaaggacgagaagactcaaagataagcaggtgagaattcctggatagatgaatgtccaggacatcaaaggacagaaagtgccaactaggttgatcagcttctgccacagtctccacattctggatggggacacaaaggcttagaagtcaccccagcctagtccacattttaattgccaattatacttcaaaccagttcttccttgacaggaggaagaaagttccacacgtggtctctccataattctctccagagtccttgaggccatgctctcagaatagaggccactcagtaaggagggtctacaacaaaagttgaaggtaaagataactgcagtgagatgaaactagcagcagaaagaatagcaacagggctatgaatggtctaagtgggagttaataaaagcagtgatgatgtagagaggaaaagtatgagctccatttcagttgtcctcctatagaagttggaataagacctttacattctgaggggccatttagcaaaatcacccaggtggggctgaccatctctctgctgtgcctcttcctggccatcctcaccttcctcctgtgccggcccatccagaacaccagcacctccctccccatctgtccagctttgccgtcctcgtggctcttgcccccaaggtaccaagattcattcctgtgaacctgtgtatgtccgagaatttgtgagttatcctcacccactttcatactattctctttccagcctggtgacaagagaacaaaacatatctgtgtctactgggagggatcagagggaggccgctggtccacggagtctataaatagcaaggaagctggcgaagccattcagagggtcaactccagggtgctgagctgaccagagaagggcagaatagatggagaataaacccacctcggtttgaaggaaaaaatttccctctctgaacctgtgttcctgacttttcgccataatcaggtgagaaacacccagggctgggtctccatgcagtacctggccactaacagtatttcttttccatttttttcttcagagagcactgcagttgccctatcacttatcaatctcttggggatattctgaatgcatccttttatattacgtagacaaaaatgctgcaccccaagcgacacacggtgtattgaacagcatttaagggatttccaagggcagttttagccattcattgcctgccgactggctgatgcatgctctgtttccatgtaatggtctgaaaaggtaacactcaatggtgtcagattgccttgaacacttccttgagaagcgtttatgtaacatggtggaataaaatgatcttccgtaaccactaactaggcattttgcagagagaagtcgggctgtcacatggggtgtgtattttgtgacatacacaaacaggactgactttcttaatgttttcagtctatgaaaccaagaggtgcaatgagacaagggagaatgcttttctggaagctggaaataacaccatggatataatgcttagctactcttgcttataaagattgcattctcccctaaccagggactccggtaccattaagtgtcctgtggaaactgcttagcttgcctatcaggaggttgacctcacaaaatgatcagctcttgtgtttataggtttacagggattcttcccaaatgcagaagttctgttcctagtgactattttttttttgggtgctctgctgtcttcaacattcaccttcactttgtgatacaagagagttgttgtactatcacacccttttaccagccaatgaaaaggggagaagagaaggaccctccataaagaccataaagacgtctgggtcctggctttgtcagggtttctgaatgatgaaatctgtgggctctcaggatcacagaccctctacaccactaattcaaagcgtggagttgagcatctggaatgcgagttttgcttctccaggaaagggtcaaatttctgaatttgatataggtaagagaggctgagtaacacgcaccttgtagcaccttaataaatatcgttgccttctgggctccccactccaacacttgtgcatattccctatttcttacatttcagtaagagtcatatctctttcaatgacatcttttctttcacagagaacaatagtcaagggacgacacagtcaaacgtggatatttgggtgagtggggtgaagcctggattttataattctactgtgcaataccttttaggcttcttagaggggaattaggatgggctgcagaattattagggaaagactcaacttgagggtgactagaagacaactttctggctggtattatagattatgatcatccggattgttacggtaagaacctccaaatcctatttccactatgaaaaatggtggggacttggggggcaacactttctcttgattcacagatattaatgaatgtgaaaccgggctggcaaagtgcaagtataaagcatattgtaggaataaagttggaggttacatagagcaccttctgcctgtgggtcccttgcttgaacctcagctctggtccattcagcacggaaggctgatgtggttgggagaggcaagacacataaacatttctgcccaaaacacataaacccatagcccaaataaaacccgtacctcttcagcagactatttgacaaagtacagccattggccaggtgtgcttgtccctgtgccaaaaggatgccaacagaggcattcctaagatgacattcttctgaccgcagcatcggtacgtggtagatgcatactgaatatttctgtttctctgcacaggggaaaggaaattgggatctggaagcttcctttggaggagggaaggagctttttcaagagacagagcatggagtctctggcactttcttctgactctgtagagaagaagctgaatttgctgctgagttttctgaaattgggaaacacagctgcaatcagatgcacttctcatacttcagaaggatgtgatgtccaaaaaagtcatctgcatttataggaaccctctctggggtaggctaaagggtcacttcgagtatctgctacatggattgacaccaaatgggtccgcacttggag

SEQ ID NO: 46 - DNA, 5000 bp, Artificial Sequence
  Nucleic acid probe
ctcatgcccctggtgtcctcgcttgatttgcttcctcaaatttggcattttctccagcaataactgcgtgtgcctgtcctagtagaaactctcctcttgaggaccatcagaaattggatttcatgaggtttatttgttccaggcattctgaccacgtcctgcttcagagagattgttcccggcgtctcagtgctatggggtccctataaccaaatacgctaagatcatcctcgcagggtgttttccttgagagagttacccagaggcagaattattgggtcaaacgagagggagagattaaagatcttcacttggctcaagattcaaaagatagaagagggtaaactgaaaagtctccttgcctcggcttcccagccacttgctttctttctctctttttccctcgttaattctcattaccactgggtaaaaggtcagtccttagcatgcaaagttgtgattacaattggcaccagggaagggacatgagagatctccataggaggcagtaatggaaggggtgagagaagcatctgcagcaaaatttgacttgcaagaggattccagtgcatgatgtgcagctgcctgcttgggaataggaggtatccagaaagtaatcaagtataactccaccaatgggacacaagattcccgcaacagaaattgatttcagtgaagtagtattgtctactgcagagattcctttattcatgaccaaagcagagggatttacatgtcaactttttcaaccggctggttatgatccacaggaggaggctggtcctctttagtgggtaacgaacaaggaaaacaagatctaaaaagatgagcgagtttcctaagtctgtagggtgaattaacagcttttccatacacctgacacccataatgtacacatcatgttatgtgatactctgaaacctgctgggatgccttatcacgcacaaagaagggggcaatggtgtggaaaggggcttaaggtgaactagaatgatcaagaaagaaaaatattctggttctggaaacattttcaaggaggcctcattacatgtccggttactttatttcttgaaagctgctatgtcggcttctatctcacaccggagctcaagataaagagcgacgtacttcctcttctgtcagttctgaaaagctaccagactcagtgagaggcaaggacttttcatctttagggtaagtcatttccccattcattttctgaggcactttgatatggtccaccatgataatgtaccacttaaattacggcagtggaaagagaactaatagaccctgcattctatcagtgtaattctcttcaatggtaccatcccaaaaaccaagaatatggatgagtcattttcccaagttggtcaactggaataggaaatgaagcaataatcgcgtgcagaattggactatgagcacacagaatgctcagaatttcagggacttcctttgggtttaggcatttcagttttacatgcagacaattttgtcagttgttgtttgccatatggaccaatccatgttccaatcagctgtggttgggaggtagagttaaataataccatgtctgacaacaagagtgcaaataacccagattctcctatccaacggcttctgcttttgttgtttcttaatttgttcattgcagctcatgagtcacgctgctgtctgtatccctttctgggacttatgtttgggaaatgaccaggccatatcaaacgttcctagaccgaacatttgctccttgggttttgaaatgggctggtttctctgacggtgacagtgtactgtaagtgcctcatgatccagaacatacagggaaacctcttaatgcacagggccggttgcagaaggatggagaacaggtgtttctcacaattgcccaaagtgatgatttatctctgaaagtgtattacctagcaggagattggggtcactgattgggtttataatgtttgttgaatgaatacatctgggaaagaccgtgtgtcaaacccgaggtgtggggcgccatcatcacagtgaagctcctgccttcagcagatcctactacttgccgccattctcccccatcacatgaaaaaaactcctatatacaccgaataaataatgatatttgcctgctctgggaaacagggagaagagaaattggattgtctggggagccaacgggagactacttgtttgtaaaatttcctggacacgtatgagtattatctcccagactagattgcttagctctgtggaggtgtgcacctcaaagcaggttcttgcaaaaacccacttccccctgcagtttttattgagtctcctttctccctctggtggagacaaatgagaccacagcggtaaaagactgtggcaagtgtagcttgatgtttatagtaagaagccacgtgggagagaaacaaggcttggcagtcaacaattcatcactctggttgccaacttccaaccacaagatgagtgagtaacactatcttagattatcttattaaaagcatttgaaaggcaacagtgtaaggagtggatggttttgtggcgagaaattgggtttcgtaaggcaaagaaggagacctttatatgtcattctccaatcttggttgaagatactgacgtggtacctggcatgacggcaacagttttgttctgtgtctggcagttgtatttagggatagtctgtctccctgcactctgttataactctcatggatatgtaagctcagtgtccacctttccattcgatgagatgtaagagagtggcattaggggagacccaaatatttctctgctggggcccctctctggactcggactcacggaatgaagcacctctcgcggagaccgaaagattctcccagacgctgcaagcgcggctcttctcacaagactggtgggtcgccccctcttccctcccaagtccaaaccaggcatagcatttcctcacccagttattcctctcaaatcccttctgcagccctagggtcccgatttctacacagtggagtcttgcaacttgcaggttctacacggtacctacctgatcatacagaagtccacggatcagtgaggataggtgaaacttgaggctcttaggaagccaagttcacagtttggagcatgtggacaaataaggggccagagtgcacgggaaaggttgtattttgaaaggtattataaagagtggtgagctgaattcaccagattcctatccagccatctggtccctaaaatgggacaagaagcttaaagctgttttcatctgacttgcttaactcatttggctggagagccattccccacaggccagttgttcgctggtacttggagccaagcagagatgggaggctctttcccattcctgcctgcagcctcacagcccccaccctccaccccctcccatcccatgctgtgggtcaggacctttcaagcctggactctgagaactgccaggaccccatgactcaccggcagttgggggcaggacgacacaggaatccacacctgccctgtgtccactcacacatctggaggcttccccacctgcctgcgatcgaccaggtccccatgcatacacagtcacccacaaagggccgtggggctccaggccactgaaaggcatcaagtccaaccttctggctgaaggaatcatgatttggaaaaaggaacatccgactcacctttagccagctggagaaaagacacatgatgtagggggactgtgcagcagacaagctctcccatgaaactgaaatcagttctgaaggtgaagaggtgtgacttcaggagcatgctcacctggacaggtacctgagagaattcctctctcttctgtcatcactttatcttcttgctccagctgggagatcagcctaggtttatcaacttggtcataggagacttaaatttcagaaaaagagatgtctttttttctcccctgaggcccaagtaaagtatgagatgacagaaagttggaagtcatgaaggtacaccattgtggattctcttatgaacagcaaggtaagatctactgctgtaggatttcccacattcactgcagccatagggtctttctccagtatgaatcttcctgactgagtgcggaactcacgaaaacactggttacatttgtacggtttttctccagtgtgaattctcaagtgcggtctgaggtatgaggaattgctgaagtattgtgagttgtcatgtgactccctgaagccatcactgaaggctttcctacagctactgcattacctgggcagctccccagtagatactttttttttggaatatgactgtttgtgtcacaggcttttcaacactgtgtatactcaacatattcctctgttgtatgatctccgaaatgtgacttaaggaatgagtgtcccaccttttgttctaagttactgcgtggccactcagttaagagatgaggcatcctaacattgattacgttcctagggcttcctctaaatgaattctctcatgatgaaaagaaaatagatgagaataagccagacggtgtctcacccagtgcgttattcctacttcttttagccagcagagtgggataaagcctgtctaagctgcagaactcgaggctggatgttgcccctcactgacctcctgtcagctcctggaatgcagccctctcctcgcttgaacaacaacttgcagttctcttcctttcggggagtatcagtgcatggacaccaccccagggtagctgagcaactgatagaattcctgggcttggatatgaactttccatccaggtgatgggcatgacaaaggaagctactgttgggataatgtcatttggtcgtattcccatcagaaaccttaagctttggccacaaccaggactcttcatataaaatggtgatggctggggtatgagtttgtcacttatgttgtagaatctggggggctgcctgttgagattctgaccattgcttgttttgggtgtcattgctggggatacttat

SEQ ID NO: 47 - DNA, 5000 bp, Artificial Sequence
  Nucleic acid probe
ctgtagacagccctttccacctgaacaagtctcagagatgcaattgatgcttatcacactcctttacttgcaccttccagattccctttacccttgactatgaggggcatctgaaggggtcatttcagggacttctgttatagttttggacgtgactgaggggtgcttgctggagtagtttcgcgatgacggaggtcttctttgccagcttgtcaggactaccatcgtcttccatgttctgagactggagtacccaacattcccctagcaggggacacttggcttgatagggctctatcaggatagcagctaccttgggtttctatatttgcttatttgtccctctctactcggtgactcattccacttggaagaaactttggactggagttccctgcccaaaagagcttgtggatttagggtggtgcaatactgccatcttcagggcatattttgaaataaaaaaagaacggcactctagatgtgtcagccgtgggaaacgattatgaaacttccattgctcactgtaatctgtgatcagaaagggagtggctcccttgtctgtggagttgaagagatttcccttgttcaaagtgagtacaagataattagcctgccatactgcagacaagtctccttgtctcatattgatctttttttcttatgggcttcttcccagctgagtcaactttacattccatgcctgtcctttcatccaaggctcctatccagtggtaagaccctgacaggctgagaaagcaaggcacaggctttggtttcatagaccacagcctcacaacgtgctggctactacccttggatctctgcctccttcttccacccttctagaagaaagaaatacttctctttcttctctttccctcttccctgctctctttcgacccccaaaatattatatttgtcttagtggctcaagtcagctcccggaagccctctgcctcattttgaaccatggctctcctatgattagttgtacgaatcaatgtgagtctcttggaaaatggaaaatgtttttcttccaaggaaagctcatcaaggaaggcatcagtctcagggagggtggacggccgtgtggtctccaaagattaaatgaggtaacattgatctctcatgctcccttcaggatggacattcccatatccccaagtccctccaagttccctgttctccaagaaattgaagttgcgtctgaattcttcacattcctccaaactctggcccagagttccgcagtatggggttgttaaattagcctgaagacacacaaagcaactaactgtgtggtgtcccggtaaggacctgtcgtgtggtcgggggatgtggctatgagttgggctgtgttaaggaaaacctcttggaatatgtaggtgcctttttttaatgggagtgaggggaggggacacaatggctttttgagcttacttttaccttctgatactagtcaaggtccagaaccagccaccagccaaatttctatctgctaatcatttctctccttcacagtgggtggaagtttagggttaaatgtcctttgaatgtcacctggtgagtccttgacaccttaggctcttcagaaacatagcagggcaggtagtccaccaagaaatgtgcttattggattgggaggtgtttatttgtagtctgctgtaacacgtgtgaaagagcaggagcgtcatcagccaagcacatcactttctctctgcatcagtgatgtcaaatacgcatcagaaaatgttcaggttttaggagctgtcctaggtgctgtttcatcattggaagccagtttcatttatttgactctactgccacagtgaaaagagcaaacagctattgggttgcaaacctcctttgacattaggaaatgttgactttgtaacaatgcagctttgataacccatgtgagagtctaggcaggaccagtggggcccaaatcttgctgctcttgtacttttaggcactgcccttgcagactcacctttttcaggctggtgatgcaccttacacacaaacacctaatgcaatgtctttttaaattctccaagtgggatgggagcatgtgagggaaattccaatccaaaatatctgatatatggggtaacccagcaaggtgtaccttcctttggtgagagatggctgccggggcaaagacgggctttgattcagagcaagcattcccaccatgtgattcaatcatggacatctttgagatttcagaattctacctggaaccggtctgaatcagggaacgtgtgtatcagctgattcgaatgccagggacctaagctggtagctacctagctctgagcctgccttcaagaacacctgtgttcatcctctgattctctgtgtgtacctcttgtggcgtttcctctcccgggtggcacaagtggcctctttgtttggtcttaaaggcatccatttctgggaatgaagccatgttcgctgctaacacttttggatgttgtgaggccacgtggagctctcgacagattcttcttgtgtcagaagtctagcctcaggtttctaccctcccttcacattggtggccaagggaggagcatttcatttggagtgattatggataagggtctgtggtcccggcgtgtgctcacgtgtgtatgcacgtgtgtgtgtccattagacacggctgatgtgtgtgcaaagtatccatgcggagtttatctttggaaggttgcacaaaacacctatagcggtagttatcttgggagaagggaactggagacctggggttgggggagagagacctgtttgcctgttattgcatagcttgtctttacatgcatgcttgcatttgcatattgcattgtggtacacacacttgagttcccatatgtgctacatgtacgcttacgtttgcagaggtgttcgttgaaagggtattgccctttacacgtgtgcttggttttgcctttcctatgtctacacgctcaccgtgtttgcatgctgtatgttacaggtcctatctgtctgctgtccgggcaggtatgtctgctctcctagaccagggctgagcacgctgcatccaggccacagggtgctgtgtgtgacatagacaccatttccacaatcttccattgctttttagactcttccatttcttgctttttcctggcacctttgtaacccaggaccacatagaggtccacctgataccacctttctcaaatcccatcctctcccacttcgcctggaaccagactctatcactgtctcatcactttctggaatgtttcattgctttccagaatctttcatcacaacctgtcttccatccccttggcactttgcagaaacagtagtctcaggtgggaagtagcatcattccatagcaagggtctgaaatcagacaagaaggatggagtgaaggatgggttccccagggaggccaagaaaagccctcttattcatttgagcttgccaaactgcccttgctgcagaaacctcattactgtgtgcatgattctgctggtaagggctgccatttgccttggggagtcttgtatgaaacacctttctgcagagtcccatgagaatctcaagctaacgtgcctcgttttcggaaaatcaggatgtggaagagcagcagagaggtttatggacaagggagggaaggaacagggtggcccacccattccaggagtggatgtgatttttgatgtgtgctttgctgccttggcgtcatttctgagcaggatcctggctgtgagctccctgcgaggggtggacactcccagatgtgcaaagctcagccaccctcctggtcttattcccaagagcctcctgtctcttctcataagttctggaattatctctagttacgtgcttatcacagggtatgtttcccactttgcaaatctcactctccacccccctcctaaatgttccagcccggttctctgcatgcactattagcttggaactgtgtcgaaaacttttcagagttttgggctcgatgcttcatcagcccggaatctctttttttgtgctggagtgcctgatctgattgtagctgtttgaggcatctggaattcatggccgggtttagaacaacttggaagcaaggagggtctgcagtggaaacgggcattgttcactctgcagaatctgtattcttgctgtcaacaatgccccgaacttcctctggaatctgctcctcacttggggattcgtgatgggaattcctaatagtgacccttgggtcccaggaacaggagacacgggccagtcaaaggcatgggaagaagctaggagaagttagttgtagtggttccaaacctcatttctttgcagtgccaaacccagaggaagacagagtgcatcccagcattcccagctagactgaggtcatgtgacatccagaaaaaggaatccccctgtatccagactagcttatgatagaggaaaagcttcttagctcccagagttgaacaactgaagagcttctcacatggctggatccatatgcctctaatccatattcatgagttactttcctctgccttttgatgtctctgtccacaatagcctttggaattatgatttgtagttgctagatagaagagattacagcaaaatgtctgatttcctggaactttgagagggagtcctcctataaccaggtggacctccctctacccctcctgggaccacatctactcattttgattccagtgcttctctcatcaggtactgtgctattagtacttaaagccttgatacctgacttcgcaggaagatgggtcagaaatgccaatctacgctgctgctccggggtccgagtgtcatgggtgggacatcaaggctgaccaaggacagattctccaccctctaagcagtgcatatctgtattttaccttatcggtggcttcccaagaccaatggacagaatccaaactcctcatcgggagggctcggccagttctcattctcatttcattccggtgccccctggccgtggt

SEQ ID NO: 48 - DNA, 5000 bp, Artificial Sequence
  Nucleic acid probe
ggtcattcctggcagtctgtattgtaatccatgttccccattgctgcaccctcctgcgctctgatctttcttcttaatcaagccttttattctccagtgtagatgtattttttcctatttaaactgatgatgtcgatcagtagcttgggccaatgagcaaatgccatttggcagggcttcctgaaaagctagactcttactttctcagtagttactcatgaagcaccaacgtcttggttattttcacgagacccatttgtgcctgtatagaaattatctgaatcttgctaatacggacatcccaggcacctgtgtggcctgatctgccctctagtggccatttgcaatactaactgtagccaagaagggtctaaaaatgtacgggcttggtccaaagcagagcgagtggtccagggtcaaagccagggtgcccttactcggacacatgtggcctccaagtgtcagagcccagtggtctgtctaatgaagttccctctgtcgtgctggagagcagatgcttcaccaattcagaaatccaatgccttcactctgaaatgaaatctgggcatgaatgtggggagaaaccttcactaacacactccgacccagcagctcctacaccatgaatggaggttgatctggaaacagaatattttcatgaaagggcgacagggtatgaacaaaagaacaccgtgtcgctgtttgacatcccagggccacctcagcaggccgtctctggggagaattttctctgatttcttccccttcccttgctggacccctgcacctgctggggaagaggaaaaatggacaaaactcagcatttgcctttcttacctctcctgagctggtctccacgtgagttccaaaacaaactccgttagacgattaacattttttagagctgtgtccgtccatctcagtggctgtaccagaggcataactgtgcttgcaccgtgggtcacaagaaccctgggatttgacttcggggcttaagacaccattcacaggaaggtacttgatacacgatttagagcttattgtctatcctcgaggcattaaggggaggagagaaaatggaatttcctttgctcagcagttaatctcaggaaaaccacagtgtaccatgtttcatgcagtggattctaggaagaggtagcaggtatcaggaatgatacctgtgcctgaacaaatatcccttgatttatctgctctgatgctttatataactccagggcgagtccagtcatcagctccgctgtaagctgttggccctgcattcaagtaaccagaaccaccccccacgcaagacttctgtgcaaagctagaaacacaactaatggttattgacaaagtcagggaaggattttggccccactgagatcctttagttgtaaggacatacatacacatttttaaacatcgtaacacaagcacgcactgaagacttgtgtcaaaaccagatttttcccatgtcttggcaccatttccctctgggatttgagatgtcagggttgacagttcctttctctttggctgttcccgagttattttttcttgtggcattagattgttgggtgagtaacatgtgaccctatggcttaggttcctttttacaatctcaccagtcgatggcatgcaaagtcaatagtgtctgcttttatcaaatgcctaaaatggaagcagacagctttcccatattagatgcgtctctccgatgtctaatgatctgcagggtctctaaactcacttggcataggtttatttgcactggagttgcacctccttccaggtcagtctttggccaaaacaggctactttgctcttccctgaactcagcttaggtgcctctgtgttatcagtatttcccctatttgcagaaattggtttctgcaaaaccaaaagcttttttatagtcattcttgagaaacaaaagtgggagcagcccaaccttggtgcccatcctccaagggtacaggctggaatttttgctaaatttgatgtactgaatactcacagtgagtaagggaaagtccttaccttgatggatttttgcgcataagtaaatcgacagatattgtaaggtcaggttgtggtctggcgtgctcctacagcacttttaggatggtcgttcagtggctcccccatggatagaaccatgctgggagtctgcctcaaaacctgaaatgaacagctcagtcgataatgctatgagtcccattgctgtcacgcacagggtagatcatggtcctgcctccatgggatatggtgtgaatcccatcactgtggggtccagcaggggtcatgttacagccgtagttgcatcacatcccacacagaacagcaaatgtcctgtctccttggagtagcaggttgaattctgccacgttggggtaactgaaggtttgtctggcctgggcctgtttagctacggctgatggtttaggggagcctcttgtgtctttgatggtcagtgggggtctctctccataagtctcttcttggcctctctcacttcttcctattaagctgaggtctcaaatttgtctacacctccatctatttcaccatcatgtctggtctgacccagtccccttgaaaaatagggccagatgttttcttccattctggtggcttatctgggaaggagttcttgatgttttcagagcagagttctgggaacccatgttaaaaggccttataaaaggcatttttccctcaccttaacaatggcctgagaaatatagcaacctcctttgattttctcactgatgatgcgggagatgctaaaacagaaacccagaaacagacacacatcaagaggccttgagctgacctgcttacgtaaagtatcttcctcttcagacttgagaattagccaagcgagagtgttggggtgggatttcctacgcacatcgcagactttctgttcgtactgtgagcttcttccatgagctattttggggccgtaactgagttgcaaaaatctgtttcctggtttctccaggaggaagagactcgcttctgcattattctcagggctttgctgttctctgaaggacagtataatcaaaccatttcctgccgtttgggtgagaaaaaaatcctccacgtggcctgtcctcactgatgtagcttcatcattgtcacataaaggatataccaaggtctttcgtgtcttccctgacctgtagcctggtatgccgcttgttaaaactgtgtgcaaacccagggttaattggctatgagtgaggtctctgctctcagatgctacttttgcaccctgttttggtcctgggcgatcattgttgtctgccttgtatattgcttccttcaattgaatttcagccctcaggttcccacagacagatcagtgttaatgatgttgataattatgatagattgcaaagtctcgcagcctctggccttgtttgcaatggttggataatttttcttcccattgtctaggccggaaggacttatgtgtttgagaggtttgtgcttgtgattttcatgtggagcttttcacattctgtctaacaatccgagttcagaggaaccagtatggggagcaagagtatgtggaaacgtccagcaccactcatcttgcatgacacgtggctttcataatgtcttcactttgcggtctctctcaccagaagaagctgctgttttgtggctagttcatctgtgtcatgccgtgagggagtgacagcccccggacggtttggcattttcctgcaagtctgccatcttgtctggcagacatagcctgggctgctgactgtccctaccccaaattctcagcagtgtatagtgaaaaagccaccgcagctgaaagtttcaggaagatgatagttgcttctgtgtgctcacccaggagcttttgggaggtttaggatcaaattgcacttgcctcttggtgcaagattcgttcctttgctttgaactgcctccctgaagcaaattggtgtttctgtgactccctctgtctagggaaaaacaaaaaagtgtcagagtgactggatttttgtgcgcaagtcatttaacaatgcagcgctgcgtctcctatttctctggcttttaaccttgctttggactgcggctcccgaagcgggaaggccctctggtactctgttccctttctagcctctatatgcgccgtatgtctccaaagagctgcgcagttctattccaaagcttgatgagcccttgcaagctcggtttctccttttccctagctttttccaaagttccatccgtggcagactcgacctgcggggtctgcagccacgaggggatggggacacacaagttttcaaatgcatgcggaatactggctggaggttaccccaaaaacgtagcctgtggattttaagaatgtatttatgagtatggtttctcaacttcctcatccatctaccataagccagaaaaatgacccccaaatgtgaatccgatggtgttctttctgcctgacatctttctttggcttctgctcccagattgtccacatgtgagcacctgtctttttggtttgctgtttctctcgtgagttgtatccagttgcccaaggtggaaagttgggcgtcatccagcactacatgaatgagaataaaaacttccaaaatgcccccaacaaacaggggtttttgcaccttgccatgagtagctttctggagcaagccggagtggttagaactctgccagctcgtgcatgtagaagctcccgggaaaactgtcaacctgtgcacattcaagaccgccatatacttgttattgaaaagccatcagtcatgtcatggctggctgtccatccgttccttagtttgattgcatggcagacatgaggaaggaaggtgagaactgagctagaaagttggcatttggtgctctggttgcataggtgccagggctcagtagcataaatccaggtagaactgggaagttccaggtatctgggctgtttgctctggtggacaaagagcagctgcaaggtg

SEQ ID NO: 49 - DNA, 5000 bp, Artificial Sequence
  Nucleic acid probe
ggtcacaatttgtaacgaaatgtgcttgcatgcccagtgctaaagatttctctctgtctaccctctaggctgtggaaaggtctgttctaaccattagcttcttttaggaacccgcagaatcaggaggtaagctaaaagtcgtctcccttttgtcacgatggaatgtgaaataggtgcattttccttctgacttatgattctgttactgtggatgagctcttctagaaacttccaagcagtttcaaaggggatttagatgtcatcaggaagtgtggttgacaacgtgatatttgggctttagagcccaaattcactggtggttcaagttcccaatcattctcggcattctataatcacaggcctcagagagtaatgctcccaatggccctcaaattatggtagtgggcggttcctcctggcagccacagaatagttacgtggcatgaggtcagtagcatctcagagggtgttgttgtctttgacttagatgagttccaagaacacggccagagggaagataggctgaggaaacagataggaaggaagttcaagggggggatttctatgtgagaaccaagaaacacctttgatctttagattttgattgctaagacacccacgacattgctcttggaattggagtagggtggatgaaatagtattcaaagatgggggtggtgcaaatgttctgtatttcaacaatctctgtttcttctgtggtcacgttcatttggaatttacttattcaacccacaaccaatagacatttccagccctttgttagtataaacagaacttctgcttggtggaatgcacgtccttttcaaataccggctcctttggaaaacacagaggatggagtgaaatccttacctggattgtactggcagtgggatgacagccggccctgaccctgcagctttcagtctgtgggagtagagtcagctgttgagcagtttactctgtccctgtgtcaatgggattgtgcatgttttggttcagtcctgccccagacaaaccgatattccctccaaagctctatttggcagaagaagtgttttcgttccctcttgcctgtggtatctcccctgataagcgtttctaagcctcacattttaaacagagctgaaaaaggactctaattgctggcctgggatccttgtgtgctgtaggaattcactgagggggcggggctggagatggcttgataaataaaggacctgcaagagagaatttatcttgagcattccttgggggagagaagcaaaggggaagtgaatgctgcataataattctttgcacttttcctgtctgaagtctttctctgctggacgtgagttgttgaaggtgttgcttttctttgtagacaattagaggcgctcatgcactgggctatggaccatccttaaaaggcatttattgtgaagctggttcatagacttctcgcccaggttgctaacagccaacctgtctgggttttgggaagttaaagtgtctgtaggcggcctagccgtcttgtcttgaagtttctcttgccttccatcatgggtttatttgttcagcaaatgtttatggagggccagcacaggggggaataagacagagaatgtcatttttccatcctgggtaaacagggtagagaagaatgaggacctcaggccaggttagagagacctcgcttggtgaccttgggaaggcttcttttccctcaggtgagaccatgagattaggaacctgtttcaattcccgtcggaatttcccaacagagctggggctttgtgattcgaatatgttcagagacctgcattttcgaacccagagcccggggacatcccccgtggttccctgttaccctctggatgtctgtcctgctactatgcacctctccccaactcctggttggaacgtgtttatttctttgctcaccgttttgcaatctctcttcccccgccacaaggcagggactcttctatctccgtcaccatcccatcctcccacaggtccctgcaacagacacccctgcagttttatcctgaatcctgggtaatttccccaggtcctgggaagcctaataaagcccctactcagatgttggattgctaactgagctttctattctcacaaactgaaagaggtcttgctaatccaaggcatgacaatcagagcaacatgtgaagcccaaagagctgcagagctccccattactcatctctggagaggaggcattcagatttgcctactcagcattcctggagatagaaccagattttgcatgggttagaatcatattttgcttgggagatcatgtgcctggtggatagtacatgcttgatagctgtaaacagttggtgctcaatatatcgtggcttgtggatcttcccagctgcttcagaaccccaaacataggctcagagtgtcttctagctttcaacccaccccagactctgacttagcctttcccaaatggtgctgtgcagaaaaatgaaaaaattgctaggagctatctgagtaaggaaacaggttcagagtcaggattaaagaccaagacagcttgaggtcatcagaggccaggaatttccatacaggctgcggtccaaagtagctggcctccctgtccctggagtaagcaaacttccttatacctatagcttcctctaaggaagcgtagttacctacagcctggccctccccaaggacacatttctttaactcatttctgtcaatggttacctccaaaaatgctcccttgtgtctttatgctgggcttctttctctagatacttccatttcttggtttggggcaaaagcccacctgctactggttagccaggaccatcacagtgtgcccaggtaaccctgcgtttcaccccaaagcagaattttacaccagagcctggacattccagtctattctggtggcttagaggagggaagatgggaaattttgggtttaatagagtaatatccatcaggttgagggctttgaagcacctgggaatacccatggcggaaagctcggccacacaccctgatgtctcgacacctttgagttgcccatgaatgggttttcagagcttcccgtgtttacttcgctgcacgcagcttcatccaaacactctttgctgtatttataaccacggtgcatttcctcgggacgcacatcttacggcttcttggccccttcgcccagggtgcactgcttctttcctctgttcccccacccccacctctgatgcagtttggaccaagttttcttcagccagctaatttagagacaagaacctggatatttattgggcgtcggggaaggggaacattagcgtttctccctgaggaatcatcttacacctataacggcaactggcccacagtaacattgatggagcaagtgtcttcttgtctgggctggggagtcaggacatagccacagggaagaaaacagaaaggtccttccctcttggaagcccctcttgaaacacctaattggtgacatggtacacgtggtttgacactgccatgctaccgacaaaaaccacgtccctagacttgggggaccaaggaatctggaggaatatgatgatagtcatttaatgaaggtggagggattgaagtgtcggaaatgctttgctgctgtcattgcatttctttacccagcatttcaattgtgctttgctttgcttttctcacttgaaattgtggcctaaaggaaattagcatcataagtctcctctggcatatatttccagcatccaagtggagctgctgagatcttgctcaagagtagcaaaattggcaggaggtagaaacattcatgtttgccatcccaagagcaaaagtcatggaatgcctgatgcttagtgttaagaggaaggaattccacacagcctgtggctagctttttgatgctaccctggtttctgtgttgaaatatgaccctagtgtgaaggtcgcggtctcagaccatcttgtttctggtatgttaagaaacagaaaaacacctcctgtgtcatcactaaagttagggtgcgtttcagaaccaccctgtaccacagtgaagagggacaaatggccttagggagcaggacccatgggggccctctgggaatctttctctccctcattgctacacctgatcaattactgagtcccatccctttaactgcttacatggccttcaaatccatcttccctacctaagctgctttgttgaaatctgtccctagatagtctctcttctgcacagacctctgaggtttttccatactgcaaagggccttgctgcaggaggaacagtctgaccaaacagcatttgcagatgcaaattcaggatccgaaattaattcttcccccctctttgctgagctgccagaatgttccaaggagatccctcctttccagttgtttctttcattgatagggtggttcccagcactcggggtcagggcttgcttgtagaagtttctgaaaagttgcagtcatgtttcatttctttctcccccttgttttcatgatgggaggatatgaaaagcagtgttttgaaatctcctccttagatcctgatactcggaactgtcgattctcccattttcattttctcctgcgagctgcaggttgaaacttgccttgcttttagactgttgactgagtttgtagttaagcagataagtgccaaaggagaagtcagggctgactgattgcaacaatcctggtctcttcaaatgatttcagggcttgtttggccaggtcaagtctgttctaggaggattttctttccatccttcctctgggaaatgaatgagtcagtgaggaatatggagggaatggagggcagtacaaaagttctccggcctggacccttgtgattctttgaaggctgagacgagagatgagactcatggctttacagccagccatttgaaaaatgagacttccccctaaccttgtgtcaaactcagagacgttgtcatcttggctttgatgtggtggaggagaggggcttc

SEQ ID NO: 50 - DNA, 5000 bp, Artificial Sequence
  Nucleic acid probe
atctgtcccgcttcccaaaacctcatttttctctgctttagcagtagcccaacactgaaaacagagtactgcttttttagaggagttctgatttggattcgactggctgtgacgtaaggcagtcagacctctccttcgaaatgggccgtgattcatctttatttttgtagggctgtgtcacgttagaaccactacagcaaaagggagaatatagtctttacctgttgatggaagaggaagaatttgtggccctgtttgccatctgccaaagaaccctggagtgggaaacagtggggctcactggctcagcactgagtgtctcagtcaacacatgaatgccagtaatgggaatctatttggattcacttaagccatgaggtgggggcagtgtgttaactggatgtgtcagctccttcctggcttctcctctctgggaacagagaacagtgcattgcagcttaagtggagtaaaatgggtgtgggtttggaggaagtggatttggcttgggaaagttgatcacaacaaagaggctattataggtacatctaatcgagaagaggaacaaggaaggagtaggtgtgttgaccggggctcagagtgaatagtccaggataatccattcttcagaacttgttcttgctaactcagttaagtgtcgaatggcgtgggttgtctactgtattttagaggacattagaaagactcagagctagggtcacactcaggttctacctcttggactgactttatttaacaagtaccatttcagcactctctgcagtcagctgttttcaaaggctttaataggctttattaccgagtgtttccaagggcttacctggggaaggaaaacatttttactcggaggtctgataaggatgggctttaaagcgctggcaagcccaggtccttcagtgatctgtttctctcaatgcgcctgtgtcagatagaaccgtgcatttcccagtaaaaggaggtgatgtgtgtgctggagtttcagcgggtagagttaaaatccatcgtaatggaaaggcggctttccctttggctgtctgctaacttggtgactggcctgacaagtttttcttttccttcctccaagctgatgggtgggcagaaagtgttttggtgggttcaaggctgcgctgaaagatggtagaccagttgaccgtgtggtcacttggctgccccagagatgagttttgtgggcaagagtgaagagggtgcctcatgccgggaagggaggcttggaccagtgcagggtccttatgggctccaatgccgtcatgaaggtcacactttcactttggagatgattggattttgtagagcaggaaagaacaagtaccctagagagtatgtttctgggctcccggctcctcttccgggggttgaagataccaccctgatttttgggtgccaacagcatgggatgctctggaaattctcagctctgggcattttgaggggaccagaagccacagggcttgcctgaccagcccaccctatctgggcagactagcttccaggtgaatatctaccagggctcagcctcatttctgactttggttttgaggcaggcagaggaacatgtatctttggacaccctctctccattatcctgcaatggatttggggcaaagataccaggagaggtccaatgcacgaagccttacatgggaagagtggagacggggtcctcttttcccccagagtcttttcagatacttctgtgaacatccgtagttgctatttttgtcttccacacttggcagcttctggaagcatcaggggaatgcttcccggggtaccgggttcctggcatgtggtatatatctcagccatccattagtagcagtatccccagggggccaaaaaatgattgttttttgaactgttgaaggatgtcctctctgtttagaaacaagtgagctgcttctagctgggacaccaggggaaacagtgaacattcttttattctattactgagttttccaaagtgagtgagtccttaaatagacctaaatagaatccttggaattgtctgtgaggactcccttggagcccccagcatgagtccttccatgggacatgttggaatctgtttgtgtgtgtgactcaacgggcgtttcgtttctctcccaaatagctaactgggggcctcagacagctgttgattatagaaatctacctctttgcgcccaggtttcctgtccatgaagccggctctgcccctgatccttctgatgcatccaccgtgcgtctgctcacctgtcttgctttctgttcattttctcttctatgttgacgcactggagacagttcccctttctccttctgcacctgccgcgttactcaatcccctgtaatcccattgtcccgactgcatcatttcagagaagtcagccagcgaggtgggaggattgacaaaacaaatacatttagctttccttgccccatttctgaccgcgggcacctttatccccgtaaattatctccctgggagggaccgccttcaggagaatgagaactcagggcatttgggcaatggagatcgggctccctgggaagccctcaccacacccaggtaaaaggtaatctgcctcccagcctgagcttggtccctgcagggttcctgcttggttcagaccccaggcagccagtgttgaggggagggtggagattaatccatagttgtgctgccttcatttctaacatcctctgggtattttctgtcccagtggtaaactgctctattgtctttcactaatagggacacatacggctttaatgaattaacttaaggatctctgacaccccagtgtgtccaagctctaatttctggaggtaataaatctgggaatggattccatctggctcagatgggagacagaacaggatttccatgtagctgacttgccgggtccagaacctgcatttgatccagacgcctccattccttctcgcagaggtagatttgaaagctattcttgcaggcattgttccaaagatgtcctcggtacatcttctgagactaatgacatgataagtttctggaaggactatgatctggatgatgagggtttcatcacagtcctcttttctcaagctcatcctccctggtgggtccatgatccctggaaattgttggtcttctccggccttttgaaattgctgccttatgaatgtccagagttggcaccagcatccgtacctgttcccccatcttcccaatgcccacgggacccacatccctgagtatcccaaagccctcacagactcaatatgctgccaactcatctcttgtttttcctcctgtcttctgtgtcctgtctgctcttgtctgtataaatttcaggactcctgaaatagcacggaagcccccttctaatcctgtggtctactctcaaggatgtatcttgttgtttccttgcagaagatcagaaaagggaggacctcatatgttccctcctaattatacccagttacacggtctccaaagtaggatatctcatcaggtttatataaaagcccctgatgtatgttcaggaaaggcaggatgctatcatgagattcttatttcctttgcctttgtgggaatcctgcggtgccacgtacagactggctctaagctcctgttggagttcaaagcggacttgtgggaatgaatgtttaatttcacatctggcattttcttcctttgaatgcaaatgccctttttgttgtattttcatctgcgtggaaaccgctgggttgataccttttggaaagttcatgagatgtccaaaaacccctggccaactcgaatgaagtgagtgaaatgaaacgctatgtctttctctttacaccgcaagagatgttaaagaaacgtgtgtcatttgctgcactgaatatacggattcttctgttttactgttctcttctccttctacctgggctaagatgttggctctctcttcccagagggtcagtgtttcctttaatcttctctcagctcactctggcttgcatctccgctgctttaccgatggctctcgaagttaatctctcatctcccagttgcgtctcctgcagcctcacctcacttgacccctctgggtgcagttattcttattctttgcttctctcctggagacctgcttcttgcccttccccattcatgcatagagaccacagggtgtgggctgagtccccagggcgatggtgaacaaaatcctgtcaaataaatgggctgtgatgcctcttgaaggcttggggaggggcaaagaacttttcagatcattaatctctaatgtcaactccctgttcttctgttccttgtagctttggtaattaattgtggatctgtgcctgggctttcagggtcagtaaatggatttaccatttctttgcacatttacagcatcttccttgggcttgacaataggcacctttggggttgtaagatgaagatggcgcaatcatttaaggaatatagcctgggtttcccactgtggggtgttctgtttccagatgtccggaggcaaattctaaagccttagcaacagcagcaattgtagcagaagtagtggcggttttttaccctatttattgtaattgctgcgccctattcgaggtctccggcgtcttctttggcgtcgtctttgccctttcagaagcgtctgcacatttttccaggtgtcatttctccaacttgaaccctcccattgagttctggctttcctatactgaaagccccttcctctcctgcagtaaggtaggtggaaccgctgtcccgccttgttggtgaatgtcgttgctcccaatcattttctaagagttttctatgttgagcccaggtaatatctgtgctgagactctaacatgaagcctcaattctgatcatggccctagcagcatattttctacctagattcgggtaactacctctctcagccagtgggaaagggtcttgtacatttgcgaaacaaatccaagaagtgcttttgagaaatgagaa

SEQ ID NO: 51 - DNA, 5000 bp, Artificial Sequence
  Nucleic acid probe
atgtttgagccatttggacttgattcaccgggcatcatggagccagtctctatcttgagtaggacagcaatctgttcaaacagcgttttaaaaagacttaaagtgctttgtaatctgatggcaattcagagctggcactgactttggggctgatgccagagatgggattagagttgaaatcatgaaaaggacaatttctcgaaaaagaaacaccgaagtaataaatgatatgtcaaaagacagcggggaaagcaggagtgatgtgtcagggagccagggaaagggacagtcgactgtgacacagctcttatctgttgaggatgatacatactggtccatgtgcttttcacagggtgagcaaaaaggattttgatctcaactaagcgcaatgaaaagctacatgatgctggttctctatggccacatacctttggtggctctgtggtcatcttgatgctgacctgcaagattgagatagggaagtctgggtgggggatggagtggcagtttgccaggaacacccagtgtcctgcaaggagatgccagggtcagtgggaatcatttcctaagccctgcctgtcctcatgctttccacctggtgtggggtccacaccaggggacaggatgaacacgagtggggatcgtggggttgaatcctccagcacctgccagaagcacaggtatagcatcaggcagaaggccgagccaccgaccgacttcatattcagcacaggcggctcgatgaagcgggagccaaggttggtgatgatgaaggaggtcacgcagaaggccacgcggtcacacagacgcctggtacctggcagcatcgccttctccggggcagcattggtataccacaggtgccagtcacgagggtactgctcaaaggagttcatgagtcagctactacatggattgtccatttggccctcccgatccaagacctggtgggagaaggggaggaagaaagggattgacttgggaatacttcaaaagaagagccctctgctggttgcctggtaataatgtctccaacctaatgactggttggtagcctcaccttcacagaagcacaaactatttactcattcattcactcaactgatgctgtgggcctgaccctctgggtctggagtccttgtcacacttgccctcatccccaaccttgaccatcccatcctgggatgtgctctctgcccactccaagattttggcacacatatgaaaactgaatagtagtttgtggcgttcgaaaagggtacggcaggtgtacctgggagaaagggacagggaagagggatggggcatgttgggggcaccctcagacctgtagacagcgtaggtgtggtagtcattcaggtagtcaatgcggtcctccagcttattgctgcggtggctttctgctcagtcacctctgtggctgtgatcttggaggtatgcagcgtgttcaccagctgcacatcatccagcagggagccggtggcctcattcagcagcctaggctgtcactccttcgaccttcaccttcctcatctctttccaccttgaaagacccctcatcctcagctgcccaacacaagcttatcccttttctcaatccatggtggtcttggctgaggtctctgggctgtagtgggggttggagagcttggtggtgatgtagaaacggaaattggtattatattccacctccttatcgccttggggacttgtcctgaccgattcgggctacagatttgttgagcatggggttcagtgtggggtccagatattcctgcacgttctgaagtagcaccgggttctaggattcgcaggtaatcgctcatctgcaggtcgatgatcttcaggccctagagagcaagggggaaggtgaaggaacactacgggagaggtggggcctccggactttggtaggattgcacaggaagttatcgatggcgaaagaaggggagcaaggaacctgaagctcccagatctggggagagaaaagccccatgcttggtctcggagcctacttgtcatgcctgagtcccatctcaccttcccgatccagatttggttgacaatctcatcccggtagttggtcaggaagggtcccataggttcaaagaggatctttccctcccctaaacctgtgtgtcctgacaactctagcctggttctaattacttttgagttctctatctctaatggcaatgttgtgcttcccagccctgcccaaacactgaagcagatccctaataatgcaaaaagcctttccttccattgctctcaccaaaggtgtgcagggggaggggaagctaaaagggatgggtctctcggaagtggcgagcagagtgaaaggacaccaggtagtacagctgatctcacctggactgtctcctcccatctggccttctctctcttcagacttcttgcgaagctcctccttctgtgccagcttctcatcatactgtttctttagcatctccagtttctcagctacctgggagagattcgaggtggcaagtgaaggccagacagcacataaatgactgccactccatgccagaggccatacctaaggtggaaccttgaaagaggaagaaaaagaaagaaattgggcagagctgtgtatactagagaaacccctctcaagtcattgccacctgtggactctgtggaggttggaatgaatatatatatattcactgaaatctggtgcctggttacagactgtatcaccgtccttttcatcatgaggaatatggggtgggggattacacatagcatttacacgatacatgttccctttcccgctgtggaaatagttccactactagaaagtagaagtagctctgccttaactgagtaattctggtaagtttcatcctttcggagcctcgccaaggtggctgcagacataaatcaaaatccaaaaggaacgaaagtgcacccggttaaggggaagtcttcctgccattcctgagcctggtcccaggccctgccctagtttcccgtagctatcaaaaattcaaaaggcctaagggcatgaggatatgaaagatcttcctcctaaccctgttccccactcagccaggttccttctttagaggcagccaaaccaagattttctctgtgtatcctcattacctcaaatgctattgggtttcttctacggtctttggcaatggtaataaccagttttttggcaacgtaatgagacttcattctgaacttaatacagcctctgaggtcctgaggaacaacatccttttccagggagaaacagcccaggttaagaaaactgatctacccagaccctcagctgagatggagcagaacaggaagttggggaggatgacttggggactttaacttcagaaggttgaagtctagatttttaactcacaaaggcaagtaatttggaatgcaaaatgggaaaaaatgggaatgttcagacttggggatacagtcgccttaaagtatattaagattctgttctgacccatttcagagccatggctaaagctatttactcaaaatttgttctgatctgcaatgtagttgggctctgagcctcttttcagacctgagcctcagtaccatgcataagctttcaggagaaatgtttgttccctgtctgctttctcttttcacttaggggttctcaagtgtgggtgtggaccctccaccaaaatacctaacactgggtgcactaccactcctaattatttagcccaggatatgattcatctaggtgaaaaccctttccaaacagattgccacacctgtgattcttcgctcttgggaaaagacagtcgcctgggttgcccagactcctcacggtaactaacccacttggcctctgggagagcccgtccccacacaacatatgcacagacaccctcacgttgttctgtcaatctttggaagggctcctgggtcgcagtgcctttgagcctttataaccctctgcaattgacttagctttctgagccagaggcagcccagtctaaactcctccttgtccagcagtttcacctccactactcacgcatagccacatttttcctgggctcagttcctaaggccaaattatctgacctggcccctagttcctcagcgagcgcggcttgcttctcccgaagctgagccaaggcagcgttcattcggattcgcttgggctccaccacccgatatagccgcccatacagctgtaagaatggaacacagagtcccggttcccattttctgattcttgttcccctctgggggcctgagggcattcctgcccttggatgcagcaatacattcttctgacagaacctagctgaggtgccacctcttcttcccacttccccagagtctttgaggacaaagaagactggcccattcccttccccggggcctcgcgttccgcgggcagtgcctcttctagatctttctgggcattgtcagccagtgcctgacacttgatttcctcaactgcaatcttttcactgttggctgttacggcctccgcttctgctgcacaatgatgaccaggtactcctcacactgcttctggaactcagccaccttcttcttggcatcctccagctccaacgacatcacttgcatcttctgggaatactgagctactgaccagtgcatagtgacaaagatctgggccaccttcctgtggatctgaggccagtgacacaggatgaaaaggcagactcacattctcctgagttcccaggtctactcctatgaggcacttctcagccacctcgagcagggcctcttggggccactctgagaaccagttgatggttgtgtaaattccgtgcacagacacagcctccaggtactggcttcccaacacgcatccactcactttctaagaacctaaaatgccccatccccttgctcctgtaaaaggatggacgactcccatgtccaccaaaggaacttcctgaaatcaggcacagcctaaggaaaggagatgggtgaggcgggcagaggagcctgctgtt

SEQ ID NO: 52 - DNA, 5000 bp, Artificial Sequence
  Nucleic acid probe
gtcctctaggaaggactcatcagctatttgggtgtccacaaaaatgaaggacgtggtcttgagctccaccccagcctggcgatacagacgcttgatatctcccagccgtaccatctcggaactcctgcttccgataatgtttggtgacctcgatctggaaggtggtgtagtcgcagatggatgaagccaggcgggccagatgaagcagaggaaccaagaggtatgagaagaagttggtgcccaaggcccaggaggcaattcaacttttaacctccaccttctctgttaaacacacactgcacttgatttgggagacctgggaagggacccctatttccttagacataccccatcactgagatgatggggaaggggataggcgtggcaggtgctcactgtgtccttcaggaaatcccctgagccatgggaagaggggatcagagtcacaaatgcctggtttgcctccccccacagggctccacacttcagaccccatgcccggtcaaagaaggagccgagcttgtcgcttatgatgcccatgaaggcttctgtgtctgccgcatcaaccagccggtcagagaagactctgtgggcagggaatccagagccgtgtgatgctggacttggtatcatggaagtccttgttggctctaagcatgccctggaacacctgggaagaggactgactgttgggaccagggcacttcagcatgggggtgacttttggtgcgcatgctagtgagtgttaaggcttagtttgagaagagaaagcctccgcggaggctgggccaagcagaaggcttcacctcttcctcaaagtcctgaagcttctgattgatcatggtgccgaatatgcggatgatctgggacttctagaaacagaccctggctcactctccaaataccttccctgttcccctgggatcccttggccccacatacttctccagttctccttcccactccataggatcaataaaatgtcatagacaggaaaaaaaccttccttagtctacttgggcttcactgtgttctctgcccattgggactacaaggcttccttgtccacagccaggaaggtacgcttccaacccgtagctctctcaaacacacatgaatatagcactaggcgagtttttggccctgagtgccagatatacacacaaacaaccccaggagccaggccgcgatggccgctgagcctgaagaacagactctagcgaggcttactcgaatgtacttgatggtctgcttcgtacggtcataccagaagccatagtcaatccagaggcggatcagcgtggtctggtgaaaaggtaccgaatgaatgtgtgggtcccctgagagaaatacgggcttagagaggagggtcaggcaggcacttggatcccaagggagccgttggccaccaagctgctcaccaggtagttgtagcgaacagtgtcgacggtgggcaccatgatcttatagaagggggcgctagggaagagcaggggccggctcttagggagcttgtcctcaaatgatgtccaactccgtattttggggtccacaaaatactcatataccgtgtcctaaggagggcatgtaggtcaggttacacacacagaccagatcatgctgaacacaaatgtcatctctaccatggtgacatagttctcgccgtcagctgggttcacctggatggagaacgagaagagtatctacaacctgctgaaagacagacaaacctgaaatttcttatagctcagcagttgccctgagcttcctcactgctaactcatgaagatgcagccatcaagcacatggggccatcccacctctctcaggcctcttggagaccacagacagcaaactcggcatccttcaaaccatccctggcaaagggatagtctgctttctgaaatgtttaaaaagtcctctcatcagaatagcctgggagcttcttctaggagaaatggtgtcccagggcctcatctacgccaaaacttatattttgaaagtgaggagcggaatgagctattttcagctcccaaagcatgggtgggaaattatagtgagatcccattgtgtacatattcaattcagaacccctagtcaagtctctgctccactcaagcctggagttcaacctgtctcagacacgccgtttcccagaagttgttctggtgaggctgtgagcaccccctgcccctgcccgtccttgcagttgtccttcttaaaggccagcatcttgttgatgagcttttcgaacatgcgttgaaggggctccacctcagcctggagttgaataagaagggcctcagttcccttcatacctttggcctcttctccagccatgactgaacatagggcttccagcccaggtcagcgtagtcagtgtagaccatcccgcagcggcattgccaggtcctccacttcaaacaggagagacacctaaggatatacgtttgtcagaacttcatctttctctgcatcccccaccttcaggaataataaagttatcgtccatgacggagttcatgttctcgatccacagtgtgtccacggggccatcgaacaggatccacttctcgtcgggtttctcatctgagtagatgcctgcccccttaggagactcaggcgtctgctctccacaccccacgatcctctggatacctgcacatgccgtccgcatgacactggacaagatgccatctgatggacaaaaacccctctttatttccagacccctcttaggtacgtgcatttgtcccttctctcatcgctccaggccaccatggtcccaggtacagctctcctggcccccaccctccattgtcttcttactactcaagtctctgcccactgactccactgtcccagccccgtactctaacaatgttgaagttagggtctccgcgggagttcttggtttcatacaactggaaaaccttggtgagggtgaacggcgtgctttgcaggcccatgtctcgaatctcctgctcaacggtctcccgcagggaaaacacagcaagcagaagaaaaaggatgaggcctttaaagggctttcaaaatctccccggtgccagagagaatacgttcaaactgttaaaaatggacaggggtgcatcaactgaagtgagcttggcgatgttcatatctctcattgagagcagcagaacctgaatgggaaaagacagagactcaggcttgaggagctccaaagggcttcggggcacgtgccaagaacaatggatacaccagttagccattagcaaagcagcgggcaacacggggatgggacgaggtgggaaggtgaagtcaggtccaaaggctgtgtccttgctctacctcttcatcagtcagatccggctgtaggcggcgcttcttgccagcatagcgcagaagggaggtgagggacagtcagagacaagaagccaggtcccaggacatgaagcagctaggaggcactctatggattctcctgaggaaattgtgtgaatgacgccattgtcagtccctaaatttttctccccacacatcctactgacaatagtctaagttccttagaggataagctgattgtgaggcccttggggagctcctctgtccagggaagaaaaggggttattggagtaccttgcagttgccaaagccctctccaaagagaatgatttctgcaatgagggtggagtcaggcaccaccatggcaattgggccatccctaactttctctccaactccaagaccacatcctttctcatctcagccccagcccttcccagtccactttggtcttctctcttatctctggtcctctctccaggttctgtgtgtatagctctcatcagtcctaatagaaatcacagctgttcctctttgttcagttcctaaaccctttcctatccctgtgctgtccacacataagtggttattaatccctttggggaaaacagtaagattgcttccccttctcccggaaagttgtgtttatccttctctcccctttgctatctttcccaggagtcccatccactccctgccacctaccaggattcatggtaatgaagatcccacaggaccacaccagatttatttcaaagccatcaaaatggaaagtgcaaggcagagtcactgggagcgagaggtgccccaagaggctagaaagcgtgattaacgcacccttccttcaaatgtttcaataggcgaggcggcctcttccatcagatggggccaatctggggtgatgctagaaaaggtgtgggttggaaagagactggggtagggaacttgtgtgcactgtctactctgctccagtactgacctgggccagacctgagtacattcggcccatggacttgtagtccaggccctcagagcagttgaccacaatgacatatatgcccagggccttgcccctttgggggagccccctcggtgcaggtgcaatgccgtggtcagtgtcatgtaacacctgagaattgaggacagaactagagtcagactcctggaagcctccagacctgtccgtcaggggggtgatgacgagccggcccgagttacccaagtactcataattatactgaaattgcgtgttggtctggcggatgacacagtcagttggctgagccagtcaaaggaattgacatccatgaggccactcttgtaaagcttctccaacacatcccgggcatgaatttctatcgtcaccagagccacagcaggcacttggtgacatcagccgtccactggatctgactggcagtgatcaccacctggggatgtgcagaagccacgtcccaaagccctgtccttggccaagtcccagctaattcaagaaccatgtgcctctcccaactgcacccaatctgctctgaaggatgggacatgggcctcaggtcaggagctaagggtccaggttgaccccagctcacctggccagcccactccttcacccatttgtccctcttgttgaggaacttcctgagggccaggtggcagttccggagaaggtcccg

SEQ ID NO: 53 - DNA, 5000 bp, Artificial Sequence
  Nucleic acid probe
cacagggaaggcacttatggagcagcacttcagggctttgtacccagagaaaagcgggaatggccaacagctggcttgcgttcctgagagaaatggaggcttgggagggcagggtcccctccaaagcttttcataccccaacaccctcccttcgccacccagcatctcactgtggccacttctactgaccttctggattcgggactggcccagaatctccagcaggtcatcattggacaagaagtagaagcgggggaaaatatgtcgcttggtctctaaatacatatccagagatttctgcgatgaatgacatttgattcatttccactgccctgggcggatctggatgccccaactctgtgttctttgccagctcttgaacagtggggcacagactgccgctttctgaagaaaggagggacactagtgagtgtgaactcagaggtgaccctcatgccttcaagcacagggtctacaggctgttggatcctttgagtcaatctgcacccccaccagaattcttctgcacaggatgcccattctaggtgcccgctgtcaaacctgactccctgattgtctaccccagtctcccccagtcgaatgggaaatgttaggagagctgggggcatcagtctttttaataaatgaagcacgcaaccatgaccaagcagagtgcctgccgaggctgtgtcagggcagggctccggagagcattgttgtccttgttcatcctgtccatgatggctttccagttgctgttgacctggtcaaataaggtcgattcattgggcagctgcttgcggagggaatctaaggtgagaggtaataactaagaatcagacagctgggtcctagatgaagagtgactttaaaggtaggagaatgaggctgataacagggaaagcgtgatgccttcatggtagacagagctacctggttatcttccagtgcctggaatacttcttctgtacctctgagacacaaggggtcaaaatcataggcccttatccttgtagggtactatgtcgagctgagtcacatcccaggtcttggcaatgttttgtaaagcctgtggagagaaattgggggacacagcagtcatccatcccaagctccacaatctgctccaaggtgaagctttcagattcctgatcaaactcccgctggatctcatcccggacctggtcccagtgcctgcaggattctgcatagttaccaattcatgattcaactcattttgctaaacaggctcgaagatgcaatcttaggcaccaggggaatacgaagattgaggtgaaactgaactgctctatttttgagcgagtggtttcaataatttcccagtttcggtcctgagcaaaagttgggggcatgagaagacccgacgacctccctgggaagccgtggtctccatggtttccgtctgcaggatcaggaaccggccagtcttccactcattccagttctcctcccagtctcgtgcgatctcccagatttgctacggacgcccaggcccttcgctgctctcgctctgtgaagcttcccctctagataatacagcggcctcctcagtccacatatttttctccactctgttgcccgctcacgtctggcaaggacagccatggctctcctatctctttgtgggcaaatctcacctctctcctcacgtgagagagggaagggaaggagagtaagagagaggtggatcaaggagtacctttgaattcgaaatcttccagaagtgtatgtgctttcttcttgaagtcatctgccgagtggatcaggcctgtcttgaatacagccaagggcctcccccgcaatctgctgctgatacgggtcccaggtaaaccaaacaccaaaggaactcacactgtcctcgactggcacctcgtactttgtgactgtctcctccttctgcacgttattagcaacttccgtgtagctgcaagagccccagatcccagctaggatgttgttttctttgcagacataccctctccaatctgccccagccatgcagctgtgactgactgtcattttcccagtcactggctataagatagcagtcaaacttcttattctggcattcaaagctctttctatttaactcccatctgccttcttagaatcaaagggctcacttctttcatcttccagatctctggaacttcttctttctcaccaggccctgccatcctcccctacccacttcccaagactctaggcctgggctcctcctgcagtgctgtgcctcctacagacagactttcaatccaccctcaccactcaccgggcaatgaggcgctggtagcgatgaatgaaggagtccttgttgatctcccagatctcccggtacatgtcccaggtcttgaggtagttctgcagcaggcttgcgttaaaggaccaagaaagatgcttctcactgacttccaccccaacccaacttctgcctgagccctgcccactgctccagaccttccactctaaaaccagcttcaaccctgtgccttgtgccctccccgttgcccactcacccacaactgtttggatgggttcacgatgtaacttgcgcttggtgagaatgtcagggaggtggcgaaggtcacacacttcagcagcaggtctgaaggccataggataaagtcaaggccaaggaccaagtttaggaattaagggaaactgacaaataagatgcaaatgcggtccagccgaatcatgtacagcatccactgctgctgaatctgatgggatggagggaggaaaaagagaacttttcagggaagacagggcacatcctactagagcacgatgtttgagaagtacacactggtaaaaaagaaaaaagggtcggggagcaagagaatggcctgatggaactgggggactgtggtttttcccatgatggtcaccacatcctggtgcaggttcatcaatttctgctgtacagctgcccgatgctctctttggtcctcttcaaattccaggtccctgtataccggccccgtcatgggggagtttcagctcgggaagagaccatctggacctcattctaccctcccacttgttctccagctagactggatgacacacaaaaaagggcatgtatacggcactccgtgatgaagaaggcactggcccccttcaaggcccagtgcagtttcttgagtcccgggtggatcttcttatccaggagccgacattcctagagaactggcccagagctacaatatatatggtacaagtgaccagtggggtgggaggggacctggttggctttactctccaccctgcagctagatgcgcaggtcctcggctcgctcagctacgttcaccacgtaatggggcgtctcaaacagcagccgctcccagtagtcaatttccgcaaagagaatcagaatttttaatatgctaaagcaaagttcagaaattcagacacctacctccaaggcaagtgtggctcaagtgtgatgataaatggcaactgacgcttggccttaattatcatgtccttcaaactggatgtcttggatttgtcactgttgataaacatctgagcaagcctacttgggcatgtatgctggccaacattgcaccaaagtttaatatgtcagtcaacaggatgaattcagacaataccttcctgatgctgagctgaaaatcctgggagaaactttgaatgatgttcagtctttaactggagagagaagctctgggaacaaaaaacagacctcagtatctcgtctcctcctctcaggaaattctactctgctccacacagggtgataatacagctccttcaggactaagagagccaggtaattccgaggaacatcagaacatttggacagtctgcttatttcaagatcttaactggagcttgaacacaaagagcaaatctaactgacttggatttctttactggtgatgagcccagactttcaaaatccttcactgtgatcgtgttctttcccttccattatttactggattgaggcagaatgtaaagtatcctataacatctctctggaaaacaatccccaaaggacagaaacaggcacctcttctatctggaagacgtgtttcgccattatttggatccttgataagctctactatcaagattatgcagaaggtattccagacaagagatgataatagcagacagagaagttgtctcctggtttatcctaactagagaaaggacagctggtgaactgtgtgccctcatatccataaacaccaagggcatctgtgcggctcccctgacacaataactttgggggtacccagctcgatgtcttgcaggttcaaccccattatgcgcttgcaaatggcgtaactaggctaactctggcctagtttctcagtcttcagaatcttatcgttgatttatgagggtggctgtgcttggccataactcacaaaaccaggaagctcagcaaagccaatctccaacaactctcactactcgaagccttttctcaaacctctcgacctttcctcatccctgcagctcccaacatccctgtcctcctcactgatgcctctgcctcacccctctcacctgacactccgctctgcttctccatcctggttcccttttgctgcccaggacaaacactcacagtctcatctgtccctgggactccgtctccacacagcacttctctggcaaagcccctcctacatttggccccttttatgagtggaatccaacactagtagccacagcctcaaagcatcttatgtggacaggaagccccactccaagagtagagctgttgacttagaaagcagtatagtatgacagtttcataacaatgaagctgtagaagaaggcatggtctgacagccccaccaacagtcaggaaagggacagggtttgggtgacttagctgtatagggaaaaggggccaggtggctcctgtacttgtcaaagttgacatccagcatgcccgccttctcctggctgattcgcagcaatggggtatccaacc

SEQ ID NO: 54 - DNA, 5000 bp, Artificial Sequence
  Nucleic acid probe
tccactcttggaaggtttttcgaaccagctcatcaatggcctggaccatctgctgataggtgtgcacactctcctttccagtcccaatacggggcaggaagcctgccaccttcctccaggggccaccatctccctgagacctacacatgcgcgcatgcgcacataccccaacaatatgcataaggcttgctatggagatgtgataacttagaaggaaggtagagtcaaaaaggaaggtgataggaagagaccaaggcgcaatgagaaaaggagtcaaaatgaggaaagagaagcacagagcagctatctgcaggctgcctaactctctaccagcaccagctgcatgatggcaggtcactacctcaacagctttcaacctttattcccaaagggtgtgtgtccatcccccatccctcctggcggtctcactgacgacagaatatggagaaggccaggcacttacggtcatgactctgtcgatgcgacgccggaggatgtgcacgtagggctccaggtctggccatttcttgttccgttcacggttcaccagggccagctcgctattgaacagcatgtagagatccaccgccttcttgtcatgtcccgcaccaactcgaaggctgaggtgatcaggttctgggtcatcacctccaggtccttgattccggcacggaacctgcaccggtggagaggcatgtggatctgccttccctgcccacgcctggcatcagcctgtggttccctcacttattgtagtcttcatgccaacaggtgttcttgacatccaggatacccccgcggatttttatgaaagatgtcctcaatctccagcaagttccgtgttatctgtggcccctgggcaccaaagaagcaaggaaggggaccctgcttgccatcttcaagtgatactgacagtcacatacctaaaaaagaagacagcatgagggttcctgctttccttccaaactccaaagtcctaagctatttggtttagcttcaaagaccccgagaaacaaccctcctgaatcccaaaggacaagactcaaggtgagaagaaccagacaggagacagagcatctaagttctaggagcagattcatgccagggcaatgagatacagtatctgtatccagacagaaaatttggccagatgtagaaactcctgggtctcggaaaatacaaccaagaggctaaacaaagtttccaagcgtgacaacagagaatgcagccttgcaggtcctccttgctggaagagacatatccctcaaagatccggtccagggagatggcgtggcagcatctgctcctcaagagccggtgctccttcctaaatcccctctcccccactgcaatctccatcttgaggaatgggaagaagagcttttccttccacacagctcaccaactcctcttctgaagcccctcttcctgcctaagccacctccacctagaaacagaaagtccagcccaggctccaatgctgagcaaccctaaagcagaagatcctctgccgaatgcattccccctgcttcagaaaacccggccaccctcataaacccctccatcccaccctcagcatatgcacacctttcggaagagcgctagagatgtccttgggcttcatgaaagccaactcctggtaaggttccttcaggattgacaaaaaggtcaggtttgactgtgcttgacgagagccatccaaccatcttcaaccccattctgacttgcctctaagaggaatccctcccatcagccctacacagtagcgatcatttcaggtctctgtgagccttcggaccaaggactccgcactaagtggatactctgttgctgtcggatcaactggccgcatatcactctcattcgtccccatagttgtcacccagttccctttcactgccccttaatgcccagctgaaattttcacctaggaagacaggtcttctatctgtattctacaggccactcttacctcctgtgtgacctagcactaacataatcaaagaaaaaaggtatttatcttgtaattcctggactgaaccacatgagctcatctttggctcccacgctgtctagtaatcctgttttgggtaatcttatcttgatgtatatttattggttaacttgatgtctctctccttagggaccctctattctactattttatactctcctgcaagtacaaggccgaaccctgggtggctccctctccttacttagctccctgtatcatcccaaccattgcccacttcttgctgagcctgaatcctttgattcgctcacaaacctggatctgctgtcaagtaggacgacttggcaaggtgcaggatggattcaacgtgcttcactcccttcttcaccagctgcttactgatgccagacaggtccatgcatcggttgctgtctccacagtctcctgggcactgagcatctcctttatctgccgggtccagtggatcatggaggctgcaggggagagtacaaaatctccttcgtttctgccaggtgatgctagcagtgccagccactcactctctagccgttgcaccagctctttgtcctttatcaccatctcaggcttcatgttcatggcctctgcattgtaccgagtgtctaaggaaaggaaagtttgtggaagtggacagaggggatgagttcttgctggccccgtaggaatcacggttgatgatcggtaggggaagaaattgccataaaataaggtcaaagatctgtatcgcgccttcaacactctctaccacgagacatgtgagtcctcccagtgtccccacagcacctatatctcctccacacaggtgctcagacgaaggacccaccacttcactagttgtacagttgggggacactaaggagaaactagtcactccggtcttcccacttacgagaagcaaaatgatttctaatgctctcaggccagcctgtgtttgcaaagatctgaggggcaaagactccaccgagcagccgaagcagggccgggatatactgatccttctctaaatggaagtgccatgaagtgcttcctgttcttttcacggatatgattccatatccagagatgtggggaggaaatgcagagagtatggaggaagcacttcagccaatggaaaaagcagaaaggagaaggactggaaggaagagacaattgtctagggttttccattttggttgcatatatttctcccttactaagattttattacaccctctcaagttcactccatgtaaccaggggagcaccattctttgactcagtgcatatgtttttcattggtctcccattttacaagacttaaggatgatgaaggaactgagagccctcggctctctgtaggaccacctttgatctgggaatgtagggtacgcacctgtacaggcatgcccacctgggcaaagtgttccagaatggcatcatgctcctgtgtccacactgcatccgccagtcctgtcagcgcagctcgggaaaggaagaggggcttctacagaatcacaagtgaggacatgggagatgagagaagccagacaggttcagaatggttaggagaaactgaaagatagctttccagctttggaagaaacagaaaagtcaaggtcatcactctattcttctcacccaggtaactatggtcatgactaggctttgatgcaacttggaaataaagaagcagaggtcatttagtgccattatccaattaggacaatatcttcccagctcattcaacttgttcaggtgttcacccgcatgatgaataagctcatcacagtttgacccaaggtggggagactgcgccttacttaaagccctcctctgtcctggcgaaggcacagatgactttgtcttttactcagtgaactggatttgtggtgagtaggctaacagcagaggacaagagagaattcctcatgtgccatccacaactgatcctttgattcaccagttttgggggaggaatatctgccacagggaagacccaagcctaccacagttggggctcataggaatcagcccttctccaaaccactggcctgctgagcattcagttctgccacacccactctgactcccataaaacccagcggctctgacctccccagaggattctccatactgaacctcactgtcttccctcccatcttcccctactcctcctagcagcgagcaatcccattttaactgctttcaccggaacctaatgcaagttcttatggcccagggacttttctcccatgcccacgcttaacctctcaacctttaattttagaaggggattctatttcacaagctcagggagatcccagcaatagcaacaactaccaaaaaccttccaaccttactggctccggctcacccacaggaaaggaatccctcaggtcaaagccacaagacaggctgagtctccatggtgatgggcttaatgggagaaaaacattatttgttttgccacccagggtgaaaatgggaaaaaggaagagatcaaaccctctgtcacttagcccgtttcctgcccagaagcaacagcgcctgctccccagccctaaaatgcctccaagcccctctgactttccccaccctccattgctaagcagcttgcctgggagcttcctcggccactcaatcgctgcttcttctcagctttgctggacatcgtgcaggcaaaacctacaggtatacaggagtggagagtaaccacaagggctctgtactccacaggggcctgggaggccttgctgtttttccctaggatatcctactgtcttcggggcactgggctaaaaatcaaagtcagtgagtgagagaggatcaaggaaggggtgcacatcccccatcctttgtcactgttcaatcattcaagcaaaccaagatgattttcctatgtacctctttcaacatctgtaaacagagaaaaacagtatttgccacctgggtctatcgctttgaagtcctgaggggcaggaacactgttttgtgtgtactgaggtctctt

SEQ ID NO: 55 - DNA, 5000 bp, Artificial Sequence
  Nucleic acid probe
tctgtgctaatggttaagatcagtcaggatcatttatttgactcaaaatgccctgagaacagattggaagaaagaaggacaaaagtcatccaatcataaagcactctcttttcattaggctgcagcctaacggcacctgtggctctccttcctcatccccacaggctgccacaggcattgatctcaggttatgaatttcaacagcaaggaggcttcctccatcagtctccagagaagcatcaagctgcgctttcctgcctcaaactagagcctgaggcgcatgcaccgtggggttcaccatttgctggatgaggagaagagaagctggcacctcagttcccccattttctgtttggcgttcacacctaacagatctcattatgccagcctggcactaccatcctgaagagcaagtggattaaccacaccctatcccagcgctgacctcaccctcccacctaccgtatctttattcccagaagtgtcaaccctttccctccggtaatcaaacatctcgctttgctaattactgctcaatccgattaaacgctgctgaagctcatcaacagaggtggaggtagggggaagggacgaaggcgggagcaaaggctgaggagggggaaagggagccagaggctaaggtctagagaccccagtccctagtggggaacagcagatttggggtgagctgggggccagaccggctgtgccctgggctggtcccgtgagaaccagagtagattactctacagcctgactcatccttctcccccagcagccccccacaggtgatttgtagcccaccgggcactgaggctgctgtttgctgagcacacagattcctcccaactcaccagctgctaccccctgaagcagctcagtctccccccgacatcacccgaacacttcgattccctgtgggaattggggtacctggggcctggtcccctcagaactcccttcagaactccactttccctgctacctttcaagtagctcttgtgacatttctcccaggtctcaaaactctcccaagccccatgtccacggtggcccatatagccccttaggaagggccatttttcattctaattaaaatttgccgttatttggaaggagggcaaagcctgagcttcactctacagctcagccccaagacagcctcaatcctatgcggtttggggatggcagggcgacagcttcctgctctggactctgccagcagctgtgccacagcccccaaaaaagctcagagcatcatccttgccctcccagagcttaaaatttagacgaggaaaactgtgtgtatttgtccccacaaagcaaataggacggcctgtgttattctgcgtggaagtcaccagcattttcaaatagctccagacacccaagtcactacatgtgcagtctgtgctgttcctctccctggctgtgcatctgtccccaggtgtcttcctgtctctcagcatgttatgatgtcttggctgctttggcgaggtgttggacccctccctttctcagtgggccctgttgctgggctctgggttgccatagtgacggttgccaagtccctgaggctgatagccagcgatggggctgtcccacttcgggcagctccagtgtgggctcagaaggcaagagtaaattccagacagcccagtacctggcctcccagctttcaactcccctcctgcggtgcctttgatcctaattgcgtcttaccctgtctctggtgctttggcttagcaagacactcagactgtccccatggatgaattggcgaccccactcctgcctgcaccaaactagccctatccctacccaccccccttggttggtttcatgcaatccaaccctcagctgaaagccattctaaccaagcctttgtcccaattcaagtttttacagtaaaactactgaggtgacggcataccccgccaccatggcaatatcaacttcctgttccccagaaggagcgattagaaaaatcaggaaggagcagcaccagagaggtgaacttgacctctggtggacaaagcaaccatcttggctggtgattgaggtggccaacctggtgcccaacaacagctttgatctaataaacagtggcctttgctaagtaccactgtcagctagaccagccagtaggccaccattttggtcactaagaagggaactgtgttgagtctgaaaggagtcagcacaagggaagggagatcttgggaagtgccactgaagactgggaaaagtccttctcggtccacctctaacctcttacagtcagtaagttccagcttcctcacctcccacctcctgatcccccagactaagccacttggagttccagccagaccacttcctgtttcagaagtgctacttcctgtcctggatgaaggcacttcatatcttgctattggccactggacaaaacgggacacttcttctcatggtagctgtcatgacccaaccaccccaaatggctggagtaagtatgtagaggagaagggctggattcaggatagccacgtgagaggagccctcataccttgtagtagatgtttggagggctctgggggggcccatcctgcacgatatacacaccgaactctgctcagaatccaggtgtccagcccttcagcttcctgccacggagtcctccccacttctctgacagcactgattgaagcactgacttggctacccgggtcccatccgatgtggctgggagagaaaagccagaggaagaggttggcactccagaatctagccctgattctggccccacaccctggtgtgaacatggccccagagattagggctatactcctggaacttgatggtgaagcggagatccaggtctgggcgatcacaagtgagaaggaggtttggggcagggggtgagatgtcagaggcctggcccccagaactttccttccctcaccccacaaccttccaggacagcaattctcagctcacgcttgcccgcctttgtctgagtggtcctaagttagccagctaatagcacagaaggcaaatgcctggtttggccacgaccatcattggggctgatgggaaccagggtctaggaacctgctgcctgtcccacactgggtcttagacttctggggcaagtgctctattgctttggaaaggaaaggagttgacttttctaaccacaaccaacacacttcaccatctccaacccctgcagccccttcctggacatcaatagctgctgagacagggtctcaggactgggtcccaggggactgctacttgatatcttcccagactcccaagccaccccacatccgaaagagcccccaggacaccctaactcccttttctctaccctgtgaaaacaagcttatgttctcaagctacacacctgatactcacaccaacagttcactgaccctctggctgtggggaagagttgggagacagactgtgaagagagtcaaggagccttggggaagttgtccccagagcccccaaacggaccaagtgctgttgttacctgggggttagtgaaaaacaagagtttaggggatacacttgggtctttcctagatggctggggggagaagcggggctgggcaggaagggctgtttccaattatcctgggggaagggaggagttgccttttcgggtgtaagagccctggctttcagcacctctgggcaggggtgacattccgctggcagtatttcttcaaagtccccccattctcagtgtttgggaaggtgttggggggaggagggaggagtcactgcttcaaatgacacactcgtcccccgagcggaggagagtgctcctgctcccctcagagctgagtaatggtccccatgctgctggagaatggcaaacagtgtggagtgtgggcgacaggcgccctgccgcccccaccttgggagtgagatttcaaacaggtttcccccttcagtttaccaagttgcctagcaaccaaccagtcaatggcatgaagccccacccccaagagatggggagggtctatcagtgccttcagtgaggacacttgctccctctgggattcacctccctccccaacccaagccccaacatctctggaattgaatcgacccagggaccacagtgggtaaccagactgccggagatcccagcacaggagctcagcatcctggagacaaggaacaggacctggagtcacccccaaaagctgctttgggctctcagggtgtgacccctaccttgcgggctcctgagaagaagcggttgccatcgggatgaaagcagaggtgggtgatgcccccttggggagatgatgccgctctggccctgcttttttgctagagagggaaggaacaaagtgggggagggggcaatggctcctggcactaaccaggctaagcagagggtcccagcaccaacatgcttggactcaacatcccccccatgctgtctgtcacaagcaggacaaagacctctgatgctccccacatctatcctcccaaaggcttcatccctctcaaatcccagttcttccttggagagatgcaagttgaaggcctacaggaaaacggaatcagctttatcaacaattttctagaaagatttcagagacgggggaatggctgagtcactcggagggaaaaggagtctgaggcgcgtgcctctagcagtgttgtggcagatggggtgtatatatggagcacagagagcaccggaaggctgtacagtgccaacgtcgtcagcgggagagagaatcagcataagcctcaagggaaaagtttcacaaagacgaaagagcaattgtggacttgcagaaagttctgtgaaaaggtggaaaagaaaggagaaaacccgagaaactatattgggaagaagactcagtacacacaagtatatggaaatacccttgaaaaacagtcccgtttgtgaacaggaaacacttggactacactttttccatctccacgaaggacttaaatgtgcagcattgatgaagaggcagtta

SEQ ID NO: 56 - DNA, 5000 bp, Artificial Sequence
  Nucleic acid probe
ttaccatttctgcatattccacctgctccccctcatggtacagctctgggggcaggttataaattcgcaagatgttatcagcactattggtcaagatgcactggactgttttacagtgaaaatctcgggggtggtcagctccctgccccgttgttatccttaccacttacagcctttcaagaagttctcaggttgggtgcggagaagctgtagttccaagcgctgaaaggaagatgggggacaataaacctgggtcgccaagcaaagggggcagaggcctggagaagtgggtctcaggacgcaggaaggcttgtattttcagaaagttcttgctcctcgattcgaggactcaactcactaggggaaccaaactctgtttccaggggagtggagagagaaataacggttgagtctccaaagtcttcatactgcagaggaagcacagcggagattagcctcagccaggatggcttcgaagttctcagggatccgacgcagagacccgcggagccgggacgtgaaaggttagaaggtttcccgttcccatcaagccctagggctcctcgtggctgctgggagttgtagtctgaacgcttctatcaaaagctttcttccttccacccttcatatttgacacaatgcaggattcctccaaaatgatttccaccaattctgccctcacagctctggcttgcagaatgattggggttttcccctcccatgtgctcaagactggcgctaaaagttttgagcttctcaaaagtctagagccaccgtccagggagcaggtagctgctgggacacgcttccctggattgggtaagctcctgactgaacttgatgagtcctctctgagtcacgggctctcggctccgtgtattttcagctcgggaaaatcgcgatgatggggatgttaggaccatccgaactcaaagttgaacgcctaggcagaggagtggagctttggggaaccttgagccggcctaaagcgtacttcttttcgaaacattgagaactcatagctgtatattttagagcccatggcatcctagtgaaaactggggctccattccgaaatgatcatttgggggtgatccgggtccaggcagcccccggctccgctagatggagaaaatccaattgaaggctgtcagtcgtggaagtgagaagtgctaaaccaggggtttgcccgccaggccgggaggaccgtcgcaatctgagaggcccggcagccctgttattgtttggctccacatttacatttctgcctcttgcagcagcatttccggtttctttttgctgctattctccgcctgcatttctttttctggattacttagttatggcctttgcaaaggcaggggtatttgttttgatgcaaacctcaatccctccccttctaccatggcgtgagacagggagggaaagaagtgtgcagaaggcaagcccggaggtattttcaagaatgagtatatctcatcttcccggaggaaaaaaaaacgtttgataatttgtcggaaaaacaatctacctgttatctagctttgggctaggccattccagttccagacgcaggctgaacgtcgtgaagcggaaggggcggaactcgaattcatttctcccgctgccccatctcttagctcgcggttgtttcattccgcagtttcttcccatgcacctgccgcgtaccggccactttgagccaagggcacctgcatttctcttggctccctggccatttggaaggcctagttcagcctggcacatttgtatcctggcccactgatgctggtacccctgacacggagattttagttgctactgaagatttgagagataaagacagggagacctgtctgtagacctgtgtccctccaagtgggattgagactttgggcccgacagcacctcctggcctgttgactgaatagatccctgaaggaggtgtacttgcattaatggagtgggggtgggagcagtaccacagatccgcactaacaaggtgcgatgatagttgcataacttcgtgcaagatgctggtccacacaggggctgccccttgctctttctcgctctcttaacctctcatataacaggcttcccttgaatttgggcttttatccatcccatcacaccctcagcatctctcctggggatgcagaacttttctttttcttcatccacgtgtattccttggcttgctagtgggttgcaggaggtgcttacgcatgtttgtttctttgctgccgtcttccagttgctttatctgttcacttgtgccctgactttcaactctgtctagcctctgcttgcctctgacccctgggcccacctcttaccgatttcttccatactactacccatccacctctcatcacatccccggcggggaatctcctttcttaacctgtggcttctcctccacctacctggagctggagcttaggctccagaaaggacaagggtggttgggagtagatggagcctggttttttaaatgggaaagtttccagtctaacactcaaaatgccgttttcttcttgactgttttacctgcaattggggcatttgccatcagggggcagtgatgcctcaaagaccctggccctactcccctgccttcctaggttggaaagccataggattccattctcatcctgccttcatggtcaaaggcagctgaccccatctcattgggtcttttagtcttcctccggttgaatcctataaccacattcttgcctcagtgtatccacagaacatccaaacccagggacgagtgtggatacttctttgccatgccaccatcttgatttgaattcccgttgtcccagccttaggcccttcaaagcattggtcagggaaaaggggcacagaccctctcactcatgtgatgtcatcttgcaataggtgtgcgtcagaagcacccaggacttccatttgctttgtcccggggctccactgaacaagttggcctgcactggtgttttgttgtggggaccttttattacatggggtctagaacttgacccccttgagggtgcttgttccctctccctgttggtcggtgggttggtagtttctacagttgggcagctggggcccagccaaaccctgtctgacaacctcttggtgaaccttagtacctaaaaggaaatctcaccccatcccacaccctggaggatttcatctcttgtatatctcaccaaggattacgggatttaaatgtctgatttagcaaggctgagcctccagggtggccatctgctccatcagaaagtggcaggatacctgggttcctaggtccttcccagcatcccctcatcctgggcctcatgccaggtagctgaatgaattgaagctttaaactctgccaggaaaacctttcaaagggcttcttctctttcttactagggaatgccaaacactctccccaggagatccagacccgcctctttcagagacttttaacttaaacatctgtccctacccagcaggcatttgacaaccatcctccaccctttcaatcccaaccttttcctagattatgtccctagctctgtttttatttctgctgtgctgcttcagatccattctgactactatataactgctggaatgaggtggatgtgacatatggagtggtagacaaaccaaggaaagcaagtaacaaatacaggagatgttctcatacaggagagtctctcttgtatggagttatcccaagatagtgcgcactaaattagtcaaggcgaagggggtagatgcttggacactggaaggaagagatcaaagctgggtgcaggaaaacatgggggcccgaagggaagaaacacagatccagctagagaaaaagggaagggtccacctgtgggtggagtgtggaggctgcagcattaatggaattttcaggctggcatgcagaggaaaaggaaggagttagtgattagttgataacttgctaaaagaagccagagaaaggatggtgggggcagatctgccagaatggccgcccctcccccagagctccctgggaagtggaacttgaaggatattctgggaaggcgaatgggggaagggagccatccataaaaacaaagcttccagtccctcatttcccagctcacagagtcaccagagggtgagaagcagaacgtgccagcaaagagggaaaaggccacagaaccaccttgtcctcaaagacctgcccaggagccagataaattcaaagtcaccaagatggcgactgtgtagacagggagaaagtatgagagatcaagggacggtggccagagcttagtagctctcacagctaaaccagaactagaaaggctcctaactcctccgtggccatcttagcccagctccctgtctgactgatctgaggttgggagaagttgggaacatgacgaagttgccgagattctcagcatcttcatctcgctgtgggggtgcaggaagatagggggtgagccagttagggatggcaggcacccaggatgtccctgtcatcattctaggaactcctgaaggctgctaatggcacgaaagcctcctactccactcctaccccaagttcttaccccatcctcaggaatcaaacagggtcccaaggacaacgaactggtggcagatggtgctaacagatcagcagctaccagacacctagaaggaactgacgggtgggggccatatgggagcggagttcaagctacagtggagcccaaaagcccactcttacccccttgtttctatggtgatggggcctctaggagcaaagaagatagggagagacaggtggtctgcaggtggtgtccccagatccttcctccattcaccctggggaccaaggtcatagatacccggttcatcttgatgaagacacagggctgcccagtgccagggcggcagacatcattcttttgggcttggatagagtcgttgtaagctaaagaggtgggagggagagactcaggggccagatgttccacttccaaggt

SEQ ID NO: 57 - DNA, 5000 bp, Artificial Sequence
  Nucleic acid probe
tcacgctccaagaacttgttgagcttctgaacatgctggtcccagctttcagtgtcactgacattgacaatgacatcaaggttctcagtcttggggcgaaacacccctctacccactctgaagttctgcactgggactgatcacagagccacagggatgtcttagagccttcctctggaggcaaaatgagacaaactggacaaaatcaaactcctggaaggaactatgagttccttctggaggtttgggggcacaagagcagttagagtagctggcagggggagcctccacactcacccgggtgaacatggcggtgaggaacccataaaaaacgaggtagaagaggaggataaaggctagggaggaggaaacagggtcagcagggcccaaggttcccacccagtgaagcccgaccttgtggaggtctgttattttgcctccaattacatttccaacttctcaaccatgcatagtcctgacaaggcacacatctgttacctaaagacctaacactagcctagctacaaatctctcagagacggcttgttagaaccttcaacagtcaccccaccctcagttcacacccagcaccctgggcaggctgttcccaccaccctggactcccaatccttccatctcagaggacacttcttccatgaagtcctctggctctcccaagcattgctggattcttctcatcacacagtgtcctctgtcccctgtgtctcaacgtcccagcttgaccatccctcttggaagaattccacccctcacctgcacctgtgccggtcaccagaccctcctgccatggtcagcctggggaggcttccaagtcatccgactgtcacagacacctggctggctgatgcacacaaatcaccacagcgaccactcctccctgagcaggggagaataacaagaggaaggggtatgaatgcagcttcaccccccaccaaaccgtctagaaacaaaacgaaatggggtgcaaaaacagacggctgatatatgctcaaaggtgggggccccaggcagggagttagaaagagactaagatacagatgcggcggaagagtccgggagcagatggggcagaatgtcagtttgaggatgtggacggcagtttggatgtgaggtgttgagaaactgggctaaggatttaaggtgacagttgaaaggagaagaaaaacctctccaaggaaatttccaggaagaaggaagtgaaagtgcaaaggctgggctttaatgagcaaagacaagatgaatacagcaagagattgaagacctaggcaggggccaaaccatactttcttcaaactcaagaaacccatagagctaccaaagatagtgcctggttaaattggcatgaacatggaaaggctccttctgcaagggacctcagagtgatacagcctctgtttgtaaagatgaaacctggtcctatcctcaaaacgtaaagcttagcaaacacagccaggggtggcctccggattcccggctcagtacaaacctccagccttttcctggagcccacctttcccaccagcatctccagaaagaaaccagggcatgaaagccttcacaccccctctttcatatctagggctgtacccgtctatctccttcccgtcagtccgtgcaattccagggacttagccgtctgggttcccaacaaaatgggtgtttgcaaagacaagaacaagttgatggtccttagtagaactagcaactaggtttcttggcatctcagatacctctgctaaagcattttacatgtctatctctctctcaagaccctggttcgttctccagcccagtaccaagttttatattcatctttagagtccctgtggtgggctacacagtatttgaccaatgtatcagcagccaatacatgttaattgacctggcaaagtcgatgtcaaagcgaagtcagtcttaatgaaagagtgtagacagctctggatggtaatcaggaatcctggaatctagggcaggctgggggttagccctgaacgtcaacaaggggccagagttaaatgtggatgccctctccattgctgaagactccactaccaacactgctggctgggtggaggcagtgggacagcagctccagtgagtgactcagcctgctgcctgggttactggtgaaaagattcttgtaagaaattaggagtcttggacactgggattctcatggagggagggatgctgggcaccagcattctctagaggtctgtggttgagagctgggtcaccgtattctttgaggggagagatattcccaaatctgtctgttttctccctgcctccacccctttttccagaacccctacccacatctctctgtgaccctctcacgccccctgctgcttcttccctgggcagcaatggccagtcatgactgagatgtcaaaatcccatctcttcccttcctcctgatctctctggaccagccacagaggcaggaaactggtctcaaagggagtagcttctgcggtcagtgccattgcctgggctctgggggcagctgtgagtccagatctcatggcttctgttcagggcctggtccacatccagcctctgaccttgtgcccaaaagggaggtagtgcaaaggtggcattagagcattaaggtggctcggatccagaatcttctccccacttttccactcctccaacctgagtttgcaaaagcacgagctgtttccaatgttgtgctcatgaacttgaacatcattctctcttacctggtaaagcccccaggaagagacgcccttgaggcttggcccagaggttatgtggcctcacccctagctgccttaattctgcctcttcgctatgcctccacctactggccattctcaccacttaaatcctgccccaattttttttggcttttaatgaaaaacaattctgtggagccagtcttgttatgttccctgggctggaaaccttcctaatattgacatgttggactcccctcttccctcattgctcatttgattctacatcacagcttctgaggctagtgggggcagatgctgagatctcggcctgtttgtccagccaggaatcccggcgcaggcagccatccaggtctgagcatttggcctcccccttgcagctctcaacccttcccttctccctaaaagtgtggacttagattcccacggccagccacccagcagtgctttaccgtggtagagggatgcccaataacttggttttgttccctttcctcagaccagctccagacatccctcccctgaggaccagagctagcttacctggtgccatagtcatccttagggttggtatccccataaaaaatcactccctctgggtcccaggttcgaacctcaaaggaggaggaggttctggaaggacacagaaggaaggactgggtcaagggctaggccaaccccatactttgtgatcttggtgaggtcaaaggtcatgacagcgataggctcttgtcctgggccattgctgaggtgtgcccaggggcccagtcacaagactcagctatcacccctctggggatcaatggggtgaaggacagagtcccctccactatcatcctccactgccgagaaactgatctaggtcctccaagatttctgtccactcggggagcctgcattcacaggcctgaggtctagaaacagtcctcccctgcgtcccaagtagcatcctgtgaactcctcccccagtcttttcccaggcctctgaggcctgtctcccaagaaggtattgtctgtgtgtatgattcagtgaatattaagaagggacctgccaaaatgcaaatgcccccaagaggtaacacttcatgctactcaagagctgcaggaaacagctttcagagctgagggtcttaaggcttacccagggtctgggccgctctctcaggctcttcaggatccaagtccgtaggccctttaaggggtctagttgccgtttgcgaggccctgggactttggtcccagacagcggggatgtacccacgccttaggagctagccgaattcgaaaaactctcggatcaggtgaagatcagtgaagaaggtgggggctcagcgcccaggttctggcggaggcggttccccaggcgacagctccaggctgggcatacacccacgtcctctttctctatctctttgtcacaactccaatctcggcagccctgagagcagatggcatcctttctatcactgtttggtagcagagattcttccagcgcccgggaagctactgggtaagagcacctggcccttagaagtccacctctccagccggccgctatgggatatactatttcatctacagtacatggaagggacgcaccatttatctggaggatatctatgtgatgccggaatatcggggtactgggcagaactgtccccagagcccagccggtgtcattttctgatccccttcagctcaaacaatagctattttttgcctctttctctccacaacaggtcaagggattggttggggcgacaagtgaggacctgaagaagcaagggtatatggaggggggacccgggttgaaagagggagtgagaaaacctttttcttttttgaccccagaaaattccgcctggccgtcctggactggaaccagagggccatggacttgtacaaggccctaggagcccaagatctgacggaagctgagggctggcacttcttcctaggaggatctctgtcttgagtttctccttccccatcagctgaagcactcttcagagactacgtccacagacactgatgctgaggcctccctggaggacatagaatatctttgcactccagtatcactccattttcacaaccacccccatgtctacatccttgggctccagcccattcatcacttcattagccatctactcttccatgattggcattaccatttactctaatccaagaagtggatggaacataggacaactcccagaatgtgaagttttcctgctactacccttattt

SEQ ID NO: 58 - DNA, 5000 bp, Artificial Sequence
  Nucleic acid probe
cagtagagagttacagattcttaaaatatcggcgatgcatacgcgaagagggtaaagctcagcgcgtgcgcaccaagcacctttttccttgcggcgactctgtgtccgttgtctggtggaagacataccttagaccttaatcacatcttccccacgatccaagcacatggagaattgtctttcttccacacggtccctttcactttcatcttatattccggttcccaagccaaggagaaatggtcagatccatttacgacgtttggcatctttgacctcaggaaccctgggttggatcagccataaggtggcatctttcgcaagtctctctccccaccacaccttgatctcaagacccactgctctaccattctactcaggaatggatttgaggtctcactcatagtcataatctctaaaaggatctctgaaaggaagcttatgaggttcccttctgtttatcttaagatttccacttcttgaagactcttggccaactaatcatctttcaaagacccttccttatttcacctccccctttgcccaggatttggttccttagaactccttataataccaccttccttatctgctctgaattttcttgcctcccaagagtttttctttgtgtgactgttggtattctgaacctcttggatttgatgcctggaattgtcctagagactctcctgatttctgtttcagttcccctcaggcctttttcccccactgtagtcctctctagtttctcatgcccttggaaatgtctttctttgactccttctctaagtcatatttttagtcattttttactcactcttcatacttacccatttttgccttacttccctcatcagatttatttctctgcggtgactgtgggtacccatgtctaacttaagctttttctcctaatatttctgtattcctgtgtaatccccaggaatcaccttattccctgtctagctgaaggaagctccctgctgatctctttttgtcatttgttgaaatagcttcctaaagtggccctccctgtttaccttggtatcttttccttcatctgggtccttaccacccccaaattctgagttccaagaaaaacgttttctgtcccacatataagtgtccagcagaaagtctctctgaatcttttccctttctcctgtgtgctgtggcagtgatagacttgaatagatgggggctgaaagccaggtaatgtggtaatgtggatctgggcaggttagcattttctgccctgttcctgagttaaaaaccacaaatccctaaattcaggctaatcagtaccctacagatacttatccgttttgaccttgctgctgatttaatattcatgactcagcaaacttgggatgggtcagggagtatttacactgcttctggcttcagtacttggtttttactcagactgttcctgctcttctcctagaaccccctcctttgctttgggagtctagaactagaagacggtgaaggtggggaaatataagggttgagtgagatttggttgaactgctgagtaatgtgagagattactccattctgagctttgcatttctgccttggaagtcatggtagctggggcaagtagaagtaaaagtgcctcagaagacttgaatggagaacagtaggagttggcattaaacctgaatgaacatgctcataactcaggttctttttgaaaaagaggccaggaagtaccagtagtaattaaagccttgggcagtagtaaggctctaagaaagaacattcagtcaagaatgatcttgactgactgcagccgacatatccctaaagacgagaaaggtggcttgtatgaagagtgaaatgcaggcttagattttcccatcgtaaggaaatagcaatctggtaagtgtttgtgttgtctagtgttccgattattgtcttaggacccatgcatcagggcagcgagtctgctcagatctcttgctgtattgcggaggtaggcccttctctctcgatcatttcctttctctgtttgactgcccatattctttccccagtgcagcctttcaggctgtctcattacaccacttgtgtccctatctaaaactcaagtttttttctaaaatccattctagggctttgtgaaggatgtccatgaagactctgtcaccatcttctttgaaaacaagtaagaccttgggaatagagaatccaggagtggagaagagggggcagctgagtcctatgaggcatgtaataggttgggagagctgagggtttgggtgggaactcatatggatcccaggagaggaaagccatgtagcctatggaaatgtaaaatgcaacagttccccttttatcactgttgtgatcagccaaactctcaggaacatccccagtttcaggcgatttgcctacaatgaaattgttaccctggagcgacttcggccagttaatcccaatccccttgcaaccaaaggcagcttcttcaaggttaccatggctgtgcccgaggtgggaatgagggccctctttacagccctaatctctggctttatttctcccaggcatcactcccactttgcagttttctctgttgttcccctggccctctagaaagccctgggagccaactgcatctttctcaacatcacaaacagtgagctcttcattctggtgagtttattctgcctgaatttcacataagccttttgatttcagaaatagagtatgtagctctcaccttatgcttgctccttgctttctctctaaaccattttccttcagtaccctgatagtcagcctttgtgggacttttctagtccctggctctcccttagtgtttattccaagccagttttaatgactttcccactggtttcctcccagtcaaccacagaagcccctgtgaagcgtacttatgtcccgcaatgaagaagctaccaagcacctagaggtaacttttagctccttttccttctgctgaagggtattgtcttcctcccttcctaccctaggctttttctgggcaattaaacctgggtcagctattttatctcagactcctccagatggcagagctctatcatggtatagtgcaattattttcaaattttttctgtgcctgtgcaatttctgggtcatgataatctttgacttcttcagggatcctgctcagggcactggcaggtaaaaattcaccattaccttcagatgtctcctttcaggtattgtttctatttggtgcttacaacatgggattcactcctctaaaacctttcctgtcttcctgcccctgaacccaggggttcctctagttatgcaaagggtcgtgtgcctgggactgtgagattctaccaccatgattcctacagtgacttttcctttcagacaagcaagcagttggcagcagccttgatgggactggcaattgggactcacggtgccaacatccagcaggcccgaaaagtacctggggtgaccgccattgagttgggtgaagagacctgcacttttggtgtgtatatacagttagaggtcagaatcaagattctctacagtgattctgagcttttatccaggattggcacctccctgtggactcccagtttcacaaaaaggctgaattcctcatttacctattccaggcaaagtgattggaaagaacgggaaagtgatccaggagattgtggataaatctggtgtggtgagggttgtgataatgacaagaagaaccccagggaggaggtatgggtacggggcaagtgggcatagagattggtttccagaagtaaatgggattgtcctctgagggcatccttccatttcattttcctttctgatccatcgtttctgctctctgttctctagggaatggttcccttcatttttgttggcacccgagagaacatcagcagaattttccttgtttagccagtgtcttccttctccctgcattcctgagaggttagatatagagggacagtcagggtgcagaagcatgaagcaaggcactgtttgggggatgtgatggatcctgaggtgggatgaatggccgtgaataaatcccattgtgttcttcaggccccagctcagatgtgtctacagcttcagagtatgggccaaaaggaaagatgatgggcacctgactgccaactcctcctcttcttcctctgaccagtgctgaaggatccagacagtaatccctacagcctaccgccgtaaccgtggtaatcggactgatggctctatcagtggagaccgccagccaggtcagccaactctggacaccagaatccttcctcagagctgagagcccagtgactgtggctgactatatctcacgagcagagtctcagagccgccagaggccacccctggaacgcactaaaccctcagaagactctctttcaggacaaatctcctactgaattgcagaggcagctgtgtctgatggagtttagaccagtgtaaactttgaataggatagagaagaaagtgtggttcacattttcttagggaatgtctcccacctgaatttctactcaccgcttcttgcttttctgttttctttcccactttcccctcagggtgactctgtcagcaagcttcctaaccctccctcttgtccaccctacccccagggtaaggggagcctctctccttccccatcagactggatgtgcctttatcctctaatgccccaatctctctcttatccctcctcccatcctgtaccccccatttctggggcctcatcactgtggaagacggggatagtaagagataagtgggtgggaggcacggggaaggttttgttttctgttcctccacatctgacttctcttcattgtctcttgtcccaaagatggtcttacttctgggaattcctgacattcctaaactccccaggcccaagtttgctggtagagggagttgggccactcctataccctgagtcacagcacaggctaggcagggctgctcagagctggagctgttcctgtagaagctga

SEQ ID NO: 59 - DNA, 5000 bp, Artificial Sequence
  Nucleic acid probe
acttggggagttgtcctgggtttcctggctcccccctcatgacttggacgactgaagctaaaacttgccataattcaatagcagtggagaaatataggtttctcacttgggaaaggggttgaatcacggcccaagcacctgctggaatgactcaggaaacaaaaatggtctccctcctcggcctttcttgccccaggggctcctctcagcacaactcgggccctgagggcacttcatccccctgtgtttgatcccattgtggaaagaattggctttaaattgcctgtgcttcagggagggcctatccgcgcctggcagctgtcggaactccggagaccacaaaggggtttatggtttaccccctccacggaaggtcggtctgatcgcagctgcgggtcggggcctcagaacacctcttcagtgccggggcaccagacagcggattttgcattcgactctccccaaagtgtgccgaacaggttggaagcaaaggctccggtcgctctaccctgtctgcccagaactggccagactcacctccaacccagggaagtgccctctaacctcttgtttctctctgcagctcttcccactgcaaacatcctctccttttctaaggatctggatccatcaaccatttgtttccactaaaagaaaaccctggctcagaaagtctgcaagtgcagaggaatggaggatgggaaacggaatgactccagtagctggctctactgcgcctttttctgcttgtggggaggctttgcattccagtagaagagcagctgggcggcgatgaggccgcaagagacagagccagggttacccttcagcatagctcaagaaaggttcccttatggacaaaggttcctcacggggcagctttggcccttagctgggagactcttgggtaagtccccagcacctttcacagtctgtcctctgtagtgcatgaccaggaagcagatggtgatcgtctggagcatcaggaataaggcttcacgatcacgcttctctacagcccccacacgggactggctcattctgaatcgtggccattctcgggatgctcaccccttctcaagatcacctccactggtgattgctgccttgtaacctccttcaccttaatccccattagagttgtgggtattacccttggggtggaagggtgggcccctcacctgaatgggaagttgttagcagcattacagactggagactcaacccttcagcactcttggctcccaggattttaaacacctggggcagctttactaggggaaagatgagaaaagtaggaccaatcttccattccctataaattaacacccaaacttggcttgacctccacggctctccattgcttccccatataaagctgagaaatgcccaagagctctggcttgcttttatgccctggccatccacaacttctctagccttgaagccttcccctcaaggaacttggtctagtgggagtctgactctcacgggtcatgagacaggggcagactgacgctccccaaacggtctgacggtgacccttgtggagctgtctcgcaatcagaaaaaggaagggaggcaaattattccgaaatcagctggaaagaaagcaatcagggtggcggagcctttgggggtgcaatgaaggcccgattcatcaggggctttgaattaagtttaaccctgctagcgatctacgctacagcacaaactccacaaagaagtaattccacccggtgacttccccactggcactaatatggtgtcctccctctaaggagcccgagacacttccgcgcagccgtttaagcggtccgtccgcctcggccgccatattgcaaagctagcttccgccagtctctccagaccgccattgactttcgttgcgcgtgcgcgctcagggaccacgttttggacgcgctgttggaattgtaaagcttgccgaagctgcacgcctgcgcggttagacctcccacgcctttgttgacgcttccagtggagcccccagtggaggtcagagtacccactgctccttcttgattcaaccccttgtaggtgggataggagcatgccaggtgctgcgagggtttaatcgggtcaggagccactggcgctgcctttgggaatgaagttccacagccccccactcattgctttggctgtctgctgcatttgaaactgtacgagggtcaggaagaacaccagctgtgccaggctttggggggtgaacccatgaggcatcgtcacacactgacattttcatccctcctcagcagctgcttccggctccctgagacggaaactggcagagtcttgtagaggatggaaagaaagctgccctgtagccatgacaagctgggagagcgagatttggggggttaagaagagagatgagtggctgcaactgaagctctgccccaggggtgcttggagatctcgaagggatgcattctgagccgagaatgtccactctggaggagtgaggaagggtcagatcacttacgtgctgcgtgggggaaggacacattgtactccaccgccatgtagctcaggtagacaaccttctgctggaggtcctgtggggcgaagaggaaggccctcccctctcatcccccagttttagctttacctctcctccaccctgggttgtgtacatgactcgaatctgaatctgggcttggaggtggacacagggctagccagcctcatggctgaaccgcctcacccaaagtccagctaggctgtctgcaccagttgctcaaccctgctccctcagccctctcttggaaaggaggaatagtcattagtttttgtaattaagttaggggactgttggccctgcttcatcagggcaaagtcaacagtgtcacatgggtgcaaacggagtcctagggcaggcttggcagttgtggcaagtagctatgggaacagctgaggtacaggcccctcccttggtggggaaatcggtttccactcctttcccctggacctctgggccagcattgtcattcacagagccgagaacatggctttccagcgtctgagaactcagatggtgacaagaaagcaacagcccaaaggtaaccatctgctggggcgtccacctcgaccgattctggaaaacaaggaaggggatgaggaatatcaggagctttaagcccagggagggccaggtgtgaaggggtaggggagtgttcagaaaacaaatatgcagcagaccaggtaggcacctgggttccaggaaccctggcttcaagtaggacacttctgacgggcagacccacaacaggctggcagccttccctagccacacctaaaactgaagcagatgcataaaaaggctcaagaatccagtatatggttcccctggcacttactatttttcaagaatcattaggaggagccaagtagtctgctctcagagtacaaagactgatcctttgcccaggaccttcccgccaggcagacaacagccctgggaagtaggacactgccaggctcttcccccagagagaaaccagcatccttgggaatgactctggcttacgctgaaagactggaaagcgtgcacaagagcaaagtgacccaggggattggagcaaccagttttatagaacaggtgatgcatatccatgtcctattgcactcctgccataggacccaagctacgaggcatgttcactctgagaaaactgagcattatccacacccagaataatcactgcccctgccacagaacatttaaaaacatgctctgacatcagtgttgcagcatgacccaggaaaaagtccagcatctgtaagatcacccccacaccaagggatctcttagcccatagtgaaggtgcttattcagaacacagtcaaacttgcccagccagccaggcttcacccaacgcacccatgtcttcctcccaaggtggtgatcagagaagacttctctggtccttggattatactcatcatccctaaatcagccctgaaggagggaatagaataaattaggagttgaatgtggtaaagagcataccctttgggatattcaagtcctaggttaatgaggtcaataagcaaccaatgaaccaagcatttcatggaccagttattaactcatctagtattgcacaccaagctggactagaaacatggctgtagatgcaaatataagctggctacaataccacctctttaaaggaagctgagctgtagtctccgagcaaacagaatctggtgcctactaacaaaagcctcagagcttacaaccctcctccctttagggaaaccagctgcttctagaaatcaatcaagaggccactctaactctatacggctgaaagttgtaatgggggaggacagaattccatttctccagtctcacctcgatgacgccttcgggcagccgggctctgtcaggagtggcctgcatgatgccatcaatctgctgtcccagatccaactcccaccagctcccaaaaaatacccaaaggaggagtttctcctcggggttaggctcagtcctggccgtcaagaaaaccagcacactgaacggccggtttcttctccccgacccgtgttttagccgcgacgaagtgactggcagcctcattggctggagggcccgcctttctcgccggtacggaagtgacatagaagagtcaggcctcgaggcctggcagccatcttggggccctgcactcttccctttacttctgctcccggtctccactggactcaaatgttatttgttttcccctgtagcagagatggattcgacactgcctggacgtgtgccagggactgagctaagcatatccactaatttgaagccccttaaggaagaccaccacttacgtatcaacaaaggatcctttattgaccagagcaggaccgtggcattaagagaggaaaggaactggagtttcttgggaagggactcccatgtctcccttcccatttatgggcttggggtctggggtacgaggctcacacagtgagtt

SEQ ID NO: 60 - DNA, 5000 bp, Artificial Sequence
  Nucleic acid probe
gggcatgtcctgctgggtgaagctgaatggctgagacagggccagatgcttgcagtactgggtgttaacaaagcaagccatgagaaaccccccgtgtcagagagaaccagtgaagccagtgttttggtgttctaaaacagattacttcctgctccacaactgactatgagccaaatgaggggatgcataacagcctcccgcccatcatctgcttacctgcaacacaaaagcaccacagtcactgtcattattctgcctggccacattctagagaagagaaagaaagcaggcccttcacataccaacaccctatacccctccctcaggaaactcaccattttgaagtaacctttccagccctggtggaaatccagtcggtctttctttaccgcctctgccttcaaaataggtgatggtgcgtcgcctcacatcaacagagatgagggaccaatgcacctccaggtggatggggattagcagtagctccttattgaagatgtagatagtgccacaggtcaggtacaacaaacagaagtcttgccttttgccatgaaggttctagagaggaacagagacacaggtaagggaaccaagtgaaagctcttgattttgaggactgaaattaaggaatgccggagccaatcatcctgcctagctgtagaaatttcctggcctttgaatgggagggcagggaaaccagggcattacacaagtggatgtgaattcaaaactcacgtttttggtccaccttttcaccccatcataaccctttggtacggagtttatcatagaagaaactatcttgttttacaatagttacaataggaacccatcccaatccttggggctggccatagcgaaaaacactaaatactggcagaaaggaaaacaactgataaagttcctctgggtactgacctatctggttgggcctaccttttcagggactgtgtccatgaccaggtctccatacatgttcatcacctggggagaagatggggacaccctgctccctgaatcccctctcaggcctgtttctctaccctttctcacctggtcattgagccagttctgtccatacaaggtccccaagtcatccataagccactcggaagcccctcaccatggcattgcctggcatccgctggtaagactggatcagctgcaacaccaggcccttcctgagtaggccaagggtggagctggggcacacagaaatacctactactccctggaatacaaattctaactgactcccttttttctggctcatgatgggtccttctgaggacctgcgaaactctagcttcctcctccccaactacagccctatggaagaaaaggcagcagtattctctccacatccccaaggccccagcccttctttcaaggagggcttttggaaaactcctgctggaaaatgtcctccagcttctctactacctcatcagtgctgaggggtatgaggctgccatacgtttgaaggaattcgtccaagatccggctttctccccaggcctctctgcctcttctgccatgcccaaatctgagccctgaaaaagctcctgggccacatggtccccgatgctgcacacattgcagggcaggggaaatggaatgggcatgagagactggcctggggaaagcacaatgggatgcacacagttgactcatgcatgcaaataagaccgcaagaaggacccactccaagaaatggggaccccataagcatgagataaacatgtccaagagattctacagggatggagagagagttcctactcatagacaggaagaaaggcctgaaactgtttggctggagttctccgaaagggaaatattctttcacaactgtaagatgatgtactccactactgggagagatcccatcccgtcacttggatttcgtgtggacaggatttttgtaaaccacatctccctttctcatctgcgctcaagaccacgggatacctacttagggcatctgtttcctagctctgggagccggcactcttccatcctactttccttaagcccagctctcaccatggcaacaggctcctggtgctccaggctcttaaccctccctaacccctcccccacccctttcccaccttacccgagtcagggtccagaggagactttggagtccacctgagtccatcttcctccatggggggcccagagggcactggtggagtcatcttccagtaccctgaaaaacaaaagatcagggtatcaaaatctaagcatcaccccaccgcagatcctgcctctcttcctggccatgcctcctctggcccagtccagagacaagggaaaatggctaagcagcaggcctcagagatcttatttctaagatcaccaggccaaacccaatagcactgccaggttctacgtgcataatagctacatctctggagatagtgatggaaactggggtccgatagatggagagaaggacgtaggataaggcttcttgtggggtgaagaggcagagtgaggaacggggagccaaacccaggaagacgcctctgttcctgcacattccctctcctttatatactcagctcttggctgtctccagtatgtacccacccagctgggagccactttttataacacaatcacagtttcacaaaccccaggaaggttccatgtggagagaggttaagtttcgcccttgcccggggaattatgtgccttctccagctccaccgttcctctgcctcctgtaaggacccggcattctgcacttcactcctcagacccgcctctgtttaggtttccaacctccctcacctgcgctatagcagctgttgtaggcccggtccgggtgggagggcatacttcttatacatcggaatagagtctcctgccttccttggccttctcgagactttggtatgaatagaggggtcaggaaccctcagtagggggccaaagaggccctctgtgaagctcagagtgtggctggggttacctggctatacagcagatcggacaccatatccttgggcctgtaggcctctcccacgcctaagagctggttgccacatcacctctgtcacatcggagtcatctggaggtgagagtgtgtttccgggatctctccccattctcccaggcttccagggcatcggaactctgctcatggaaagagggtcaaggttaggttaagatgcgagggtcccagccagccctccaccctccctcagcctcaggagaattaggttccagtccctctaggaaggacagggctgccagtgacacccaggaggaacaggcagtgcgcaggaaccgggtgtgttggaataaggcctgcactactccctgtgctgaaattggcaagcaggacttcctgtttcccgagaaaagctcttacataaggctctggacgcgcaggaggaagtctgcctcctaggccaaggaggccgcttctgctgtaccttgggcggaggattgatttttttcctgggcagtgtgccttcctacctctgggcctaggcccagtctctttggcttcctgccgccaggtccagcccctcttagtgcctgaagtggggtcttctggacccatccttgcccacagagtacagttgtaagataagagtgggaatactgtatttatgtgggatggaaaacacgtgaacaggcccaggcagcctctagagggaggggcaggtctggagcaaagagccaactcaccctcccagccctgtagcttgtcccccactctcctggcaaacccttcgccactctcttactgcgttacccatgcagccagatggggcttacctgctggagctgttgattctggcttcctcccagccactcactgtcccgtccacacctgaacacggatgctcagagataaggaaacgtctctctacaggactcggccagagacattcagggcagattgtcacctgcagtcacttctggggagaagagtagaactgaaggagaagcgtgaggggatttaatgttttacttcatgaccatctgaacagcaatagcccctcaaactcatctcaaccaacactgagctgacccccctcatcccactcttgctttactgtctgttttccacatttggcctcacagtggaaagacattctggtgagggggatggtatgtgcaaagtccagaagaggcacaccgggagcaggggtgtcagcgcagcaaacatgaagacagcagaccacgaagagactggaagccatgtagagagccaataccacaagaaaagtgaagtttaggggctcagcatggcagcacaggtcagatggagtggaggtagagggtggggtagaaatggaaaagatgagcagacactagcagcatttagagaaaaagatcatcagaacgtggaatatggaggagacagaagaaaaaagctttcatggggtcccacgcctgggtcacagagagaaccaagaccttcatgaactggcttctgtggttctgagactgatggccgggaagggagtgggaggtcccaggcctctgactgccaggcctagtctccatcaggatcccagtagttcttctaactcatggccaccctgccttcctgtttggggtagggtctgaaggataaatgcaaataaagaatttcacgtagtctgtaagggtagggactgtgtatgctttgcaggcattgtatttccagggtcagcaaaagtttgttgaatgaaaggtaagattcagcccagagctggggctgagtatcagaaggcgctgtattctggggattggtggggcgtgtgccctggacactctctgcttggcctggctattaaccctttggccagtgatgggtgggaaatggggtatctgggttctggtctgcactggcttctggcttttggcaaggccttgagtgggccccccagcttttctggaagaagacacagaaactggtaccagtggcttcctctgtggaagggaaattgggtatgtggggtctggtgagagaattttcaccatacactcat

SEQ ID NO: 61 - DNA, 79 bp, Artificial Sequence
  Nucleic acid probe
aattcgttggaaacgggataatttcagctgactaaacagaagcagtctcagaatcttctttgtgatgtttgcattcaaa

SEQ ID NO: 62 - DNA, 79 bp, Artificial Sequence
  nucleic acid probe
cttcgttcgaaacgggtatatcttcacatgccatctagacagaagcatcctcagaagcttctctgtgatgactgcattc

SEQ ID NO: 63 - DNA, 79 bp, Artificial Sequence
  nucleic acid probe
tgaactctccttttgagagcgcagttttgaaactctctttctgtggcatctgcaaggggacatgtagacctctttgaag

SEQ ID NO: 64 - DNA, 79 bp, Artificial Sequence
  nucleic acid probe
tttcgttggaaacggaatcatcttcacataaaaactacacagatgcattctcaggaactttttggtgatgtttgtattc

SEQ ID NO: 65 - DNA, 83 bp, Artificial Sequence
  nucleic acid probe
cctatggtagtaaagggaatagcttcatagaaaaactagacagaagcattctcagaaaatactttgtgatgattgagtttaac

SEQ ID NO: 66 - DNA, 87 bp, Artificial Sequence
  nucleic acid probe
cacagagctgaacattcctttggatggagcaggtttgagacactctttttgtacaatctacaagtggatatttggacctctctgagg

SEQ ID NO: 67 - DNA, 71 bp, Artificial Sequence
  nucleic acid probe
gtttcacattgcttttcatagagtagttctgaaacatgcttttcgtagtgtctacaagtggacatttggag

SEQ ID NO: 68 - DNA, 58 bp, Artificial Sequence
  nucleic acid probe
cctgtggtggaaaacgaattatcgtcacgtaaaaactagagagaagcattgtcagaaa

SEQ ID NO: 69 - DNA, 65 bp, Artificial Sequence
  nucleic acid probe
tgcattcaactcacagagttgaaggttccttttcaaagagcagtttccaatcactctttgtgtgg

SEQ ID NO: 70 - DNA, 71 bp, Artificial Sequence
  nucleic acid probe
cattccctttgacagagcagtttggaaactctctttgtgtagaatctgcaagtggagatatggaccgcttt

SEQ ID NO: 71 - DNA, 80 bp, Artificial Sequence
  nucleic acid probe
cctatggtagtaaaggaaatagcttcatataaaagctagacagtagcattcacagaaaactcttggtgacgactgagttt

SEQ ID NO: 72 - DNA, 80 bp, Artificial Sequence
  nucleic acid probe
atttcgttggaaacgggataaaccgcacagaactaaacagaagcattctcagaaccttcttcgtgatgtttgcattcaac

SEQ ID NO: 73 - DNA, 80 bp, Artificial Sequence
  nucleic acid probe
cgtagtaaaggaaataacttcctataaaaagaagacagaagctttctcagaaaattctttgggatgattgagttgaactc

SEQ ID NO: 74 - DNA, 79 bp, Artificial Sequence
  nucleic acid probe
acagagctgagcattccttgcgatgtagcagtttagaaacacactttctgcagaatctgcaattgcatatttggacctt

(19) United States
(12) Patent Application Publication
(10) Pub. No.: US 2017/0212122 A1
(43) Pub. Date: Jul. 27, 2017

(54) MULTIPLEX TP53/CEN17/B CELL GENE-PROTEIN CO-DETECTION ASSAY AND UNIQUELY SPECIFIC PROBES FOR 19q12, INSR, ATM, DLEU2, TP53, AND 13q12

(71) Applicant: Ventana Medical Systems, Inc., Tucson, AZ (US)

(72) Inventors: Nelson R. Alexander, Marana, AZ (US); Thomas M. Grogan, Tucson, AZ (US); Leigh A. Henricksen, Oro Valley, AZ (US); Brian D. Kelly, Tucson, AZ (US); Hiro Nitta, Tucson, AZ (US); Stacey Stanislaw, Tucson, AZ (US); Alisa Tubbs, Phoenix, AZ (US)

(21) Appl. No.: 15/472,146

(22) Filed: Mar. 28, 2017

Related U.S. Application Data
(63) Continuation of application No. PCT/EP2015/072231, filed on Sep. 28, 2015.
(60) Provisional application No. 62/057,164, filed on Sep. 29, 2014.

(51) Int. Cl. G01N 33/574; G01N 1/30; C12Q 1/68

(52) U.S. Cl. CPC G01N 33/57492; C12Q 1/6886; G01N 1/30; C12Q 2600/158; G01N 2333/70503; G01N 2001/302

(57) ABSTRACT

Disclosed herein are multiplex methods for co-detecting a B cell marker, TP53 nucleic acid, and Chromosome 17 centromere DNA in a single sample. Samples stained for the B cell marker (e.g., CD79a protein), TP53 nucleic acid, and Chromosome 17 centromere DNA may allow for the identification of the subtype of chronic lymphocytic leukemia (CLL) with the 17p deletion. The methods feature staining the B cell marker (e.g., CD79a) a first distinct color, TP53 in a second distinct color, and chromosome 17 centromere DNA in a third distinct color. Further disclosed are nucleic acid probes specific for 19q12, INSR, ATM, DLEU2, TP53, and 13q12.

### CROSS-REFERENCE TO RELATED APPLICATIONS

This patent application is a continuation of International Patent Application No. PCT/EP2015/072231 filed Sep. 28, 2015, which claims priority to and the benefit of U.S. Provisional Application No. 62/057,164 filed Sep. 29, 2014. Each of the above patent applications is incorporated herein by reference as if set forth in its entirety.

### BACKGROUND OF THE INVENTION

Field

This disclosure relates to immunohistochemistry and in situ hybridization, particularly to the detection of CD79a protein, TP53 nucleic acid, and chromosome 17 centromere DNA in a single sample. The disclosure also relates to nucleic acid probes, particularly useful for the in situ hybridization detection of 19q12, INSR, ATM, DLEU2, TP53, and 13q12.

Background

Many cancers are characterized by genetic changes that lead to aberrant control of cellular processes, or to uncontrolled growth and proliferation of cells. These genetic changes include gain or loss of function (for example, including amplification or deletion of all or a portion of a gene), gene rearrangement, and changes in sequence (for example, substitution, addition, or deletion or one or more bases). Such changes are known to occur in the genetic regions such as 19q12, and with regions associated with various genes, including ATM, DLEU2, AND TP53 genes. In addition to their well-known applicability to genetic abnormalities associated with cancer, abnormalities in these regions, and others such as 13q12 and INSR, have been associated with autism spectrum disorders, metabolism, motoneuron specification, and cardiovascular disease. Detection of genetic changes in these regions can provide diagnostic and prognostic information for patients and in some cases, inform treatment decisions.

Chronic lymphocytic leukemia (‘CLL’) is an indolent disease of the bone marrow, which begins to produce too many lymphocytes (white blood cells). It is the most common leukemia in the Western world, with approximately 25,000 (roughly 13 cases per 100,000 age 65+) new cases in the Western world diagnosed per year. Patients have variable courses, from indolent disease, to rapid progression with limited response to treatment and clinical studies have described CLL as a clinically heterogeneous disease. Because not all patients benefit from the current standard of care, there has been rising research interest to identify the molecular subgroups with prognostic and potential therapy-predictive signatures within a specific clinical stage of CLL progression. Abnormal cytogenetics are found in the majority of patients with CLL, and each subtype is associated with differentiated frequency, outcome, and suggested treatments.

Del 17p is associated with aggressive clinical course and short OS. Some CLL patients present originally with only 13q aberration, but then progress over time and years of treatments to carry the more aggressive aberrations. Until most recently, patients testing 17p-deleted were evaluated for novel agents or stem cell transplantation, with limited results. However, in the past year, 4 novel therapeutics have been FDA approved for treatment of CLL patients, and other trials are still awaiting final results. Obinutuzumab has shown improved results compared to the standard CD20 therapies in all subtypes, and idelalisib has proven effective for patients who have failed prior treatment (many of these patients progress to harbor more aggressive aberrations after treatment). Ibrutinib received updated approval for 17p-patients specifically, and each CLL clinical trial will need to show efficacy in this most aggressive subtype specifically. These new types of highly specific treatments meet an unmet medical need in targeting these specific CLL patients whose clinical course is most grim.

The current standard of care to accurately diagnose and subtype these patients is a fluorescence in situ hybridization (FISH) panel testing for the 4 aberrations (13q, trisomy 12, 17p, and 11q). Some laboratories have begun incorporating CGH array testing or next generation sequencing technology to analyze for these CLL subtypes, but all of these diagnostic technology options are very costly, require a large capital investment in platforms and lab-specific requirements. As a result, the standard FISH testing is typically centralized among a few Heme-specific academic or reference laboratories per region, and turnaround times for results may be as long as 3 weeks. Further, FISH does not incorporate the context of tissue for the reader/scorer.

Workers in the field did not believe it was possible to perform a gene-protein assay to co-detect TP53, chromosome 17, and a B cell marker (e.g., CD79a, etc.) using bright field microscopy. One of the reasons is because scoring a deleted entity (e.g., 17p) using bright field staining was thought to be not possible or at most would result in inaccurate scores. Another reason it was thought not to be possible to perform such a gene-protein assay was because the protease treatment used in in situ hybridization was thought to destroy tissue morphology, and thus also destroy the staining of the B cells. In other words, the in situ process would affect the proper reading of the slide.

As such, prior to the present invention, workers in the field believed that a multiplex assay for co-staining a B cell protein marker (e.g., CD79a protein), TP53 DNA, and chromosome 17 centromere DNA would not be possible and at most would not result in accurate scoring.

### SUMMARY

Disclosed herein are nucleic acid probes that include a plurality of segments of uniquely specific nucleic acid sequences. The disclosed probes are useful for detecting presence of a target nucleic acid in a sample. The uniquely specific nucleic acid sequences are designed to occur only once each in the haploid genome of an organism, and provide high levels of sensitivity and specificity for the detection of a target nucleic acid in a sample. In some embodiments, the probes are useful for detecting presence and location of 19q12, 13q12, ATM, DLEU2, INSR, or TP53.

In some embodiments, the disclosed probes include isolated nucleic acid molecules comprising the sequences provided herein as any one of SEQ ID NOs: 1-60, or nucleic acids having at least 70% (such as at least 75%, at least 80%, at least 85%, at least 90%, at least 91%, at least 92%, at least 93%, at least 94%, at least 95%, at least 96%, at least 97%, at least 98%, at least 99%, or 100%) sequence identity with any one of SEQ ID NOs: 1-60. In other embodiments, the disclosed probes include isolated nucleic acid molecules comprising at least 70% (such as at least 75%, at least 80%, at least 85%, at least 90%, at least 91%, at least 92%, at least 93%, at least 94%, at least 95%, at least 96%, at least 97%, at least 98%, at least 99%, or 100%) sequence identity with at least 250 contiguous nucleotides of any one of SEQ ID NOs: 1-60. In some examples, the nucleic acid probes include nucleic acid molecules consisting of the sequence of any one of SEQ ID NOs: 1-60 or at least 250 contiguous nucleotides of any one of SEQ ID NOs: 1-60. In some examples, the disclosed probes include a detectable label.

Also disclosed herein are probe sets for detecting a target nucleic acid molecule. A probe set includes two or more (for example, 2, 3, 4, 5, 6, 7, 8, 9, 10, or more) of the disclosed nucleic acid probes. In some examples a probe set useful for detecting 19q12 includes two or more nucleic acid molecules selected from SEQ ID NOs: 1-10. In other example, a probe set useful for detecting 13q12 includes two or more nucleic acid molecules selected from SEQ ID NOs: 11-20. In another example, a probe set useful for detecting ATM includes two or more nucleic acid molecules selected from SEQ ID NOs: 21-30. In a still further example, a probe set useful for detecting DLEU2 includes two or more nucleic acid molecules selected from SEQ ID NOs: 31-40. In another example, a probe set useful for detecting INSR includes two or more nucleic acid molecules selected from SEQ ID NOs: 41-50. In another example, a probe set useful for detecting TP53 includes two or more nucleic acid molecules selected from SEQ ID NOs: 51-60.

Also disclosed are kits including one or more of the probes or probe sets disclosed herein.

Methods of using the disclosed probes include, for example, detecting (and in some examples quantifying) a target nucleic acid. For example, the method can include contacting one or more of the disclosed probes or probe sets with a sample containing nucleic acid molecules under conditions sufficient to permit hybridization between the nucleic acid molecules in the sample and the one or more probes. Resulting hybridization is detected, wherein the presence of hybridization indicates the presence (and in some examples, the quantity) of the target nucleic acid sequence. In some embodiments, methods of using the disclosed probes include detecting copy number of the target nucleic acid.

Also disclosed herein are multiplex gene-protein assays for co-staining a B cell protein marker, TP53 DNA, and chromosome 17 centromere DNA, which may help quickly identify patients with the 17p-aberration.

The assays use bright field detection technology and co-detect TP53 DNA, chromosome 17 centromere DNA, and the B-cell protein marker on a single slide. The staining of the B cells helps identify the cells that should be scored for the two DNA markers (TP53 and chromosome 17).

In some embodiments, the methods comprise staining the B cell protein marker by contacting the sample (e.g., blood) with a B cell protein marker-specific antibody and contacting the sample with a first chromogen component for the B cell protein marker-specific antibody, the first chromogen component is adapted to emit or make visible a first color, wherein the presence of the first color indicates the presence of the B cell protein marker. The methods may further comprise staining TP53 genomic DNA and staining chromosome 17 centromere DNA by contacting the sample (e.g., blood) with a TP53 DNA-specific nucleic acid probe and with a chromosome 17 centromere DNA-specific nucleic acid probe, and contacting the sample with a second chromogen component for the TP53 DNA-specific nucleic acid probe and with a third chromogen component for the chromosome 17 centromere DNA-specific nucleic acid probe, wherein the second chromogen component is adapted to emit or make visible a second color and the third chromogen component is adapted to emit or make visible a third color, wherein the presence of the second color indicates the presence of TP53 genomic DNA and the presence of the third color indicates the presence of chromosome 17 centromere DNA. The step of staining the protein is performed before the step of staining the DNA, e.g., the staining of the B cell protein marker is performed before the staining TP53 genomic DNA and staining chromosome 17 centromere DNA.

In some embodiments, the B cell protein marker comprises CD79a protein and the B cell protein marker-specific antibody comprises a CD79a protein-specific antibody. In some embodiments, the first chromogen component comprises fast red, the second chromogen component comprises silver, and the third chromogen component comprises a green chromogen component.

In some embodiments, the sample is subjected to a protease treatment after the step of staining the B cell protein marker but before the step of staining TP53 genomic DNA and staining chromosome 17 centromere DNA. The protease treatment is effective to allow for hybridization of the nucleic acid probes to their respective DNA targets. The protease treatment does not eliminate the first color, and tissue morphology is sufficiently maintained so as to allow for the detection of the first color. In some embodiments, the sample is subjected to a heat treatment after the step of staining the B cell protein marker but before the protease treatment.

The foregoing and other features of the disclosure will become more apparent from the following detailed description, which proceeds with reference to the accompanying figures.

### BRIEF DESCRIPTION OF THE DRAWINGS

FIGS. 1 and 2 show dual ISH assays for 17p-(TP53/Chr17), 13q-(DLEU/13q), Trisomy 12, and 11q-(ATM/Chr11). Ovals indicate exemplary stained cells. Dashed arrows indicate red dots, which correspond to Chr17, 13q, Chr12, and 11q probes labeled with DIG. Solid arrows indicate black dots, which correspond with TP53, DLEU, and ATM probes labeled with DNP.

FIG. 3 is a Whole Blood Smear 17p-dual Chromogenic ISH assay. Solid arrows indicate black dots, which are TP53 nucleic acid probes labeled with DNP. Dashed arrows indicate red dots, which are Chr15 nucleic acid probes labeled with DIG.

FIG. 4 shows a 19q12 Dual Chromogenic ISH assay. Ovals indicate exemplary stained cells. Dashed arrows indicate red dots, which correspond to Chr17, 13q, Chr12, and 11q probes labeled with DIG. Solid arrows indicate black dots, which correspond with 19q12 nucleic acid probes labeled with DNP. Dashed arrows indicate red spots, which correspond with INSR nucleic acid probes labeled with DIG.

FIG. 5 shows a round shape defined by a simple closed curve fitting within a first region. The first region is an area on and between an inner concentric circle and an outer concentric circle. The inner concentric circle has an inner radius (Rᵢₙ and the outer concentric circle has an outer radius (Rₒᵤₜ). Rᵢₙ is ≧50% of Rₒᵤₜ. The simple closed curve has a radius Rₛᵢₘₚₗₑ wherein Rᵢₙ≦Rₛᵢₘₚₗₑ≦Rₒᵤₜ.

FIG. 6 shows a schematic representation of various steps used to stain CD79a, TP53 DNA, and centromere 17 DNA. The present invention is not limited to the markers, reagents, steps, or order of steps shown in FIG. 6.

FIG. 7 shows a gene-protein assay in normal bone marrow staining for CD79a (arrows pointing to regions of red stain), TP53 (arrows pointing to black dots), and chromosome 17 DNA (arrows pointing to diffuse blue/green dots).

FIG. 8 shows a gene-protein assay in a peripheral blood smear staining for CD79a (solid arrow, pointing to region of red stain), TP53 (dashed arrows pointing to black dots), and chromosome 17 DNA (dotted arrows pointing to diffuse blue/green dots). This sample has a homozygous 17p deletion.

### SEQUENCES

This application hereby incorporates-by-reference a sequence listing submitted herewith in a computer-readable format, having a file name of 31819_US2_ST25, created on Mar. 28, 2017, which is 404,175 bytes in size.

The nucleic acid sequences provided herein are shown using standard letter abbreviations for nucleotide bases, as defined in 37 C.F.R. 1.822. Only one strand of each nucleic acid sequence is shown, but the complementary strand is understood as included by any reference to the displayed strand. In the provided sequences:

SEQ ID NOs: 1-10 are nucleic acid sequences of probes including linked uniquely specific nucleic acid segments complementary to region 19q12 of the human genome.

SEQ ID NOs: 11-20 are nucleic acid sequences of probes including linked uniquely specific nucleic acid segments complementary to region 13q12 of the human genome.

SEQ ID NOs: 21-30 are nucleic acid sequences of probes including linked uniquely specific nucleic acid segments complementary to the human ATM gene.

SEQ ID NOs: 31-40 are nucleic acid sequences of probes including linked uniquely specific nucleic acid segments complementary to the human DLEU2 gene.

SEQ ID NOs: 41-50 are nucleic acid sequences of probes including linked uniquely specific nucleic acid segments complementary to the human INSR gene.

SEQ ID NOs: 51-60 are nucleic acid sequences of probes including linked uniquely specific nucleic acid segments complementary to the human TP53 gene.

SEQ ID NOs: 61-74 are nucleic acid sequences of probes to the human chromosome 17 centromere DNA.

### DETAILED DESCRIPTION

### I. Abbreviations

aCGH array comparative genomic hybridization

ATM ataxia telangiectasia mutated

AP alkaline phosphatase

CGH comparative genomic hybridization

CISH chromogenic in situ hybridization

DLEU2 deleted in lymphocytic leukemia 2 (non-protein coding)

DNP 2,4-dinitrophenyl

FISH fluorescent in situ hybridization

HRP horseradish peroxidase

INSR insulin receptor

ISH in situ hybridization

SISH silver in situ hybridization

TP53 tumor protein p53

### II. Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety by reference herein.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Antibody: A polypeptide that includes at least a light chain or heavy chain immunoglobulin variable region and specifically binds an epitope of an antigen (such as CD79a protein). Antibodies include monoclonal antibodies, polyclonal antibodies, or fragments of antibodies. An antibody can be conjugated or otherwise labeled with a detectable label, such as an enzyme, hapten, or fluorophore.

Detectable label: A compound or composition that is conjugated directly or indirectly to another molecule (such as a nucleic acid probe) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable). Exemplary labels in the context of the probes disclosed herein are described below. Methods for labeling nucleic acids, and guidance in the choice of labels useful for various purposes, are discussed, e.g., in Sambrook and Russell, in Molecular Cloning: A Laboratory Manual, 3rd Ed., Cold Spring Harbor Laboratory Press (2001) and Ausubel et al., in Current Protocols in Molecular Biology, Greene Publishing Associates and Wiley-Intersciences (1987, and including updates).

Detectable labels may include chromogenic, fluorescent, phosphorescent and/or luminescent molecules, catalysts (such as enzymes) that convert one substance into another substance to provide a detectable signal (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Particular examples of detectable labels include: enzymes, such as horseradish peroxidase, alkaline phosphatase, acid phosphatase, glucose oxidase, β-galactosidase or β-glucuronidase; fluorophores, such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles, such as quantum dots (U.S. Pat. Nos. 6,815,064, 6,682,596 and 6,649,138, each of which is incorporated in its entirety by reference herein); metal chelates, such as DOTA and DPTA chelates of radioactive or paramagnetic metal ions like Gd3+; and liposomes, for example, liposomes containing trapped fluorescent molecules. Where the detectable label includes an enzyme, a detectable substrate such as a chromogen, a fluorogenic compound, or a luminogenic compound is used in combination with the enzyme to generate a detectable signal (a wide variety of such compounds are commercially available, for example, from Life Technologies, Carlsbad, Calif.).

Alternatively, an enzyme can be used in a metallographic detection scheme. In some examples, metallographic detection methods include using an enzyme, such as alkaline phosphatase, in combination with a water-soluble metal ion and a redox-inactive substrate of the enzyme. The substrate is converted to a redox-active agent by the enzyme, and the redox-active agent reduces the metal ion, causing it to form a detectable precipitate (see, for example, U.S. Pat. Nos. 7,642,064; 7,632,652; each of which is incorporated by reference herein). In other examples, metallographic detection methods include using an oxido-reductase enzyme (such as horseradish peroxidase) along with a water soluble metal ion, an oxidizing agent and a reducing agent, again to form a detectable precipitate (see, for example, U.S. Pat. No. 6,670,113, which is incorporated in its entirety by reference herein). Haptens are small molecules that can be bound by antibodies. Exemplary haptens include dinitrophenyl (DNP), biotin, digoxigenin (DIG), and fluorescein. Additional haptens include oxazole, pyrazole, thiazole, nitroaryl, benzofuran, triperpene, urea, thiourea, rotenoid, coumarin and cyclolignan haptens, such as those disclosed in U.S. Pat. No. 7,695,929, which is incorporated in its entirety by reference herein.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. The presence of a chemical which decreases hybridization (such as formamide) in the hybridization buffer will also determine the stringency (Sadhu et al., J. Biosci. 6:817-821, 1984). Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). Hybridization conditions for ISH are also discussed in Landegent et al., Hum. Genet. 77:366-370, 1987; Lichter et al., Hum. Genet. 80:224-234, 1988; and Pinkel et al., Proc. Natl. Acad. Sci. USA 85:9138-9142, 1988.

Immunohistochemistry (IHC): A method of determining the presence or distribution of an antigen in a sample by detecting interaction of the antigen with a specific binding agent, such as an antibody. A sample is contacted with an antibody under conditions permitting antibody-antigen binding. Antibody-antigen binding can be detected by means of a detectable label conjugated to the antibody (direct detection) or by means of a detectable label conjugated to a secondary antibody, which binds specifically to the primary antibody (e.g., indirect detection).

In situ hybridization (ISH): A method of determining the presence or distribution of a nucleic acid in a sample using hybridization of a labeled nucleic acid probe to localize a specific DNA or RNA sequence in a portion or section of tissue (in situ), or, if the tissue is small enough (e.g., plant seeds, Drosophila embryos), in the entire tissue (whole mount ISH). DNA ISH can be used to determine the structure of chromosomes, such as for use in medical diagnostics to assess chromosomal integrity and/or to determine gene copy number in a sample. RNA ISH measures and localizes mRNAs and other transcripts within tissue sections or whole mounts.

For ISH, sample cells and tissues are usually treated to fix the target nucleic acids in place and to increase access of the probe to the target molecule. The detectably labeled probe hybridizes to the target sequence at elevated temperature, and then the excess probe is washed away. Solution parameters, such as temperature, salt and/or detergent concentration, can be manipulated to remove any non-identical interactions (e.g., so only exact sequence matches will remain bound). Then, the labeled probe is localized and potentially quantitated in the tissue using either autoradiography, fluorescence microscopy or immunohistochemistry, respectively. ISH can also use two or more probes, which are typically differently labeled to simultaneously detect two or more nucleic acids.

Intron: An intron is any nucleotide region or sequence within a gene that is removed by RNA splicing during generation of a final mature RNA product of a gene. The term intron may refer to both the DNA sequence within a gene or the corresponding sequence in unprocessed RNA transcripts. Introns are found in the genes of most organisms, and can be located in a wide range of genes, including those that generate proteins, ribosomal RNA (rRNA), and transfer RNA (tRNA). When proteins are generated from intron-containing genes, RNA splicing takes place as part of the RNA processing pathway that follows transcription and precedes translation. The length of intron sequences is highly variable, ranging from less than 100 base pairs to tens of thousands or even hundreds of thousands of base pairs.

Isolated: An “isolated” biological component (such as a nucleic acid molecule, protein, or cell) has been substantially separated or purified away from other biological components in a preparation, a cell of an organism, or the organism itself, in which the component occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. In some examples, the nucleic acid probes disclosed herein are isolated nucleic acid probes.

TP53 Tumor Protein p53

The protein encoded by this gene is a tumor suppressor protein containing transcriptional activation, DNA binding, and oligomerization domains. It responds to cellular stresses and regulates expression of target genes with the effect of inducing cell cycle arrest, apoptosis, senescence, DNA repair, or changes in metabolism. A variety of human cancers are associated with mutations in this gene. A TP53 probe may be used with a Chromosome 17 centromere probe.

ATM Ataxia-Telangiectasia Mutated Gene

The protein encoded by this gene is a member of the phosphatidylinositol 3-kinase family. These proteins respond to DNA damage by phosphorylating substrates involved in DNA repair and/or cell cycle control. Reference is made to Savitsky et al. (Science 268: 1749-1753, 1995) which suggested that an increased risk of cancer may be associated with aberrations at this gene.

DLEU2 Deleted in Lymphocytic Leukemia 1 (Non-Protein Coding)

The DLEU2 gene is located in region 13q14 and is a non-coding region originally identified as a potential tumor suppressor gene and that may be deleted in patients with B-cell chronic lymphocytic leukemia.

INSR Insulin Receptor

The INSR gene encodes for an insulin receptor comprising a tetramer of two alpha and two beta subunits. The insulin receptor is a transmembrane receptor that is activated by insulin, IGF-I, IGF-II. The insulin receptor plays a key role in the regulation of glucose homeostasis, a functional process that under degenerate conditions may result in a range of clinical manifestations including diabetes and cancer.

19q12 Amplicon

The 19q12 locus is amplified in many cancer types including ovarian, breast and colon. Several genes are encoded within the region including CCNE1 and URI, which have been implicated as potential “drivers” of cancer cell survival. Each of these targets may provide valuable prognostic/predictive information for patient care.

13q12 Region

The 13q12 region was selected as a highly conserved and uniquely distinct region of chromosome 13 that can serve as a surrogate for a centromere probe for chromosome 13. Chromosome 13 is known to being highly repetitive with other centromeres and thus a probe specific to a typically unamplified region near the centromere of chromosome 13 is desirable.

Probe: A nucleic acid molecule that is capable of hybridizing with a target nucleic acid molecule (e.g., genomic target nucleic acid molecule) and, when hybridized to the target, is capable of being detected either directly or indirectly. Thus probes permit the detection, and in some examples quantification, of a target nucleic acid molecule. In particular examples, a probe includes at least two segments complementary to uniquely specific nucleic acid sequences of a target nucleic acid molecule and are thus capable of specifically hybridizing to at least a portion of the target nucleic acid molecule. Generally, once at least one segment or portion of a segment has (and remains) hybridized to the target nucleic acid molecule other portions of the probe may (but need not) be physically constrained from hybridizing to those other portions' cognate binding sites in the target (e.g., such other portions are too far distant from their cognate binding sites); however, other nucleic acid molecules present in the probe can bind to one another, thus amplifying signal from the probe. A probe can be referred to as a “labeled nucleic acid probe,” indicating that the probe is coupled directly or indirectly to a detectable moiety or “label,” which renders the probe detectable.

Sample: A specimen containing DNA (for example, genomic DNA), RNA (including mRNA), protein, or combinations thereof, obtained from a subject. Examples include, but are not limited to, chromosomal preparations, peripheral blood, urine, saliva, tissue biopsy, fine needle aspirate, surgical specimen, bone marrow, amniocentesis samples, and autopsy material. In one example, a sample includes genomic DNA. In some examples, the sample is a cytogenetic preparation, for example which can be placed on microscope slides. In particular examples, samples are used directly, or can be manipulated prior to use, for example, by fixing (e.g., using formalin).

Sequence identity: The identity (or similarity) between two or more nucleic acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8:155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology and on the Internet, for use in connection with the sequence analysis programs blastp, blastn, blastx, tblastn and tblastx. Additional information can be found at the NCBI web site.

BLASTN may be used to compare nucleic acid sequences, while BLASTP may be used to compare amino acid sequences. If the two compared sequences share homology, then the designated output file will present those regions of homology as aligned sequences. If the two compared sequences do not share homology, then the designated output file will not present aligned sequences.

The BLAST-like alignment tool (BLAT) may also be used to compare nucleic acid sequences (Kent, Genome Res. 12:656-664, 2002). BLAT is available from several sources, including Kent Informatics (Santa Cruz, Calif.) and on the Internet (genome.ucsc.edu).

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 15 consecutive nucleotides from an identified sequence as follows contains a region that shares 75 percent sequence identity to that identified sequence (that is, 15÷20*100=75).

Subject: Any multi-cellular vertebrate organism, such as human or non-human mammals (e.g., veterinary subjects).

Target nucleic acid sequence or molecule: A defined region or particular portion of a nucleic acid molecule, for example a portion of a genome (such as a gene or a region of mammalian genomic DNA containing a gene of interest). In an example where the target nucleic acid sequence is a target genomic sequence, such a target can be defined by its position on a chromosome (e.g., in a normal cell), for example, according to cytogenetic nomenclature by reference to a particular location on a chromosome; by reference to its location on a genetic map; by reference to a hypothetical or assembled contig; by its specific sequence or function; by its gene or protein name; or by any other means that uniquely identifies it from among other genetic sequences of a genome. In some examples, the target nucleic acid sequence is mammalian genomic sequence (for example human genomic sequence).

In some examples, alterations of a target nucleic acid sequence (e.g., genomic nucleic acid sequence) are “associated with” a disease or condition. In some examples, detection of the target nucleic acid sequence can be used to infer the status of a sample with respect to the disease or condition. For example, the target nucleic acid sequence can exist in two (or more) distinguishable forms, such that a first form correlates with absence of a disease or condition and a second (or different) form correlates with the presence of the disease or condition. The two different forms can be qualitatively distinguishable, such as by polynucleotide polymorphisms, and/or the two different forms can be quantitatively distinguishable, such as by the number of copies of the target nucleic acid sequence that are present in a cell.

Topoisomerase II alpha (TOP2A): DNA topoisomerases (EC 5.99.1.3) are enzymes that control and alter the topologic states of DNA in both prokaryotes and eukaryotes. Topoisomerase II from eukaryotic cells catalyzes the relaxation of supercoiled DNA molecules, catenation, decatenation, knotting, and unknotting of circular DNA. The reaction catalyzed by topoisomerase II likely involves the crossing-over of two DNA segments. The gene encoding topoisomerase II in humans is present at cytogenetic location: 17q21.2 and has genomic coordinates (GRCh37): 17:38, 544, 772-38, 574, 201. Tsai-Pflugfelder et al. determined the entire coding sequence of the human TOP2 gene as early as 1988 (Proc. Natl. Acad. Sci. USA 85:7177-7181, 1988). Lang et al. reported the complete structures of the human INSR and TOP2B genes in 1998 (Gene 221:255-266, 1998). The human INSR gene spans approximately 30 kb and contains 35 exons.

Uniquely specific sequence: A nucleic acid sequence (for example, a sequence of at least of at least 20 bp (such as at least 20 bp, 30 bp, 40 bp, 50 bp, 60 bp, 70 bp, 80 bp, 90 bp, 100 bp, or more) that is present only one time in a haploid genome of an organism. In a particular example, a uniquely specific nucleic acid sequence is a nucleic acid sequence from a target nucleic acid that has 100% sequence identity with the target nucleic acid and has no significant identity to any other nucleic acid sequences present in the specific haploid genome that includes the target nucleic acid.

Vector: Any nucleic acid that acts as a carrier for other (“foreign”) nucleic acid sequences that are not native to the vector. When introduced into an appropriate host cell a vector may replicate itself (and, thereby, the foreign nucleic acid sequence) or express at least a portion of the foreign nucleic acid sequence. In one context, a vector is a linear or circular nucleic acid into which a nucleic acid sequence of interest is introduced (for example, cloned) for the purpose of replication (e.g., production) and/or manipulation using standard recombinant nucleic acid techniques (e.g., restriction digestion). A vector can include nucleic acid sequences that permit it to replicate in a host cell, such as an origin of replication. A vector can also include one or more selectable marker genes and other genetic elements known in the art. Common vectors include, for example, plasmids, cosmids, phage, phagemids, artificial chromosomes (e.g., BAC, PAC, HAC, YAC), and hybrids that incorporate features of more than one of these types of vectors. Typically, a vector includes one or more unique restriction sites (and in some cases a multi-cloning site) to facilitate insertion of a target nucleic acid sequence.

### III. Multiplex Gene-Protein Assays

Disclosed herein are methods for co-detecting multiple target molecules, such as at least one gene and at least one protein (e.g., one protein and two genes, etc.), in a single sample. In particular embodiments, the methods of the present invention disclose multiplex gene-protein assays for detecting a B cell protein marker, TP53 genomic DNA, and chromosome 17 centromere DNA.

The present invention is not limited to detecting a B cell marker and/or TP53 and/or chromosome 17; other gene-protein marker combinations may be detected using methods of the present invention. For example, in some embodiments, the methods of the present invention feature detecting a B cell marker, DLEU2 DNA, and chromosome 13 DNA; or, the methods may feature detecting a different protein marker and different gene pairs altogether; or, the methods may feature detecting two protein markers and one or more genes, etc. The present invention is also not limited to the B cell marker comprising CD79a. Any appropriate B cell marker may be used. In one embodiment, one or more B-cell markers selected from the group consisting of CD79a, CD79b, BCL-2, CD19, CD22, MUM1, PAX5, CD20, Oct2, and Bob.1 is used. In another embodiment, the B cell marker is CD79a. In another embodiment, the B cell marker is CD79b. In another embodiment, the B cell marker is BCL-2. In another embodiment, the B cell marker is CD19. In another embodiment, the B cell marker is CD22. In another embodiment, the B cell marker is MUM1. In another embodiment, the B cell marker is PAX5. In another embodiment, the B cell marker is CD20. In another embodiment, the B cell marker is Oct2. In another embodiment, the B cell marker is Bob.1.

In some embodiments of the methods, a sample is stained for the B cell marker by contacting the sample with a B cell marker-specific antibody, and then the sample is contacted with a means (e.g., a first chromogen component) for detecting the B cell marker-specific antibody corresponding to the B cell marker. In one embodiment, the sample is stained for one or more B-cell markers selected from the group consisting of CD79a, CD79b, BCL-2, CD19, CD22, MUM1, PAX5, CD20, Oct2, and Bob.1 by contacting the sample with an antibody specific for the one or more B cell markers, and then the sample is contacted with a first chromogen component for visualizing the B cell marker, e.g., the first chromogen component is adapted to emit or make visible a first color corresponding to the B cell marker. In another embodiment, the B cell marker is CD79a and the antibody is a CD79a-specific antibody. In another embodiment, the B cell marker CD79b and the antibody is a CD79b-specific antibody. In another embodiment, the B cell marker is BCL-2 and the antibody is a BCL-2-specific antibody. In another embodiment, the B cell marker is CD19 and the antibody is a CD19-specific antibody. In another embodiment, the B cell marker is CD22 and the antibody is a CD22-specific antibody. In another embodiment, the B cell marker is MUM1 and the antibody is a MUM1-specific antibody. In another embodiment, the B cell marker is PAX5 and the antibody is a PAX5-specific antibody. In another embodiment, the B cell marker is CD20 and the antibody is a CD20-specific antibody. In another embodiment, the B cell marker is Oct2 and the antibody is an Oct-2-specific antibody. In another embodiment, the B cell marker is Bob.1 and the antibody is a Bob.1-specific antibody.

In some embodiments of the methods, the sample is stained for TP53 DNA and chromosome 17 DNA by contacting the sample with a TP53 DNA-specific nucleic acid probe and a chromosome 17 DNA-specific nucleic acid probe (e.g., chromosome 17 centromere probe). The sample may then be contacted with a second chromogen component for visualizing the TP53 DNA and a third chromogen component for visualizing chromosome 17 DNA. For example, the second chromogen component is adapted to emit or make visible a second color corresponding to TP53 DNA, and the third chromogen component is adapted to emit or make visible a third color corresponding to the chromosome 17 DNA.

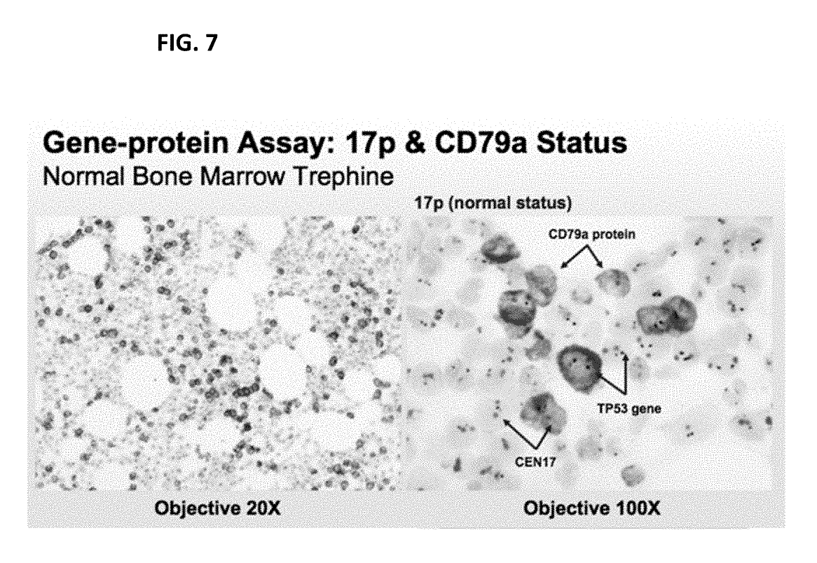

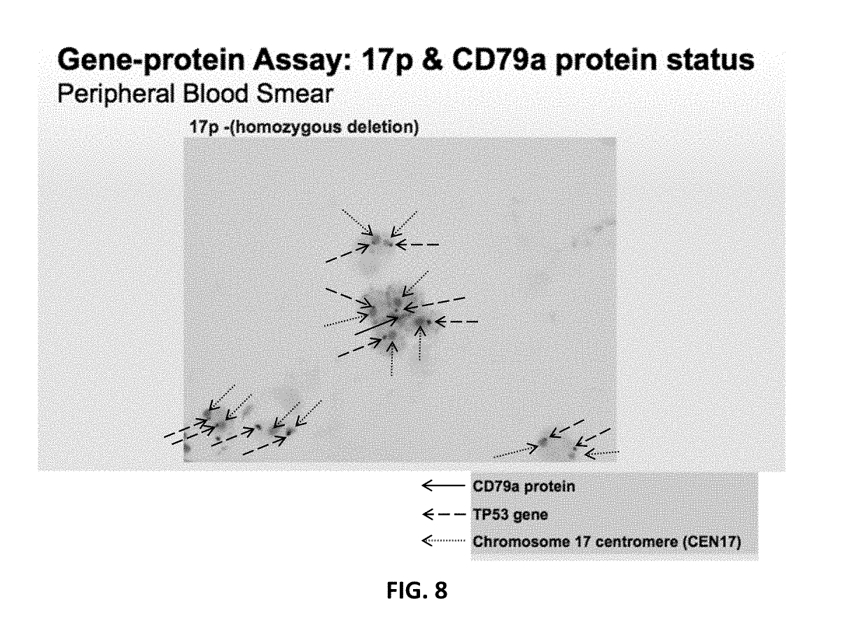

FIG. 7 shows a gene-protein assay of a normal bone marrow sample stained for CD79a (red), TP53 (black), and chromosome 17 (blue/green). FIG. 8 shows a gene-protein assay of a blood sample stained for CD79a (red), TP53 (black), and chromosome 17 (blue/green). That sample shows a homozygous 17p deletion.

Samples may be blood samples, however, the samples are not limited to blood. In some embodiments, the sample comprises tissue, bone marrow, or any other appropriate sample material. Samples are further discussed below.

The methods may utilize different detectable labels and/or detection systems for each of the targets such that each can be individually detected in a single sample. For example, in some embodiments, the B cell marker is stained with a first chromogen (producing a first color), TP53 is stained with a second chromogen (producing a second color), and chromosome 17 is stained with a third chromogen (producing a third color). The proteins/DNA may be detected by the chromogens using additional reagents such as secondary antibodies specific for the primary antibodies. The first color is transparent enough to allow visualization of the second color and/or third color. In some embodiments, the first color blocks no more than 50%, no more than 40%, no more than 30%, no more than 20%, no more than 10%, no more than 8%, no more than 6%, no more than 4%, no more than 2%, or none of the intensity of the second color and/or the third color. Detection includes but is not limited to bright field microscopy.

In one embodiment, the first chromogen component comprises fast red, the second chromogen component comprises silver, and the third chromogen component comprises a green chromogen component.

In some embodiments, the step of staining protein is performed before the step of staining DNA. For example, the step of staining the B cell marker is performed before the step of staining TP53 genomic DNA and staining chromosome 17 centromere DNA.

In one embodiment, the sample is stained for CD79a by contacting the sample with a CD79a-specific antibody. CD79a antibodies are known in the art and are commercially available. In one example, the sample is contacted with an anti-CD79a rabbit monoclonal antibody, such as the anti-CD79a SP18 rabbit monoclonal antibody (Ventana Medical Systems, Inc., Tucson, Ariz., e.g., catalog number 790-4432). In some embodiments, the CD79a-specific antibody is detectably labeled (the label may be referred to as the first chromogen component), allowing for detection of CD79a protein in the sample. In some embodiments, after contacting the sample with the CD79a-specific antibody (the primary antibody), the sample is contacted with a detectably labeled secondary antibody against the primary antibody, such as a secondary antibody conjugated to an enzyme (e.g., alkaline phosphatase (AP), horseradish peroxidase (HRP), etc.) or a secondary antibody conjugated to a hapten that can be detected with a further reagent conjugated to an enzyme. The presence of the CD79a protein is detected by contacting the enzyme with a chromogen and/or substrate composition, which produced a colored precipitate (first color) in the vicinity of the CD79a-specific antibody.

In some embodiments, the sample is contacted with a CD79a-specific antibody; the sample is then contacted with an AP-conjugated secondary antibody that specifically binds the primary antibody, for example under conditions sufficient for the secondary antibody to specifically bind to the primary antibody. The sample is then contacted with a naphthol phosphate and Fast Red chromogen, which produces a red precipitate near the anti-CD79a antibody (and CD79a protein) that can be visually detected by light microscopy. In one example, the reagents (except for the anti-CD79a antibody) are included in a kit, such as the ULTRAVIEW Universal Alkaline Phosphatase Red Detection Kit (Ventana Medical Systems, Tucson, Ariz., catalog number 760-501). One of ordinary skill in the art can select alternative detection reagents (such as alternative secondary antibodies, enzymes, substrates, and/or chromogens) including those that produce a different color precipitate for detection of the CD79a.

In some examples of the disclosed methods, the sample is contacted with a nucleic acid probe that specifically binds to TP53 genomic DNA. Methods of constructing TP53-specific nucleic acid probes are known to one of ordinary skill in the art, and TP53-specific nucleic acid probes are disclosed herein (below). In one example, the sample is contacted with a hapten-labeled TP53 nucleic acid probe, for example under conditions specific for the probe to specifically bind to (hybridize with) TP53 genomic DNA in the sample. The sample is then contacted with an antibody that specifically binds to the hapten, for example, under conditions sufficient for the antibody to specifically bind to the hapten. The antibody may be conjugated to an enzyme (such as AP or HRP) or alternatively, the sample may be contacted with a second antibody that specifically binds the anti-hapten antibody, where the second antibody is conjugated to an enzyme. The presence of TP53 genomic DNA is detected by contacting the enzyme with a chromogen and/or substrate composition to produce a colored precipitate in the vicinity of the TP53 nucleic acid probe. In some examples, the number of TP53 DNA copies in the sample is scored by a slide reader by counting the number of areas of precipitate (“spots”) in the nuclei of the tumor cells.

In one particular example, the method includes contacting the sample with a TP53 genomic DNA probe conjugated to dinitrophenyl (DNP), for example under conditions sufficient for the TP53 probe to specifically bind to TP53 genomic DNA in the sample. The sample is then contacted with an anti-hapten antibody that specifically binds DNP, for example under conditions sufficient for the anti-hapten antibody to specifically bind to the DNP. The sample is then contacted with an HRP-conjugated secondary antibody that specifically binds to the anti-hapten antibody, for example under conditions sufficient for the secondary antibody to specifically bind to the anti-DNP antibody. The sample is then contacted with silver acetate, hydroquinone, and hydrogen peroxide. The silver ions are reduced by hydroquinone to metallic silver ions, which can be visually detected by light microscopy as black spots. In one example, the reagents (except for the TP53 probe) are included in a kit, such as the ULTRAVIEW SISH DNP Detection Kit (Ventana Medical Systems, Tucson, Ariz., catalog number 760-098). One of ordinary skill in the art can select alternative detection reagents (such as alternative haptens, antibodies, enzymes, substrates, and/or chromogens) including those that produce a different color precipitate for detection of TP53 genomic DNA.

Examples of TP53 probes are disclosed herein (see below). Briefly, in some embodiments, the TP53 DNA-specific nucleic acid probe comprises a nucleic acid molecule having at least 90% sequence identity with the sequence according to any one of SEQ ID NOs: 51-60; or a nucleic acid molecule having at least 90% sequence identity with at least 250 contiguous nucleotides of any one of SEQ ID NOs: 51-60. In some embodiments, the TP53 DNA-specific nucleic acid probe comprises a nucleic acid molecule having at least 95% sequence identity with the sequence according to any one of SEQ ID NOs: 51-60; or a nucleic acid molecule having at least 95% sequence identity with at least 250 contiguous nucleotides of any one of SEQ ID NOs: 51-60. In some embodiments, the TP53 DNA-specific nucleic acid probe comprises a nucleic acid molecule having at least 99% sequence identity to the sequence according to any one of SEQ ID NOs: 51-60; or a nucleic acid molecule having at least 99% sequence identity with at least 250 contiguous nucleotides of any one of SEQ ID NOs: 51-60. In some embodiments, the TP53 DNA-specific nucleic acid probe comprises a nucleic acid molecule consisting of the sequence according to any one of SEQ ID NOs: 51-60; or a nucleic acid molecule consisting of at least 250 contiguous nucleotides of any one of SEQ ID NOs: 51-60. In some embodiments, the TP53 DNA-specific nucleic acid probe comprises a nucleic acid molecule with at least 90% sequence identity to at least 400 contiguous of any one of SEQ ID NOs: 51-60, at least 500 contiguous nucleotides of any one of SEQ ID NOs: 51-60, at least 1000 contiguous nucleotides of any one of SEQ ID NOs: 51-60, at least 2500 contiguous nucleotides of any one of SEQ ID NOs: 51-60, etc. In some embodiments, the TP53 DNA-specific nucleic acid probe comprises two or more portions, wherein the first portion comprises at least 250 contiguous nucleotides of a nucleic acid sequence with at least 90% sequence identity to one of SEQ ID NOs: 51-60; and the second portion comprises at least 250 contiguous nucleotides of a nucleic acid with at least 90% sequence identity to one of SEQ ID NOs: 51-60, wherein the first and second portions are different from one another. In some embodiments, the TP53 DNA-specific nucleic acid probe is at least 500 nucleotides in length, at least 1000 nucleotides in length, at least 5000 nucleotides in length, etc. In some embodiments, the TP53 DNA-specific nucleic acid probe comprises at least two of the probes disclosed herein.

In some examples of the disclosed methods, the sample is contacted with a nucleic acid probe that specifically binds to chromosome 17 centromere DNA. Methods of constructing chromosome 17 centromere-specific nucleic acid probes are known to one of ordinary skill in the art. In addition, chromosome 17 centromere nucleic acid probes may also be commercially available. For example, a chromosome 17 centromere probe suitable for use in the disclosed methods includes the chromosome 17 centromere probe included in the INFORM HER2 Dual ISH Probe Cocktail (Ventana Medical Systems, Tucson, Ariz., catalog number 780-4422). In one example, the sample is contacted with a hapten-labeled chromosome 17 centromere nucleic acid probe, for example under conditions specific for the probe to specifically bind to (hybridize with) chromosome 17 centromere genomic DNA in the sample. The sample is then contacted with an antibody that specifically binds to the hapten, for example, under conditions sufficient for the antibody to specifically bind to the hapten. The antibody may be conjugated to an enzyme (such as AP or HRP) or alternatively, the sample may be contacted with a second antibody that specifically binds the anti-hapten antibody, where the second antibody is conjugated to an enzyme. The presence of chromosome 17 centromere genomic DNA is detected by contacting the enzyme with a chromogen and/or substrate composition to produce a colored precipitate (third color) in the vicinity of the chromosome 17 centromere nucleic acid probe. In some examples, the gene copy number of chromosome 17 centromere DNA in the sample is scored by a slide reader by counting the number of areas of precipitate (“spots”) in the nuclei of the tumor cells.

In a particular example, the method comprises contacting the sample with a chromosome 17 centromere DNA probe conjugated to digoxigenin (DIG), for example under conditions sufficient for the chromosome 17 centromere probe to specifically bind to chromosome 17 centromere DNA in the sample. The sample is then contacted with an anti-hapten antibody that specifically binds DIG, for example under conditions sufficient for the anti-DIG antibody to specifically bind to the DIG. The sample is then contacted with a HRP-conjugated secondary antibody that specifically binds to the anti-DIG antibody, for example under conditions sufficient for the secondary antibody to specifically bind to the anti-DIG antibody. The sample is then contacted with a green chromogen component, producing a green/blue precipitate which is deposited in the nuclei near the chromosome 17 centromere probe (and the chromosome 17 centromere DNA) and can be visually detected by light microscopy as green/blue spots. In one example, the green chromogen component is HRP-Green (42 Lifescience, Bremerhaven, Germany, catalog number S-99056). One of ordinary skill in the art can select alternative detection reagents (such as alternative haptens, antibodies, enzymes, substrates, and/or chromogens) including those that produce a different color precipitate for detection of chromosome 17 centromere DNA.

In some embodiments, the chromosome 17 centromere-specific nucleic acid probe comprises a set of two or more single-stranded oligonucleotide control probes. The chromosome 17 oligonucleotide control probes are specific for two or more (between 2 and 14, e.g., 2, 3, 4, 5, 6, 7, 8, 9, 10, 11, 12, 13, 14, ≧4, ≧6, ≧8, etc.) distinct monomers of the alpha satellite control region of chromosome 17. In some embodiments, the chromosome 17 oligonucleotide probes (control probes) each comprise between 50 to 100 nucleotides.

In some embodiments, the chromosome 17 oligonucleotide control probes (each control probe) may comprise one of SEQ ID NOs: 61-74 (or complements thereof) (see below in Table 1). In some embodiments, the chromosome 17 oligonucleotide control probes (each control probe) may comprise a truncated version of one of SEQ ID NOs: 61-74 (or complements thereof). The truncated version may be at least 30 contiguous base pairs of said sequence, at least 40 contiguous base pairs of said sequence, or at least 50 contiguous base pairs of said sequence. In some embodiments, the chromosome 17 oligonucleotide control probes (each control probe) may comprise a sequence that has at least 70%, at least 80%, at least 90%, or at least 95% sequence identity to one of SEQ ID NOs: 61-74 (or complements thereof).

TABLE 1
Single-stranded Oligonucleotide Probes for
Chromosome 17

| Oligo | | |
|---|---|---|
| name | Sequences | Length |
| SEQ ID. | AATTCGTTGGAAACGGGATAATTTCAGCTGACTAA | 79 |
| NO. 61 | ACAGAAGCAGTCTCAGAATCTTCTTTGTGATGTTTG | |
| | CATTCAAA | |
| SEQ ID. | CTTCGTTCGAAACGGGTATATCTTCACATgcCATCT | 79 |
| NO. 62 | AGACAGAAGCATcCTCAGAAgCTTcTCTGTGATGAC | |
| | TGCATTC | |
| SEQ ID. | TGAACtCTCCTTTTGAGAGCGCAGTTTTGAAACTCT | 79 |
| NO. 63 | CTTTCTgTGGcATCTGCAAGGGGACATGtAGACCTCT | |
| | TTGAAG | |
| SEQ ID. | TTTCGTTGGAAACGGAATCATCTTCACATAAAAAcT | 79 |
| NO. 64 | ACACAGAtGCATTCTCAGGAACTttTTGGTGATGTTT | |
| | GTATTC | |
| SEQ ID. | CCTATGGTAGTAAAGGGAAtAGCTTCAtAgAAAAaC | 83 |
| NO. 65 | TAGACAGAAGCATTCTCAGAAAATACTTTGTGATG | |
| | ATTGAGTTTAAC | |
| SEQ ID. | CACAGAGCTGAACATTCCTTTGGATGGAGCAGGTT | 87 |
| NO. 66 | TGAgACACtCTTTtTGTAcAATCTaCaAGTGGATATTT | |
| | GGACCTCTCTGAGG | |
| SEQ ID. | GTTTCACaTTGCTTTTCATAGAGtAGtTctGAAACATG | 71 |
| NO. 67 | CTTTTCGTAGtGTCTaCAAGTGGACATTTGGAG | |
| SEQ ID. | CCTGTGGTGGAAAACGAATTATcGTCACgTAAAAA | 58 |
| NO. 68 | CTaGAGAGAAGCATTGTCAGAAA | |
| SEQ ID. | TGCATTCAACTCACAGAGTTGAAGGTTCCTTTTCAA | 65 |
| NO. 69 | AgAGCAGTTTCCAAtCACTCTTTgTGTGG | |
| SEQ ID. | CATTCCCTTTgACAGAGCAgTTTGGAAACTCTCtTTG | 71 |
| NO. 70 | TGTAGAATCTGCAAGTGGAGATATGGACCGCTTT | |
| SEQ ID. | CCTATGGTAGTAAAGGAAAtAGCTTCATATAAAAgC | 80 |
| NO. 71 | TAGACAGtAGCATTCaCAGAAAACTCTTgGTGACGA | |
| | CTGAGTTT | |
| SEQ ID. | ATTTCGTTGGAAACGGGATAAACCGCACAGAACTA | 80 |
| NO. 72 | AACAGAAGCATTCTCAGAACCTTCTTCGTGATGTTT | |
| | GCATTCAAC | |
| SEQ ID. | CGTAGTAAAGGAAATAACTTCCTATAAAAAgAAGA | 80 |
| NO. 73 | CAGAAGCTTTCTCAGAAAATTCTtTGGGATGATTGA | |
| | GTTGAACTC | |
| SEQ ID. | ACAGAGCTGAgCATT | 80 |
| NO. 74 | CCTTgcGATGtAGcAGT | |
| | TTaGAAACACACTTTC | |
| | TGcAGAATCTGCaAtT | |
| | GcATATTTGGACCTT | |

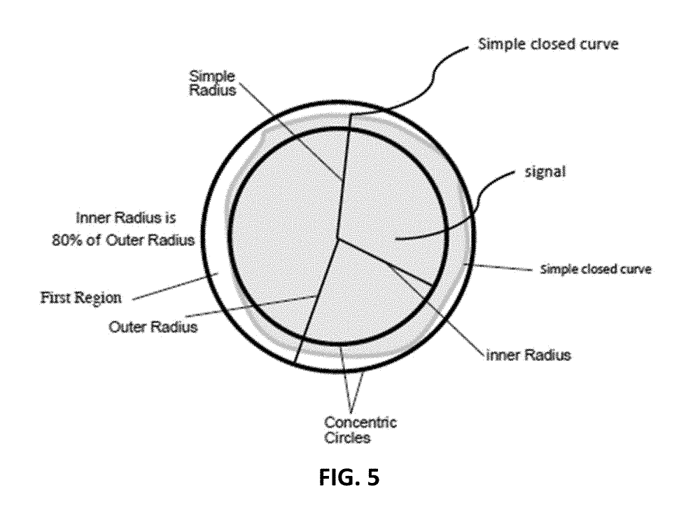

The chromosome 17 centromere oligonucleotide probes (control probes) can achieve an enumerable signal when hybridized to its DNA target. An enumerable signal has a generally round shape. In some embodiments, a round shape is a shape defined by a simple closed curve (see FIG. 5) fitting within a first region. The first region is an area on and between an inner concentric circle and an outer concentric circle. The inner concentric circle has an inner radius (Rᵢₙ) and the outer concentric circle has a outer radius (Rₒᵤₜ). Rᵢₙ is ≧50% of Rₒᵤₜ, and the simple closed curve has a radius Rₛᵢₘₚₗₑ wherein Rᵢₙ≦Rₛᵢₘₚₗₑ≦Rₒᵤₜ.

The chromosome 17 oligonucleotide control probes may be hybridized under conditions for a period of time less than about 3 hours, less than about 2 hours, 1 hour, or less than about an hour. The chromosome 17 centromere oligonucleotide probes (control probes) may achieve at least two enumerable signals per cell, e.g., with a staining intensity of ≧2 and staining coverage of ≧50% of the number of total nuclei within 3 hours of hybridization (or within 2 hours of hybridization, or within 1 hour of hybridization). In some embodiments, the chromosome 17 centromere oligonucleotide probes are configured to hybridize uniquely and specifically to a portion of the control region of human chromosome 17 so that other chromosomes or portions thereof are not evidently labeled without the influence of blocking DNA. In some embodiments, the chromosome 17 oligonucleotide control probes each comprise between 50 to 100 nucleotides.

The chromosome 17 oligonucleotide control probes may each comprise a detectable label, e.g., a hapten (e.g., dinitrophenyl, digoxigenin, biotin, or fluorescein, etc.). The labeled chromosome 17 oligonucleotide probes may be detected as previously described, e.g., with a secondary antibody directed to the hapten and/or with other detection components and reagents. For example, in a particular example, the method comprises contacting the sample with a chromosome 17 oligonucleotide control probes conjugated to digoxigenin (DIG), for example under conditions sufficient for the chromosome 17 oligonucleotide control probes to specifically bind to chromosome 17 centromere DNA in the sample. The sample is then contacted with an anti-hapten antibody that specifically binds DIG, for example under conditions sufficient for the anti-DIG antibody to specifically bind to the DIG. The sample is then contacted with a HRP-conjugated secondary antibody that specifically binds to the anti-DIG antibody, for example under conditions sufficient for the secondary antibody to specifically bind to the anti-DIG antibody. The sample is then contacted with a green chromogen component, producing a green/blue precipitate which is deposited in the nuclei near the chromosome 17 oligonucleotide control probes (and the chromosome 17 centromere DNA) and can be visually detected by light microscopy as green/blue spots. In one example, the green chromogen component comprises HRP-Green (42 Lifescience, Bremerhaven, Germany, catalog number S-99056). One of ordinary skill in the art can select alternative detection reagents (such as alternative haptens, antibodies, enzymes, substrates, and/or chromogens) including those that produce a different color precipitate for detection of chromosome 17 centromere DNA.

The disclosed methods are directed to detection of multiple protein and/or nucleic acid targets in a single sample. As a result, the detectable signal for each member of the assay must be individually distinguishable. Therefore, in some examples, the visual signal generated by the detection assay for each member of the assay is a different color. In one specific example, the methods result in a red staining for CD79a protein, black staining for TP53 DNA (for example, black spots in the nucleus, such as individually distinguishable black spots or clusters of black spots), and green/blue staining for chromosome 17 centromere DNA (for example, green/blue spots in the nucleus, such as individually distinguishable green/blue spots or clusters of green/blue spots). However, other combinations may be used.

The methods disclosed herein may also include steps for pre-treatment of samples (e.g., blood samples) prior to or between the steps including contacting the sample with a CD79a-specific antibody, a TP53-specific nucleic acid probe, and a chromosome 17 centromere-specific nucleic acid probe. These steps are known to one of ordinary skill in the art and may include deparaffinization of a sample (such as a FFPE sample), cell conditioning, washes, and so on. One of skill in the art can make adjustments to these conditions (for example, minor adjustments to times and/or temperatures of incubations, wash steps, etc.).

In some embodiments, the sample is subjected to a protease treatment (proteinase K, pepsin, collagenase, dispase, a combination thereof, etc.) after the step of staining the B cell marker but before the step of staining TP53 genomic DNA and staining chromosome 17 centromere DNA. The protease treatment is effective to allow for hybridization of the nucleic acid probes to their respective DNA targets. The protease treatment does not eliminate the first color and tissue morphology is sufficiently maintained so as to allow for the detection of the first color. In some embodiments, the sample is subjected to a heat treatment after the step of staining the B cell marker but before the protease treatment.

The present invention also features a slide (single slide) comprising a sample of cells chromogenically stained for CD79a protein, TP53 DNA, and chromosome 17 DNA. The markers may each be stained with a different chromogen, e.g., CD79a protein is stained with a first chromogen, TP53 DNA is stained with a second chromogen, and chromosome 17 is stained with a third chromogen. In some embodiments, the first chromogen comprises fast red, the second chromogen comprises silver, and the third chromogen comprises a green chromogen component. In some embodiments, more than 50% of the nuclei have enumerable signals for chromosome 17. An enumerable signal may have a generally round shape. In some embodiments, a round shape is a shape defined by a simple closed curve fitting within a first region. The first region is an area on and between an inner concentric circle and an outer concentric circle. The inner concentric circle has an inner radius (Rᵢₙ) and the outer concentric circle has a outer radius (Rₒᵤₜ). Rᵢₙ is ≧50% of Rₒᵤₜ, and the simple closed curve has a radius Rₛᵢₘₚₗₑ wherein Rᵢₙ≦Rₛᵢₘₚₗₑ≦Rₒᵤₜ.

The disclosed methods can be automated. Systems for automated IHC and/or ISH are commercially available, such as the BENCHMARK ULTRA slide staining system, the BENCHMARK XT slide staining system, and the DISCOVERY XT slide staining system (Ventana Medical Systems, Tucson, Ariz.), BOND-MAX and BOND-III slide stainers (Leica Biosystems, Buffalo Grove, Ill.), and the IQ Kinetic slide stainer (Biocare Medical, Concord, Calif.). Ventana Medical Systems, Inc. is the assignee of a number of United States patents disclosing systems and methods for performing automated analyses, including U.S. Pat. Nos. 5,650,327; 5,654,200; 6,296,809; 6,352,861; 6,582,962; 6,827,901 and 6,943,029, each of which is incorporated in its entirety herein by reference.

Non-limiting examples of chromogens that may be used in the disclosed methods include (but are not limited to) those shown in Table 2. While not exhaustive, Table 2 provides insight into the varieties of presently available chromogens. Further illustrative chromogens include those described in U.S. Pat. Publ. 2013/0260379 and U.S. Prov. Pat. App. No. 61/831,552, filed Jun. 5, 2013; both of which are incorporated by reference herein in their entirety.

TABLE 2
Examples of commercially available chromogen/substrate systems

| Abbr. | Name | Color | Enzyme |
|---|---|---|---|
| DAB | 3,3′-diamino-benzidine + | brown-black | peroxidase |
| | H₂O₂ | | |
| AEC | 3-amino-9-ethyl- | red | peroxidase |
| | carbazole + H₂O₂ | | |
| CN | 4-chloro-1-naphthol + | blue | peroxidase |
| | H₂O₂ | | |
| BCIP/NBT | 5-bromo-4-chloro-3- | indigo-black | alkaline |
| | indolyl-phosphate + | | phosphatase |
| | nitroblue tetrazolium | | |
| FAST RED | 4-chloro-2- | red | alkaline |
| | methylbenzenediazonium + | | phosphatase |
| | 3-hydroxy-2-naphthoic | | |
| | acid 2,4-dimethylanilide | | |
| | phosphate | | |
| FAST BLUE | Naphthol AS-MX | blue | alkaline |
| | phosphate disodium salt + | | phosphatase |
| | fast blue BB salt | | |
| | hemi(zinc chloride) salt | | |
| FUCHSIN | Naphthol AS-BI + New | red | alkaline |
| | Fuchsin | | phosphatase |
| NBT | nitroblue tetrazolium + | blue-purple | dehydrogenase |
| | phenazine methosulfate | | |
| ALK | 3-methyl-1-phenyl-1H- | yellow-gold | alkaline |
| GOLD† | pyrazol-5-yl dihydrogen | | phosphatase |
| | phosphate + fast blue BB | | |

†International Pat. Publ. No. WO 2012/024185, incorporated herein by reference

### IV. Samples

Exemplary samples include, without limitation, blood smears, cytocentrifuge preparations, cytology smears, core biopsies, and/or fine-needle aspirates. In some examples, the samples include tissue sections (e.g., cryostat tissue sections and/or paraffin-embedded tissue sections). In particular embodiments, the samples include tumor cells, such as breast tumor cells or ovarian tumor cells. Methods of obtaining a biological sample from a subject are known in the art. For example, methods of obtaining breast tissue or breast cells are routine. Exemplary biological samples may be isolated from normal cells or tissues, or from neoplastic cells or tissues. A solid support can hold the biological sample and permit the convenient detection of components (e.g., proteins and/or nucleic acid molecules) in the sample. Exemplary supports include microscope slides (e.g., glass microscope slides or plastic microscope slides), cover slips (e.g., glass cover slips or plastic cover slips), tissue culture dishes, multi-well plates, membranes (e.g., nitrocellulose or polyvinylidene fluoride (PVDF)) or BIACORE™ chips.

The samples described herein can be prepared using any method now known or hereafter developed in the art. Generally, tissue samples are prepared by fixing and embedding the tissue in a medium. In other examples, samples include a cell suspension which is prepared as a monolayer on a solid support (such as a glass slide) for example by smearing or centrifuging cells onto the solid support. In further examples, fresh frozen (for example, unfixed) tissue sections may be used in the methods disclosed herein.

The process of fixing a sample can vary. Fixing a tissue sample preserves cells and tissue constituents in as close to a life-like state as possible and allows them to undergo preparative procedures without significant change. Fixation arrests the autolysis and bacterial decomposition processes that begin upon cell death, and stabilizes the cellular and tissue constituents so that they withstand the subsequent stages of tissue processing, such as for ISH or IHC.

Tissues can be fixed by any suitable process, including perfusion or by submersion in a fixative. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum.

The most commonly used fixative in preparing samples is formaldehyde, generally in the form of a formalin solution (4% formaldehyde in a buffer solution, referred to as 10% buffered formalin). In one example, the fixative is 10% neutral buffered formalin.

In some examples an embedding medium is used. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics. Many embedding media are hydrophobic; therefore, the inert material may need to be removed prior to histological or cytological analysis, which utilizes primarily hydrophilic reagents. The term deparaffinization or dewaxing is broadly used herein to refer to the partial or complete removal of any type of embedding medium from a biological sample. For example, paraffin-embedded tissue sections are dewaxed by passage through organic solvents, such as toluene, xylene, limonene, or other suitable solvents.

### V. Nucleic acid probes to 19q12, 13q12, ATM, DLEU2, INSR, TP53

Also disclosed herein are nucleic acid probes that include a plurality of segments of uniquely specific nucleic acid sequences. The disclosed probes are useful for detecting presence of a target nucleic acid in a sample. The uniquely specific nucleic acid sequences are designed to occur only once each in the haploid genome of an organism, and provide high levels of sensitivity and specificity for the detection of a target nucleic acid in a sample. In some embodiments, the probes are useful for detecting presence and location of 19q12, 13q12, ATM, DLEU2, INSR, or TP53.

Briefly, the disclosed probes may include isolated nucleic acid molecules comprising the sequences provided herein as any one of SEQ ID NOs: 1-74, or nucleic acids having at least 70% (such as at least 75%, at least 80%, at least 85%, at least 90%, at least 91%, at least 92%, at least 93%, at least 94%, at least 95%, at least 96%, at least 97%, at least 98%, at least 99%, or 100%) sequence identity with any one of SEQ ID NOs: 1-74. In other embodiments, the disclosed probes include isolated nucleic acid molecules comprising at least 70% (such as at least 75%, at least 80%, at least 85%, at least 90%, at least 91%, at least 92%, at least 93%, at least 94%, at least 95%, at least 96%, at least 97%, at least 98%, at least 99%, or 100%) sequence identity with at least 250 contiguous nucleotides of any one of SEQ ID NOs: 1-74. In some examples, the nucleic acid probes include nucleic acid molecules consisting of the sequence of any one of SEQ ID NOs: 1-74 or at least 250 contiguous nucleotides of any one of SEQ ID NOs: 1-74. In some examples, the disclosed probes include a detectable label.

Also disclosed herein are probe sets for detecting a target nucleic acid molecule. A probe set includes two or more (for example, 2, 3, 4, 5, 6, 7, 8, 9, 10, or more) of the disclosed nucleic acid probes. In some examples a probe set useful for detecting 19q12 includes two or more nucleic acid molecules selected from SEQ ID NOs: 1-10. In other example, a probe set useful for detecting 13q12 includes two or more nucleic acid molecules selected from SEQ ID NOs: 11-20. In another example, a probe set useful for detecting ATM includes two or more nucleic acid molecules selected from SEQ ID NOs: 21-30. In a still further example, a probe set useful for detecting DLEU2 includes two or more nucleic acid molecules selected from SEQ ID NOs: 31-40. In another example, a probe set useful for detecting INSR includes two or more nucleic acid molecules selected from SEQ ID NOs: 41-50. In another example, a probe set useful for detecting TP53 includes two or more nucleic acid molecules selected from SEQ ID NOs: 51-74.

The uniquely specific nucleic acid probes may be complementary to target nucleic acid molecules of interest, e.g., 19Q12, 13q12, ATM, DLEU2 and INSR. The probes may include a plurality of uniquely specific nucleic acid segments, which were designed and synthesized utilizing the methods described in U.S. Pat. App. Publ. No. 2011/0160076 and International Pat. Publ. No. WO 2011/062293, both of which are incorporated herein by reference in their entirety. The nucleic acid segments included in each probe are each uniquely specific (occur only once in the haploid human genome) and are from non-contiguous portions of the human genome.

Surprisingly, many of the segments are located in introns in the human genome. While not being bound by theory, it is generally believed in the field that the intronic regions of the human genome evolve at a faster rate than coding regions. In illustrative embodiments, the present probes have sequences that are exon-free or substantially exon-free. In one embodiment, the sequences are selected from non-coding gene regions. The development and use of intronic probes is counter-intuitive as most of the field is concerned with genetic abnormalities associated with the protein coding region of the gene. The present probes provide researchers and clinicians with unique and distinct data heretofore unavailable. It is unexpected that introns include uniquely specific sequences due to the belief in the field that introns are not as highly conserved as the exon-containing DNA sequences. The identification of uniquely specific sequences in intronic regions indicates that some of these regions may be highly conserved. The present probes provide a means to study these highly-conserved intronic regions of these very important genes.

### A. 19q12

Disclosed herein are isolated nucleic acid probes including linked uniquely specific nucleic acid segments complementary to region 19q12 of the human genome. In some embodiments, the nucleic acid probes include or consist of nucleic acid molecules with at least 70%, at least 75%, at least 80%, at least 85%, at least 90% or more sequence identity to one or more of SEQ ID NOs: 1-10.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 1-10. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 1-10. In some examples, the nucleic acid probes include or consist of the sequences set forth in SEQ ID NOs: 1-10. In one example, the probe includes or consists of a contiguous nucleic acid molecule comprising two or more of SEQ ID NOs: 1-10 (such as 2, 3, 4, 5, 6, 7, 8, 9, or 10 of SEQ ID NOs: 1-10) in a single nucleic acid molecule.

### B. 13q12

Disclosed herein are isolated nucleic acid probes including linked uniquely specific nucleic acid segments complementary to region 13q12 of the human genome. In some embodiments, the isolated nucleic acid probes include or consist of nucleic acid molecules with at least 70%, at least 75%, at least 80%, at least 85%, at least 90%, or more sequence identity to one of more of SEQ ID NOs: 11-20.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 11-20. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 11-20. In some examples, the nucleic acid probes include or consist of the sequences set forth in SEQ ID NOs: 11-20. In one example, the probe includes or consists of a contiguous nucleic acid molecule comprising two or more of SEQ ID NOs: 11-20 (such as 2, 3, 4, 5, 6, 7, 8, 9, or 10 of SEQ ID NOs: 11-20) in a single nucleic acid molecule.

### C. ATM

Disclosed herein are isolated nucleic acid probes including linked uniquely specific nucleic acid segments complementary to the human ATM gene (e.g., Gene ID No. 472; Chromosome: 11; NC_000011.9 (108093559 . . . 108239829), incorporated herein by reference as present in GENBANK® on SEP 25, 2013). In some embodiments, the isolated nucleic acid probes include or consist of nucleic acid molecules with at least 70%, at least 75%, at least 80%, at least 85%, at least 90%, or more sequence identity to one or more of SEQ ID NOs: 21-30.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 21-30. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 21-30. In some examples, the nucleic acid probes include or consist of the sequences set forth in SEQ ID NOs: 21-30. In one example, the probe includes or consists of a contiguous nucleic acid molecule comprising two or more of SEQ ID NOs: 21-30 (such as 2, 3, 4, 5, 6, 7, 8, 9, or 10 of SEQ ID NOs: 21-30) in a single nucleic acid molecule.

### D. DLEU2

Disclosed herein are isolated nucleic acid probes including linked uniquely specific nucleic acid segments complementary to the human DLEU2 gene (e.g., Gene ID No. 8847; NC_000013.10 (50549491 . . . 50699680), incorporated herein by reference as present in GENBANK® on SEP 25, 2013). In some embodiments, the isolated nucleic acid probes include or consist of nucleic acid molecules with at least 70%, at least 75%, at least 80%, at least 85%, at least 90%, or more sequence identity to one or more of SEQ ID NOs: 31-40.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 31-40. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 31-40. In some examples, the nucleic acid probes include or consist of the sequences set forth in SEQ ID NOs: 31-40. In one example, the probe includes or consists of a contiguous nucleic acid molecule comprising two or more of SEQ ID NOs: 31-40 (such as 2, 3, 4, 5, 6, 7, 8, 9, or 10 of SEQ ID NOs: 31-40) in a single nucleic acid molecule.

### E. INSR

Disclosed herein are isolated nucleic acid probes including linked uniquely specific nucleic acid segments complementary to the human INSR gene (e.g., Gene ID No. 3643; NC_000019.9 (7112266 . . . 7294011, complement), incorporated herein by reference as present in GENBANK® on Sep. 25, 2013). In some embodiments, the isolated nucleic acid probes include or consist of nucleic acid molecules with at least 70%, at least 75%, at least 80%, at least 85%, at least 90%, or more sequence identity to one or more of SEQ ID NOs: 41-50.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 41-50. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 41-50. In some examples, the nucleic acid probes include or consist of the sequences set forth in SEQ ID NOs: 41-50. In one example, the probe includes or consists of a contiguous nucleic acid molecule comprising two or more of SEQ ID NOs: 41-50 (such as 2, 3, 4, 5, 6, 7, 8, 9, or 10 of SEQ ID NOs: 41-50) in a single nucleic acid molecule.

### F. TP53

Disclosed herein are isolated nucleic acid probes including linked uniquely specific nucleic acid segments complementary to the human TP53 gene (e.g., Gene ID No. 7157; NC_000017.10 (7571720 . . . 7590868, complement), incorporated herein by reference as present in GENBANK® on SEP 25, 2013). In some embodiments, the isolated nucleic acid probes include or consist of nucleic acid molecules with at least 70%, at least 75%, at least 80%, at least 85%, at least 90%, or more sequence identity to one or more of SEQ ID NOs: 51-60.

In some embodiments, the nucleic acid probe can have a sequence with at least 70%, at least 75%, at least 80%, at least 85%, or at least 90% sequence identity (for example at least 91%, 92%, 93%, 94%, 95%, 96%, 97%, 98%, 99%, or 100% identity) to one of the nucleotide sequences set forth in SEQ ID NOs: 41-50. In other embodiments, the nucleic acid probe can include a sequence with at least 70%, at least 75%, at least 80%, at least 85%, or at least 90% sequence identity (for example at least 91%, 92%, 93%, 94%, 95%, 96%, 97%, 98%, 99%, or 100% identity) to at least 250 contiguous nucleotides (such as at least 400, at least 500, at least 750, at least 1000, at least 2000, at least 3000, at least 4000, or at least 5000 contiguous nucleotides) of any one of SEQ ID NOs: 41-50. In some examples, the nucleic acid probes include or consist of the sequences set forth in SEQ ID NOs: 41-50. In one example, the probe includes or consists of a contiguous nucleic acid molecule comprising two or more of SEQ ID NOs: 41-50 (such as 2, 3, 4, 5, 6, 7, 8, 9, or 10 of SEQ ID NOs: 41-50) in a single nucleic acid molecule.

In some embodiments, the disclosed probes have a length of at least 250 contiguous nucleotides (such as at least 300, at least 400, at least 500, at least 750, at least 1000, at least 2000, at least 3000, at least 4000, at least 5000, at least 6000, at least 7000, at least 8000, at least 9000, at least 10,000, at least 12,000, at least 15,000, at least 20,000, at least 25,000, at least 30,000, at least 35,000, at least 40,000, at least 45,000, at least 50,000, or more contiguous nucleotides). In other embodiments, the disclosed probes have a length of about 250-50,000 nucleotides (for example, about 500-50,000, about 1000-40,000, about 5000-25,000, about 7000-20,000, or about 10,000 to 15,000 nucleotides). The probes can include all or a portion of one or more of SEQ ID NOs: 1-74, or all or a portion of a nucleic acid having at least 70% sequence identity (for example, at least 75%, at least 80%, at least 85%, at least 90%, at least 91%, at least 92%, at least 93%, at least 94%, at least 95%, at least 96%, at least 97%, at least 98%, at least 99%, or 100% identity) to one of more of SEQ ID NOs: 1-74. In some examples, the probe is a contiguous nucleic acid molecule comprising up to 10 of the disclosed sequences (such as SEQ ID NOs: 1-10, SEQ ID NOs: 11-20, SEQ ID NOs: 21-30, SEQ ID NOs: 31-40, SEQ ID NOs: 41-50, or SEQ ID NOs: 51-60, or SEQ ID NOs: 61-74).

In other examples, the probe is a contiguous nucleic acid molecule including two or more portions or segments, wherein the first portion includes at least 250 contiguous nucleotides at least 70% identical (for example, at least 75%, at least 80%, at least 85%, at least 90%, at least 91%, at least 92%, at least 93%, at least 94%, at least 95%, at least 96%, at least 97%, at least 98%, at least 99%, or 100% identical) to the corresponding portion of any one of SEQ ID NOs: 1-74 and the second portion includes at least 250 contiguous nucleotides at least 70% identical (for example, at least 75%, at least 80%, at least 85%, at least 90%, at least 91%, at least 92%, at least 93%, at least 94%, at least 95%, at least 96%, at least 97%, at least 98%, at least 99%, or 100% identical) to the corresponding portion of any one of SEQ ID NOs: 1-74, where the first and second portions are different.

Each of the disclosed sequences of SEQ ID NOs: 1-74 include a plurality of 100 bp uniquely specific nucleic acid segments, which were designed and synthesized utilizing the methods described in U.S. Pat. App. Publ. No. 2011/0160076 and International Pat. Publ. No. WO 2011/062293, both of which are incorporated herein by reference in their entirety. The disclosed probes are exemplary, and it is understood that in some examples, at least one (such as at least two, at least three, at least four, at least five, or more) of the 100 bp segments (for example nucleotides 1-100, 101-200, 201-300, and so on) or portions thereof could be removed from the disclosed sequences to provide additional nucleic acid probes for 13q12, 19q12, ATM, DLEU2, INSR, or TP53. In other examples, the order of at least one (such as at least two, at least three, at least four, at least five, or more) of the 100 bp segments (for example nucleotides 1-100, 101-200, 201-300, and so on) could be rearranged from the order in the disclosed probes to provide additional nucleic acid probes for 13q12, 19q12, ATM, DLEU2, INSR, or TP53. One of skill in the art could make and test such modified probes, for example by synthesizing a modified probe and assessing its hybridization to samples known to contain or not to contain the target nucleic acid.

In some examples, the individual uniquely specific segments are produced (for example by oligonucleotide synthesis or by amplification of the sequences from the genomic target nucleic acid) and joined together. In other examples, the nucleic acid probe is synthesized as a series of oligonucleotides (such as individual oligonucleotides of about 100 bp), which are joined together. For example, the segments may be joined or ligated to one another enzymatically (e.g., using a ligase). For example, the segments can be joined in a blunt-end ligation or at a restriction site. In another example, the segments may be synthesized with complementary nucleic acid overhangs (such as at least a 3 bp overhang), annealed, and joined to one another, for example with a ligase. Chemical ligation and amplification can also be used to join the uniquely specific segments. In another example, the entire nucleic acid probe is synthesized and the individual uniquely specific segments are directly joined during synthesis.

In some embodiments, the disclosed nucleic acid probes are included in a vector, such as a plasmid or an artificial chromosome (e.g., yeast artificial chromosome (YAC), P1 based artificial chromosome (PAC), or bacterial artificial chromosome (BAC)). In some examples, any one of SEQ ID NOs: 1-74 disclosed herein are introduced into a plasmid vector, for example to allow replication and/or labeling of the nucleic acid probe by standard molecular biology techniques. In one example, the vector is a pUC plasmid vector. In other examples, two or more of the disclosed sequences (such as 2, 3, 4, 5, 6, 7, 8, 9, 10, or more of the disclosed sequences) are introduced into a vector, for example to allow replication and/or labeling of the nucleic acid probe by standard molecular biology techniques. The two or more sequences can be introduced into a vector in any order. One of skill in the art can determine whether the sequences the overlap the junctions between the individual sequences (for example, a window of about 100 bp) are uniquely specific, for example, utilizing the teachings of U.S. Pat. App. Publ. No. 2011/0160076 and International Pat. Publ. No. WO 2011/062293, each of which is incorporated in its entirety by reference herein. If a sequence that is not uniquely specific is introduced, the sequences can be reordered and reanalyzed in order to select an order that does not produce any non-uniquely specific sequences. In particular examples, all of SEQ ID NOs: 1-10 are introduced into a single vector, all of SEQ ID NOs: 11-20 are introduced into a single vector, all of SEQ ID NOs: 21-30 are introduced into a single vector, all of SEQ ID NOs: 31-40 are introduced into a single vector, all of SEQ ID NOs: 41-50 are introduced into a single vector, or all of SEQ ID NOs: 51-60 are introduced into a single vector. In one example, the vector is an artificial chromosome (such as a YAC, BAC or PAC).

### VI. Probe Sets

In some embodiments, the disclosed probes are included in a set of probes, for example as set of two or more (such as 2, 3, 4, 5, 6, 7, 8, 9, 10, or more) of the disclosed probes. In some examples, a probe set includes two or more probes specific for a particular target nucleic acid molecule (such as two or more probes for 13q12, 19q12, ATM, DLEU2, INSR, or TP53). In some examples, a probe set includes probes specific for two or more target nucleic acid molecules (such as probes specific for two or more of 13q12, 19q12, ATM, DLEU2, INSR, and TP53).

One exemplary probe set includes two or more probes specific for human 19q12. The probe set includes two or more (such as 2, 3, 4, 5, 6, 7, 8, 9, 10, or more) probes selected from probes including or consisting of the nucleic acid sequence having at least at least 70%, at least 75%, at least 80%, at least 85%, or 90% sequence identity with SEQ ID NOs: 1-10 or at least 250 contiguous nucleotides of any one of SEQ ID NOs: 1-10. In one example, a probe set for 19q12 includes probes including or consisting of nucleic acid sequences having at least 70%, at least 75%, at least 80%, at least 85%, or at least 90% sequence identity with each of SEQ ID NOs: 1-10. In a specific example, the probe set includes each of SEQ ID NOs: 1-10.

Another exemplary probe set includes probes specific for human 13q12. The probe set includes two or more (such as 2, 3, 4, 5, 6, 7, 8, 9, 10, or more) probes selected from probes including or consisting of the nucleic acid sequence having at least 70%, at least 75%, at least 80%, at least 85%, or at least 90% sequence identity with any one of SEQ ID NOs: 11-20 or at least 250 contiguous nucleotides of any one of SEQ ID NOs: 11-20. In one example, a probe set for 13q12 includes probes including or consisting of nucleic acid sequences having at least 70%, at least 75%, at least 80%, at least 85%, or at least 90% sequence identity with each of SEQ ID NOs: 11-20. In a particular example, the probe set includes each of SEQ ID NOs: 11-20.

A further exemplary probe set includes probes specific for human ATM. The probe set includes two or more (such as 2, 3, 4, 5, 6, 7, 8, 9, 10, or more) probes selected from probes including or consisting of the nucleic acid sequence having at least 70%, at least 75%, at least 80%, at least 85%, or at least 90% sequence identity with any one of SEQ ID NOs: 21-30 or at least 250 contiguous nucleotides of any one of SEQ ID NOs: 21-30. In one example, a probe set for ATM includes probes including or consisting of nucleic acid sequences having at least 70%, at least 75%, at least 80%, at least 85%, or at least 90% sequence identity with each of SEQ ID NOs: 21-30. In a particular example, the probe set includes each of SEQ ID NOs: 21-30.

A still further exemplary probe set includes probes specific for human DLEU2. The probe set includes two or more (such as 2, 3, 4, 5, 6, 7, 8, 9, 10, or more) probes selected from probes including or consisting of the nucleic acid sequence having at least 70%, at least 75%, at least 80%, at least 85%, or at least 90% sequence identity with any one of SEQ ID NOs: 31-40 or at least 250 contiguous nucleotides of any one of SEQ ID NOs: 31-40. In one example, a probe set for DLEU2 includes probes including or consisting of nucleic acid sequences having at least 70%, at least 75%, at least 80%, at least 85%, or at least 90% sequence identity with each of SEQ ID NOs: 31-40. In a particular example, the probe set includes each of SEQ ID NOs: 31-40.

An additional exemplary probe set includes probes specific for human INSR. The probe set includes two or more (such as 2, 3, 4, 5, 6, 7, 8, 9, 10, or more) probes selected from probes including or consisting of the nucleic acid sequence having at least 70%, at least 75%, at least 80%, at least 85%, or at least 90% sequence identity with any one of SEQ ID NOs: 41-50 or at least 250 contiguous nucleotides of any one of SEQ ID NOs: 41-50. In one example, a probe set for INSR includes probes including or consisting of nucleic acid sequences having at least 70%, at least 75%, at least 80%, at least 85%, or at least 90% sequence identity with each of SEQ ID NOs: 41-50. In a particular example, the probe set includes each of SEQ ID NOs: 41-50.

An additional exemplary probe set includes probes specific for human TP53. The probe set includes two or more (such as 2, 3, 4, 5, 6, 7, 8, 9, 10, or more) probes selected from probes including or consisting of the nucleic acid sequence having at least 70%, at least 75%, at least 80%, at least 85%, or at least 90% sequence identity with any one of SEQ ID NOs: 51-60 or at least 250 contiguous nucleotides of any one of SEQ ID NOs: 51-60. In one example, a probe set for INSR includes probes including or consisting of nucleic acid sequences having at least 70%, at least 75%, at least 80%, at least 85%, or at least 90% sequence identity with each of SEQ ID NOs: 51-60. In a particular example, the probe set includes each of SEQ ID NOs: 51-60.

### VII. Kits

Also disclosed are kits including one or more of the disclosed nucleic acid probes (for example, one or more of SEQ ID NOs: 1-74). For example, kits can include at least one probe (such as at least 1, 2, 3, 4, 5, 6, 7, 8, 9, 10, or more probes) or at least one probe set (such as at least 1, 2, 3, 4, or 5 probe sets) as described herein. In one example, the kit includes probes including the nucleic acid sequence of at least 1, 2, 3, 4, 5, 6, 7, 8, 9, or all of SEQ ID NOs: 1-10 (or sequences at least 70%, at least 75%, at least 80%, at least 85%, or at least 90% identical to SEQ ID NOs: 1-10) or one or more nucleic acids including at least 250 contiguous nucleotides of any one of sequences at least 70%, at least 75%, at least 80%, at least 85%, or at least 90% identical to SEQ ID NOs: 1-10. In another example, the kit includes probes including the nucleic acid sequence of at least 1, 2, 3, 4, 5, 6, 7, 8, 9, or all of SEQ ID NOs: 11-20 (or sequences at least 70%, at least 75%, at least 80%, at least 85%, or at least 90% identical to SEQ ID NOs: 11-20) or one or more nucleic acids including at least 250 contiguous nucleotides of any one of sequences at least 70%, at least 75%, at least 80%, at least 85%, or at least 90% identical to SEQ ID NOs: 11-20. In a further example, the kit includes probes including the nucleic acid sequence of at least 1, 2, 3, 4, 5, 6, 7, 8, 9, or all of SEQ ID NOs: 21-30 (or sequences at least 70%, at least 75%, at least 80%, at least 85%, or at least 90% identical to SEQ ID NOs: 21-30) or one or more nucleic acids including at least 250 contiguous nucleotides of any one of sequences at least 70%, at least 75%, at least 80%, at least 85%, or at least 90% identical to SEQ ID NOs: 21-30. In a still further example, the kit includes probes including the nucleic acid sequence of at least 1, 2, 3, 4, 5, 6, 7, 8, 9, or all of SEQ ID NOs: 31-40 (or sequences at least 70%, at least 75%, at least 80%, at least 85%, or at least 90% identical to SEQ ID NOs: 31-40) or one or more nucleic acids including at least 250 contiguous nucleotides of any one of sequences at least 70%, at least 75%, at least 80%, at least 85%, or at least 90% identical to SEQ ID NOs: 31-40. In an additional example, the kit includes probes including the nucleic acid sequence of at least 1, 2, 3, 4, 5, 6, 7, 8, 9, or all of SEQ ID NOs: 41-50 (or sequences at least 70%, at least 75%, at least 80%, at least 85%, or at least 90% identical to SEQ ID NOs: 41-50) or one or more nucleic acids including at least 250 contiguous nucleotides of any one of sequences at least 70%, at least 75%, at least 80%, at least 85%, or at least 90% identical to SEQ ID NOs: 41-50. In another example, the kit includes probes including the nucleic acid sequence of at least 1, 2, 3, 4, 5, 6, 7, 8, 9, or all of SEQ ID NOs: 51-60 (or sequences at least 70%, at least 75%, at least 80%, at least 85%, or at least 90% identical to SEQ ID NOs: 51-60) or one or more nucleic acids including at least 250 contiguous nucleotides of any one of sequences at least 70%, at least 75%, at least 80%, at least 85%, or at least 90% identical to SEQ ID NOs: 51-60. In some examples, the probes are present in separate containers. In other examples, the probes (or the probe set) are in a single container.

In one particular embodiment, the kit includes ten plasmids, each plasmid including one of SEQ ID NOs: 1-10. The plasmids are pooled and labeled (for example, labeled with DNP) and provided in a vial at a concentration of about 60 μg/ml.

In another particular embodiment, the kit includes ten plasmids, each plasmid including one of SEQ ID NOs: 11-20. The plasmids are pooled and labeled (for example, labeled with DNP) and provided in a vial at a concentration of about 60 μg/ml.

In a further particular embodiment, the kit includes ten plasmids, each plasmid including one of SEQ ID NOs: 21-30. The plasmids are pooled and labeled (for example, labeled with DNP) and provided in a vial at a concentration of about 60 μg/ml.

In another particular embodiment, the kit includes ten plasmids, each plasmid including one of SEQ ID NOs: 31-40. The plasmids are pooled and labeled (for example, labeled with DNP) and provided in a vial at a concentration of about 70 μg/ml.

In an additional particular embodiment, the kit includes ten plasmids, each plasmid including one of SEQ ID NOs: 41-50. The plasmids are pooled and labeled (for example, labeled with DNP) and provided in a vial at a concentration of about 60 μg/ml.

In one particular embodiment, the kit includes ten plasmids, each plasmid including one of SEQ ID NOs: 51-60. The plasmids are pooled and labeled (for example, labeled with DNP) and provided in a vial at a concentration of about 60 μg/ml.

The kits can also include one or more reagents for detecting a target nucleic acid molecule in a sample (for example, by in situ hybridization or CGH assay), or for producing a detectably labeled probe. For example, a kit can include at least one of the disclosed nucleic acid probes or probe sets, along with one or more buffers, labeled dNTPs, a labeling enzyme (such as a polymerase), primers, nuclease free water, and instructions for producing a labeled probe. In another example, the kit includes one or more of the disclosed nucleic acid probes (unlabeled or labeled) along with buffers and other reagents for performing in situ hybridization. For example, if one or more unlabeled probes are included in the kit, labeling reagents can also be included, along with specific detection agents (for example, fluorescent, chromogenic, luminescent and/or radiometric) and other reagents for performing an in situ hybridization assay, such as paraffin pretreatment buffer, protease(s) and protease buffer, prehybridization buffer, hybridization buffer, wash buffer, counterstain(s), mounting medium, or combinations thereof. In some examples, such kit components are present in separate containers. The kit can optionally further include control slides (such as positive or negative controls, such as those known to contain or not contain the target sequence(s), such as 19Q12, 13q12, ATM, DLEU2, INSR, or TP53 nucleic acid sequences) for assessing hybridization and signal of the probe(s).

In certain examples, the kits include avidin, antibodies, and/or receptors (or other anti-ligands). Optionally, one or more of the detection agents (including a primary detection agent, and optionally, secondary, tertiary or additional detection reagents) are labeled, for example, with a hapten or fluorophore (such as a fluorescent dye or quantum dot). In some instances, the detection reagents are labeled with different detectable moieties (for example, different fluorescent dyes, spectrally distinguishable quantum dots, different haptens, etc.). For example, a kit can include two or more nucleic acid probes or probe sets that correspond to and are capable of hybridizing to different target nucleic acids (for example, any of the target nucleic acids disclosed herein). The first probe or probe set can be labeled with a first detectable label (e.g., hapten, fluorophore, etc.), the second probe or probe set can be labeled with a second detectable label, and any additional probes or probe sets (e.g., third, fourth, fifth, etc.) can be labeled with additional detectable labels. The first, second, and any subsequent probes or probe sets can be labeled with different detectable labels, although other detection schemes are possible. If the probe(s) are labeled with indirectly detectable labels, such as haptens, the kits can include detection agents (such as labeled avidin, antibodies or other specific binding agents) for some or all of the probes. In one embodiment, the kit includes probes and detection reagents suitable for multiplex ISH.

In one example, the kit also includes an antibody conjugate, such as an antibody conjugated to a label (e.g., an enzyme, fluorophore, or fluorescent nanoparticle).

In some examples, the antibody is conjugated to the label through a linker, such as PEG, 6X-His, streptavidin, or GST.

In another example, the kit includes one or more of the disclosed nucleic acid probes affixed to a solid support (such as an array) along with buffers and other reagents for performing CGH. Reagents for labeling sample and control DNA can also be included, along with other reagents for performing an aCGH assay, prehybridization buffer, hybridization buffer, wash buffer, or combinations thereof. The kit can optionally further include control slides for assessing hybridization and signal of the labeled DNAs.

### VIII. Detectable Labels and Methods of Labeling

The nucleic acid probes disclosed herein can include one or more labels, for example to permit detection of a target nucleic acid molecule. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or quantity (for example, gene copy number) of a target nucleic acid (to which the labeled uniquely specific nucleic acid molecule is bound or hybridized) in a sample. The disclosure is not limited to the use of particular labels, although examples are provided.

A label associated with one or more nucleic acid molecules (such as the disclosed probes) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies, e.g., see, The Handbook—A Guide to Fluorescent Probes and Labeling Technologies. Examples of particular fluorophores that can be attached (for example, chemically conjugated) to a nucleic acid molecule (such as a uniquely specific binding region) are provided in U.S. Pat. No. 5,866,366 to Nazarenko et al. (which is incorporated in its entirety by reference herein), such as 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DAB ITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); 2′, 7′-difluorofluorescein (OREGON GREEN®); fluorescamine; IR144; IR1446; Malachite Green isothiocyanate; 4-methylumbelliferone; ortho-cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™ diethylaminocoumarin, fluorescein chlorotriazinyl, naphthofluorescein, 4,7-dichlororhodamine and xanthene (as described in U.S. Pat. No. 5,800,996 to Lee et al., which is incorporated in its entirety by reference herein) and derivatives thereof. Other fluorophores known to those skilled in the art can also be used, for example those available from Life Technologies (Carlsbad, Calif.) and including the ALEXA FLUOR® series of dyes (for example, as described in U.S. Pat. Nos. 5,696,157, 6,130,101 and 6, 716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912), the disclosures of which are incorporated in their entirety herein by reference.

In addition to the fluorochromes described above, a fluorescent label can be a fluorescent nanoparticle, such as a semiconductor nanocrystal, e.g., a quantum dot (obtained, for example, from Life Technologies); see also, U.S. Pat. Nos. 6,815,064; 6,682,596; and 6,649,138, the disclosures of which are incorporated in their entirety herein by reference). Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:2013-2016, 1998; Chan et al., Science 281:2016-2018, 1998; and U.S. Pat. No. 6,274,323, the disclosures of which are incorporated in their entirety herein by reference.

Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. WO 99/26299, the disclosures of which are incorporated in their entirety herein by reference. Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlsbad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules (such as the disclosed probes) also include enzymes, for example horseradish peroxidase (HRP), alkaline phosphatase (AP), acid phosphatase, glucose oxidase, β-galactosidase, β-glucuronidase, or β-lactamase. Where the detectable label includes an enzyme, a chromogen, fluorogenic compound, or luminogenic compound can be used in combination with the enzyme to generate a detectable signal (numerous of such compounds are commercially available, for example, from Life Technologies). Particular examples of chromogenic compounds include diaminobenzidine (DAB), 4-nitrophenylphosphate (pNPP), fast red, fast blue, bromochloroindolyl phosphate (BCIP), nitro blue tetrazolium (NBT), BCIP/NBT, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-a-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, and tetrazolium violet.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922 the disclosures of which are incorporated in their entirety herein by reference). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

In non-limiting examples, the disclosed nucleic acid probes are labeled with dNTPs covalently attached to hapten molecules (such as a nitro-aromatic compound (e.g., 2,4-dinitrophenyl (DNP)), biotin, fluorescein, digoxigenin, etc.). Additional haptens suitable for labeling the disclosed probes include nitropyrazole, 3-hydroxyquinoxaline, thiazolesulfonamide, nitrocinnamic acid, rotenone, 7-(diethylamino)coumarin-3-carboxylic acid, benzodiazepine, or benzofuran haptens (see, e.g., International Pat. Publ. No. WO 2012/003476. incorporated herein by reference). Methods for conjugating haptens and other labels to dNTPs (e.g., to facilitate incorporation into labeled probes) are well known in the art. For examples of procedures, see, e.g., U.S. Pat. Nos. 5,258,507, 4,772,691, 5,328,824, and 4,711,955, the disclosures of which are incorporated in their entirety herein by reference. Indeed, numerous labeled dNTPs are available commercially, for example from Life Technologies (Carlsbad, Calif.). A label can be directly or indirectly attached to a dNTP at any location on the dNTP, such as a phosphate (e.g., α, β or γ phosphate) or a sugar.

Detection of labeled nucleic acid molecules can be accomplished by contacting the hapten-labeled nucleic acid molecules bound to the genomic target nucleic acid with a primary anti-hapten antibody. In one example, the primary anti-hapten antibody (such as a mouse anti-hapten antibody) is directly labeled with an enzyme. In another example, a secondary anti-antibody (such as a goat anti-mouse IgG antibody) conjugated to an enzyme is used for signal amplification. In CISH a chromogenic substrate is added, for SISH, silver ions and other reagents as outlined in the referenced patents/applications are added.

In some examples, a probe is labeled by incorporating one or more labeled dNTPs using an enzymatic (polymerization) reaction. For example, the disclosed nucleic acid probes (for example, incorporated into a plasmid vector) can be labeled by nick translation (using, for example, biotin, DNP, digoxigenin, etc.) or by random primer extension with terminal transferase (e.g., 3′ end tailing). In some examples, the nucleic probe is labeled by a modified nick translation reaction where the ratio of DNA polymerase Ito deoxyribonuclease I (DNase I) is modified to produce greater than 100% of the starting material. In particular examples, the nick translation reaction includes DNA polymerase Ito DNase I at a ratio of at least about 800:1, such as at least 2000:1, at least 4000:1, at least 8000:1, at least 10,000:1, at least 12,000:1, at least 16,000:1, such as about 800:1 to 24,000:1 and the reaction is carried out overnight (for example, for about 16-22 hours) at a substantially isothermal temperature, for example, at about 16° C. to 25° C. (such as room temperature). If the probe is included in a probe set (for example, multiple plasmids, such as 2, 3, 4, 5, 6, 7, 8, 9, 10, or more plasmids), the plasmids may be mixed in an equal molar ratio prior to performing the labeling reaction (such as nick translation or modified nick translation).

In other examples, chemical labeling procedures can also be employed. Numerous reagents (including hapten, fluorophore, and other labeled nucleotides) and other kits are commercially available for enzymatic labeling of nucleic acids, including the disclosed nucleic acid probes. As will be apparent to those of skill in the art, any of the labels and detection procedures disclosed above are applicable in the context of labeling a probe, e.g., for use in in situ hybridization reactions. For example, the Amersham MULTIPRIME® DNA labeling system, various specific reagents and kits available from Molecular Probes/Life Technologies, or any other similar reagents or kits can be used to label the nucleic acids disclosed herein. In particular examples, the disclosed probes can be directly or indirectly labeled with a hapten, a ligand, a fluorescent moiety (e.g., a fluorophore or a semiconductor nanocrystal), a chromogenic moiety, or a radioisotope. For example, for indirect labeling, the label can be attached to nucleic acid molecules via a linker (e.g., PEG or biotin). Additional methods that can be used to label probe nucleic acid molecules are provided in U.S. Application Pub. No. 2005/0158770, the disclosure of which is incorporated in its entirety herein by reference.

### VI. Methods of Detecting a Target Nucleic Acid

The disclosed probes can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH). Exemplary uses are discussed below.

In some embodiments, the disclosed methods of detecting a target nucleic acid include comparing the signal or gene copy number detected in a sample utilizing one or more of the disclosed probes or probe sets in a sample with a control or reference value. In some examples, a change in signal from a probe or probe set relative to a control (such as an increase of about 1.5-fold, 2-fold, 3-fold, 5-fold, 10-fold, 20-fold, or more relative to a control sample or reference value) indicates the presence, expression, or gene copy number of a target nucleic acid (such as 19Q12, 13q12, ATM, DLEU2, INSR, and/or TP53) in the sample. In some examples, change in expression of a target nucleic acid (such as 19Q12, 13q12, ATM, DLEU2, INSR and/or TP53) compared to a control (such as an increase or decrease of about 1.5-fold, 2-fold, 3-fold, 5-fold, 10-fold, 20-fold, or more relative to a control sample or reference value) indicates presence or prognosis of a tumor. In other examples, the gene copy number of a target nucleic acid (such as 19Q12, 13q12, ATM, DLEU2, INSR, and/or TP53) is determined and an increase in gene copy number (such as a gene copy number greater than about 2, 3, 4, 5, 10, 20, or more) indicates the presence or prognosis of a tumor.

In other examples, the probes may be used to analyze copy number changes associated with autism (e.g., cultured peripheral blood lymphocytes as described by van Daalen et al., Neurogenetics 12:315-323, 2011, the disclosures of which are incorporated in their entirety herein by reference). In another example, the probes may be used to evaluate signaling pathways associated with leptins and metabolism (e.g., Donato et al., Arq. Bras. Endocrinol. Metab. 54(7):591-602, 2010 and Williams et al., J. Neurosci. 31(37):13147-13156, 2011, the disclosures of which are incorporated in their entirety herein by reference).

### A. In Situ Hybridization

In situ hybridization (ISH) involves contacting a sample containing a target nucleic acid (e.g., a genomic target nucleic acid) in the context of a metaphase or interphase chromosome preparation (such as a cell or tissue sample mounted on a slide) with a labeled probe specifically hybridizable or specific for the target nucleic acid (for example, one or more of the probes disclosed herein). The slides are optionally pretreated, e.g., to remove paraffin or other materials that can interfere with uniform hybridization. The chromosome sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat anti-avidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). The enzyme reaction can be performed in, for example, AP buffer containing NBT/BCIP and stopped by incubation in 2×SSC. For a general description of in situ hybridization procedures, see, e.g., U.S. Pat. No. 4,888,278, the disclosures of which are incorporated in their entirety herein by reference.

Numerous procedures for FISH, CISH, and SISH are known in the art. For example, procedures for performing FISH are described in U.S. Pat. Nos. 5,447,841; 5,472,842; and 5,427,932, the disclosures of which are incorporated in their entirety herein by reference; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970, the disclosures of which are incorporated in their entirety herein by reference. Additional detection methods are provided in U.S. Pat. No. 6,280,929, the disclosures of which are incorporated in their entirety herein by reference.

Numerous reagents and detection schemes can be employed in conjunction with FISH, CISH, and SISH procedures to improve sensitivity, resolution, or other desirable properties. As discussed above, probes labeled with fluorophores (including fluorescent dyes and quantum dots) can be directly optically detected when performing FISH. Alternatively, the probe can be labeled with a non-fluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., quantum dot) or with another indirectly detectable moiety, or can be contacted with one or more additional specific binding agents (e.g., secondary or specific antibodies), which can in turn be labeled with a fluorophore. Optionally, the detectable label is attached directly to the antibody, receptor (or other specific binding agent). Alternatively, the detectable label is attached to the binding agent via a linker, such as a hydrazide thiol linker, a polyethylene glycol linker, or any other flexible attachment moiety with comparable reactivities. For example, a specific binding agent, such as an antibody, a receptor (or other anti-ligand), avidin, or the like can be covalently modified with a fluorophore (or other label) via a heterobifunctional polyalkyleneglycol linker such as a heterobifunctional polyethyleneglycol (PEG) linker. A heterobifunctional linker combines two different reactive groups selected, e.g., from a carbonyl-reactive group, an amine-reactive group, a thiol-reactive group and a photo-reactive group, the first of which attaches to the label and the second of which attaches to the specific binding agent.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153, the disclosures of which are incorporated in their entirety herein by reference.

In further examples, a signal amplification method is utilized, for example, to increase sensitivity of the probe. For example, CAtalyzed Reporter Deposition (CARD), also known as Tyramide Signal Amplification (TSA™) may be utilized. In one variation of this method a biotinylated nucleic acid probe detects the presence of a target by binding thereto. Next a streptavidin-peroxidase conjugate is added. The streptavidin binds to the biotin. A substrate of biotinylated tyramide (tyramine is 4-(2-aminoethyl)phenol) is used, which presumably becomes a free radical when interacting with the peroxidase enzyme. The phenolic radical then reacts quickly with the surrounding material, thus depositing or fixing biotin in the vicinity. This process is repeated by providing more substrate (biotinylated tyramide) and building up more localized biotin. Finally, the “amplified” biotin deposit is detected with streptavidin attached to a fluorescent molecule. Alternatively, the amplified biotin deposit can be detected with avidin-peroxidase complex, that is then fed 3,3′-diaminobenzidine to produce a brown color. It has been found that tyramide attached to fluorescent molecules also serve as substrates for the enzyme, thus simplifying the procedure by eliminating steps.

In other examples, the signal amplification method utilizes branched DNA (bDNA) signal amplification. In some examples, target-specific oligonucleotides (label extenders and capture extenders) are hybridized with high stringency to the target nucleic acid. Capture extenders are designed to hybridize to the target and to capture probes, which are attached to a microwell plate. Label extenders are designed to hybridize to contiguous regions on the target and to provide sequences for hybridization of a preamplifier oligonucleotide. Signal amplification then begins with preamplifier probes hybridizing to label extenders. The preamplifier forms a stable hybrid only if it hybridizes to two adjacent label extenders. Other regions on the preamplifier are designed to hybridize to multiple bDNA amplifier molecules that create a branched structure. Finally, alkaline phosphatase (AP)-labeled oligonucleotides, which are complementary to bDNA amplifier sequences, bind to the bDNA molecule by hybridization. The bDNA signal is the chemiluminescent product of the AP reaction See, e.g., Tsongalis, Microbiol. Inf. Dis. 126:448-453, 2006; U.S. Pat. No. 7,033,758, the disclosures of which are incorporated in their entirety herein by reference.

In further examples, the signal amplification method utilizes polymerized antibodies. In some examples, the labeled probe is detected by using a primary antibody to the label (such as an anti-DIG or anti-DNP antibody). The primary antibody is detected by a polymerized secondary antibody (such as a polymerized HRP-conjugated secondary antibody or an AP-conjugated secondary antibody). The enzymatic reaction of AP or HRP leads to the formation of strong signals that can be visualized.

It will be appreciated by those of skill in the art that by appropriately selecting labeled probe-specific binding agent pairs, multiplex detection schemes can be produced to facilitate detection of multiple target nucleic acids (e.g., genomic target nucleic acids) in a single assay (e.g., on a single cell or tissue sample or on more than one cell or tissue sample). For example, a first probe that corresponds to a first target nucleic acid can be labeled with a first hapten, such as biotin, while a second probe that corresponds to a second target nucleic acid can be labeled with a second hapten, such as DNP. Following exposure of the sample to the probes, the bound probes can be detected by contacting the sample with a first specific binding agent (in this case avidin labeled with a first fluorophore, for example, a first spectrally distinct quantum dot, e.g., that emits at 585 nm) and a second specific binding agent (in this case an anti-DNP antibody, or antibody fragment, labeled with a second fluorophore (for example, a second spectrally distinct quantum dot, e.g., that emits at 705 nm). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Additional details regarding certain detection methods, e.g., as utilized in CISH and SISH procedures, can be found in Bourne, The Handbook of Immunoperoxidase Staining Methods, published by Dako Corporation, Santa Barbara, Calif.

In some embodiments, the disclosed probes can be used in methods of determining the copy number of a target nucleic acid (such as 19Q12, 13q12, ATM, DLEU2, or INSR) in a biological sample (such as a tissue sample). Methods of determining the copy number of a gene or chromosomal region are well known to those of skill in the art. In some examples, the methods include in situ hybridization (such as fluorescent, chromogenic, or silver in situ hybridization), comparative genomic hybridization, or polymerase chain reaction (such as real-time quantitative PCR). In some examples, methods of determining gene copy number include counting the number of ISH signals (such as fluorescent, colored, or silver spots) for the target nucleic acid in one or more individual cells. The methods may also include counting the number of ISH signals (such as fluorescent, colored, or silver spots) for a reference (such as a chromosome-specific probe) in the cells. In particular examples, the number of copies of the gene (or chromosome) may be estimated by the person (or computer, in the case of an automated method) scoring the slide. In some examples, an increased copy number relative to a control (such as an increase of about 1.5-fold, 2-fold, 3-fold, 5-fold, 10-fold, 20-fold, or more relative to a control sample or reference value) indicates an increase in the target nucleic acid copy number.

In some examples, the method includes counting the number of copies per cell or nucleus of a reference, such as a chromosomal locus known not to be abnormal, for example a centromere. In some examples, the reference is on the same chromosome as the gene of interest. Exemplary reference chromosomes that can be used for particular human genes of interest are provided in Table 1. In particular examples, the reference locus is detected by using a centromere-specific probe. Such probes are known in the art and are commercially available, for example, Vysis CEP probes (Abbott Molecular, Des Plaines, Ill.) and SPOTLIGHT centromeric probes (Invitrogen, Carlsbad, Calif.). In some examples, a ratio of target nucleic acid copy number to reference copy number greater than about two (such as greater than about 2, 3, 4, 5, 10, 20, or more), indicates an increase in the target nucleic acid copy number.

### B. Microarray Applications

Comparative genomic hybridization (CGH) is a molecular-cytogenetic method for the analysis of copy number changes (gain/loss) in the DNA content of cells. The contribution of genome structural variation to human disease is found in rare genomic disorders (for example, Trisomy 21, Prader-Willi Syndrome) and a broad range of human diseases, such as genetic diseases, autism, schizophrenia, cancers, and autoimmune diseases. In one example, the method is based on the hybridization of differently fluorescently labeled sample DNA (for example, labeled with fluorescein-FITC) and normal DNA (for example, labeled with rhodamine or Texas red) to normal human metaphase preparations. Using methods known in the art, such as epifluorescence microscopy and quantitative image analysis, regional differences in the fluorescence ratio of sample versus control DNA can be detected and used for identifying abnormal regions in the sample cell genome. CGH detects unbalanced chromosomes changes (such as increase or decrease in DNA copy number). See, e.g., Kallioniemi et al., Science 258:818-821, 1992; U.S. Pat. Nos. 5,665,549 and 5,721,098, the disclosures of which are incorporated in their entirety herein by reference.

Genomic DNA copy number may also be determined by array CGH (aCGH). See, e.g., Pinkel and Albertson, Nat. Genet. 37:S11-S17, 2005; Pinkel et al., Nat. Genet. 20:207-211, 1998; Pollack et al., Nat. Genet. 23:41-46, 1999, the disclosures of which are incorporated in their entirety herein by reference. Similar to standard CGH, sample and reference DNA are differentially labeled and mixed. However, for aCGH, the DNA mixture is hybridized to a slide containing hundreds or thousands of defined DNA probes (such as probes that specifically hybridize to a genomic target nucleic acid of interest). The fluorescence intensity ratio at each probe in the array is used to evaluate regions of DNA gain or loss in the sample, which can be mapped in finer detail than CGH, based on the particular probes which exhibit altered fluorescence intensity.

In general, CGH (and aCGH) does not provide information as to the exact number of copies of a particular genomic DNA or chromosomal region. Instead, CGH provides information on the relative copy number of one sample (such as a tumor sample) compared to another (such as a reference sample, for example a non-tumor cell or tissue sample). Thus, CGH is most useful to determine whether genomic DNA copy number of a target nucleic acid is increased or decreased as compared to a reference sample thereby determining the copy number variation of a target nucleic acid sample relative to a reference sample.

In a particular example, the disclosed probes may be utilized for aCGH. For example, an unlabeled probe disclosed herein (such as one or more of any one of SEQ ID NOs: 1-50 or a portion thereof) may be immobilized on a solid surface (such as nitrocellulose, nylon, glass, cellulose acetate, plastics (for example, polyethylene, polypropylene, or polystyrene), paper, ceramics, metals, and the like). Methods of immobilizing nucleic acids on a solid surface are well known in the art (see, e.g., Bischoff et al., Anal. Biochem. 164:336-344, 1987; Kremsky et al., Nuc. Acids Res. 15:2891-2910, 1987, the disclosures of which are incorporated in their entirety herein by reference). As discussed above, differently fluorescently labeled sample DNA (for example, labeled with fluorescein-FITC) and reference DNA (for example, labeled with rhodamine or Texas red) is hybridized to the probe array and regional differences in the fluorescence ratio of sample versus reference DNA can be detected and used for identifying abnormal regions in the sample cell genome.

In another example, disclosed probes are synthesized in situ on a solid surface (such as nitrocellulose, nylon, glass, cellulose acetate, plastics (for example, polyethylene, polypropylene, or polystyrene), paper, ceramics, metals, and the like). For example, the probes are utilized for printing, in situ, on a solid support utilizing computer based microarray printing methodologies, such as those described in U.S. Pat. Nos. 6,315,958; 6,444,175; and 7,083,975 and U.S. Pat. Application Nos. 2002/0041420, 2004/0126757, 2007/0037274, and 2007/0140906, the disclosures of which are incorporated in their entirety herein by reference. In some examples, using a maskless array synthesis (MAS) instrument, oligonucleotides synthesized in situ on the microarray are under software control resulting in individually customized arrays based on the particular needs of an investigator. The number of probes synthesized on a microarray varies, for example presently anywhere from 50,000 to 2.1 million probes, in various configurations, can be synthesized on a single microarray slide (for example, Roche NimbleGen CGH microarrays contain from 385,000 to 4 million or more probes/array).

Probe sequences are synthesized either in situ by MAS instruments, or alternatively by utilizing photolithographic methods as described in U.S. Pat. Nos. 5,143,854; 5,424,186; 5,405,783; and 5,445,934, the disclosures of which are incorporated in their entirety herein by reference. Utilizing the disclosed probes for microarray applications is not limited by their method of manufacture, and a skilled artisan will understand additional methods of creating microarrays with uniquely specific oligonucleotide probes thereon that are equally applicable. For example, historical methods of spotting nucleic acid sequences onto solid supports are also contemplated, such that historically utilized nucleic acid probes are replaced by uniquely specific probes as described herein. Regardless of method used to place probes on a microarray, the uniquely specific probes can be used to target one or more nucleic acid samples, either individually or on the same array.

Applications of the probes disclosed herein that are in situ synthesized or otherwise immobilized on a microarray slide can be utilized for aCGH as well as other microarray based genomic target enrichment applications such as those described in U.S. Pat. Publication Nos. 2008/0194413, 2008/0194414, 2009/0203540, and 2009/0221438, the disclosures of which are incorporated in their entirety herein by reference. Utilizing uniquely specific probes for generating in situ synthesized microarrays provides many improvements over current microarray probe designs. For example, use of uniquely specific probes allows for more specific binding of target sequences as compared to current probes, therefore not as many probes are needed per target and/or in conjunction more can be added to capture additional targets. Further, the need for blocking DNA (for example, Cot-1™ DNA) typically utilized in microarray experiments is reduced or eliminated when utilizing uniquely specific oligonucleotide probes.

For CGH applications, typically both target and reference genomic DNA are hybridized on one array for comparison on one microarray substrate. The CGH Analysis User's Guide (version 5.1, Roche NimbleGen, Madison, Wis.; available on the World Wide Web at nimblegen.com) describes methods for performing CGH analysis utilizing microarrays. In general, two genomic DNA samples, a target sample and a reference sample, are fragmented and labeled with different detection moieties (for example, Cy-3 and Cy-5 fluorescent moieties). The two labeled samples are mixed and hybridized to a microarray support, in this case a microarray comprising uniquely specific oligonucleotide probes, and the microarray is subsequently assayed for both detection moieties. The microarrays are scanned and detection data captured, for example by scanning a microarray with a microarray scanner (for example, a MS200 Microarray Scanner; Roche NimbleGen). The data is analyzed using analysis software (for example, NimbleScan; Roche NimbleGen). The target genomic sequence data is compared to the reference and DNA copy number gains and losses in target samples are thereby characterized. The target genomic sequences can be, for example, from targeted region(s) of one or more chromosome(s), one whole chromosome, or the total genomic complement of an organism (for example, a eukaryotic genome, such as a mammalian genome, for example a human genome).

For genomic enrichment (also known as sequence capture), typically a genomic sample is hybridized to a microarray support comprising targeted sequence specific probes for specific target enrichment prior to downstream applications, such as sequencing. The Sequence Capture User's Guide (version 3.1, Roche NimbleGen, incorporated by reference herein) describes methods for performing genomic enrichment. In general, a genomic DNA sample is prepared for hybridization to a microarray support, in this case a microarray comprising the disclosed uniquely specific oligonucleotide probes designed to capture targeted sequences from a genomic sample for enrichment. The captured genomic sequences are then eluted from the microarray support and sequenced, or used for other applications.

### C. Blocking DNA

Genome-specific blocking DNA (such as human DNA, for example, total human placental DNA or Cot-1™ DNA) is usually included in a hybridization solution (such as for in situ hybridization) to suppress probe hybridization to repetitive DNA sequences or to counteract probe hybridization to highly homologous (frequently identical) off target sequences when a probe complementary to a human genomic target nucleic acid is utilized. In hybridization with standard probes, in the absence of genome-specific blocking DNA, an unacceptably high level of background staining (for example, non-specific binding, such as hybridization to non-target nucleic acid sequence) is usually present, even when a “repeat-free” probe is used. The disclosed nucleic acid probes exhibit reduced background staining, even in the absence of blocking DNA. In particular examples, the hybridization solution including the disclosed probes does not include genome-specific blocking DNA (for example, total human placental DNA or Cot-1™ DNA, if the probe is complementary to a human genomic target nucleic acid). This advantage is derived from the uniquely specific nature of the target sequences included in the nucleic acid probe; each labeled probe sequence binds only to the cognate uniquely specific genomic sequence. This results in dramatic increases in signal to noise ratios for ISH techniques.

In some examples the hybridization solution may contain carrier DNA from a different organism (for example, salmon sperm DNA or herring sperm DNA, if the genomic target nucleic acid is a human genomic target nucleic acid) to reduce non-specific binding of the probe to non-DNA materials (for example to reaction vessels or slides) with high net positive charge which can non-specifically bind to the negatively charged probe DNA.

The disclosure is further illustrated by the following non-limiting Examples.

### IX. Examples

The following examples are provided to illustrate certain specific features of working embodiments and general protocols. The scope of the present invention is not limited to those features exemplified by the following examples.

### Example 1: CLL Analysis

Abnormal cytogenetics are found in the majority of patients with CLL, and each subtype is associated with differentiated frequency, outcome, and suggested treatments (see Table 3).

Example 1 demonstrates that probes of the present disclosure can detect such abnormalities via in situ hybridization (ISH) on formalin fixed paraffin embedded bone marrow/trephine samples.

A. Preparation of Reagents:

DNA probes were labeled with either DNP or DIG using standard reaction conditions. Specificity of each probe was verified using CGH Target Metaphase spread. Functionality of each probe was determined by standard dual ISH using the U Dual Color ISH Open Probe procedure on standard cancer tissues supplied by Ventana Medical Systems, Inc.

B. Reagents

(if commercially available from Ventana Medical Systems Inc., reagents are listed with the associated part number).

TP53/Chr17:
TP53-DNP [30 ug/ml] (mixture of 10 probes according to SEQ ID NOs: 51-60);
ASR CEN-17-DIG [1.25 ug/ml] (Ventana Medical Systems, Inc., cat. #760-1224);

DLEU/13q12.11:
DLEU-DNP [30 ug/ml], (mixture of 10 probes according to SEQ ID NOs: 31-40);
13q12.11-DIG [30 ug/ml], (mixture of 10 probes according to SEQ ID NOs: 11-20);
1 mg/ml sonicated fsDNA;

ATM/13q12.11:
ATM-DNP [30 ug/ml], (mixture of 10 probes according to SEQ ID NOs: 21-30)
CEN11-DIG [4 ug/ml], 1 mg/ml placental DNA

Trisomy 12:
ASR CEN-12-DNP [0.75 ug/ml] (Ventana Medical Systems, Inc., cat. #760-1221);
ISH Probe Dispenser Card

C. Detection Reagents
ISH Protease 2 (780-4148)
ISH Protease 3 (780-4149)
HYB READY (780-4409)
ULTRAVIEW SISH DNP Detection Kit (760-098)
ULTRAVIEW Red ISH DIG Detection Kit (760-505)
Hematoxylin II (790-2208)
Bluing Reagent (760-2037)

D. Bulk Reagents
EZ PREP Concentrate (950-102)
Ultra LSC (650-210)
SSC Concentrate (950-110)
Reaction Buffer (950-300)
Ultra CC1 (950-224)
Ultra CC2 (950-223)
ULTRAVIEW SilverWash II (780-003)

E. Tissue—Suggested Protocols

The following staining protocols were performed on formalin-fixed paraffin-embedded bone marrow trephine samples and blood clot using a BenchMark XT or BenchMark ULTRA automated slide staining platform:

TABLE 4
17p-(TP53/CHR17)
17p-(TP53/Chr17)

| | Procedure | | |
|---|---|---|---|
| | U Dual Color Open Probe | | |
| | Protocol | | |
| | 1st Pass | 2nd Pass | 3rd Pass |
| Baking | 63° C., 20 min | | |
| Depar | Extended II; 69° C., 16 min | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | TP53-DNP [30 ug/ml] |
| | Chr17-DIG [1.25 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 4-8 min |
| Counterstain | Hematoxylin II, 8 min |
| | BluingReagent, 8 min |

TABLE 5
13 q-(DLEU/13q12.11)
13 q-(DLEU/13q12.11)

| | Procedure | | |
|---|---|---|---|
| | U Dual Color Open Probe | | |
| | Protocol | | |
| | 1st Pass | 2nd Pass | 3rd Pass |
| Baking | 63° C., 20 min | | |
| Depar | Extended II; 69° C., 16 min | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | DLEU-DNP [30 ug/ml] |
| | 13q12.11 [30 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 12-16 min |
| Counterstain | Hematoxylin II, 8 min |
| | BluingReagent, 8 min |

TABLE 6
11p-(ATM/CEN11)
11p-(ATM/CEN11)

| | Procedure | | |
|---|---|---|---|
| | U Dual Color Open Probe | | |
| | Protocol | | |
| | 1st Pass | 2nd Pass | 3rd Pass |
| Baking | 63° C., 20 min | | |
| Depar | Extended II, 69° C., 16 min | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | ATM-DNP [30 ug/ml] |
| | CEN11-DIG [4 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 12-16 min |
| Counterstain | Hematoxylin II, 8 min |
| | BluingReagent, 8 min |

TABLE 7
Trisomy 12 (CEN12)
Trisomy 12 (CEN12)

| | Procedure | | |
|---|---|---|---|
| | U Dual Color Open Probe | | |
| | Protocol | | |
| | 1st Pass | 2nd Pass | 3rd Pass |
| Baking | 63° C., 20 min | | |
| Depar | Extended II; 69° C., 16 min | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | CEN12-DIG [0.75 ug/ml] |
| Hybridzation | 47° C., 6 hr |
| Stringency Wash | 68° C.*, 8 min |
| Silver Detection | NOT SELECTED |
| ISH DNP HRP | |
| ISH DNP CHRC | |
| Red Detection | |
| Red ISH DIG AP | 24 min |
| Red ISH DIG FR | 4-8 min |
| Counterstain | Hematoxylin II, 8 min |
| | BluingReagent, 8 min |

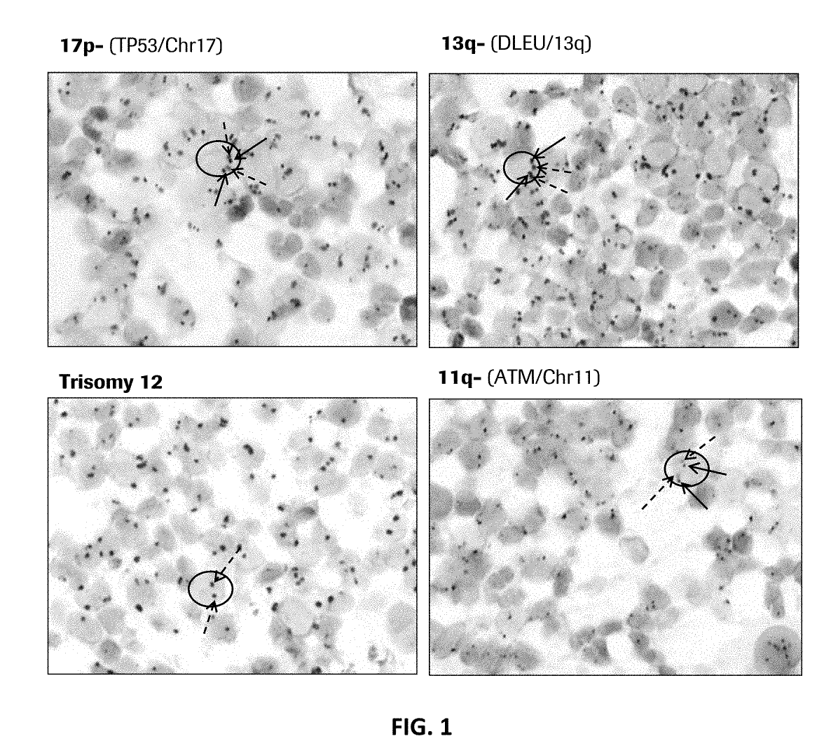

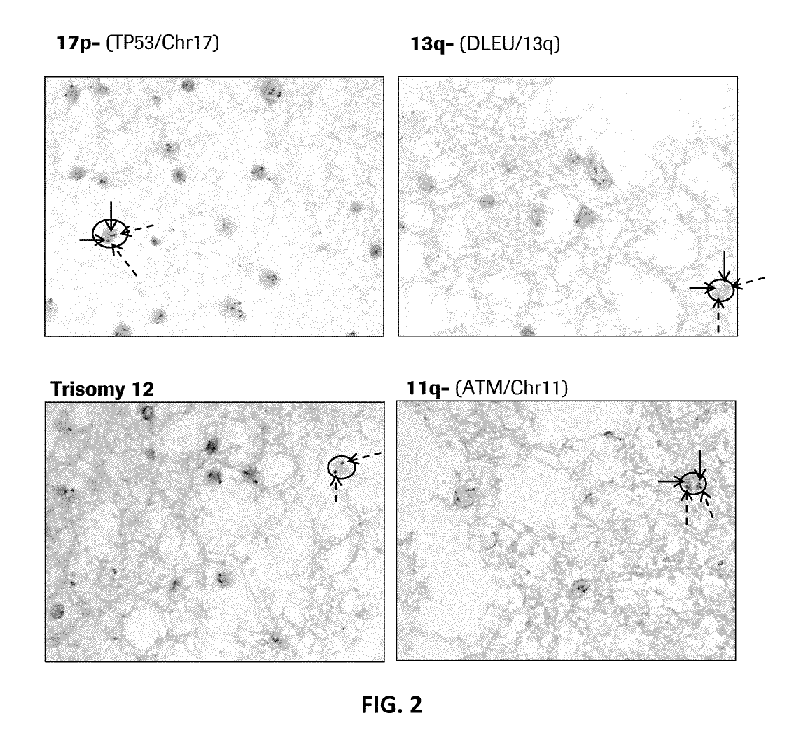

Exemplary stained slides are illustrated at FIG. 1 (bone marrow/trephine samples) and FIG. 2 (blood clot). Ovals indicate an exemplary cell. Dashed arrows indicate staining corresponding to the centromere/chromosome specific probe, which is red in color images. Solid arrows indicate staining corresponding to the gene-specific probe, which is black in color images.

F. Blood—Suggested Protocols

The following staining protocols were performed on formalin-fixed whole blood smears using a BenchMark XT or BenchMark ULTRA automated slide staining platform:

TABLE 8
17p-(TP53/CHR17)
17p-(TP53/Chr17)

| | Procedure | | |
|---|---|---|---|
| | U Dual Color Open Probe | | |
| | Protocol | | |
| | 1st Pass | 2nd Pass | 3rd Pass |
| Baking | Not Selected | | |
| Depar | Not Selected | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | TP53-DNP [30 ug/ml] |
| | Chr17-DIG [1.25 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 4-8 min |
| Counterstain | Hematoxylin II, 8 min |
| | BluingReagent, 8 min |

TABLE 9
13q-(DLEU/13q12.11)
13q-(DLEU/13q12.11)

| | Procedure | | |
|---|---|---|---|
| | U Dual Color Open Probe | | |
| | Protocol | | |
| | 1st Pass | 2nd Pass | 3rd Pass |
| Baking | Not Selected | | |
| Depar | Not Selected | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | DLEU-DNP [30 ug/ml] |
| | 13q12.11 [30 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 12-16 min |
| Counterstain | Hematoxylin II, 8 min |
| | BluingReagent, 8 min |

TABLE 10
11p-(ATM/CEN11)
11p-(ATM/CEN11)

| | Procedure | | |
|---|---|---|---|
| | U Dual Color Open Probe | | |
| | Protocol | | |
| | 1st Pass | 2nd Pass | 3rd Pass |
| Baking | Not Selected | | |
| Depar | Not Selected | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | ATM-DNP [30 ug/ml] |
| | CEN11-DIG [4 ug/ml] |
| Hybridzation | 47° C., 6 hr |
| Stringency Wash | 68° C.*, 8 min |
| Silver Detection | |
| ISH DNP HRP | 24 min |
| ISH DNP CHRC | 8-12 min |
| Red Detection | |
| Red ISH DIG AP | 24 min |
| Red ISH DIG FR | 12-16 min |
| Counterstain | Hematoxylin II, 8 min |
| | BluingReagent, 8 min |

TABLE 11
Trisomy 12 (CEN12)
Trisomy 12 (CEN12)

| | Procedure | | |
|---|---|---|---|
| | U Dual Color Open Probe | | |
| | Protocol | | |
| | 1st Pass | 2nd Pass | 3rd Pass |
| Baking | Not Selected | | |
| Depar | Not Selected | | |
| Cell Conditioning, | 3 Cycles, 82° C. | | |
| CC2 | | | |
| ISH Protease | ISH Protease | ISH Protease | ISH Protease |
| | 3, 16 min | 3, 24 min | 2, 8 min |

| | |
|---|---|
| Probes | CEN12-DIG [0.75 ug/ml] |
| Hybridzation | 47° C., 6 hr |
| Stringency Wash | 68° C.*, 8 min |
| Silver Detection | NOT SELECTED |
| ISH DNP HRP | |
| ISH DNP CHRC | |
| Red Detection | |
| Red ISH DIG AP | 24 min |
| Red ISH DIG FR | 4-8 min |
| Counterstain | Hematoxylin II, 8 min |
| | BluingReagent, 8 min |

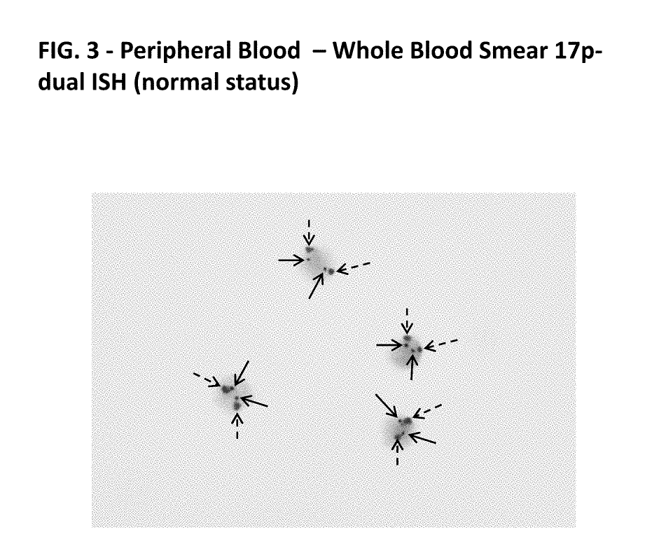

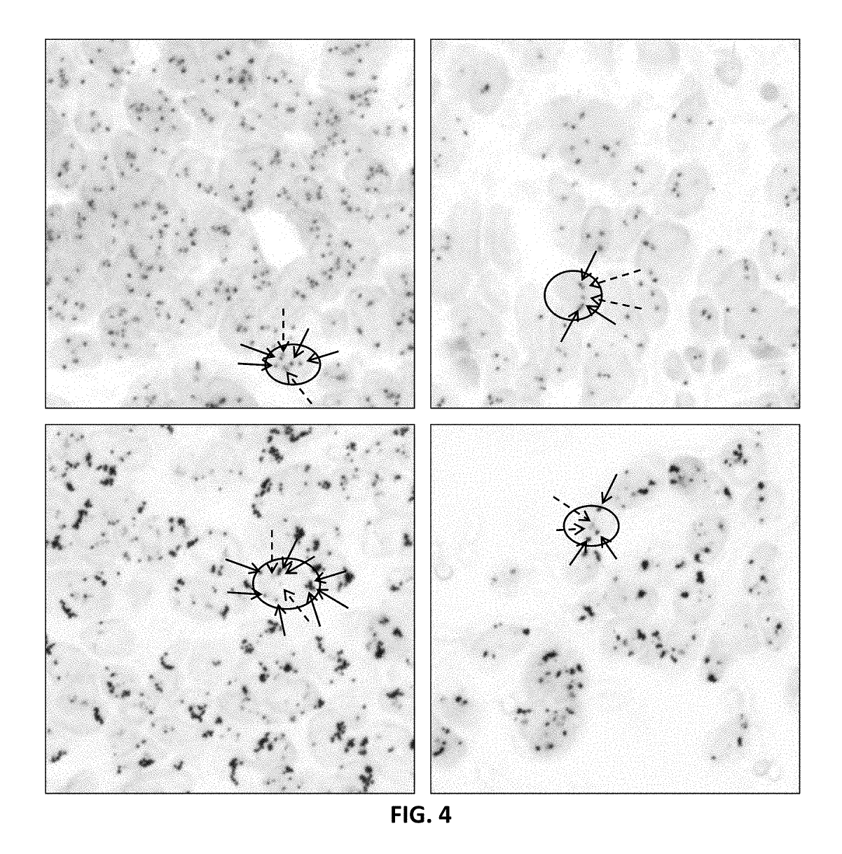

An exemplary slide stained for TP53 and chromosome 17 is illustrated at FIG. 3. Dashed arrows indicate staining corresponding to the chromosome 17 specific probe, which is red in color images. Solid arrows indicate staining corresponding to the TP53-specific probe, which is black in color images.

G. Cell Fixation Method for Cell Preparation
Prepare blood Sample as desired (whole blood smear/Cytospin/etc)
Air-dry.
Immerse slides in 10% NBF (Neutral Buffered Formalin); incubate for 20 min at room temperature.
Rinse slides 3 times in PBS at room temperature.
Rinse slides in ddH20 at room temperature.
Air dry.
Store slides at −20° C. freezer in airtight container.

H. Examining 19q12 Amplification Using Chromogenic ISH

Preparation of Materials: DNA probes were label with either DNP or DIG by Ventana Pilot Plant using standard reaction conditions as per Genomics Technology and Applied Research (GTAR). Specificity of CCNE1-DNP and INSR-DIG probes was verified using CGH Target Metaphase spread. Functionality of each probe was determined by standard dual ISH using the Dual Color ISH Open Probe procedure on standard cancer tissues supplied by Ventana TSM. These protocols are sufficient for both DNP and DIG signals but not deemed “robust”.

I. Dual ISH Scoring Criteria

TABLE 12

| | Procedure | | | | |
|---|---|---|---|---|---|
| | U Dual Color Open Probe | | | | |
| | Protocol | | | | |
| | | | | | 4/5th |
| | 0 Pass | 1st Pass | 2nd Pass | 3rd Pass | Pass |
| Baking | 63° C., 20 min | | | | |
| Depar | Extended II; 69° C., 16 min | | | | |
| Cell Conditioning, | 3 Cycles, 82° C. | | | | 4 Cycles, |
| CC2 | | | | | 82° C. |
| ISH Protease | ISH | ISH | ISH | ISH | ISH |
| | Protease | Protease | Protease | Protease | Protease |
| | 3, 16 | 3, 24 | 3, 32 | 2, 8 | 3, 24 |
| | min | min | min | min | min/ISH |
| | | | | | Protease |
| | | | | | 2, 8 min |

| | |
|---|---|
| Probes | 19q12-DNP (20 ug/ml) |
| | INSR-DIG (20 ug/ml) |
| Hybridzation | 47° C., 6 hr |
| Stringency Wash | 68° C.*, 8 min |
| Silver Detection | |
| ISH DNP HRP | 24 min |
| ISH DNP CHRC | 8 min* |
| Red Detection | |
| Red ISH DIG AP | 24 min |
| Red ISH DIG FR | 12 min* |
| Counterstain | Hematoxylin II, 8 min |
| | BluingReagent, 8 min |

*Adjust Stringency Wash to 72° C. to reduce excessive blush. Adjust ISH DNP CHR C to 12 min to increase DNP silver signal size, although increasing silver times may result in silver “dusting”.

For a more detailed explanation of scoring, please refer to the INFORM HER2 DualISH PMA ULTRA Interpretation guide.)

Review the entire section/specimen looking for tumor regions with good staining (both red and black) that have the highest number of signals.

Select an area of interest and count 17 cells and classify signals with the most information (highest signals).

Select a second area of interest and count 17 cells with the most information (highest signals).

Select a third area of interest and count 16 cells with the most information (highest signals).

Total number of cells=50

### Example 2—Multiplex Gene-Protein Assay for CD79a/TP53/CEN17

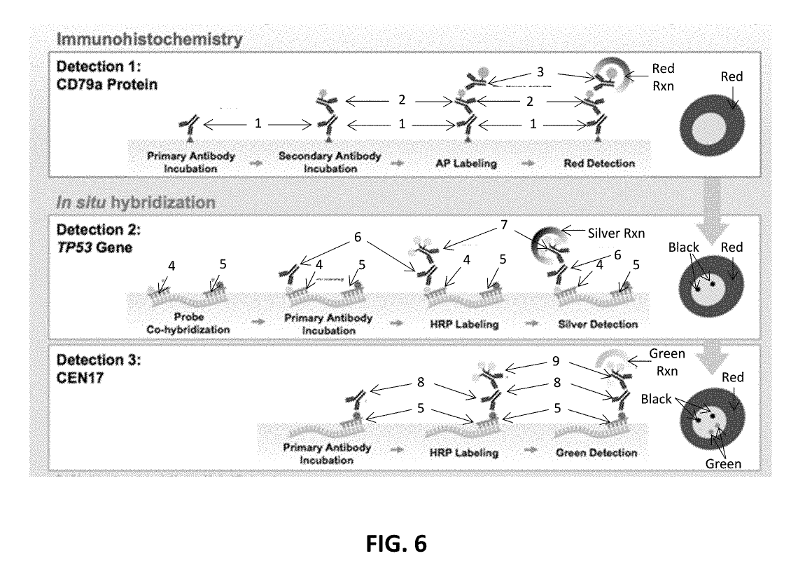

This example describes a multiplex gene-protein assay for detection of CD79a protein, TP53 DNA, and chromosome 17 centromere DNA in a single sample. The staining strategy consists of essentially three staining steps: (1) CD79a protein; (2) TP53 Gene; and (3) CEN17. An overview of the staining strategy is illustrated at FIG. 6.

In the first detection, an antibody specific for CD79a 1 is contacted with the sample. A species-specific secondary antibody 2 conjugated to a first antibody-reactive group (such as a hapten) is then applied to the sample, which binds to the primary antibody 1. A tertiary antibody 3 is then applied to the sample. The tertiary antibody 3 is specific for the antibody-reactive subunit of the secondary antibody 2. The tertiary antibody 3 also is labeled with a detectable label. After reaction to visualize the detectable label, regions at which the primary antibody has bound appear as a first color. In the example illustrated in FIG. 6, the detectable label is alkaline phosphatase (AP), and the first color is red.

In the second detection, a nucleic acid probe specific for the TP53 Gene (TP53 Probe) 4 is hybridized to the sample. The TP53 probe 4 is modified to contain a second antibody-reactive group that is distinct from the first antibody-reactive group (such as a different hapten). The TP53 probe 4 may be hybridized by itself, or may be co-hybridized to the sample with a probe specific for the centromere region of chromosome 17 (CEN17 probe) 5 that is modified to contain a third antibody-reactive group that is distinct from the first and second antibody-reactive groups (such as a different hapten). FIG. 6 illustrates a co-hybridization reaction. A primary antibody specific for the second antibody-reactive group 6 is then reacted with the sample, which binds to the second antibody reactive group. A species specific secondary antibody 7 is then reacted with the sample, which binds to the primary antibody 6. The secondary antibody 7 is conjugated to a detectable label. After reaction to visualize the detectable label, regions at which the primary antibody has bound appear as a second color that is visually distinct from the first color. In the example illustrated in FIG. 6, the detectable label is horseradish peroxidase (HRP), and the second color is silver/black.

In the third detection, a nucleic acid probe specific for the CEN17 probe 5 as described above is applied (if sequential hybridization is used) and/or detected. FIG. 6 illustrates a co-hybridization reaction, so a separate hybridization step is not necessary in that specific example. A primary antibody specific for the third antibody-reactive group 8 is then reacted with the sample, which binds to the third antibody reactive group. A species specific secondary antibody 9 is then reacted with the sample, which binds to the primary antibody 8. The secondary antibody 7 is conjugated to a detectable label. After reaction to visualize the detectable label, regions at which the primary antibody has bound appear as a third color that is visually distinct from the first and second colors. In the example illustrated in FIG. 6, the detectable label is horseradish peroxidase (HRP), and the third color is green.

An example of this reaction scheme was run on blood smear glass slides. The blood smear glass slides were re-fixed in neutral buffered formalin at room temperature overnight. After fixation, the glass slides were rinsed in distilled water several times and air-dried completely. Prior to the staining protocol, the glass slides were soaked in 10% nonfat dry milk (Carnation) in Reaction Buffer (Ventana) for 10 minutes at room temperature. Blood smear glass slides were placed onto VENTANA BenchMark XT automated slide stainer for a TP53 gene-protein assay. Blood samples were heat pretreated with Cell Conditioning 1 (CC1, Ventana) for 48 minutes at 95° C. Then, after rinsing the slides with Reaction Buffer (Ventana), CONFIRM anti-CD79a (SP18) Rabbit Monoclonal Primary Antibody (Ventana, catalog#790-4432) was applied for 28 minutes at 37° C. followed by Reaction Buffer washing steps. A red chromogen was used for detecting CD79a protein as red staining. Blood samples were heat-pretreated with EZ Prep (Ventana) diluted Cell Conditioning 2 (CC2) for two cycles of an 8 minute incubation at 82° C. Then, the samples were digested with ISH-Protease 3 (Ventana) for 20 minutes at 37° C. for completing the pretreatment for the ISH assay part of gene-protein assay. A cocktail of DNP-labeled TP53 and DIG-labeled CEN17 probes was applied onto glass slides with SSC diluted HybReady solution (Ventana). After denaturing step for 8 minutes at 80° C., the hybridization step was performed for 6 hours. A sequential stringency wash and ISH detection step was conducted for TP53 gene and CEN17 signal visualization. For the first stringency and TP53 ISH detection, three cycles of stringency wash steps were performed at 68° C. using SSC followed by ultraView SISH DNP Detection Kit (Ventana, catalog#760-098) and TP53 gene signal were visualized as black dots. For the second stringency and CEN17 ISH detection, three cycles of stringency wash steps were performed at 76° C. using SSC and a HRP-Green signal detection protocol was applied for CEN17 ISH signal visualization. Anti-DIG antibody (from ultraView Red ISH DIG Detection Kit, Ventana, catalog#760-505) was applied to glass slides for 12 minutes at 37° C. followed by HRP-conjugated anti-mouse antibody (UltraMap, Ventana, catalog#760-4313) incubation for 12 minutes at 37° C. CEN17 ISH detection was completed with HRP-Green detection (42 Lifescience). Finally the glass slides were counterstained with diluted Mayer's hematoxylin (1:4 in water) for 4 minutes. The glass slides were washed and air-dried followed by coverslipping.

As illustrated at FIG. 7 and FIG. 8, the protocol results in red staining of CD79a protein, black staining of the TP53 genomic DNA (which appears as dark black dots in the grayscale image of FIGS. 7 & 8), and green/blue staining of chromosome 17 centromere DNA (which appear as more diffuse and lighter dots in the grayscale image of FIGS. 7 & 8). FIG. 8 illustrates an example having a p17 homozygous deletion. CD79a protein staining is illustrated by solid arrows, TP53 gene staining by dashed arrows, and CEN17 staining by dotted arrows. All steps were performed on a BENCHMARK XT automated IHC/ISH staining instrument (Ventana Medical Systems, Tucson, Ariz., Catalog #: N750-BMKXT-FS).

### Further Embodiments

### Embodiment 1

A multiplex method for co-detecting a B cell marker protein, TP53 genomic DNA, and chromosome 17 centromere DNA in a sample on a single slide, said method comprising:

staining the B cell marker protein by contacting the sample with a B cell marker protein-specific antibody and contacting the sample with a first chromogen component for the B cell marker protein-specific antibody, the first chromogen component is adapted to emit or make visible a first color, wherein the presence of the first color indicates the presence of the B cell marker protein; and

staining TP53 genomic DNA and staining chromosome 17 centromere DNA by contacting the sample with a TP53 genomic DNA-specific nucleic acid probe and with a chromosome 17 centromere DNA-specific nucleic acid probe, and contacting the sample with a second chromogen component for the TP53 genomic DNA-specific nucleic acid probe and with a third chromogen component for the chromosome 17 centromere DNA-specific nucleic acid probe, the second chromogen component is adapted to emit or make visible a second color and the third chromogen component is adapted to emit or make visible a third color, wherein the presence of the second color indicates the presence of TP53 genomic DNA and the presence of the third color indicates the presence of chromosome 17 centromere DNA.

### Embodiment 2

The method of embodiment 1, wherein the sample is a blood sample.

### Embodiment 3

The method of embodiment 1 or 2, wherein the B cell marker protein comprises one or more B cell marker proteins selected from the group consisting of CD79a protein, CD79b protein, BCL-2 protein, CD19 protein, CD22 protein, MUM1 protein, PAX5 protein, CD20 protein, Oct2 protein, and Bob.1 protein and the B cell marker protein-specific antibody comprises a CD79a protein-specific antibody, CD79b protein-specific antibody, BCL-2 protein-specific antibody, CD19 protein-specific antibody, CD22 protein-specific antibody, MUM1 protein-specific antibody, PAX5 protein-specific antibody, CD20 protein-specific antibody, Oct2 protein-specific antibody, or Bob.1 protein-specific antibody.

### Embodiment 4

The method of embodiment 3, wherein the B cell marker protein comprises CD79a and the B cell marker protein-specific antibody comprises a CD79a protein-specific antibody.

### Embodiment 5

The method of embodiment 3, wherein the B cell marker protein comprises CD79b or CD20 and the B cell marker protein-specific antibody comprises a CD79b protein-specific antibody or a CD20-specific antibody.

### Embodiment 6

The method of any of embodiments 1 to 5, wherein the first chromogen component comprises fast red, the second chromogen component comprises silver, and the third chromogen component comprises a green chromogen component.

### Embodiment 7

The method of any of embodiments 1 to 5, wherein the first color is transparent enough to allow visualization of the second color and the third color.

### Embodiment 8

The method of any of embodiments 1 to 7 further comprising visualizing the colors using bright-field microscopy.

### Embodiment 9

The method of any of embodiments 1 to 8, wherein the method is automated.

### Embodiment 10

The method of any of embodiments 1 to 9, wherein the step of staining the B cell protein marker is performed before the step of staining TP53 genomic DNA and staining chromosome 17 centromere DNA.

### Embodiment 11

The method of any of embodiments 1 to 10, wherein the sample is subjected to a protease treatment after the step of staining the B cell protein marker but before the step of staining TP53 genomic DNA and staining chromosome 17 centromere DNA, wherein the protease treatment is effective to allow for hybridization of the TP53 DNA-specific nucleic acid probe to the TP53 genomic DNA and for hybridization of the chromosome 17 centromere DNA-specific nucleic acid probe to the chromosome 17 centromere DNA.

### Embodiment 12

The method of embodiment 11, wherein the sample is subjected to a heat treatment after the step of staining the B cell protein marker but before the protease treatment.

### Embodiment 13

The method of embodiment 11 or 12, wherein the protease comprises proteinase K, pepsin, collagenase, dispase, or a combination thereof.

### Embodiment 14

The method of any of embodiments 11 to 13, wherein the protease treatment does not eliminate the first color and tissue morphology is sufficiently maintained so as to allow for the detection of the first color.

### Embodiment 15

The method of embodiment 4, wherein the CD79a protein-specific antibody comprises a polyclonal antibody or a monoclonal antibody that specifically binds to the CD79a protein.

### Embodiment 16

The method of embodiment 15, wherein the CD79a protein-specific monoclonal antibody comprises a rabbit monoclonal antibody.

### Embodiment 17

The method of embodiment 16, wherein the rabbit monoclonal antibody is an anti-CD79a SP18 rabbit monoclonal antibody.

### Embodiment 18

The method of embodiment 4, wherein the CD79a protein-specific antibody comprises a first label, and the first chromogenic component comprises an inducing component for inducing the first label to emit the first color.

### Embodiment 19

The method of embodiment 18, wherein the first label comprises an enzyme.

### Embodiment 20

The method of embodiment 19, wherein the inducing component comprises a substrate for the enzyme of the first label.

### Embodiment 21

The method of embodiment 18, wherein the first label comprises biotin.

### Embodiment 22

The method of embodiment 21, wherein the inducing component comprises streptavidin conjugated to an enzyme.

### Embodiment 23

The method of embodiment 4, wherein the first chromogen component comprises a detectably labeled secondary antibody that specifically binds to the CD79a protein-specific antibody.

### Embodiment 24

The method of embodiment 23, wherein the detectably labeled secondary antibody comprises alkaline phosphatase and the first chromogen component further comprises fast red.

### Embodiment 25

The method of any of embodiments 1 to 24, wherein the TP53 DNA-specific nucleic acid probe comprises:
a) a nucleic acid molecule having at least 90% sequence identity with the sequence according to any one of SEQ ID NOs: 51-60; or
b) a nucleic acid molecule having at least 90% sequence identity with at least 250 contiguous nucleotides of any one of SEQ ID NOs: 51-60.

### Embodiment 26

The method of embodiment 25, wherein the TP53 DNA-specific nucleic acid probe comprises:
(a) a nucleic acid molecule having at least 95% sequence identity with the sequence according to any one of SEQ ID NOs: 51-60; or
(b) a nucleic acid molecule having at least 95% sequence identity with at least 250 contiguous nucleotides of any one of SEQ ID NOs: 51-60.

### Embodiment 27

The method of embodiment 26, wherein the TP53 DNA-specific nucleic acid probe comprises:
a) a nucleic acid molecule having at least 99% sequence identity to the sequence according to any one of SEQ ID NOs: 51-60; or
b) a nucleic acid molecule having at least 99% sequence identity with at least 250 contiguous nucleotides of any one of SEQ ID NOs: 51-60.

### Embodiment 28

The method of embodiment 27, wherein the TP53 DNA-specific nucleic acid probe comprises:
(a) a nucleic acid molecule consisting of the sequence according to any one of SEQ ID NOs: 51-60; or
(b) a nucleic acid molecule consisting of at least 250 contiguous nucleotides of any one of SEQ ID NOs: 51-60.

### Embodiment 29

The method of embodiment 25, wherein the TP53 DNA-specific nucleic acid probe comprises a nucleic acid molecule with at least 90% sequence identity to at least 400 contiguous of any one of SEQ ID NOs: 51-60.

### Embodiment 30

The method of embodiment 29, wherein the TP53 DNA-specific nucleic acid probe comprises a nucleic acid molecule with at least 90% sequence identity to at least 500 contiguous nucleotides of any one of SEQ ID NOs: 51-60.

### Embodiment 31

The method of embodiment 30, wherein the TP53 DNA-specific nucleic acid probe comprises a nucleic acid molecule with at least 90% sequence identity to at least 1000 contiguous nucleotides of any one of SEQ ID NOs: 51-60.

### Embodiment 32

The method of embodiment 31, wherein the TP53 DNA-specific nucleic acid probe comprises a nucleic acid molecule with at least 90% sequence identity to at least 2500 contiguous nucleotides of any one of SEQ ID NOs: 51-60.

### Embodiment 33

The method of any of embodiments 1 to 24, wherein the TP53 DNA-specific nucleic acid probe comprises two or more portions, wherein:
(c) the first portion comprises at least 250 contiguous nucleotides of a nucleic acid sequence with at least 90% sequence identity to one of SEQ ID NOs: 51-60; and
(d) the second portion comprises at least 250 contiguous nucleotides of a nucleic acid with at least 90% sequence identity to one of SEQ ID NOs: 51-60, wherein the first and second portions are different from one another.

### Embodiment 34

The method of embodiment 25, wherein the TP53 DNA-specific nucleic acid probe is at least 500 nucleotides in length.

### Embodiment 35

The method of embodiment 34, wherein the TP53 DNA-specific nucleic acid probe is at least 1000 nucleotides in length.

### Embodiment 36

The method of embodiment 35, wherein the TP53 DNA-specific nucleic acid probe is at least 5000 nucleotides in length.

### Embodiment 37

The method of any of embodiments 1 to 24, wherein the TP53 DNA-specific nucleic acid probe comprises at least two of the probes of any one of embodiments 25 through 36.

### Embodiment 38

The method of any of embodiments 1 to 37, wherein the TP53 DNA-specific nucleic acid probe comprises a detectable label.

### Embodiment 39

The method of embodiment 38, wherein the detectable label is a hapten.

### Embodiment 40

The method of embodiment 39 wherein the hapten comprises dinitrophenyl, digoxigenin, biotin, or fluorescein.

### Embodiment 41

The method of embodiment 38, wherein the second chromogen component comprises a primary antibody that specifically binds to the detectable label.

### Embodiment 42

The method of embodiment 41, wherein the second chromogen component further comprises a secondary antibody that specifically binds to the primary antibody.

### Embodiment 43

The method of embodiment 42, wherein the secondary antibody is conjugated to an enzyme.

### Embodiment 44

The method of embodiment 43, wherein the second chromogen component further comprises a substrate for the enzyme of the secondary antibody and a metal.

### Embodiment 45

The method of embodiment 44, wherein the enzyme of the secondary antibody comprises horseradish peroxidase, the substrate comprises hydrogen peroxidase, and the metal comprises silver.

### Embodiment 46

The method of any of embodiments 1 to 45, wherein the chromosome 17 centromere-DNA specific nucleic acid probe comprises a set of two or more single-stranded oligonucleotide control probes specific for X distinct monomers of an alpha satellite control region of chromosome 17, wherein X=2-14.

### Embodiment 47

The method of embodiment 46, wherein X≧4.

### Embodiment 48

The method of embodiment 46, wherein X≧6.

### Embodiment 49

The method of embodiment 46, wherein X≧8.

### Embodiment 50

The method of embodiment 46, wherein the control probes are configured to achieve at least two enumerable signals per cell with a staining intensity of ≧2 and staining coverage of ≧50% of the number of total nuclei within 3 hours of hybridization.

### Embodiment 51

The method of embodiment 46, wherein each control probe comprises:
a sequence selected from the group consisting of SEQ ID NOs: 61-74; or
a sequence selected from the group consisting of a truncated version of SEQ ID NOs: 61-74, the truncated version being at least 40 contiguous bp of said SEQ ID NOs:61-74; or
a sequence selected from the group consisting of a sequence that has at least 70% sequence identity to one of SEQ ID NOs: 61-74, or complements thereof.

### Embodiment 52

The method of any of embodiments 46 to 51, wherein the step of contacting the sample with the chromosome 17 centromere-DNA specific nucleic acid probe comprises hybridizing the probe under conditions for a period of time less than about 3 hours.

### Embodiment 53

The method of embodiment 46, wherein the method is free from the use of blocking DNA.

### Embodiment 54

The method of embodiment 46, wherein an amount of blocking DNA is used in one or more steps of the method.

### Embodiment 55

The method of any of embodiments 46 to 54, wherein each of the two or more single-stranded oligonucleotide control probes can achieve an enumerable signal when hybridized to chromosome 17.

### Embodiment 56

The method of embodiment 55, wherein each enumerable signal has a generally round shape, a round shape is a shape defined by a simple closed curve fitting within a first region, the first region is an area on and between an inner concentric circle and an outer concentric circle, the inner concentric circle having an inner radius (Rᵢₙ) and the outer concentric circle having a outer radius (Rₒᵤₜ) wherein Rᵢₙ is ≧50% of Rₒᵤₜ, and the simple closed curve has a radius Rₛᵢₘₚₗₑ wherein Rᵢₙ≦Rₛᵢₘₚₗₑ≦Rₒᵤₜ.

### Embodiment 57

The method of any of embodiments 46 to 56, wherein the two or more single-stranded oligonucleotide control probes are configured to hybridize uniquely and specifically to a portion of the control region of human chromosome 17 so that other chromosomes or portions thereof are not evidently labeled without the influence of blocking DNA.

### Embodiment 58

The method of embodiment 46, wherein the two or more single-stranded oligonucleotide control probes each comprise between 50 to 100 nucleotides.

### Embodiment 59

The method of any of embodiments 1 to 58, wherein the chromosome 17 centromere DNA-specific nucleic acid probe comprises a detectable label.

### Embodiment 60

The method of embodiment 59, wherein the detectable label is a hapten.

### Embodiment 61

The method of embodiment 60 wherein the hapten comprises dinitrophenyl, digoxigenin, biotin, or fluorescein.

### Embodiment 62

The method of embodiment 59, wherein the third chromogen component comprises a primary antibody that specifically binds to the detectable label.

### Embodiment 63

The method of embodiment 62, wherein the third chromogen component further comprises a secondary antibody that specifically binds to the primary antibody.

### Embodiment 64

The method of embodiment 63, wherein the secondary antibody is conjugated to an enzyme.

### Embodiment 65

The method of embodiment 64, wherein the third chromogen component further comprises a substrate for the enzyme of the secondary antibody.

### Embodiment 66

The method of embodiment 65, wherein the enzyme of the secondary antibody comprises horseradish peroxidase and the substrate comprises a green chromogen component.

### Embodiment 67

A multiplex method for co-detecting CD79a protein, TP53 genomic DNA, and chromosome 17 (CHR17) centromere DNA in a sample on a single slide, said method comprising:
(a) contacting the sample with a CD79a protein-specific primary antibody; contacting the sample with an alkaline phosphatase-conjugated secondary antibody that specifically binds to the CD79a protein-specific primary antibody; and contacting the sample with fast red, thereby producing a red color in the vicinity of the CD79a protein; and
(b) contacting the sample with a TP53 DNA-specific nucleic acid probe conjugated to dinitrophenyl; contacting the sample with a primary antibody that specifically binds to dinitrophenyl; contacting the sample with a horseradish peroxidase-conjugated secondary antibody that specifically binds to the primary antibody; contacting the sample with silver acetate, hydroquinone, and hydrogen peroxide, thereby producing a black precipitate in the nuclei indicating the presence of TP53 DNA; and
(c) contacting the sample with a chromosome 17 (CHR17) centromere DNA-specific nucleic acid probe conjugated to digoxigenin; contacting the sample with a primary antibody that specifically binds to digoxigenin; contacting the sample with HRP-conjugated secondary antibody that specifically binds to the primary antibody; contacting the sample with a green chromogen component, thereby producing a green or blue precipitate in the vicinity of the chromosome 17 centromere DNA.

### Embodiment 68

The method of embodiment 67, wherein bright-field microscopy is used to determine the presence and/or amount of the CD79a protein, TP53 genomic DNA, and chromosome 17 centromere DNA in the sample.

### Embodiment 69

A single slide comprising a sample of cells chromogenically stained for CD79a protein, TP53 DNA, and chromosome 17 DNA.

### Embodiment 70

The slide of embodiment 69, wherein each of CD79a protein, TP53 DNA, and chromosome 17 DNA are stained with a different chromogen.

### Embodiment 71

The slide of embodiment 69 or 70, wherein CD79a protein is stained with a first chromogen, TP53 DNA is stained with a second chromogen, and chromosome 17 is stained with a third chromogen.

### Embodiment 72

The slide of embodiment 70, wherein the first chromogen comprises fast red, the second chromogen comprises silver, and the third chromogen comprises a green chromogen component.

### Embodiment 73

The slide of any of embodiments 69 to 72, wherein more than 50% of the nuclei have enumerable signals for chromosome 17.

### Embodiment 74

The slide of embodiment 73, wherein each enumerable signal is a generally round shape, a round shape is a shape defined by a simple closed curve fitting within a first region, the first region is an area on and between an inner concentric circle and an outer concentric circle, the inner concentric circle having an inner radius (Rᵢₙ) and the outer concentric circle having a outer radius (Rₒᵤₜ) wherein Rᵢₙ is ≧50% of Rₒᵤₜ, and the simple closed curve has a radius Rₛᵢₘₚₗₑ wherein Rᵢₙ≦Rₛᵢₘₚᵢₑ≦Rₒᵤₜ.

### Embodiment 75

An isolated nucleic acid probe comprising:
(a) a nucleic acid molecule having at least 90% sequence identity with the sequence according to any one of SEQ ID NOs: 1-60; or
(b) a nucleic acid molecule having at least 90% sequence identity with at least 250 contiguous nucleotides of any one of SEQ ID NOs: 1-60.

### Embodiment 76

The nucleic acid probe of embodiment 75, wherein the probe comprises:
(a) a nucleic acid molecule having at least 95% sequence identity with the sequence according to any one of SEQ ID NOs: 1-60; or
(b) a nucleic acid molecule having at least 95% sequence identity with at least 250 contiguous nucleotides of any one of SEQ ID NOs: 1-60.

### Embodiment 77

The nucleic acid probe of embodiment 76, wherein the probe comprises:
(a) a nucleic acid molecule having at least 99% sequence identity to the sequence according to any one of SEQ ID NOs; 1-60; or

(b) a nucleic acid molecule having at least 99% sequence identity with at least 250 contiguous nucleotides of any one of SEQ ID NOs: 1-60.

### Embodiment 78

The nucleic acid probe of embodiment 77, wherein the probe comprises:
(a) a nucleic acid molecule consisting of the sequence according to any one of SEQ ID NOs: 1-60; or
(b) a nucleic acid molecule consisting of at least 250 contiguous nucleotides of any one of SEQ ID NOs: 1-60.

### Embodiment 79

The nucleic acid probe of embodiment 75, wherein the nucleic acid probe comprises a nucleic acid molecule with at least 90% sequence identity to at least 400 contiguous of any one of SEQ ID NOs: 1-60.

### Embodiment 80

The nucleic acid probe of embodiment 79, wherein the nucleic acid probe comprises a nucleic acid molecule with at least 90% sequence identity to at least 500 contiguous nucleotides of any one of SEQ ID NOs: 1-60.

### Embodiment 81

The nucleic acid probe of embodiment 80, wherein the nucleic acid probe comprises a nucleic acid molecule with at least 90% sequence identity to at least 1000 contiguous nucleotides of any one of SEQ ID NOs: 1-60.

### Embodiment 82

The nucleic acid probe of embodiment 80, wherein the nucleic acid probe comprises a nucleic acid molecule with at least 90% sequence identity to at least 2500 contiguous nucleotides of any one of SEQ ID NOs: 1-60.

### Embodiment 83

An isolated nucleic acid probe comprising two or more portions, wherein:
the first portion comprises at least 250 contiguous nucleotides of a nucleic acid sequence with at least 90% sequence identity to one of SEQ ID NOs: 1-60; and
the second portion comprises at least 250 contiguous nucleotides of a nucleic acid with at least 90% sequence identity to one of SEQ ID NOs: 1-60, wherein the first and second portions are different from one another.

### Embodiment 84

The nucleic acid probe of any of embodiments 75 to 83, wherein the probe is at least 500 nucleotides in length.

### Embodiment 85

The nucleic acid probe of embodiment 84, wherein the probe is at least 1000 nucleotides in length.

### Embodiment 86

The nucleic acid probe of embodiment 85, wherein the probe is at least 5000 nucleotides in length.

### Embodiment 87

The nucleic acid probe of any of embodiments 75 to 86, further comprising a detectable label.

### Embodiment 88

A vector comprising the nucleic acid probe of any of embodiments 75 to 87.

### Embodiment 89

The vector of embodiment 88, wherein the vector is a plasmid vector.

### Embodiment 90

A probe set comprising at least two of the nucleic acid probes of any of embodiments 75 to 87 or at least two of the vectors of embodiment 88 or embodiment 89.

### Embodiment 91

The probe set of embodiment 90, wherein the at least two nucleic acid probes are selected from the group consisting of SEQ ID NOs: 1-10 and a nucleic acid molecule comprising at least 250 contiguous nucleotides of SEQ ID NOs: 1-10.

### Embodiment 92

The probe set of embodiment 91, wherein probe set comprises:
at least one probe comprising SEQ ID NO: 1;
at least one probe comprising SEQ ID NO: 2;
at least one probe comprising SEQ ID NO: 3;
at least one probe comprising SEQ ID NO: 4;
at least one probe comprising SEQ ID NO: 5;
at least one probe comprising SEQ ID NO: 6;
at least one probe comprising SEQ ID NO: 7;
at least one probe comprising SEQ ID NO: 8;
at least one probe comprising SEQ ID NO: 9; and
at least one probe comprising SEQ ID NO: 10.

### Embodiment 93

The probe set of embodiment 90, wherein the at least two nucleic acid probes are selected from the group consisting of SEQ ID NOs: 51-60 and a nucleic acid molecule comprising at least 250 contiguous nucleotides of SEQ ID NOs: 51-60.

### Embodiment 94

The probe set of embodiment 93, wherein the probe set comprises:
at least one probe comprising SEQ ID NO: 51;
at least one probe comprising SEQ ID NO: 52;
at least one probe comprising SEQ ID NO: 53;
at least one probe comprising SEQ ID NO: 54;
at least one probe comprising SEQ ID NO: 55;
at least one probe comprising SEQ ID NO: 56;
at least one probe comprising SEQ ID NO: 57;
at least one probe comprising SEQ ID NO: 58;
at least one probe comprising SEQ ID NO: 59; and
at least one probe comprising SEQ ID NO: 60.

### Embodiment 95

The probe set of embodiment 90, wherein the at least two nucleic acid probes are selected from the group consisting of SEQ ID NOs: 21-30 and a nucleic acid molecule comprising at least 250 contiguous nucleotides of SEQ ID NOs: 21-30.

### Embodiment 96

The probe set of embodiment 95, wherein the probe set comprises:
at least one probe comprising SEQ ID NO: 21;
at least one probe comprising SEQ ID NO: 22;
at least one probe comprising SEQ ID NO: 23;
at least one probe comprising SEQ ID NO: 24;
at least one probe comprising SEQ ID NO: 25;
at least one probe comprising SEQ ID NO: 26;
at least one probe comprising SEQ ID NO: 27;
at least one probe comprising SEQ ID NO: 28;
at least one probe comprising SEQ ID NO: 29; and
at least one probe comprising SEQ ID NO: 30.

### Embodiment 97

The probe set of embodiment 90, wherein the at least two nucleic acid probes are selected from the group consisting of SEQ ID NOs: 31-40 and a nucleic acid molecule comprising at least 250 contiguous nucleotides of SEQ ID NOs: 31-40.

### Embodiment 98

The probe set of embodiment 97, wherein the probe set comprises:
at least one probe comprising SEQ ID NO: 31;
at least one probe comprising SEQ ID NO: 32;
at least one probe comprising SEQ ID NO: 33;
at least one probe comprising SEQ ID NO: 34;
at least one probe comprising SEQ ID NO: 35;
at least one probe comprising SEQ ID NO: 36;
at least one probe comprising SEQ ID NO: 37;
at least one probe comprising SEQ ID NO: 38;
at least one probe comprising SEQ ID NO: 39; and
at least one probe comprising SEQ ID NO: 40.

### Embodiment 99

The probe set of embodiment 90, wherein the at least two nucleic acid probes are selected from the group consisting of SEQ ID NOs: 41-50 and a nucleic acid molecule comprising at least 250 contiguous nucleotides of SEQ ID NOs: 41-50.

### Embodiment 100

The probe set of embodiment 99, wherein the probe set comprises:
at least one probe comprising SEQ ID NO: 41;
at least one probe comprising SEQ ID NO: 42;
at least one probe comprising SEQ ID NO: 43;
at least one probe comprising SEQ ID NO: 44;
at least one probe comprising SEQ ID NO: 45;
at least one probe comprising SEQ ID NO: 46;
at least one probe comprising SEQ ID NO: 47;
at least one probe comprising SEQ ID NO: 48;
at least one probe comprising SEQ ID NO: 49; and
at least one probe comprising SEQ ID NO: 50.

### Embodiment 101

A kit comprising one or more of the nucleic acid probes of any of embodiments 75 to 87, the vector of embodiment 88 or embodiment 89, or one or more probe sets of any of embodiments 90 to 100.

### Embodiment 102

A method of detecting a target nucleic acid molecule in a sample, comprising:
contacting the sample with the nucleic acid probe of any of embodiments 75 to 87 or the probe set of any of embodiments 90 to 100 under conditions sufficient to permit hybridization between the probe or the probe set and the target nucleic acid; and
detecting the hybridization, wherein presence of hybridization indicates presence of the target nucleic acid in the sample.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A multiplex method for co-detecting a B cell marker protein, TP53 genomic DNA, and chromosome 17 centromere DNA in a sample on a single slide, said method comprising:
staining the B cell marker protein by contacting the sample with a B cell marker protein-specific antibody and contacting the sample with a first chromogen component for the B cell marker protein-specific antibody, the first chromogen component is adapted to emit or make visible a first color, wherein the presence of the first color indicates the presence of the B cell marker protein; and
staining TP53 genomic DNA and staining chromosome 17 centromere DNA by contacting the sample with a TP53 genomic DNA-specific nucleic acid probe and with a chromosome 17 centromere DNA-specific nucleic acid probe, and contacting the sample with a second chromogen component for the TP53 genomic DNA-specific nucleic acid probe and with a third chromogen component for the chromosome 17 centromere DNA-specific nucleic acid probe, the second chromogen component is adapted to emit or make visible a second color and the third chromogen component is adapted to emit or make visible a third color, wherein the presence of the second color indicates the presence of TP53 genomic DNA and the presence of the third color indicates the presence of chromosome 17 centromere DNA.

2. The method of claim 1, wherein the sample is a blood sample.

3. The method of claim 1, wherein the B cell marker protein comprises one or more B cell marker proteins selected from the group consisting of CD79a protein, CD79b protein, BCL-2 protein, CD19 protein, CD22 protein, MUM1 protein, PAX5 protein, CD20 protein, Oct2 protein, and Bob.1 protein and the B cell marker protein-specific antibody comprises a CD79a protein-specific antibody, CD79b protein-specific antibody, BCL-2 protein-specific antibody, CD19 protein-specific antibody, CD22 protein-specific antibody, MUM1 protein-specific antibody, PAX5 protein-specific antibody, CD20 protein-specific antibody, Oct2 protein-specific antibody, or Bob.1 protein-specific antibody.

4. The method of claim 3, wherein the B cell marker protein comprises CD79a and the B cell marker protein-specific antibody comprises a CD79a protein-specific antibody.

5. The method of claim 3, wherein the B cell marker protein comprises CD79b or CD20 and the B cell marker protein-specific antibody comprises a CD79b protein-specific antibody or a CD20-specific antibody.

6. The method of claim 1, wherein the first chromogen component comprises fast red, the second chromogen component comprises silver, and the third chromogen component comprises a green chromogen component.

7. The method of claim 1, wherein the first color is transparent enough to allow visualization of the second color and the third color.

8. The method of claim 1 further comprising visualizing the colors using bright-field microscopy.

9. The method of claim 1, wherein the method is automated.

10. The method of claim 1, wherein the step of staining the B cell protein marker is performed before the step of staining TP53 genomic DNA and staining chromosome 17 centromere DNA.

11. The method of claim 1, wherein the sample is subjected to a protease treatment after the step of staining the B cell protein marker but before the step of staining TP53 genomic DNA and staining chromosome 17 centromere DNA, wherein the protease treatment is effective to allow for hybridization of the TP53 DNA-specific nucleic acid probe to the TP53 genomic DNA and for hybridization of the chromosome 17 centromere DNA-specific nucleic acid probe to the chromosome 17 centromere DNA.

12. The method of claim 11, wherein the sample is subjected to a heat treatment after the step of staining the B cell protein marker but before the protease treatment.

13. The method of claim 11, wherein the protease comprises proteinase K, pepsin, collagenase, dispase, or a combination thereof.

14. The method of claim 11, wherein the protease treatment does not eliminate the first color and tissue morphology is sufficiently maintained so as to allow for the detection of the first color.

15. The method of claim 4, wherein the CD79a protein-specific antibody comprises a polyclonal antibody or a monoclonal antibody that specifically binds to the CD79a protein.

16. The method of claim 15, wherein the CD79a protein-specific monoclonal antibody comprises a rabbit monoclonal antibody.

17. The method of claim 16, wherein the rabbit monoclonal antibody is an anti-CD79a SP18 rabbit monoclonal antibody.

18. The method of claim 4, wherein the CD79a protein-specific antibody comprises a first label, and the first chromogenic component comprises an inducing component for inducing the first label to emit the first color.

19. The method of claim 18, wherein the first label comprises an enzyme.

20. The method of claim 19, wherein the inducing component comprises a substrate for the enzyme of the first label.

21. The method of claim 18, wherein the first label comprises biotin.

22. The method of claim 21, wherein the inducing component comprises streptavidin conjugated to an enzyme.

23. The method of claim 4, wherein the first chromogen component comprises a detectably labeled secondary antibody that specifically binds to the CD79a protein-specific antibody.

24. The method of claim 23, wherein the detectably labeled secondary antibody comprises alkaline phosphatase and the first chromogen component further comprises fast red.

25. The method of claim 1, wherein the TP53 DNA-specific nucleic acid probe comprises:
(a) a nucleic acid molecule having at least 90% sequence identity with the sequence according to any one of SEQ ID NOs: 51-60; or
(b) a nucleic acid molecule having at least 90% sequence identity with at least 250 contiguous nucleotides of any one of SEQ ID NOs: 51-60.

26. The method of claim 25, wherein the TP53 DNA-specific nucleic acid probe comprises:
(a) a nucleic acid molecule having at least 95% sequence identity with the sequence according to any one of SEQ ID NOs: 51-60; or
(b) a nucleic acid molecule having at least 95% sequence identity with at least 250 contiguous nucleotides of any one of SEQ ID NOs: 51-60.

27. The method of any of claim 1, wherein the chromosome 17 centromere-DNA specific nucleic acid probe comprises a set of two or more single-stranded oligonucleotide control probes specific for X distinct monomers of an alpha satellite control region of chromosome 17, wherein X=2-14.

28. The method of claim 27, wherein X≧4.

29. The method of claim 27, wherein X≧6.

30. The method of claim 27, wherein X≧8.

31. The method of claim 27, wherein the control probes are configured to achieve at least two enumerable signals per cell with a staining intensity of ≧2 and staining coverage of ≧50% of the number of total nuclei within 3 hours of hybridization.

32. The method of claim 27, wherein the step of contacting the sample with the chromosome 17 centromere-DNA specific nucleic acid probe comprises hybridizing the probe under conditions for a period of time less than about 3 hours.

33. The method of claim 27, wherein the method is free from the use of blocking DNA.

34. The method of claim 27, wherein an amount of blocking DNA is used in one or more steps of the method.

35. The method of claim 27, wherein the two or more single-stranded oligonucleotide control probes are configured to hybridize uniquely and specifically to a portion of the control region of human chromosome 17 so that other chromosomes or portions thereof are not evidently labeled without the influence of blocking DNA.

36. The method of claim 27, wherein the two or more single-stranded oligonucleotide control probes each comprise between 50 to 100 nucleotides.

37. A multiplex method for co-detecting CD79a protein, TP53 genomic DNA, and chromosome 17 (CHR17) centromere DNA in a sample on a single slide, said method comprising:
contacting the sample with a CD79a protein-specific primary antibody; contacting the sample with an alkaline phosphatase-conjugated secondary antibody that specifically binds to the CD79a protein-specific primary antibody; and contacting the sample with fast red, thereby producing a red color in the vicinity of the CD79a protein; and
contacting the sample with a TP53 DNA-specific nucleic acid probe conjugated to dinitrophenyl; contacting the sample with a primary antibody that specifically binds to dinitrophenyl; contacting the sample with a horseradish peroxidase-conjugated secondary antibody that specifically binds to the primary antibody; contacting the sample with silver acetate, hydroquinone, and hydrogen peroxide, thereby producing a black precipitate in the nuclei indicating the presence of TP53 DNA; and
contacting the sample with a chromosome 17 (CHR17) centromere DNA-specific nucleic acid probe conjugated to digoxigenin; contacting the sample with a primary antibody that specifically binds to digoxigenin; contacting the sample with HRP-conjugated secondary antibody that specifically binds to the primary antibody; contacting the sample with a green chromogen component, thereby producing a green or blue precipitate in the vicinity of the chromosome 17 centromere DNA.

38. The method of claim 37, wherein bright-field microscopy is used to determine the presence and/or amount of the CD79a protein, TP53 genomic DNA, and chromosome 17 centromere DNA in the sample.

39. An isolated nucleic acid probe comprising:
(a) a nucleic acid molecule having at least 90% sequence identity with the sequence according to any one of SEQ ID NOs: 1-60; or
(b) a nucleic acid molecule having at least 90% sequence identity with at least 250 contiguous nucleotides of any one of SEQ ID NOs: 1-60.

40. The isolated nucleic acid probe of claim 39, wherein the isolated nucleic acid probe comprises two or more portions, wherein:
the first portion comprises at least 250 contiguous nucleotides of a nucleic acid sequence with at least 90% sequence identity to one of SEQ ID NOs: 1-60; and
the second portion comprises at least 250 contiguous nucleotides of a nucleic acid with at least 90% sequence identity to one of SEQ ID NOs: 1-60, wherein the first and second portions are different from one another.

41. The nucleic acid probe of claim 39, wherein the probe is at least 500 nucleotides in length.

42. The nucleic acid probe of claim 39, wherein the probe is at least 1000 nucleotides in length.

43. The nucleic acid probe of claim 39, wherein the probe is at least 5000 nucleotides in length.

44. The nucleic acid probe of claim 39, further comprising a detectable label.

45. A vector comprising the nucleic acid probe of claim 39.

46. The vector of claim 45, wherein the vector is a plasmid vector.

47. A probe set comprising at least two of the nucleic acid probes of 39.

48. The probe set of claim 47, wherein the at least two nucleic acid probes are selected from the group consisting of SEQ ID NOs: 1-10 and a nucleic acid molecule comprising at least 250 contiguous nucleotides of SEQ ID NOs: 1-10.

49. The probe set of claim 48, wherein probe set comprises:
at least one probe comprising SEQ ID NO: 1;
at least one probe comprising SEQ ID NO: 2;
at least one probe comprising SEQ ID NO: 3;
at least one probe comprising SEQ ID NO: 4;
at least one probe comprising SEQ ID NO: 5;
at least one probe comprising SEQ ID NO: 6;
at least one probe comprising SEQ ID NO: 7;
at least one probe comprising SEQ ID NO: 8;
at least one probe comprising SEQ ID NO: 9; and
at least one probe comprising SEQ ID NO: 10.

50. The probe set of claim 47, wherein the at least two nucleic acid probes are selected from the group consisting of SEQ ID NOs: 51-60 and a nucleic acid molecule comprising at least 250 contiguous nucleotides of SEQ ID NOs: 51-60.

51. The probe set of claim 50, wherein the probe set comprises:
at least one probe comprising SEQ ID NO: 51;
at least one probe comprising SEQ ID NO: 52;
at least one probe comprising SEQ ID NO: 53;
at least one probe comprising SEQ ID NO: 54;
at least one probe comprising SEQ ID NO: 55;
at least one probe comprising SEQ ID NO: 56;
at least one probe comprising SEQ ID NO: 57;
at least one probe comprising SEQ ID NO: 58;
at least one probe comprising SEQ ID NO: 59; and
at least one probe comprising SEQ ID NO: 60.

52. The probe set of claim 47, wherein the at least two nucleic acid probes are selected from the group consisting of SEQ ID NOs: 21-30 and a nucleic acid molecule comprising at least 250 contiguous nucleotides of SEQ ID NOs: 21-30.

53. The probe set of claim 52, wherein the probe set comprises:
at least one probe comprising SEQ ID NO: 21;
at least one probe comprising SEQ ID NO: 22;
at least one probe comprising SEQ ID NO: 23;
at least one probe comprising SEQ ID NO: 24;
at least one probe comprising SEQ ID NO: 25;
at least one probe comprising SEQ ID NO: 26;
at least one probe comprising SEQ ID NO: 27;
at least one probe comprising SEQ ID NO: 28;
at least one probe comprising SEQ ID NO: 29; and
at least one probe comprising SEQ ID NO: 30.

54. The probe set of claim 47, wherein the at least two nucleic acid probes are selected from the group consisting of SEQ ID NOs: 31-40 and a nucleic acid molecule comprising at least 250 contiguous nucleotides of SEQ ID NOs: 31-40.

55. The probe set of claim 54, wherein the probe set comprises:
at least one probe comprising SEQ ID NO: 31;
at least one probe comprising SEQ ID NO: 32;
at least one probe comprising SEQ ID NO: 33;
at least one probe comprising SEQ ID NO: 34;
at least one probe comprising SEQ ID NO: 35;
at least one probe comprising SEQ ID NO: 36;
at least one probe comprising SEQ ID NO: 37;
at least one probe comprising SEQ ID NO: 38;
at least one probe comprising SEQ ID NO: 39; and
at least one probe comprising SEQ ID NO: 40.

56. The probe set of claim 47, wherein the at least two nucleic acid probes are selected from the group consisting of SEQ ID NOs: 41-50 and a nucleic acid molecule comprising at least 250 contiguous nucleotides of SEQ ID NOs: 41-50.

57. The probe set of claim 56, wherein the probe set comprises:
at least one probe comprising SEQ ID NO: 41;
at least one probe comprising SEQ ID NO: 42;
at least one probe comprising SEQ ID NO: 43;
at least one probe comprising SEQ ID NO: 44;
at least one probe comprising SEQ ID NO: 45;
at least one probe comprising SEQ ID NO: 46;
at least one probe comprising SEQ ID NO: 47;
at least one probe comprising SEQ ID NO: 48;
at least one probe comprising SEQ ID NO: 49; and
at least one probe comprising SEQ ID NO: 50.

58. A kit comprising one or more probe sets of claim 47.

59. A method of detecting a target nucleic acid molecule in a sample, comprising:
contacting the sample with the nucleic acid probe of claim 39 under conditions sufficient to permit hybridization between the probe or the probe set and the target nucleic acid; and
detecting the hybridization, wherein presence of hybridization indicates presence of the target nucleic acid in the sample.
